SEQUENCE LISTING (135 sequences)

SEQ ID NO: 1 - DNA, 972 bp, Rubus occidentalis
  source 1..972: mol_type = other DNA
atggactgcggcggcccaacaacaaacaaggacaagctcctcgtcattatgggagccaccggcgccggcaagtcgcggctatccctcgacctcgccgcttgcttccctccggcaaccttcgaagtcatcaacgcggataaaatgcaggtctacaagggactcgacatcaccaccaacaagctccccgctccggagcgactcggcgtcccgcaccacttgctcggcgagttcgactcgctcgacggcgaggttactcccgccaagtttcgcgagctcgcggctcgggtggtttccggcgttagtgctcggaggaaagtgcctgtgcttgtgggtgggtccaactcattcatccacgcgctggtcgtcgaccggttcgaaccaggttttaatgtctttgacggcgactctgtgacagtatcgatctcatcggagctccggtacaattgttgttttctctgggttgatgtgtcgtcggtggtgttgacggagtacttgtcgaagcgagtagacgaaatgctagactcggggatgttactcgagctggccgagttttacaactcggccgaggaagactcggtggttcgaacggggataagaaagtccatcggagtagcggagttcagccggtattttaggaagcacccgccgccgggaggtagcgaggaggtgggggacgatgacccggtacggagagaagcatataaggacgccgtgagggaaattaagctgaacacgtgtcagctggcgaagaggcagattggaaaaatctcacggctgaggggggcggggtggaacctacggaggttagatgcgacggaggcgtttagggcggcggtgaagtcagatgaaaaagacgggaataggtggtcggagatttgggaaagagaggtggttggaccaagcgtgaagattgtgaatcatttcttggaggaggagattcaatcaacaaaagtacaaaatcaaaagatttag

SEQ ID NO: 2 - DNA, 972 bp, Rubus occidentalis
  source 1..972: mol_type = other DNA
atggattgcggcggacctactaccaataaggataagctgttggttattatgggcgctaccggcgccggaaagtctagactgtcattggatcttgctgcctgttttccaccagctacattcgaagttattaatgctgataagatgcaggtttacaaaggactggatatcacaactaacaaactgcctgcaccagagcgcttgggtgttcctcatcaccttctgggcgaatttgattctcttgatggagaggtgaccccagctaagttcagagaactggcagctcgcgtggtttctggcgtttcagccagacgcaaagtgcctgttctggtgggtggctctaattcttttattcatgccttggtggttgatagattcgaaccaggctttaatgtgttcgatggcgattctgttactgtgtcaatctcttcagagcttcgctataattgctgttttctgtgggttgatgtgtcttcagtggttcttaccgaatacctgtctaagagggtggatgagatgttggattcaggcatgttgcttgaacttgctgagttctacaactctgccgaggaagattcagtggttcgcacaggcattaggaagtctatcggagttgctgaattttcaaggtatttcagaaaacatccaccaccaggcggctctgaggaagttggagatgatgatcctgtgaggagagaagcctacaaggatgcagtgcgcgagatcaaattgaatacatgccagcttgccaagaggcagattggtaaaatctcaagattgcgcggtgcaggctggaacctgcgcaggttggatgcaactgaggcttttagagccgctgttaagtctgatgaaaaagatggtaacaggtggtcagagatttgggaaagagaggtggttggcccatctgtgaagattgtgaatcacttcctggaggaagagatccagtcaactaaggtgcagaaccagaaaatctga

SEQ ID NO: 3 - DNA, 1074 bp, Rubus occidentalis
  source 1..1074: mol_type = other DNA
atgacacttgattccttcccaactcttcattattcttattataatattaccccccattctctctcctcgccggcggtcaccctcactcccccaccatctcttccccaaaagccgcgctgggcccgaatggactccggcccagcaacaactacaaccgtcccactccggcacaaggacaagcttgttgtcatcatgggagccaccggagccggaaagtcccgtctctcaatcgacctcgccactcacttcccttcttgcttcgaaatcatcaactcggacaaaatgcaggtctacaagggactcgacatcaccaccaacaagattccagttcccgaccgtatcggagttccccaccacctcctcggtcagttcgacccgctcgacggggagctcacccccgccgagtttcgacaactcgccggtcaagccgtttccgccattacttctcggcggaaagtgcccatgcttgtgggtgggtccaactctctaatacacgccctcctcgttgaccggttcgaaccgggtgtagatgtcttcgaaggcggcgccgccatatcgtcggagctcaggtacaattgctgctttctctgggttgatgtctccttgacggttttagcggactacttgtctaagcgagtagacgaaatgctcgactcgggaatgttggaggagttggccgagttttgcgacccggaagatgacgacttggcgattcgaaccgggttgagaaaggctattggagtaccggagttcagccggtttttcaaaaaatacccgccgatttgtcagagtcagaaggcgaggcccgatgatccggtgcggagaggagcatataaagaagcggttagggccattaaggacaacacgtgtcagctggccaagacgcagattggaaagattctacggcttagagtggcgggagggtgggacctacagaggctagatgcgaccgaggcgttcagggcggtggtgacgtcagaagacgatgccggaaagaggtggtcagagatatggcagaaacaggtggtggaagcaagcgtgaaaattgtgaagcgattcttggagcaggagtag

SEQ ID NO: 4 - DNA, 1074 bp, Rubus occidentalis
  source 1..1074: mol_type = other DNA
atgacattggattcttttcctactcttcattactcatactacaacatcactcctcactcactgtcttcaccagcagtgactctgactccaccaccatctctgcctcagaagccaagatgggctcgcatggattctggacctgccactaccacaactgttccacttagacataaggataaactggtggttattatgggtgcaacaggcgctggaaaatctcgcctgtcaatcgatttggccactcactttccttcttgcttcgagattatcaattcagataagatgcaggtttacaaaggcctggatattaccacaaacaagatccctgtgccagatagaattggagttccacatcacctgttgggtcaatttgatcctcttgatggcgagctgaccccagctgaattccgccagttggccggtcaggcagtgtctgctattacatcaagacgcaaagtgcctatgctggttggcggatctaattcattgatccatgcccttctggttgataggtttgagccaggcgtggatgttttcgaaggtggcgctgccatctcttcagagcttagatataactgctgtttcctgtgggtggatgtttctctgactgtgttggccgattacttgtcaaagagagttgatgaaatgcttgattctggaatgcttgaggaactggcagagttttgtgatcctgaagatgatgatttggcaattaggaccggtcttagaaaggctatcggcgtgccagagttctcaagatttttcaagaagtatcctccaatctgccagtctcagaaagcccgccctgatgatccagtgaggagaggcgcatacaaggaagccgttagggcaatcaaagataacacctgtcagcttgctaagacacagattggaaaaatcttgcgccttagggttgctggcggctgggatttgcagcgccttgatgctactgaggcctttagggcagtggttacctcagaagatgatgcaggcaagcgctggtctgagatttggcagaaacaggtggttgaagcttctgtgaagatcgttaaaaggttcttggagcaggaatga

SEQ ID NO: 5 - DNA, 1125 bp, Prunus avium
  source 1..1125: mol_type = other DNA
atgacacttcatcccttcccaactcactcccaccaccattattctaatctcaatttctattcttatatctctcgtttccacttgttcccgccggtcaaccacctctgccccaccactccttcccatcatcatcctctcccccacatcaagccccgccgcggggcccacatatcctccggccccaccaccccacgccaacaacaacagcgcaaggacaagcttctcattatcttgggcgccaccggcgccggcaagtcccgcctctccctcgacctcgccacccgcttccctttcttcgaaatcgtgaactccgacaaaatgcaactctacgccggcctcgacatcaccaccaacaagcttcccatccccgaccgcctcggcgtcccccaccatctcctcggcgagttcgaccctcgccacggcgattttacgcccgcccaattccgcgccgttgccggtcaagccatttccagcattacgaatcgaaggaaggtgccgatgctcgtcggcggctccaactccttcattcatgcgctcctcgtggaccggttcgaaccgggctccaatgtcttcgaaccgggtttcaatggctctgtctcttccgagctcaggtataattgctgttttctgtgggtggacgtgtcgttgacggtcttgacggagtacttgtgcaagcgagtcgacgaaatgctcgactcggggatgttggacgagttggccgagttctgcgacccggatcgccaggacgaagacgaattgacggcgagtcagacagcgttgagaaaggcgattggcgtgcccgagttcactcggtattttgaaaaatatccaccacaggggaggggcggagagggtgatgatcgggagcggagggaagcatacgaagaggcggtgagggcgatcaaggacaacacgtgtcagctggcgaagaggcagatagggaagatcctacggctgaagggaggagggtgggacctacggaggctagatgcaacggacgcgtttagggcggtggttgcgacgacgtcgtcggataaggacgacggaaagaggtggtcagagatatgggagaggcaggttgtcaaaccaagcgtgaaggttgtgaagcgtttcttggacgagtag

SEQ ID NO: 6 - DNA, 1125 bp, Prunus avium
  source 1..1125: mol_type = other DNA
atgactcttcatccatttcctacccactctcatcaccattactcaaatctgaacttctactcttacatttcacgctttcacctgtttccaccagttaatcacttgtgcccaactaccccttctcaccatcacccattgcctcatattaagccaagacgcggagcccacatctcttcaggtccaacaactccaagacagcagcagcagagaaaggataaactgttgattatccttggtgcaacaggagctggcaagtctcgcctgtcattggatcttgcaactaggtttccatttttcgagatcgtgaactcagataagatgcagctttacgctggcctggatattaccacaaacaagttgccaatccctgataggcttggcgttcctcatcaccttctgggagaattcgatccaagacatggcgattttacccctgcccagttccgcgctgtggctggacaggcaatctcttcaatcacaaacaggagaaaagtgcctatgctggttggcggatctaactcttttattcacgccttgcttgtggatcgcttcgagccaggttctaatgtttttgaacctggcttcaacggatcagtgtcttcagagcttaggtataattgctgttttctgtgggtggatgtttctcttacagttctgactgagtacctgtgcaagagagtggatgaaatgttggattcaggcatgttggatgagcttgcagaattctgtgatccagatcgccaggatgaggatgaattgaccgcctctcagacagcacttaggaaagctattggagttcctgagtttactagatatttcgaaaagtacccacctcagggtcgcggtggcgaaggcgatgatagggagcgcagggaagcttatgaggaagccgtgagagcaatcaaagataacacttgtcagctggccaagagacagattggcaaaatcctgagactgaagggcggcggctgggatctgagaagactggatgctaccgatgcctttagggcagtggttgctactacctcttcagataaggatgatggaaaaaggtggtctgagatctgggaaagacaggtggttaagccatcagttaaggtggttaaaagattcttggatgagtga

SEQ ID NO: 7 - DNA, 1173 bp, Rubus occidentalis
  source 1..1173: mol_type = other DNA
atggaggaatacaataacagtaatgaaatggatcatgagagttcaggtactcccagaggaaatttcctgtacgcatctccaaatctcggaggtaactatggtagaactgccgcaggtgatcataatcagcagatcaacacctttcatcttcaatcaagcggtggcgagtgttttcagtccggaggaacagcacagcccaccgtgaagaccgaagcagcaggtagcacttcccaacatgctccccacaaatttcatcatcagtttcctcctccctcattaatattgtcaagaggtcagaatgatcagctaccagttcagcaacaacagcacatagaaaacgagaatgaagtggaggccatcaaggctaaaatcatcgctcaccctcactactctaatctcttgcaagcttacatggattgccaaagggtgggagctccgtctgaagtcgtggctcgtctcgcagctgcgcgccaggagtttgaagcacgacagcgatctttagtcagttcgagagatgcttcttcgaaagacccagaactcgatcagttcatggaagcttactatgatatgttggttaaatatcgggaggaactaacaaggcccatacaagaagcaatggatttcatgaggaagattgaaactcagcttaacatgctcggcaatgacagtagcactactcctcttcgcatcttctcggcctcggcgcaagaggacaaatgtgacggaaatggttcatctgaagaggaccaggacaacaatagtggtggagaaactgaagtggccgagatcgatccgagagccgaagacagagaactcaagaatcacctcttgagaaagtacagtggttacttaagtagcctcaagcaagaactctccaagaaaaagaagaaaggcaaattgcccaaagatgccaggcagaagcttctgagttggtgggagttgcactacaagtggccatatccttcggagtcggagaaggtggcattggcggagtcgacaggtttggatcagaagcagataaacaattggttcataaatcaaaggaagcggcactggaagccatcggaggacatgcagttcatggtgatggatgggttgcacccacagaatgcagcaactctctacatggatggacactacattggtgatggtcattaccgtctcggtccctaa

SEQ ID NO: 8 - DNA, 1362 bp, Rubus occidentalis
  source 1..1362: mol_type = other DNA
atggaggaatacaataacagtaatgaaatggatcatgagagttcaggtactcccaggtaagtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaatttgttgatgtgcaggggaaatttcctgtacgcatctccaaatctcggaggtaactatggtagaactgccgcaggtgatcataatcagcagatcaacacctttcatcttcaatcaagcggtggcgagtgttttcagtccggaggaacagcacagcccaccgtgaagaccgaagcagcaggtagcacttcccaacatgctccccacaaatttcatcatcagtttcctcctccctcattaatattgtcaagaggtcagaatgatcagctaccagttcagcaacaacagcacatagaaaacgagaatgaagtggaggccatcaaggctaaaatcatcgctcaccctcactactctaatctcttgcaagcttacatggattgccaaagggtgggagctccgtctgaagtcgtggctcgtctcgcagctgcgcgccaggagtttgaagcacgacagcgatctttagtcagttcgagagatgcttcttcgaaagacccagaactcgatcagttcatggaagcttactatgatatgttggttaaatatcgggaggaactaacaaggcccatacaagaagcaatggatttcatgaggaagattgaaactcagcttaacatgctcggcaatgacagtagcactactcctcttcgcatcttctcggcctcggcgcaagaggacaaatgtgacggaaatggttcatctgaagaggaccaggacaacaatagtggtggagaaactgaagtggccgagatcgatccgagagccgaagacagagaactcaagaatcacctcttgagaaagtacagtggttacttaagtagcctcaagcaagaactctccaagaaaaagaagaaaggcaaattgcccaaagatgccaggcagaagcttctgagttggtgggagttgcactacaagtggccatatccttcggagtcggagaaggtggcattggcggagtcgacaggtttggatcagaagcagataaacaattggttcataaatcaaaggaagcggcactggaagccatcggaggacatgcagttcatggtgatggatgggttgcacccacagaatgcagcaactctctacatggatggacactacattggtgatggtcattaccgtctcggtccctaa

SEQ ID NO: 9 - DNA, 1068 bp, Prunus avium
  source 1..1068: mol_type = other DNA
atggaagagtacaaccaaatggatcatgagagctcaggtggtagaggaaatttcctgtacgcctcaccaaatcttggaggcaactatgggagggcaagccatgatcatcagatgggcatcaacgcctttcatcttcaatcgagcggtggtggtgatttccactctccaggaggagcacacccaattaatgtcaagactgaagccagcacatcccatcatggccatcacaaatttcagtacaacaacaacaacaataataataataataatcctcttctgtcatcaagagggcaccaaccagtagttcaacaacagcataatctggaccgtcaaaacgatgatcacaccctgagctccaacgaagtggaagccatcaaagccaagatcatcgcccaccctcagcagcggtcttcagtggcttcaagagaggcttcaaaagatccagaactcgatcagttcatggaagcttactatgatatgttggtgaaatatcgagaggagctaacaaggcccatacaagaagccatggatttcatgcggaggattgaaactcagctcaacatgcttggcaacaataatgctcctcctcttcgcatcttctcaccctctgaggacaaatgtgaaggaattggttcatctgaagaggagcaggagaatagtggtggagaaacagaagtgcctgagattgatccaagagctgaagacagagagctcaagaaccacctgttgaaaaagtatagtggttacttaagtagcctgaagcaagagctttccaagaaaaagaagaaagggaaattgcccaaagatgccaggcagaagcttctcagttggtgggagttgcattacaagtggccatatccttcagaatcggagaaggtggcattggcggagtcaaccggtttggatcagaaacaaataaacaattggttcataaatcagaggaagaggcactggaaaccctcagaggacatgcagttcatggtgatggatggcctacaccctcagaatgcggccctgtatatggatggacactacataggcgatggccactaccgtctggggccatga

SEQ ID NO: 10 - DNA, 1068 bp, Prunus avium; identical to SEQ ID NO: 9
  source 1..1068: mol_type = other DNA

SEQ ID NO: 11 - DNA, 2103 bp, Rubus occidentalis
  source 1..2103: mol_type = other DNA
atgaacatgaactggttgggtttctctctttcccctcaagaagatgatgctccaatatcacaccagctggctgaccaagagacgttagcctctcgccttggcttcaactccaatgatcaaatccaaggtgccggcggtgaggcagattggaatatgaagggtgtagacatgaactcatcagatggcaataactacaaaaggacctcatcatcatccgatctgtcgcctatgctaatgggaagcagtactactactagtacttcatgcagcattatcagccagccaaaccttgaaaatcatcatcaacaacaacaactaccaaagcttgaaaacttccttggccgacattcttttgcggatcatgaccaaagtagtactagtcatgattacatgttcgatatgaataatggaccgggtgcaattagcagtaatgttatgaatacttctaatactaatagtaacaatattgggctgtccatgatcaagacttggctgaggaatcaacctcctcagccgcgcgataacaaccaccagctggagcaggagagcaagactactaaggagagcagaaatcaaccacagagtagtctatcgctctcaatgagtactggatcgcttcatacagtaacaactgcaactgcaggtggtgccagtgccagtgccggtgccggggaaactacctcatcatcaaataataatagaaagcagtcgccagttttgacagccacaacaactgatccccaaactcaaactactggtggtgccattgaggctgtgcccagaaaggccattgatacatttggccagaggacttctatataccgtggtgtaaccaggcacagatggacaggtagatatgaagctcatctatgggataatagctgccgaagagagggacaaactcgcaagggaaggcaaggtggttatgacaaagaagacaaggcagctagagcttatgacttagcagcattgaaatattggggcaccacaaccactaccaactttccgatcagtacctatgaaaaagagatagatgaaatgaagccgatgacaagacaggagtatgttgcatctctgagaaggaagagtagcgggttttctcgaggtgcatccatttatcgaggggtaaccaggcaccaccagcatgggagatggcaagcaagaattggtagagttgcagggaacaaagatctgtaccttggaactttcagtacccaagaggaagcagctgaggcgtatgacattgctgccataaagttccgaggactcaatgcggtaacaaactttgacatgagcagatatgatgtcaagtccattctggagagcagcgcattgcccattactactggtgcctctgcaaagcgattgaaggatgttcagcagcagcctcctcctccggcagatcatcatcatcagatcatgttgtcgtctgtgctggatcatcacggacagatgatcagatcttcatcgtcaacagagcatgatatcatgggcaatatctttcatcaggatgatgatcatctccgtcaacaacttcagatgggaggcacccacaatttcttccatgatcatgatcaccagcagcgtagcggccttatgggattaatggattcttcggcagcagcagcttctatggaacacagctcaggctctaattctgtcatatatagtgttggggatcaccatggtaataataatggatatggaagcaatgctagagggactggtagtggctatataatgcccatggctatgagtacagttgtggctaatcatggccaaaatcaagctgatggaaataatatcaatggtttcggagatggtgatggtgatgatcaggaagtgaatattaaggcgcagcagcttggttatgatcatcatcaaaatgtgtttctgggttcaagtaatactacagatcccgcttatcagcatcacgcaagcaacaggaacttgtattatcaacttccagtacatgatgaccagcatgaatcgtcatcagaagcagtagctactagtagtactacatgtaacatgaattgggttccaacagctgttccaactcatcttgctcattctacttttacagtctggaacgacacatag

SEQ ID NO: 12 - DNA, 2103 bp, Rubus occidentalis
  source 1..2103: mol_type = other DNA
atgaatatgaactggttgggcttttctctttctccacaggaagatgatgctcctatttctcatcagctggccgatcaggaaactctggcatctagattgggcttcaattcaaacgatcagatccagggagctggcggagaggccgattggaatatgaagggtgtggatatgaactcttcagatggcaataactataagcgcacctcttcatcttcagatctttctccaatgctgatgggttcttcaactaccacatctacatcatgctctattatctcacagccaaatttggagaaccatcaccagcagcagcagttgcctaagcttgaaaattttcttggcagacattctttcgccgatcacgatcagtcttcaacctcacacgattacatgtttgatatgaataacggacctggtgcaatctcttcaaacgttatgaacacttctaacaccaactcaaataacatcggactgtctatgatcaaaacatggttgaggaatcagccacctcagccaagagataataaccatcagctggagcaggaatctaagactaccaaagagtcaaggaaccagcctcagtcttcactgtcattgtctatgtcaactggttctttgcacaccgtgacaactgctacagccggtggcgcatctgcttcagctggcgctggcgagactacatcttcatctaataacaatagaaagcagtcaccagttttgaccgctactaccacagatcctcagacacagactaccggaggtgccattgaagcagtgccaaggaaagccatcgatactttcggacagagaacctctatctatcgcggtgttacacgccataggtggactggcagatacgaggctcacctttgggataattcatgtagacgcgaaggacagactagaaagggtcgccagggcggatatgataaggaagataaagctgctagagcttatgatctggcagctttgaaatactggggcacaactaccacaactaacttcccaatctctacttatgagaaggaaatcgatgagatgaaacctatgaccagacaggaatacgtggcttcactgaggagaaagtcatctggattctctcgcggtgcctcaatctatcgcggagtgactaggcatcaccagcatggtaggtggcaggctaggatcggcagagttgccggaaataaggatctttatctgggcacattttctactcaggaagaggctgcagaggcatacgatattgctgccatcaagtttcgcggacttaatgctgtgaccaacttcgatatgtctagatacgatgttaaatcaatcttggaatcatctgcccttccaatcaccacaggcgcttctgccaagcgcctgaaagatgtgcagcagcagccaccaccaccagctgatcatcaccatcagattatgttgtcatctgttcttgatcaccatggacagatgatcaggtcatcttcatctactgagcatgatattatgggtaatatcttccatcaggatgatgatcacctgagacagcagttgcagatgggtggcacccacaactttttccacgatcatgatcaccagcagcgctctggtcttatgggcctgatggattcatctgcagctgccgcatctatggaacattcatctggatctaattctgtgatctattcagttggcgatcatcatggcaacaataacggttatggctctaacgcccgcggaacaggttcaggctacatcatgcctatggcaatgtctactgtggttgctaatcacggccagaaccaggcagatggaaataacattaatggctttggcgatggcgatggcgatgatcaggaagtgaacatcaaggctcagcagcttggttatgatcaccatcagaatgttttcctgggctcatctaacactaccgatccagcataccagcaccatgcttctaataggaacctttattaccagctgcctgtgcatgatgatcagcacgagtcatcttcagaagcagttgctacatcttcaacaacttgcaatatgaactgggtgccaaccgccgttcctacacatttggcacactcaacctttacagtttggaatgatacttga

SEQ ID NO: 13 - DNA, 1599 bp, Prunus avium
  source 1..1599: mol_type = other DNA
atgggttccatgaactggttgggtttctctctttcccctcaagaagtcgttcctgctgatccatccatatctgagggtgatcatcatcatcaacatcatgacggccatgagttatctgatgatcagaccgccacactagtctctcgccttggcttcaagtactccgatgacatctccggtaccgatgtcaccggaggctgctttgatctcacttctcatgatgactcatccgctactactactgctgctgctgttcctaataatcattctcacaaccacctcatccctccaccttttggcatgcttcatcaagctgctgcattcaacaccaccaatattcactcccaaggtgcagattggagtaatatgaactcatcagatagcagcacctacaagacgacttcagaccacctgtctatgctgatgggaagcaacagttcgtacagaagccaaaaccttgaaaataatcatcaacagccaaagcttgaaaacttccttggccgacactccttcgttgcggatgagcaccacatcggcggccagagtagtagtggcacgcatgcatacaataataatagcaccagcaatacaatacagactctttcgctttcgatgagtactggatctcacactcatcataatactactagtactgctgctgctggagaaacttgttcatcagatcgagataatacttgtaataataataacaagctggcagttgttaggaccaccgcctcaaccgctcctccccctgggatcgatagccagactactagtactactgctactgctattgaggccgtgcccagaaagtccgttgatacttttggacagaggacttctatataccgtggcacagatggacaggtagatatgaagctcatctctgggataatagttgccggagagaaggacaaactcgcaagggaaggcaagatcagcaactatgagaaagagatagacgaaatgaagcacatgacgagacaggagtacgttgcatctttaagaaggaagagtagtggcttttctcgtggtgcatccatttatcgaggggtaacaagacaccaccagcatgggagatggcaagcaaggattggtcgagtcgcagggaacaaagatctctacctgggaacatttagctccaactctgtcatgtatagcactgctactactactaatggaggaggaggaggaggaggaggagatcatggtggctatataatcccaatgggtacagttatatctaatgatcacaatgatggttttggagatggcactaatcatgatcatcaggtgaaggcggtggcgcttggatttgaaaatgtgtttggctcaacttctgcttctgttaatgctacaaatgttactagggatgcttataataatcatgcaaggaacttgtattatcttccgcagcagctgccgcaatcgtcatcagtgtcatcagtatcatcagcaggtgtggccaagggcggcagcgcatatgatattcatgaagcagggcaatgtaacaattggatgcccacagcagttccaacgtcgactaacagtaacatcaacatgcctcctactttcactgtctggaatgacacataa

SEQ ID NO: 14 - DNA, 1599 bp, Prunus avium
  source 1..1599: mol_type = other DNA
atgggctctatgaattggcttggattttctctgtctccacaggaagtggtgccagccgatccttctatttcagaaggcgatcatcaccatcagcaccatgatggacatgagctgtctgatgatcagactgcaaccttggtgtcacgccttggttttaaatactctgatgatatctcaggcactgatgttaccggcggatgcttcgatttgacctctcacgatgattcttcagcaactaccacagctgccgcagttcctaataaccactcacataatcaccttattccacctccatttggtatgctgcatcaggctgccgcattcaatactaccaacatccactctcagggcgctgattggtcaaacatgaactcttcagattcttctacttataagacaacttctgatcatttgtcaatgcttatgggctctaattcttcatacagatcacagaacttggagaataaccatcagcagccaaagctggaaaactttttgggacgccactctttcgtggcagatgaacaccatattggtggccagtcttcatctggcactcatgcttacaataacaattctacatcaaacactatccagaccctgtctttgtcaatgtctaccggatctcacacacaccataacaccacatcaacagctgctgctggcgagacttgctcatctgatcgcgataatacatgtaacaataacaataagctggctgtggttaggactaccgcctcaactgcacctccacctggcattgattctcagacaacttcaaccacagctaccgccatcgaagctgtgccaagaaagtctgttgatactttcggacagagaacctcaatctatcgcggaacagatggtcaggtggatatgaagttgatctctggaattatcgttgccggtgagaaggataaacttgcaagagaaggcaagatttcaaactacgagaaggaaatcgatgagatgaagcatatgaccagacaggaatacgttgcttctctgagaagaaagtcttctggcttttctcgcggagcctcaatctatcgcggagtgacaaggcaccatcagcacggtaggtggcaggctaggatcggtagagttgccggcaataaggatctttatctgggcaccttctcatctaactctgtgatgtactcaacagccactaccactaatggcggcggcggcggcggcggcggcgatcatggcggatacattatcccaatgggcactgttatttctaatgatcacaacgatggatttggcgatggcactaaccatgatcaccaggtgaaagcagttgctcttggttttgagaatgtgttcggctcaacttctgcatcagtgaatgctacaaacgttactagggatgcctataacaatcatgcaagaaacctgtattacttgccacagcagctgcctcagtcatcttcagtgtcttcagtttcttcagctggcgtggccaagggtggctctgcatacgatattcacgaagctggacagtgtaacaattggatgccaacagctgttcctacatctactaactcaaatatcaacatgccacctaccttcacagtgtggaatgatacttga

SEQ ID NO: 15 - DNA, 894 bp, Rubus occidentalis
  source 1..894: mol_type = other DNA
atggaaccacaatcccagcaaccaactgaggatggaggaagcaacaaagcagctgggagtgctaacatgctttgcaggcagagcagtaccaggtggactcccactactgatcagataagaatcctcaaggagctttactacaacaagggagttaggtccccaagtgcagagcagattcagaggatctgtctccagctgaaacggtacggaaagatcgagggaaagaatgtcttttactggttccagaaccacaaggctcgagagaggcagaagaagaggttcacttcggatgttcatgtgcccatgcaaagatcagggcttgttgggaacagtaacgtagctaattggaaacctgaggatcagtatctcaacatgaacaacaagtactccaacatttgtcaaggggtttcttcttctgcatctgcttctgcttcttcagctggtgtggtttcttttaatgggcagatggggaactatggtgtttatggatctatgaacatggagaagagttttagggactgttcaatctcagctggaggaggtaccagtactactggtgttggaattggtggatccattagtcacaattacgggtcatgggtcggtggtagtgttgacccgtattcctcctcatcgtacaccactactagtactactttctttgacaaccaaaaacaaattttcatggaagaagaagaagaggatcaccaggagattgaaacccttcccctgttccccatgcatggtgaggacatctttggcaacatcaagacaacttccgaaggcggcggcggcgactacggctactactccagcggctggggcggtagctctcgcacttcccttgagctcagcctcaattcctacggctacaacaaccccaactattactga

SEQ ID NO: 16 - DNA, 894 bp, Rubus occidentalis
  source 1..894: mol_type = other DNA
atggaaccacagtctcagcagcctactgaggatggcggatcaaataaggctgccggatctgctaacatgctgtgcaggcagtcttcaacaagatggactccaactaccgatcagatcaggatcttgaaggaactttactacaataagggcgtgagatcaccttctgccgagcagattcagcgcatctgtctgcagttgaagagatatggcaagattgaaggcaagaatgtgttttattggttccagaaccataaggcaagagagcgccagaagaagagattcacttctgatgtgcacgttccaatgcagcgctcaggtctggtgggcaattctaacgttgctaattggaagcctgaagatcagtatttgaacatgaataacaagtactcaaacatttgccagggtgtgtcttcatctgcatcagcttctgcctcatctgccggcgtggtgtcttttaatggccagatgggtaactatggcgtttacggatctatgaatatggagaaatctttccgcgattgttcaatctctgcaggtggcggaacttcaacaactggcgtgggaattggtggctcaatctctcataattatggctcatgggtgggaggttctgttgatccatattcatcttcatcttacaccacaacttctaccacatttttcgataaccagaagcagattttcatggaggaagaggaagaggatcatcaggaaatcgagactcttccactgtttcctatgcacggagaagatattttcggtaatatcaagactacctcagagggcggaggtggcgattacggctattactcatctggatggggaggttcatctagaacatctttggaactttcactgaattcttatggctacaataaccctaactattactga

SEQ ID NO: 17 - DNA, 918 bp, Prunus avium
  source 1..918: mol_type = other DNA
atggaacctcaacaaacccaacaacaaccaaatgaagatggcagcaacaaagggactagtggtactaattttctgtgcaggcaaagcagtacaaggtggacacccacaagtgatcagattagaatattgaaggacctttactacaacaatggtgtgaggtccccaagtgcagagcagattcagaggatctctgctcggctgagacagtacggcaagattgaaggcaagaacgttttctattggtttcagaaccacaaggctcgtgagaggcagaagaaaaggttcattgatgtaccagccccacccatcatgcaaagatcagggcttgggattaataataatgctcctactgcttatgaacccattaatcacagcaacaagtaccccaacatttctgcttctgctggggtttcttctgcatctgcttcttcagctggtgtgattgctgttgggcaaatggggagctatggctatggatctatgaccatggagaagagttttagggactgctcaatatcagctgcaggaggtactagtagtggtcatgttggtggatctaataataatattggtcacaacgttggatcatgggttggtgttgatccatattcttcaccctacactctctttgacaagagatcatcatcaagacaagtgtttggtgaccaagaaaatatgatggatgaagaagatcatgaataccaagaaaacctgcaagattccccagagattgagaccctccctctcttccccatgcatggtgaagacatccatggctttggcaacatcaagtccacctccgacagctactactccggctggtaccgctccgacgagggcaacaatggccgcacttcccttgagctcagcctcaattcctacggccacatgacccctgattatttcagctcatattaa

SEQ ID NO: 18 - DNA, 1107 bp, Prunus avium
  source 1..1107: mol_type = other DNA
atggaacctcaacaaacccaacaacaaccaaatgaagatggcagcaacaaaggtaagtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaatttgttgatgtgcagggactagtggtactaattttctgtgcaggcaaagcagtacaaggtggacacccacaagtgatcagattagaatattgaaggacctttactacaacaatggtgtgaggtccccaagtgcagagcagattcagaggatctctgctcggctgagacagtacggcaagattgaaggcaagaacgttttctattggtttcagaaccacaaggctcgtgagaggcagaagaaaaggttcattgatgtaccagccccacccatcatgcaaagatcagggcttgggattaataataatgctcctactgcttatgaacccattaatcacagcaacaagtaccccaacatttctgcttctgctggggtttcttctgcatctgcttcttcagctggtgtgattgctgttgggcaaatggggagctatggctatggatctatgaccatggagaagagttttagggactgctcaatatcagctgcaggaggtactagtagtggtcatgttggtggatctaataataatattggtcacaacgttggatcatgggttggtgttgatccatattcttcaccctacactctctttgacaagagatcatcatcaagacaagtgtttggtgaccaagaaaatatgatggatgaagaagatcatgaataccaagaaaacctgcaagattccccagagattgagaccctccctctcttccccatgcatggtgaagacatccatggctttggcaacatcaagtccacctccgacagctactactccggctggtatcgctccgacgagggcaacaatggccgcacttcccttgagctcagcctcaattcctacggccacatgacccctgattatttcagctcatattaa

SEQ ID NO: 19 - protein, 323 aa, Rubus occidentalis
MDCGGPTTNKDKLLVIMGATGAGKSRLSLDLAACFPPATFEVINADKMQVYKGLDITTNKLPAPERLGVPHHLLGEFDSLDGEVTPAKFRELAARVVSGVSARRKVPVLVGGSNSFIHALVVDRFEPGFNVFDGDSVTVSISSELRYNCCFLWVDVSSVVLTEYLSKRVDEMLDSGMLLELAEFYNSAEEDSVVRTGIRKSIGVAEFSRYFRKHPPPGGSEEVGDDDPVRREAYKDAVREIKLNTCQLAKRQIGKISRLRGAGWNLRRLDATEAFRAAVKSDEKDGNRWSEIWEREVVGPSVKIVNHFLEEEIQSTKVQNQKI

SEQ ID NO: 20 - protein, 357 aa, Rubus occidentalis
MTLDSFPTLHYSYYNITPHSLSSPAVTLTPPPSLPQKPRWARMDSGPATTTTVPLRHKDKLVVIMGATGAGKSRLSIDLATHFPSCFEIINSDKMQVYKGLDITTNKIPVPDRIGVPHHLLGQFDPLDGELTPAEFRQLAGQAVSAITSRRKVPMLVGGSNSLIHALLVDRFEPGVDVFEGGAAISSELRYNCCFLWVDVSLTVLADYLSKRVDEMLDSGMLEELAEFCDPEDDDLAIRTGLRKAIGVPEFSRFFKKYPPICQSQKARPDDPVRRGAYKEAVRAIKDNTCQLAKTQIGKILRLRVAGGWDLQRLDATEAFRAVVTSEDDAGKRWSEIWQKQVVEASVKIVKRFLEQE

SEQ ID NO: 21 - protein, 374 aa, Prunus avium
MTLHPFPTHSHHHYSNLNFYSYISRFHLFPPVNHLCPTTPSHHHPLPHIKPRRGAHISSGPTTPRQQQQRKDKLLIILGATGAGKSRLSLDLATRFPFFEIVNSDKMQLYAGLDITTNKLPIPDRLGVPHHLLGEFDPRHGDFTPAQFRAVAGQAISSITNRRKVPMLVGGSNSFIHALLVDRFEPGSNVFEPGFNGSVSSELRYNCCFLWVDVSLTVLTEYLCKRVDEMLDSGMLDELAEFCDPDRQDEDELTASQTALRKAIGVPEFTRYFEKYPPQGRGGEGDDRERREAYEEAVRAIKDNTCQLAKRQIGKILRLKGGGWDLRRLDATDAFRAVVATTSSDKDDGKRWSEIWERQVVKPSVKVVKRFLDE

SEQ ID NO: 22 - protein, 446 aa, Rubus occidentalis
MEEYNNSNEMDHESSGTPRVSASTFDIYIIIIINYNISNIFFKIKECSIQLLFCSLVCIFFITFLIYDQNLLMCRGNFLYASPNLGGNYGRTAAGDHNQQINTFHLQSSGGECFQSGGTAQPTVKTEAAGSTSQHAPHKFHHQFPPPSLILSRGQNDQLPVQQQQHIENENEVEAIKAKIIAHPHYSNLLQAYMDCQRVGAPSEVVARLAAARQEFEARQRSLVSSRDASSKDPELDQFMEAYYDMLVKYREELTRPIQEAMDFMRKIETQLNMLGNDSSTTPLRIFSASAQEDKCDGNGSSEEDQDNNSGGETEVAEIDPRAEDRELKNHLLRKYSGYLSSLKQELSKKKKKGKLPKDARQKLLSWWELHYKWPYPSESEKVALAESTGLDQKQINNWFINQRKRHWKPSEDMQFMVMDGLHPQNAATLYMDGHYIGDGHYRLGP

SEQ ID NO: 23 - protein, 355 aa, Prunus avium
MEEYNQMDHESSGGRGNFLYASPNLGGNYGRASHDHQMGINAFHLQSSGGGDFHSPGGAHPINVKTEASTSHHGHHKFQYNNNNNNNNNNPLLSSRGHQPVVQQQHNLDRQNDDHTLSSNEVEAIKAKIIAHPQQRSSVASREASKDPELDQFMEAYYDMLVKYREELTRPIQEAMDFMRRIETQLNMLGNNNAPPLRIFSPSEDKCEGIGSSEEEQENSGGETEVPEIDPRAEDRELKNHLLKKYSGYLSSLKQELSKKKKKGKLPKDARQKLLSWWELHYKWPYPSESEKVALAESTGLDQKQINNWFINQRKRHWKPSEDMQFMVMDGLHPQNAALYMDGHYIGDGHYRLGP

SEQ ID NO: 24 - protein, 700 aa, Rubus occidentalis
MNMNWLGFSLSPQEDDAPISHQLADQETLASRLGFNSNDQIQGAGGEADWNMKGVDMNSSDGNNYKRTSSSSDLSPMLMGSSTTTSTSCSIISQPNLENHHQQQQLPKLENFLGRHSFADHDQSSTSHDYMFDMNNGPGAISSNVMNTSNTNSNNIGLSMIKTWLRNQPPQPRDNNHQLEQESKTTKESRNQPQSSLSLSMSTGSLHTVTTATAGGASASAGAGETTSSSNNNRKQSPVLTATTTDPQTQTTGGAIEAVPRKAIDTFGQRTSIYRGVTRHRWTGRYEAHLWDNSCRREGQTRKGRQGGYDKEDKAARAYDLAALKYWGTTTTTNFPISTYEKEIDEMKPMTRQEYVASLRRKSSGFSRGASIYRGVTRHHQHGRWQARIGRVAGNKDLYLGTFSTQEEAAEAYDIAAIKFRGLNAVTNFDMSRYDVKSILESSALPITTGASAKRLKDVQQQPPPPADHHHQIMLSSVLDHHGQMIRSSSSTEHDIMGNIFHQDDDHLRQQLQMGGTHNFFHDHDHQQRSGLMGLMDSSAAAASMEHSSGSNSVIYSVGDHHGNNNGYGSNARGTGSGYIMPMAMSTVVANHGQNQADGNNINGFGDGDGDDQEVNIKAQQLGYDHHQNVFLGSSNTTDPAYQHHASNRNLYYQLPVHDDQHESSSEAVATSSTTCNMNWVPTAVPTHLAHSTFTVWNDT

SEQ ID NO: 25 - protein, 532 aa, Prunus avium
MGSMNWLGFSLSPQEVVPADPSISEGDHHHQHHDGHELSDDQTATLVSRLGFKYSDDISGTDVTGGCFDLTSHDDSSATTTAAAVPNNHSHNHLIPPPFGMLHQAAAFNTTNIHSQGADWSNMNSSDSSTYKTTSDHLSMLMGSNSSYRSQNLENNHQQPKLENFLGRHSFVADEHHIGGQSSSGTHAYNNNSTSNTIQTLSLSMSTGSHTHHNTTSTAAAGETCSSDRDNTCNNNNKLAVVRTTASTAPPPGIDSQTTSTTATAIEAVPRKSVDTFGQRTSIYRGTDGQVDMKLISGIIVAGEKDKLAREGKISNYEKEIDEMKHMTRQEYVASLRRKSSGFSRGASIYRGVTRHHQHGRWQARIGRVAGNKDLYLGTFSSNSVMYSTATTTNGGGGGGGGDHGGYIIPMGTVISNDHNDGFGDGTNHDHQVKAVALGFENVFGSTSASVNATNVTRDAYNNHARNLYYLPQQLPQSSSVSSVSSAGVAKGGSAYDIHEAGQCNNWMPTAVPTSTNSNINMPPTFTVWNDT

SEQ ID NO: 26 - protein, 297 aa, Rubus occidentalis
MEPQSQQPTEDGGSNKAAGSANMLCRQSSTRWTPTTDQIRILKELYYNKGVRSPSAEQIQRICLQLKRYGKIEGKNVFYWFQNHKARERQKKRFTSDVHVPMQRSGLVGNSNVANWKPEDQYLNMNNKYSNICQGVSSSASASASSAGVVSFNGQMGNYGVYGSMNMEKSFRDCSISAGGGTSTTGVGIGGSISHNYGSWVGGSVDPYSSSSYTTTSTTFFDNQKQIFMEEEEEDHQEIETLPLFPMHGEDIFGNIKTTSEGGGGDYGYYSSGWGGSSRTSLELSLNSYGYNNPNYY

SEQ ID NO: 27 - protein, 305 aa, Prunus avium
MEPQQTQQQPNEDGSNKGTSGTNFLCRQSSTRWTPTSDQIRILKDLYYNNGVRSPSAEQIQRISARLRQYGKIEGKNVFYWFQNHKARERQKKRFIDVPAPPIMQRSGLGINNNAPTAYEPINHSNKYPNISASAGVSSASASSAGVIAVGQMGSYGYGSMTMEKSFRDCSISAAGGTSSGHVGGSNNNIGHNVGSWVGVDPYSSPYTLFDKRSSSRQVFGDQENMMDEEDHEYQENLQDSPEIETLPLFPMHGEDIHGFGNIKSTSDSYYSGWYRSDEGNNGRTSLELSLNSYGHMTPDYFSSY

SEQ ID NO: 28 - DNA, 723 bp, Agrobacterium tumefaciens
  source 1..723: mol_type = other DNA
atggatctgcgtctaattttcggtccaacttgcacaggaaagacgtcgaccgcggtagctcttgcccagcagactgggcttccagtcctttcgctcgatcgggtccaatgttgtcctcagctgtcaaccggaagcggacgaccaacagtggaagaactgaaaggaacgagccgtctataccttgatgatcggcctctggtgaagggtatcatcgcagccaagcaagctcatgaaaggctgatgggggaggtgtataattatgaggcccacggcgggcttattcttgagggaggatctatctcgttgctcaagtgcatggcgcaaagcagttattggagtgcggattttcgttggcatattattcgccacgagttagcagacgaagagaccttcatgaacgtggccaaggccagagttaagcagatgttacgccctgctgcaggcctttctattatccaagagttggttgatctttggaaagagcctcggctgaggcccatactgaaagagatcgatggatatcgatatgccatgttgtttgctagccagaaccagatcacatccgatatgctattgcagcttgacgcagatatggaggataagttgattcatgggatcgctcaggagtatctcatccatgcacgccgacaagaacagaaattccctcgagttaacgcagccgcttacgacggattcgaaggtcatccattcggaatgtattag

SEQ ID NO: 29 - DNA, 723 bp, Agrobacterium tumefaciens; identical to SEQ ID NO: 28
  source 1..723: mol_type = other DNA

SEQ ID NO: 30 - DNA, 957 bp, Arabidopsis thaliana
  source 1..957: mol_type = other DNA
atgaagtgtaatgacaaaatggttgtgatcatgggtgccaccggttctggcaagtcatcactctctgttgatctcgctttacattttaaagccgagatcatcaactctgacaaaatgcagttctacgatggcttgaagatcaccacgaatcaatcgaccattgaagatcgacgtggagtgccacatcaccttctcggtgaactaaacccggaggctggagaagtcacagcggcagaatttcgcgttatggcggctgaagccatctccgagattactcaacgtaaaaagctcccaatccttgccggtggatccaactcatacattcatgctctccttgcaaaatcttatgaccctgaaaactatccgttttctgatcacaagggctcaatctgctccgagttgaaatatgattgttgtttcatttggatagatgtggatcagtctgtgttattcgagtatctttctttacgtttggatcttatgatgaagtcaggtatgttcgaggagatcgctgagttccaccgctctaagaaggccccgaaagagccattggggatatggaaggctataggagtgcaagagtttgatgactacctcaaaatgtacaagtgggacaatgacatggataaatgggaccctatgagaaaggaggcttatgagaaggcggtgagagccatcaaagaaaacacatttcagctcacaaaggatcaaatcacgaagatcaacaagctgagaaatgccgggtgggacataaagaaggtggatgctacagcatcgtttcgagaggcaattagggcagccaaggaaggcgaaggtgtagccgagatgcagagaaagatatggaacaaggaagtgttggaaccgtgtgtgaagattgtcaggagccacttggaccaaccgatcaactattattattattacttttatttactaaaaagatttttaagtcttaactag

SEQ ID NO: 31 - DNA, 956 bp, Arabidopsis thaliana
  source 1..956: mol_type = other DNA
atgaagtgcaatgataaaatggtggttattatgggtgcaacaggttctggcaagtcttcactgtcagttgatctggctttgcatttcaaggccgaaattatcaattctgataagatgcagttctacgatggcctgaagattactaccaaccagtcaactatcgaggatagacgcggtgtgccacatcacctgttgggcgaattgaaccctgaggctggagaagtgaccgctgccgagtttagagttatggcagctgaggctatttctgaaatcacacagcgcaagaaacttccaattctggccggcggatctaattcatatatccatgcacttctggctaagtcttacgatccagaaaactatcctttctcagatcacaaaggatctatttgttcagagttgaagtacgattgctgttttatttggatcgatgtggatcagtctgttcttttcgagtatctttcactgcgcttggatcttatgatgaaatctggaatgtttgaggaaatcgcagagttccataggtcaaagaaagctccaaaggaacctcttggcatttggaaagcaatcggagtgcaggaattcgatgattacctgaagatgtataagtgggataatgatatggataagtgggatcctatgcgcaaggaagcttacgaaaaagccgttagggcaattaaagagaacacattccagttgactaaggatcagatcactaagattaataagcttagaaacgccggatgggatattaagaaagtggatgctaccgcctcattcagggaagccatcagagccgcaaaggaaggcgagggtgtggcagagatgcagaggaagatctggaataaggaagtgctggaaccatgcgtgaaaattgttagatctcacttggatcagcctatcaattactactactactacttctatttgcttaagcgcttcctgtcattgaactg

SEQ ID NO: 32 - DNA, 7908 bp, Zea mays
  source 1..7908: mol_type = genomic DNA
agtgcactgtctctctccaaaggcagctgcattggcctccagccttttccctactgtgccgcgcgccctcccttctctctaatgatagcatagggagagaaggcatactccgaggcatccttctcctttccctctccttccccaaacccttttcctctttccctcgccccaagaacttcatctcatctccaggcgccccttttgcgcttgcgcaggaggagctcacggggacagtgggcggagagctcgatcgctgcaccactacttcactggaggtccgcccactcccatggaggagatcacccaacactttggagttggcgcaagcagccacggccatggccacggccagcaccaccatcatcaccaccaccaccacccgtgggcatcctccctcagcgccgtcgtagcgccgctgccgccgcaaccgccaagcgcaggcctgccgctgaccctgaacacggtggcggccactgggaacagcggcggtagcggcaacccggtgctgcagcttgccaacggtggcggcctcctcgacgcatgcgtcaaggcgaaggagccctcgtcgtcgtctccctacgcaggcgacgtcgaggccatcaaggccaagatcatctcgcacccacactactactcgctcctcactgcctacctcgagtgcaacaaggcaagcaagactagtagccgcagtctaacaatagctctcctgcttccggttcagctactgctgctagcgaagtttggccacatctgatgtcaagagcatggcgcatgcacatactttctgcttgcttttacatagcatactatagtttatgcatgctgctcatgtagacatgtgcatctggtgttcggaactttctcaaacggccgtgggtgtttgcaggtgggggcaccaccggaggtgtcggcgaggctgacggagatagcgcaggaggtggaggcgcggcagcgcacggcgctcggcggcctggccgctgcgacggagccggagctggaccagttcatggtgaggcatctttaggtttatttcacgcgcgcgcagcgcaccattattagtcgttttcagctcgggctcggatttaatttgcgtgtatatatggtaggaggcgtaccacgagatgctggtgaagttcagggaggagctgacgaggccgctgcaggaggcgatggagttcatgcgaagggtggagtcgcagctgaactcgctttccatctccggaaggtcgctgcgcaacatcctttcatctggtactgaagttgctgcggcccatgcccctcttattttatattagatgatttcttggatcggttgtcgcctcatgcagcgatgcatgccctccctcttgttaaaatcttcctgtcctctccttcgtcttgctgtcctctttcttggttgagcatatgttgatgggtttttttttttgcatttcaaaagggttaaatttttccttgatgtggtttctcagcaattaattttggcatctgtgtagttccctttttaagagcatcttgctatgcatgcatattttgagtatagatagctgcggaacgcagatgaatgtcttgtctgcgcttcatgttttttaatgcgcggcagcattcctaatcaggggtttcgaaagagaatcttctccctgcttgcttttcctggccatgcatctgtgagattcttctctctggcttccgtcgatctttgcttctgctggaaaggaaatagtcctcagtcacacatgtgcaggagcgcagcttcaatctagcatggctgaaagctctctggttctttccatacactccatcctgcacattccttgcatatattctgctgttaaactgcttagcgacgaggatgaatgaaaacacctgtcttttttactagtgggagtactaatttggctgctgttgccatggatgcgttgttcatcgtcctccattatattgatcgattccatttggaatggaacatgtgcatgtgttctgcgagtagttcctggctcagcgcagccatcttttcccatgatgggaaccccagggagttcccttgttctagggtttgggaaagggccacagtgtactgggtgctgtggcattgtgcaagcacagtctatagccgaggagtctcactggcgtatgtcagtgaaacatagtagtatacacgttgtagtagtgggagatcagagagagagagagatggggagtcatcccccatctgtcacgcaagcttgtggccagaacattcttgtcatctttctccccctcaagagatgtagctacctgttgtcctagaatctatagtgttgtggtgtggtcttccaggtgcgcacacagatctggccactgctggccttagtatttgctaattttaaaatgaacacatcaggtagatcgagacgatccatggcaagcgtctttctaatgacctatcacaaggctatagtaacaactcgtaccaccacagttacgcacagcccagagtttttcacttctcggttctgttcttcttagagcactggctatggctagtatatatgcatggcagcaacacacacacacacacacaatcacttcattactggagcaagttagcgaagaagctgccatctgatgtcgtagaatgctgcaaaaatgaaaggttcagaggcaggcatacgggttgaaatggagcaccgtatgcggcggcccagttttttgtgttctgaccggcgcagtggacaaaatggcctgtgtgcctcgagaaccatagagacagttgcctagcgcatgagcgctgagcggcccctattatttgatgtgagatctctctatcaatggatgtgtgatctgctaggtttatattatatatgcgtctactatatccctaccagtccctgtattttctgacagatagacttctcccccgcgcgttctctcataaataaatgacggtcaaggaacattggatgctttccgaggcgagctagtacaagtttcatctgcgtttgcgtttgtatttttcagctgtcaactcagatttctcctcagctccaactgttctttgaccttcttggtactgttttcttggaagaaactatttgttgaccatttttggttataataaggccacagacaaactgttgaatttaatcacaaaacaacactactgcgttttttcttttcttttctgactgatgttgcacatgtactccaagattcttggttgcatgcactctttcaaggtcatgcaaaagcagggacaccgggtgtcacgagtttccgtttgtctcgaggtaagaccaaaaaagataccggaagcaaacaacataaaacaaagggtaggatggaaaagagctgatggattttacaatgtatcgccagagtggaaaagacaacgcgactagaggaaaggaacgatctggcacttccaactgtgtgaattcttccatttgatgtgcgtctcgtgcatgtttattttcttggttactggaaatcgggtgcatccatttttattcaggattcaattattggttactggagccaggtccgttcgtttttattttccttttgagattgactggatcgattctctggttctagtcacttatgctgttggttttatcgttttcatggatttctattctatctctcccaatattcagtatcacataaaatcctgtgactagatttctttatcacatacaatcttgtttttttattttctttaatttaacactggatttgtttgataatatgttctgaaaaacgttaataaatttaagcacaacagccttttaaaaatatgtataattatttaccttgaaacccagcatcggccgggaacaaggaaaagaactcaaggttttaaatgcattgaaatgaaacattaatggagtgtttggtttgaataatgatgtagtccatcatcttctcactcctcacttttttgtttggtttatggaatggagtgagttaatccatcaacacctcattcctcatagttagttgtttagtactaatatgtggaatgaagtcatcccaccaaatttgaagaatggactcatgatgcaccacttcattttagatagagtgattcatcaaaccaaacacctcataagggcatgtttggatcctaggagctaaaagaaaagtgactaaagtttagtcactttaggagctaaagatctactaaataggaaactaaaagtgactagaatagtaaaaggtatctttttagtcatttttagctcctaagaaggagctaaattttagttagtttggtttagctcttggatccaaacaggccctaagaaaatgtttcgacaacatttgggacaataaaacagttcctaaggatttctttggcaagattaggccttctttggaacaaagaaaaatgaaggaatcttgaaggattgaaatcctataggaagctttcatatgcaaagaattgtgttcctaggatgatttctaacaagaggctcatccccttgaaaattgttctttgtgtctatctctctcctctaattcatgtgttcttatgttgcattgaaacactattagaaaattttcatgtgttttaatttatgtatgattgtaagtgtcaagcagcactattcctacattttttctattcctgtgttttatcgatactgcatcccaatgaaggcctaagcgtatcatgcaatcttttccagaagttttttagattgctgtataatgacaaggtgcaatgctcttcgttttcttttttggttttcctcattttcagcaaggccaccaaatttttccatgatgttcttgtcttattcctgtagtatccctcgaaaaaatttcatagctttctacatccatgttatctaatgatgttcttcatttcaaagacagacacatttggcataagttctgattggtcactaaaactttgtggtacggtcatttacttttactatacacatgtactgtattcatgcgtacattgtactttgtccattggggcctttcctctttttaatacaacgggcatcgtttcaaaaaaaatgtgtcagtcgtaactgcacacttctttagttttccccagttaacagcatgacggaatagagttacagagtctcatgtcaagtcacatatatcctcaagtcgtttctaaaattaaagtaatattttcattgttgcttccgtaagaatgcaccacaaacacaaaatatcatttcctttatgcaaatatatatatatgaatgtttgtttctggcagtggtgagaagcttctcactgagccaaaggttttagggatgccatgtgctctttttcttttttaagtgtagattaagcaaacctttataatttttaaactccaaccagataccttttctaaattcataaatgctctggactatcttcacgaaaaaggctttggtaatatgttcacaagcctcctttatctgttatcctgggctagacataggatgtgttgaagcaatacaagcggagttgtttcgttaaagcaaaaaaaaaactgtaactttatattggagaatatacatcgttctcccatatttgttacagtgctcataaatagacaaagttttttttttgaaatcttaagccctgtgtttctttggttatcttatctctatattgcttccaaatatgttgtcatcaagatggatatagataattgtcaaaacgaaaagtgtgcatggttcatttatgccaattctgaaaagcataagttaaatattaagataccaataaagaaaaacatatgcagtgttgactgttgggaaaagaacaaggtctgcacatacacttgcaatataaatctttttacccagcaataaaaaaacgatcaaatatcacgcaaacaaagtaatacgtccagaaacacccatacttttgaaattcgtctttgaaatgcagtaggttcacaaaataaaatggtgcaactgcacatgtcctttatatctgtactcaatgggttttctggagatgttagattgattggtgggacaatatcctaatgcaactcgcaaattcccaaggccgaactcaagtgggagtttggatgattttttttgtaaagaagcagatggctgcgctagtttacatagaccatcacacttcacaattcacattcatgaagtcataactttgttcacttcttgtttaactatggcatataaaaacatactgtgtgttgttttgcttgtatggaacatgtattttagttttttagtaaataagttcagtaaatgtctttgccggacaaatttcaccaatctggctacgaatgatgcttgatcacttcttttgttttatctaaaagttccatgtcttatgcttgaaggctcttctgaggaggatcaagaaggtagcggaggagagaccgagctccctgaagttgatgcacatggtgtggaccaagagctgaagcaccatctcctgaagaaatacagtggctatctaagctcgctcaagcaagaactgtcaaagaagaagaagaaagggaagctccccaaggaggctcgccagcagctccttagctggtgggatcagcactacaaatggccttacccctcagtacgtcttctttttattcttccattttaactattgttggtgacacatgatttagacgatgccaattcttcatgaacttttcatagccagctacccaatgttagtactgactgcacattgtaattcaagggtaagtatatatatacataaatcacatttggcaaatctaagctacatatgggtctttgatcttccatgacggtctgttgatctctgatttgcatatcggcatataaaaagtgagccaaaatatgtcagagtctaataatattgatcagggagtggcaggtgattattggtattaatttaaccttatttaaggtattttgaaacttctgtagcgttcttactaaataccattgattttaatttaagcaactatatatttatctggtgaaaaatgaagccttttctgatatacaaattgaagagtctacaatggtttcacttacatggctgaaacagaaaatcatagtgccctgaattgtgtgttgatactcataagcgcagattcaaatttgtaattttcaagtttagggttctaagtgaaaaaaaaacattgagtccaggagcatacactgaactttttttttatcatatcttcattttgttggatgttttgtatacggcatatagcctgtgcttccctactggatatgaattaaccaactcttcccatcggtgagcaggagactcagaaggtggcactggctgagtctaccgggcttgacctgaagcagatcaacaactggttcatcaaccagcggaagcggcactggaagccatccgaggagatgcaccacctgatgatggacgggtaccacaccaccaatgccttctacatggacggccacttcatcaacgacggcgggctgtaccggctcggctagccaccggtatctcgcttccatttcacaccccacggcctagctataaagactaatggttccaggtgtctgaagtactgaagacaggggggctagctatctaatgtttgtgccgcacgcatgagctgtaaggaggccatgcttaattattctgttgccgttgctactctatctatatgcgcctatgcctccgtgcatgaactatgctttaggtggttgctgctccacactgtggtggtgtgcttttgcttttgtgtggtcgtattgtatgcgtaacctgacagatggatccctgattgctacatgtttgaataatttgcatgatctagctagtttctgcctaatctaatggtacggctcatgtcttgtcaggtggatttgttatgtgttttttttttcaaattgaggcttgtttgttttgctctcaatccatccatgtgaattgggtggaattaaatgagtttaaatt

SEQ ID NO: 33 - DNA, 1077 bp, Zea mays
  source 1..1077: mol_type = other DNA
atggaggagatcacccaacactttggagttggcgcaagcagccacggccatggccacggccagcaccaccatcatcaccaccaccaccacccgtgggcatcctccctcagcgccgtcgtagcgccgctgccgccgcaaccgccaagcgcaggcctgccgctgaccctgaacacggtggcggccactgggaacagcggcggtagcggcaacccggtgctgcagcttgccaacggtggcggcctcctcgacgcatgcgtcaaggcgaaggagccctcgtcgtcgtctccctacgcaggcgacgtcgaggccatcaaggccaagatcatctcgcacccacactactactcgctcctcactgcctacctcgagtgcaacaaggtgggggcaccaccggaggtgtcggcgaggctgacggagatagcgcaggaggtggaggcgcggcagcgcacggcgctcggcggcctggccgctgcgacggagccggagctggaccagttcatggaggcgtaccacgagatgctggtgaagttcagggaggagctgacgaggccgctgcaggaggcgatggagttcatgcgaagggtggagtcgcagctgaactcgctttccatctccggaaggtcgctgcgcaacatcctttcatctggctcttctgaggaggatcaagaaggtagcggaggagagaccgagctccctgaagttgatgcacatggtgtggaccaagagctgaagcaccatctcctgaagaaatacagtggctatctaagctcgctcaagcaagaactgtcaaagaagaagaagaaagggaagctccccaaggaggctcgccagcagctccttagctggtgggatcagcactacaaatggccttacccctcagagactcagaaggtggcactggctgagtctaccgggcttgacctgaagcagatcaacaactggttcatcaaccagcggaagcggcactggaagccatccgaggagatgcaccacctgatgatggacggataccacaccaccaatgccttctacatggacggccacttcatcaacgacggcgggctgtaccggctcggc

SEQ ID NO: 34 - DNA, 1755 bp, Arabidopsis thaliana
  source 1..1755: mol_type = other DNA
atgaactcgatgaataactggttaggcttctctctctctcctcatgatcaaaatcatcaccgtacggatgttgactcctccaccaccagaaccgccgtagatgttgccggagggtactgttttgatctggccgctccctccgatgaatcttctgccgttcaaacatcttttctttctcctttcggtgtcaccctcgaagctttcaccagagacaataatagtcactcccgagattgggacatcaatggtggtgcatgcaataacattaacaataacgaacaaaatggaccaaagcttgagaatttcctcggccgcaccaccacgatttacaataccaacgagaccgttgtagatggaaatggcgattgtggaggaggagacggtggtggtggcggctcactaggcctttcgatgataaaaacatggctgagtaatcattcggttgctaatgctaatcatcaagacaatggtaacggtgcacgaggcttgtccctctctatgaattcatctactagtgatagcaacaactacaacaacaatgatgatgtcgtccaagagaagactattgttgatgtcgtagaaactacaccgaagaaaactattgagagttttggacaaaggacgtctatataccgcggtgttacaaggcatcggtggacaggtagatacgaggcacatttatgggacaatagttgcaaaagagaaggccagactcgcaaaggaagacaagtttatctgggaggttatgacaaagaagaaaaagcagctagggcttacgatttagccgcactaaagtattggggaaccaccactactactaacttccccttgagtgaatatgagaaagaggtagaagagatgaagcacatgacgaggcaagagtatgttgcctctctgcgcaggaaaagtagtggtttctctcgtggtgcatcgatttatcgaggagtaacaaggcatcaccaacatggaaggtggcaagctaggatcggaagagtcgccggtaacaaagacctctacttgggaactttcggcacacaggaagaggctgctgaggcttatgacattgcagccattaaattcagaggattaagcgcagtgactaacttcgacatgaacagatacaatgttaaagcaatcctcgagagcccgagtctacctattggtagttctgcgaaacgtctcaaggacgttaataatccggttccagctatgatgattagtaataacgtttcagagagtgcaaataatgttagcggttggcaaaacactgcgtttcagcatcatcagggaatggatttgagcttattgcagcaacagcaggagaggtacgttggttattacaatggaggaaacttgtctaccgagagtactagggtttgtttcaaacaagaggaggaacaacaacacttcttgagaaactcgccgagtcacatgactaatgttgatcatcatagctcgacctctgatgattctgttaccgtttgtggaaatgttgttagttatggtggttatcaaggattcgcaatccctgttggaacatcggttaattacgatccctttactgctgctgagattgcttacaacgcaagaaatcattattactatgctcagcatcagcaacaacagcagattcagcagtcgccgggaggagattttccggtggcgatttcgaataaccatagctctaacatgtactttcacggggaaggtggtggagaaggggctccaacgttttcagtttggaacgacacttag

SEQ ID NO: 35 - DNA, 1755 bp, Arabidopsis thaliana
  source 1..1755: mol_type = other DNA
atgaattctatgaataactggttgggcttttctctttcaccacatgatcagaaccatcacaggactgatgtggattcttcaactaccagaaccgccgtggatgttgcaggcggatactgctttgatttggctgccccatcagatgaatcttcagcagtgcagacttcttttctgtcacctttcggcgttactcttgaggctttcaccagagataataactctcactcacgcgattgggatattaatggtggcgcctgcaataacatcaataacaatgagcagaatggccctaagctggaaaacttcttgggaagaacaactaccatctataatactaacgaaaccgtggttgatggtaatggcgattgtggaggtggcgatggaggtggcggaggttctcttggtctgtcaatgatcaaaacctggctgtctaatcattcagtggctaatgccaaccaccaggataatggcaacggtgctcgcggcttgtctctttctatgaattcttcaacatctgattcaaacaattacaacaataacgatgatgtggttcaggagaagactattgtggatgtggttgaaacaactccaaagaaaacaattgagtcttttggtcagcgcacttcaatctacaggggcgttacaaggcatagatggactggacgctatgaagctcacctgtgggataattcttgtaagagggagggccagactcgcaaaggaaggcaggtttacttgggcggatatgataaggaagagaaagcagctagggcctacgatcttgccgcactgaaatattggggcaccacaactaccacaaattttcctctttctgaatacgagaaggaagtggaggagatgaagcatatgacccgccaggagtatgttgcttctctgagaagaaagtcttcaggtttctctaggggcgcctcaatctataggggtgtgacaagacatcaccagcacggcagatggcaggctagaatcggacgcgttgccggtaataaggatctgtacttgggcacatttggaactcaggaagaggctgccgaagcatatgatattgcagctatcaagtttagaggcttgtctgctgtgactaacttcgatatgaatcgctataacgttaaagccattcttgagtctccatcactgcctatcggatcttcagccaagcgccttaaagatgtgaataacccagttcctgcaatgatgatctctaataacgtgtctgaatcagcaaataacgtttcaggatggcagaataccgctttccagcatcaccagggtatggatctttcactgttgcagcagcagcaggaaagatatgtgggatattacaatggtggcaacctgtctaccgagtcaacaagggtttgctttaagcaggaagaggagcagcagcatttcttgagaaattctccatcacacatgacaaacgttgatcatcactcttcaacctctgatgattcagtgacagtttgtggcaatgtggtttcttacggaggttatcagggttttgcaattccagtgggcacctcagttaactacgatcctttcacagccgcagagatcgcatacaacgctagaaaccattactactacgcccagcaccagcagcagcagcagattcagcagtctccaggcggagatttccctgtggcaatctcaaataaccattcttcaaacatgtacttccacggagagggtggcggagaaggtgctcctactttctctgtttggaacgatacctga

SEQ ID NO: 36 - DNA, 1986 bp, Triticum aestivum
  source 1..1986: mol_type = other DNA
atggcttctggaaacgactggttggggttctcgctctccggccaagaaaacccacagccgcatcaggatagcccgccgcccgccggcatcgacatctccggcgccagtgacttctatggcgtacccacgcagccagctcccgacgctcaactcggcgtgccaggtcatcagccttcatacgggatcacggaggacttcaataggggagctcatgaaacccacgattggaacatgaggggtgatctggactataacggcggtgcgtccgagctctcgatgctcgtcgggtcgagcgcagtcggcggcaagagggcggccatggaggccgagaccgagcccaagcttgaggacttcctcggttgcaactcgttcgtctccgagcaagatcaggccggcggcttcttgttctccggagtcccgatgaccaccagcagcaacagcaacaccgggagcaacaccatggagctctctatgatcaagagctggctccgtgacaaccagctaccccaagcgccgcacccgcagtcgcagaccggggcgcccgcgcagcagccgcaaccccacgaggagatgggcaccgacgcgagcagcttcgatccgcgggggagaaacggcgcgttggtggtggcggggagctcgcagagcctggcgctctcgatgagcacgggttcaggatcgcacttgccaatggctgtggtcagcggcagtgccagtgggggagtctcagagagcacctcctcggagaacaagcgagccagcggcgccatggattcgccgggcggtgcggtagaagctgtcgcgaggaagtccatcgacacattcgggcaaaggacgtcgatatatcgaggtgtaacaagacatagatggacaggccgttatgaggctcatctctgggacaatagctgcagaagagaagggcagagtcgcaagggccgacaagtttatcttggtggctatgacaaggaggacaaggcagcaagggcttatgatttggcagcactcaagtattggggtacaacaacaactacaaatttcccaattaatacctacgaaaaagaggtggatgaaatgaaacatatgactaggcaggaatatatcgcatacttaagaaggaatagcagcggattttctcgtggtgcgtcaaaatatcgtggtgtaactaggcaccatcagcatgggagatggcaagcaagaattgggcgggttgcaggaaacaaagatctctacttaggcaccttcagcaccgaggaggaggcggcggaggcctacgacatcgcggcgatcaagttccgcgggctcaacgccgtcaccaacttcgacatgagtcgctacgacgtcaagaccatcctcgagagcagcaccctgccggtgggtggcgcggctaggcgcctcaaggaggccgctgatcatccggaggccggcgccacgatctggcgagccggcatggacggcgccatcatctcccagctgaccgacgtcgggatgggcgcgtacgcgtcgtaccaccacggctggccgaccatcgcattccagcagccgtcgccgctctcggtgcactacccatacgtcgcccagccgccccgcgggtggtgcaagcccgagcaggacgccaccgtcgccacgcaaagcctgcaggacctccagcagctgcacctcgggaccgccgctcacaactatttccaggcatcgtcgagctcaacggtctacaacggcggcggcgtcggcgggtaccagcagggcaccggtggcaacgccttcttgatgccggctagcaccgttgtggatgaacaggggcacagcagcactgccgccaaccagggaagcacctgcagctacggggacgaagaggggaagctcaatcttgggtacgatgccatggcgatggcgagcaccggcgccgacccatacgcggcggcgtcaactgtgagcatcgcgagggcgaacgggtactccaacaactggagctcgccgttcagtggcatgtga

SEQ ID NO: 37 - DNA, 1986 bp, Triticum aestivum
  source 1..1986: mol_type = other DNA
atggcctcaggaaatgattggcttggtttttctctgtcaggccaggagaaccctcagccacatcaggattctccacctccagcaggaattgatatctcaggtgcttctgatttctatggcgtgcctactcagcctgccccagatgcacagttgggagttcctggtcaccagccatcttacggtattactgaggattttaatagaggcgctcatgaaacccacgattggaatatgcgcggagatcttgattataacggcggagcctcagagctttctatgctggtgggttcttcagccgttggtggcaagcgcgctgccatggaagcagagactgaacctaaattggaggattttcttggctgcaactcattcgtgtctgaacaggatcaggccggaggttttctgttctcaggcgttccaatgactacctcttcaaactcaaacacaggatctaacactatggaattgtcaatgatcaagtcttggctgagagataaccagttgcctcaggctcctcatccacagtctcagaccggtgctccagcacagcagcctcagccacacgaggaaatgggcacagatgcatcttcattcgatcctagaggccgcaatggcgcccttgtggttgcaggctcttcacagtcactggccttgtctatgtcaacaggctctggatcacatctgccaatggccgtggtttctggctcagcatctggcggagtgtcagagtctacttcttcagaaaacaaacgcgcttcaggagccatggattctcctggtggcgctgtggaggcagttgctaggaagtcaattgatacctttggacagcgcacatctatctataggggtgtgactaggcatagatggaccggcagatacgaggcccacttgtgggataattcatgtagacgcgaaggccagtctcgcaaaggaaggcaggtttatcttggaggttacgataaggaagataaagcagctcgcgcttatgatttggccgcacttaagtactggggcacaactaccacaactaacttcccaattaacacttacgagaaggaagttgatgagatgaaacatatgacccgccaggaatatatcgcttacctgaggagaaattcttcaggattttcaaggggtgcctctaaatacaggggagtgaccagacatcaccagcacggtagatggcaggctagaattggccgcgttgccggaaacaaggatctttatctgggcactttttctacagaggaagaggctgccgaggcatacgatattgcagctatcaagtttaggggcttgaatgctgtgacaaacttcgatatgtctagatacgatgttaaaacaatccttgaatcttcaactctgcctgtgggaggagcagcaaggaggttgaaggaggctgccgatcatccagaagccggcgcaactatctggagggctggtatggatggcgccattatctcacagcttaccgatgttggtatgggcgcttatgcctcttaccatcacggatggcctactatcgcctttcagcagccttcaccactgtctgtgcactatccttacgttgcacagcctccaagaggatggtgcaaaccagagcaggatgcaactgtggctacccagtctctgcaggatttgcagcagttgcatcttggtacagcagctcacaattactttcaggcctcttcatcttcaactgtgtataacggtggcggagttggtggctaccagcagggcaccggtggtaatgcattcctgatgccagcttctacagtggttgatgaacagggccactcttcaaccgccgcaaatcagggatcaacatgttcttatggcgatgaagagggcaagctgaacttgggttacgatgcaatggctatggcctctacaggcgctgatccttatgctgccgcatcaactgtttctattgcaagagctaatggctactcaaataactggtcttcaccattctctggtatgtga

SEQ ID NO: 38 - DNA, 1980 bp, Triticum aestivum
  source 1..1980: mol_type = other DNA
atggcttctgggaacgactggttggggttctcgctctccggccaagagaacccacagccgcatcaggatagctcgccgcccgccgacatcgacatcgctggcgccagtggcttctatggcctacccacgcagccagctcccgacgctcaactcggtgtgccaggtcatcatcagccttcatacgggatcacggaggccttcaataggggagctcatgaaatccacgattggaacatgaggggtgatctggactacaacggcggtgcgtccgagctctcgatgctcgtcgggtcgagcgcagttggcggcaagagggcggccatggaggccgagaccgagcccaagcttgaggacttcctcgttggcaactcgttcgtctccgagcaagatcaggccggtggcttcttgttctccggtgtcccgatggcgaccagcaccaacagcaacagcaggagcaacaccatggagctctctatgatcaagagctggctccgtgacaaccaggtaccccaagcgccgcacccgcagtcgcagaccggggcgcccgcgcagcagccgcaaccccacgaggagatgggcaccgacgcgagcagcttcgatccgctggggagaaacggcgcgttggtggtggcggggagctcgcagagcctggcgctctcgatgagcacgggctcaggatcgcacttgccgatggctgtgctcggcggcagtcccagtgggggagtctcagagagcacctcctcggagaacaagcgggccagcggcgccatggattcgccgggcggtgcggtagaagctgtcgcgaggaagtccatcgacacattcgggcaaaggacgtcgatatatcgaggtgtaacaagacatagatggacagggcgttatgaggctcatctctgggacaatagctgcagaagagaagggcagagtcgcaagggtaggcaagtttatctgggtggctatgacaaggaggacaaggcagcaagggcttatgatttggcagcactcaagtattggggtacaacaacaacaacaaatttcccaattaatacctacgaaaaagaggtggatgaaatgaaacatatgactaggcaggaatacatcgcatacctaagaaggaatagcagcggattttcccgtggtgcgtcaaaatatcgtggtgtaactaggcaccatcagcatgggagatggcaagcaagaattgggagggttgcaggaaacaaggatctctacttaggcaccttcagcaccgaggaggaggcggcggaggcctacgacatcgcggcgatcaagttccgtgggctgaacgccgtcaccaacttcgacatgagtcgctacgacgtcaagaccatcctcgagagcagcaccctgccggtgggtggcgcggcgaggcgccccaaggaggtcgctgatcatccagaggctggcgccacgatctggcgggccggcatggacggcggcgtcatctcccagctgaccgacgtcgggatgggcacgtacgcgtcgtaccaccacggctggccaaccatcgcattccagcagccctcgccgctgtcggtgcactacccatacgtcgcccagccgtctcgcgggtggtgcaagcccgagcaggacgccagcgtcgccacgcaaagcctgcaggacctccagcagctgcacctcgggaccgccgctcacaactatttccaggcatcgtcgagctccacggtctacaacggcggcgggtaccagcaaggcaccagtggcaacgccttcttgatgccggctagcaccgtggtggatgaacaggggcacagcagcactgccaccaaccagggaagcacatgcagctacggggacgaagaggggaagctcaatctcgggtacgatgccatggcgatggcgagcaccggcgccgacccatacgcggcggcgccaactgtgagcattgcgagggcgaacgggtactccaacaactggagctcgccgttcaatggcatgtga

SEQ ID NO: 39 - DNA, 1980 bp, Triticum aestivum
  source 1..1980: mol_type = other DNA
atggcttctggaaatgattggcttggtttttctctgtcaggccaggagaacccacagcctcatcaggattcttcaccacctgccgatattgatatcgcaggagcttcaggtttctatggccttccaactcagccagctcctgatgcccagctgggagtgccaggtcatcaccagccttcttacggtattaccgaggcttttaatcgcggcgctcatgaaatccacgattggaatatgaggggcgatctggattataacggcggagcctcagagctttctatgctggtgggctcttcagcagttggtggcaagcgcgctgccatggaagctgagactgaaccaaaattggaggattttcttgtgggaaactcattcgtttctgaacaggatcaggctggaggttttctgttctcaggtgtgcctatggccacatctactaattctaactcaaggtctaataccatggaactgtcaatgattaagtcttggttgagagataaccaggttccacaggccccacatcctcagtcacagaccggagcacctgcacagcagccacagcctcacgaggaaatgggaacagatgcatcttcatttgatccattgggcagaaatggcgctcttgtggttgccggctcttcacagtctctggcattgtcaatgtctacaggctcaggatctcacctgccaatggctgtgttgggcggatcaccttctggtggcgtttcagagtctacttcttcagaaaacaaacgcgcatcaggcgctatggattctcctggaggtgctgtggaggctgttgccaggaagtcaattgatacattcggacagaggacttctatctatagaggtgtgaccagacatcgctggacaggcagatacgaggcccacttgtgggataattcatgcagacgcgaaggacagtctaggaaaggtagacaggtttatcttggcggatacgataaggaagataaagcagctcgcgcttatgatttggccgcacttaagtactggggcactaccacaactaccaacttcccaattaacacttacgagaaggaagtggatgagatgaaacatatgacccgccaggaatatatcgcctacttgaggagaaattcttcaggattctcaaggggtgcatctaaatacagaggagtgacacgccatcaccagcacggtagatggcaggctcgcattggcagggttgccggaaacaaggatctttatctgggcactttttcaaccgaggaagaggctgccgaggcttacgatattgcagctatcaagtttagaggactgaatgccgtgactaacttcgatatgtctcgctacgatgttaaaactatccttgaatcttcaaccctgcctgtgggtggagcagcacgcaggccaaaggaggttgcagatcatcctgaagcaggcgctaccatttggagggctggcatggatggaggtgtgatctcacagctgacagatgttggtatgggcacttatgcctcttaccatcacggctggccaacaattgcattccagcagccatcacctttgtctgtgcactatccatacgttgcccagccttctagaggttggtgcaaacctgagcaggatgcctcagtggcaacacagtctctgcaggatttgcagcagttgcatcttggaactgctgcccacaattactttcaggcttcttcatcttcaactgtttataatggcggaggttaccagcagggcacttctggcaacgccttcctgatgccagcatctacagtggttgatgaacagggccattcttcaaccgcaacaaatcagggatcaacctgttcttatggcgatgaagagggcaagctgaacttgggttacgatgccatggcaatggcttctaccggcgccgatccatatgcagctgcccctacagtttcaatcgccagagcaaatggctactctaataactggtcttcacctttcaacggtatgtga

SEQ ID NO: 40 - DNA, 2112 bp, Sorghum bicolor
  source 1..2112: mol_type = other DNA
atggctactgtgaacaactggctcgctttctccctctccccgcaggagctgccgcccacccagacggactccaccctcatctctgccgccaccaccgacgatgtctccggcgatgtctgcttcaacatcccccaagattggagcatgaggggatccgagctttcggcgctcgtcgccgagccgaagctggaggacttcctcggcggaatctccttctccgagcagcaccacaaggccaactgcaacatgatccccagcactagcagcacagcttgctacgcgagctcgggtgctaccgccggctaccatcaccagctgtaccaccagcccaccagctccgcgctccacttcgctgactccgtcatggtggcctcctcggccggcggcgtccacgacggaggtgccatgctcagcgcggccagcgctaatggtagcgctggcgctggcgctgccagtgccaatggcagcggcagcatcgggctgtccatgatcaagaactggctgcggagccaaccagctcccatgcagccgagggtggcggcggctgagagcgtgcaggggctctctttgtccatgaacatggcgggggcgacgcaaggcgccgctggcatgccacttcttgctggagagcgcggccgggcgcccgagagtgtctcgacgtcggcacagggtggagccgtcgtcacggctccaaaggaggatagcggtggcagcggtgttgccgccaccggcgccctagtagccgtgagcacggacacgggtggcagcggcgcgtcggctgacaacacggcaaggaagacggtggacacgttcgggcagcgcacgtcgatttaccgtggcgtgacaaggcatagatggactgggagatatgaagcacatctgtgggacaacagttgcagaagggaaggacaaactcgcaagggtcgtcaagtctatttaggtggctatgataaagaggagaaagctgctagggcttatgatctggctgctcttaagtactggggtcccacgacaacaacaaattttccagtgaataactacgaaaaggagctggaggatatgaagcacatgacaaggcaggagtttgtagcgtctctgagaaggaagagcagtggtttctccagaggtgcatccatttacaggggagtgactaggcatcaccagcatggaagatggcaagcacggattggacgagttgcagggaacaaggatctctacttgggcaccttcagcacgcaggaggaggcagcggaggcatacgacattgcggcgatcaagttccgcggcctcaacgccgtcacaaacttcgacatgagccgctacgacgtcaagagcatcctggacagcagtgcgctccccatcggcagcgccgccaagcgtctcaaggaggccgaggccgccgcgtccgcacagcaccatgccggcgtggtgagctacgacgtcggccgcatagcctcacagctcggcgacggcggcgccctggcggcggcgtacggcgcgcactaccatggcgcctggccgaccatcgcgttccagccgagcgcggccacgggcctgtaccacccgtacgcgcagccgatgcgcgggtggtgcaagcaggagcaggaccacgcggtgatcgcggccgcgcacagcctgcaggagctccaccacctgaacctgggtgctgccgccggcgcgcacgacttcttctcggcggggcagcaggcggcgatgcacggcctgggtagcatggacaatgcatcactcgagcacagcaccggctccaactccgtcgtgtacaacggtgttggtgatagcaacggcagcaccgtcgtcggcagtggtggctacatgatgcctatgagcgctgccacggcgacggctaccacggcaatggtgagccacgagcaggtgcatgcacgggcacagggtgatcaccacgacgaagccaagcaggctgctcagatggggtacgagagctacctggtgaacgcagagaactatggcggcgggaggatgtctgcggcctgggcgactgtctcagcgccaccggcggcaagcagcaacgataacatggcggacgtcggccatggcggcgcacagctcttcagtgtctggaacgatacttaa

SEQ ID NO: 41 - DNA, 2112 bp, Sorghum bicolor
  source 1..2112: mol_type = other DNA
atggccacagtgaataactggcttgcattttctctgtcaccacaggaacttccacctacacagactgattcaactctgatttctgctgccactaccgatgatgtgtcaggcgatgtttgcttcaatatcccacaggattggtctatgcgcggctcagaactttctgcactggttgctgaacctaagttggaggattttcttggcggaatttctttctcagagcagcatcacaaagccaattgcaacatgatcccatcaacatcttcaactgcctgttatgcatcttcaggtgcaacagctggctatcatcaccagttgtaccatcagcctacttcttcagcccttcactttgcagattctgtgatggttgcttcttcagccggtggcgttcatgatggaggtgctatgctttcagcagcttctgcaaatggctcagcaggtgcaggagcagcatctgcaaacggatctggttcaattggtttgtcaatgatcaagaattggcttcgctctcagccagctcctatgcagccaagggtggctgccgcagaatctgttcagggtctttctctgtctatgaatatggctggcgccacacagggagctgccggtatgccactgttggcaggagaaaggggaagggcacctgagtctgtgtcaacctctgctcagggcggagccgtggttacagcacctaaggaggattcaggtggctctggagttgcagctactggtgcattggtggctgtttctaccgatacaggaggttcaggagcatctgctgataataccgcccgcaaaaccgtggatacattcggccagaggacatcaatctatagaggagttacaagacatcgctggactggaagatacgaagctcacctttgggataactcttgcagacgcgagggccagaccaggaagggaagacaggtgtatctgggcggatacgataaggaggaaaaagccgcaagggcttatgatttggctgcccttaaatactggggcccaacaactaccacaaattttcctgtgaataactatgagaaggaactggaggatatgaaacacatgaccaggcaggaatttgttgcttcattgaggagaaagtcttcaggattctcaagaggtgcctctatctatagaggcgtgacacgccatcaccagcatggaaggtggcaggctaggatcggaagggttgcaggaaacaaagatttgtatcttggcactttctctacccaggaggaagcagctgaggcatacgatattgccgcaatcaagtttagaggattgaatgctgtgactaacttcgatatgtcacgctacgatgttaaatctatcctggattcttcagccttgccaatcggctcagctgccaagaggcttaaagaagctgaagcagctgcctctgctcagcatcacgccggcgtggtttcatacgatgtgggaagaattgcctctcagcttggcgatggtggcgcactggcagctgcctatggtgcccattaccacggcgcatggccaactatcgcttttcagccttcagcagctaccggactgtatcacccatacgcccagcctatgagaggttggtgtaagcaggaacaggatcatgctgttatcgccgcagctcactctctgcaggagttgcatcacctgaatttgggtgccgcagctggcgcacatgatttcttttctgctggtcagcaggccgcaatgcacggcctgggatctatggataatgcctcattggagcattctactggctcaaattctgtggtttataacggtgtgggcgattcaaacggatctaccgtggttggttcaggaggttacatgatgcctatgtctgctgccactgccaccgcaactaccgctatggtgtcacatgaacaggttcacgctagagctcagggcgatcatcacgatgaggccaaacaggcagctcagatgggatatgaatcttacctggtgaatgctgagaactacggaggtggtaggatgtcagcagcatgggcaactgtttctgctccacctgctgcctcttcaaatgataacatggcagatgtgggccatggcggagctcagttgttctctgtttggaatgatacctga

SEQ ID NO: 42 - DNA, 927 bp, Triticum aestivum
  source 1..927: mol_type = other DNA
atggacaagcagagcgtcatgtggagacagctgcagcaccagcaccaccaggccagcggcgcggcgggtggcaatgccgtcgctgctgccggtgctggtgcgacgacgacgaccgcgcccgtccggccgagcggcgcgcggtggacgcccacgccggagcaggtcaagatcctcaaggacctctactacgactgcggcatccggtcgcccacggcggagcagatccagcggatcgcggcccggctccggcagtacggccggatcgagggcaagaacgtcttctactggttccagaaccacaaggcccgcgagcgccagaagaagcgcctcggcgtcgacgtcaacggctcccccctcgccaccgccaccgccgccgacgtcctcgccctctccccctccggtgcggcggccggcttgtacggcgccggcagctgcgccggtagaggcgcggctgtccatccagatgcgagcgccactactactacttgctggggagacagcaccctgcaggactacatgggcgcgaggagcacggcgggagcgggcaaccacggcggcgccgccgcagcagcacctacgccgtggccagctgcgagcttccctttctccaccaaccagacgccgccaatgcccccgccgcgggagctcccgctcttcccgaccggcggaggccggcaagaaagcgccgacgacttcaacggcagcagctaccatctccagcccaacagctcgcagtggtgggaagctgccgccgccagcaacgccaacgccatggcagtcgtccatcatcagctgctgcaagaacagcacgagcagcagcacagcttttacaacagcagcggcaaccagcagcagatgatgatgatgatgcccatccaggacgccggcacctccccggagctcactctccgcgccccttacatgtga

SEQ ID NO: 43 - DNA, 927 bp, Triticum aestivum
  source 1..927: mol_type = other DNA
atggataagcagtcagttatgtggaggcagcttcagcatcagcaccaccaggcatctggagctgccggcggaaatgcagtggcagctgccggagccggtgcaactaccacaactgcaccagttcgcccttcaggagctaggtggacacctactccagagcaggttaagattcttaaagatctgtactacgattgcggtatcagatctccaactgctgaacagattcagcgcatcgcagctagactgcgccagtatggccgcattgagggaaagaatgtgttttactggttccagaaccacaaagcaagggaaagacagaagaaaaggttgggagtggatgttaacggttcaccacttgctaccgccacagccgcagatgttcttgccctgtctccttcaggtgctgccgcaggcctgtatggcgctggatcttgtgctggtagaggcgctgctgtgcatcctgatgcatctgctaccacaactacctgttggggcgattcaaccttgcaggattacatgggagctcgctctacagccggagcaggtaatcatggtggcgcagctgccgcagctcctactccatggcctgccgcatcttttccattctcaactaaccagaccccacctatgccaccaccaagagagttgccactttttcctactggcggcggcagacaggaatcagccgatgatttcaatggctcttcatatcatttgcagcctaactcttcacagtggtgggaggctgccgcagcttcaaatgctaacgccatggcagtggttcatcaccagctgttgcaggagcagcatgaacagcagcactctttttataattcttcaggtaaccagcagcagatgatgatgatgatgccaatccaggatgctggcacatctccagaactgactttgagagccccttacatgtga

SEQ ID NO: 44 - DNA, 984 bp, Sorghum bicolor
  source 1..984: mol_type = other DNA
atggcggccaatgtgggcgcgggcaggagtgccgtcggcggcggcggtggtggtggagcgggtggcactggcactgctgctgccagcggcagcgtggcgacgacagcggtgtgccgccccatcggctcgcggtggacgccgacgcccgagcagatcaggatactcaaggagttctactacggctgcggcatccggtcgcccaactcggagcagatccagcgcatcaccgccatgctgcgccagcacggcaagatcgagggcaagaacgtcttctactggttccagaaccacaaggcccgcgaacgccagaagcgccgcctcaccaacctcgacgtcaacgtgcccaccgccgcagccgccggcgcggccgacgccagcacccacctaggcgtcctctcgctgtcgtcgccttcaggcgcggcgcctccctcgcccaccctcggcttctacgccggcaatggcggcgccggctcgaccgtgctgctggacacgagttccgactgcgctgccatggccactgagacatgcttcctgcaggactacatgggcgtgatgggcacgggcagcgcggccgccgcgtcgccgtgggcatgcttctcgtcgtcgaacactatggcggcggccgcggcacgggcaccgacggtgacgcgggcgcccgagacgctccctctcttcccgacgggcggcgacgacagccagccccggcggccgcggcacggagtcccagttccagttgcagcaggcgaggccatccgcggcggcagcagcagcagcaggtacctgccgttctggggtgccgcgcccacaactgccagtgccacttccattgggatccagcagcaacaccagctgctgcagctgcaagagcagtacagcttttacagcaacgccatgcccggcaccggcagccaagatgcatcagcagcatccctggagctcagcctcagctcctggtgctccccttaccctgcagggaccatgtga

SEQ ID NO: 45 - DNA, 984 bp, Sorghum bicolor
  source 1..984: mol_type = other DNA
atggcggcgaacgtgggggcaggtcggtcagcggtcgggggagggggagggggtggagcgggtgggactggcacagcggcagcgtcaggctcagttgccactaccgcagtgtgcagaccaattggatctcgctggaccccaacacctgagcagattaggatcctgaaggaattttattacggctgtggaattagatcacctaattctgagcagattcagaggatcactgctatgttgagacagcatggcaagatcgagggaaaaaatgtgttttattggttccagaaccacaaggcccgcgaaaggcagaaaagacgccttacaaatctggatgtgaatgttccaacagctgccgcagctggcgccgcagatgcatcaactcatttgggagtgttgtcactttcttcaccatctggtgctgccccaccttcacctacccttggtttttatgctggcaatggcggagccggatctaccgttctgttggatacatcttcagattgcgcagctatggcaactgaaacctgctttctgcaggattacatgggcgtgatgggcactggatcagccgcagctgcctctccttgggcttgcttctcttcatctaacacaatggcagctgccgcagctcgcgcaccaacagttactagggctcctgagactctgccattgttccctaccggtggcgatgattctcagccaagaagaccaagacatggagtgccagttcctgtggctgctggcgaggctatcagaggcggctcatcttcatctcgctaccttccattttggggtgctgcccctacaactgcatcagctacatctattggcatccagcagcagcaccagcttctgcagctgcaggagcagtattcattctactctaacgccatgccaggaactggttcacaggatgcatctgccgcatctcttgaattgtccttgtcctcctggtgctctccctatcccgctggaactatgtga

SEQ ID NO: 46 - DNA, 783 bp, Arabidopsis thaliana
  source 1..783: mol_type = other DNA
atggaaaacgaagtaaacgcaggaacagcaagcagttcaagatggaacccaacgaaagatcagatcacgctactggaaaatctttacaaggaaggaatacgaactccgagcgccgatcagattcagcagatcaccggtaggcttcgtgcgtacggccatatcgaaggtaaaaacgtcttttactggttccagaaccataaggctaggcaacgccaaaagcagaaacaggagcgcatggcttacttcaatcgcctcctccacaaaacctcccgtttcttctacccccctccttgctcaaacgtgggttgtgtcagtccgtactatttacagcaagcaagtgatcatcatatgaatcaacatggaagtgtatacacaaacgatcttcttcacagaaacaatgtgatgattccaagtggtggctacgagaaacggacagtcacacaacatcagaaacaactttcagacataagaacaacagcagccacaagaatgccaatttctccgagttcactcagatttgacagatttgccctccgtgataactgttatgccggtgaggacattaacgtcaattccagtggacggaaaacactccctctttttcctcttcagcctttgaatgcaagtaatgctgatggtatgggaagttccagttttgcccttggtagtgattctccggtggattgttctagcgatggagccggccgagagcagccgtttattgatttcttttctggtggttctacttctactcgtttcgatagtaatggtaatgggttgtaa

SEQ ID NO: 47 - DNA, 783 bp, Arabidopsis thaliana; identical to SEQ ID NO: 46
  source 1..783: mol_type = other DNA

SEQ ID NO: 48 - DNA, 978 bp, Arabidopsis thaliana
  source 1..978: mol_type = other DNA
atgtcctcctcaaacaaaaattggccaagcatgttcaaatccaaaccttgcaacaataatcatcatcatcaacatgaaatcgatactccatcttacatgcactactctaattgcaacctatcatcttccttttcctcagatcggataccagatcctaaaccgagatggaatcctaaaccggagcagattaggatactcgaatcaatcttcaattccggtactattaacccacctagagaggagattcaaagaatccggatccggcttcaagaatatggtcaaatcggtgacgcaaacgtgttttactggtttcaaaaccggaaatctcgagcaaaacacaagcttcgtgttcatcacaaaagccctaaaatgtcaaagaaggacaagacggttattcctagtactgacgctgatcattgttttggttttgttaaccaagaaaccggattatatccggttcaaaacaatgagttggtggtaaccgaaccggccggttttctatttccggttcataatgatccgagcgctgctcaatcagcgtttggttttggcgattttgttgtaccggtggtaacggaagaagggatggcattctctaccgttaataacggcgttaatttggagactaacgaaaattttgataaaattccggcgatcaatttatacggcggagatggaaatggcggtggaaattgttttcctcctttgactgttccattaaccatcaatcaatctcaagaaaaacgagatgtaggattatccggtggtgaagacgtcggagataatgtttatccggtgagaatgacggtgtttattaacgagatgcctatcgaagtagtgtctggattattcaacgttaaggcagctttcggaaacgatgccgttttgatcaactcgtttggccagcctattcttacagatgaatttggtgttacttatcaacctctccaaaatggcgcaatctattatcttatttag

SEQ ID NO: 49 - DNA, 978 bp, Arabidopsis thaliana; identical to SEQ ID NO: 48
  source 1..978: mol_type = other DNA

SEQ ID NO: 50 - protein, 240 aa, Agrobacterium tumefaciens
MDLRLIFGPTCTGKTSTAVALAQQTGLPVLSLDRVQCCPQLSTGSGRPTVEELKGTSRLYLDDRPLVKGIIAAKQAHERLMGEVYNYEAHGGLILEGGSISLLKCMAQSSYWSADFRWHIIRHELADEETFMNVAKARVKQMLRPAAGLSIIQELVDLWKEPRLRPILKEIDGYRYAMLFASQNQITSDMLLQLDADMEDKLIHGIAQEYLIHARRQEQKFPRVNAAAYDGFEGHPFGMY

SEQ ID NO: 51 - protein, 318 aa, Arabidopsis thaliana
MKCNDKMVVIMGATGSGKSSLSVDLALHFKAEIINSDKMQFYDGLKITTNQSTIEDRRGVPHHLLGELNPEAGEVTAAEFRVMAAEAISEITQRKKLPILAGGSNSYIHALLAKSYDPENYPFSDHKGSICSELKYDCCFIWIDVDQSVLFEYLSLRLDLMMKSGMFEEIAEFHRSKKAPKEPLGIWKAIGVQEFDDYLKMYKWDNDMDKWDPMRKEAYEKAVRAIKENTFQLTKDQITKINKLRNAGWDIKKVDATASFREAIRAAKEGEGVAEMQRKIWNKEVLEPCVKIVRSHLDQPINYYYYYFYLLKRFLSLN

SEQ ID NO: 52 - protein, 359 aa, Zea mays
MEEITQHFGVGASSHGHGHGQHHHHHHHHHPWASSLSAVVAPLPPQPPSAGLPLTLNTVAATGNSGGSGNPVLQLANGGGLLDACVKAKEPSSSSPYAGDVEAIKAKIISHPHYYSLLTAYLECNKVGAPPEVSARLTEIAQEVEARQRTALGGLAAATEPELDQFMEAYHEMLVKFREELTRPLQEAMEFMRRVESQLNSLSISGRSLRNILSSGSSEEDQEGSGGETELPEVDAHGVDQELKHHLLKKYSGYLSSLKQELSKKKKKGKLPKEARQQLLSWWDQHYKWPYPSETQKVALAESTGLDLKQINNWFINQRKRHWKPSEEMHHLMMDGYHTTNAFYMDGHFINDGGLYRLG

SEQ ID NO: 53 - protein, 584 aa, Arabidopsis thaliana
MNSMNNWLGFSLSPHDQNHHRTDVDSSTTRTAVDVAGGYCFDLAAPSDESSAVQTSFLSPFGVTLEAFTRDNNSHSRDWDINGGACNNINNNEQNGPKLENFLGRTTTIYNTNETVVDGNGDCGGGDGGGGGSLGLSMIKTWLSNHSVANANHQDNGNGARGLSLSMNSSTSDSNNYNNNDDVVQEKTIVDVVETTPKKTIESFGQRTSIYRGVTRHRWTGRYEAHLWDNSCKREGQTRKGRQVYLGGYDKEEKAARAYDLAALKYWGTTTTTNFPLSEYEKEVEEMKHMTRQEYVASLRRKSSGFSRGASIYRGVTRHHQHGRWQARIGRVAGNKDLYLGTFGTQEEAAEAYDIAAIKFRGLSAVTNFDMNRYNVKAILESPSLPIGSSAKRLKDVNNPVPAMMISNNVSESANNVSGWQNTAFQHHQGMDLSLLQQQQERYVGYYNGGNLSTESTRVCFKQEEEQQHFLRNSPSHMTNVDHHSSTSDDSVTVCGNVVSYGGYQGFAIPVGTSVNYDPFTAAEIAYNARNHYYYAQHQQQQQIQQSPGGDFPVAISNNHSSNMYFHGEGGGEGAPTFSVWNDT

SEQ ID NO: 54 - protein, 661 aa, Triticum aestivum
MASGNDWLGFSLSGQENPQPHQDSPPPAGIDISGASDFYGVPTQPAPDAQLGVPGHQPSYGITEDFNRGAHETHDWNMRGDLDYNGGASELSMLVGSSAVGGKRAAMEAETEPKLEDFLGCNSFVSEQDQAGGFLFSGVPMTTSSNSNTGSNTMELSMIKSWLRDNQLPQAPHPQSQTGAPAQQPQPHEEMGTDASSFDPRGRNGALVVAGSSQSLALSMSTGSGSHLPMAVVSGSASGGVSESTSSENKRASGAMDSPGGAVEAVARKSIDTFGQRTSIYRGVTRHRWTGRYEAHLWDNSCRREGQSRKGRQVYLGGYDKEDKAARAYDLAALKYWGTTTTTNFPINTYEKEVDEMKHMTRQEYIAYLRRNSSGFSRGASKYRGVTRHHQHGRWQARIGRVAGNKDLYLGTFSTEEEAAEAYDIAAIKFRGLNAVTNFDMSRYDVKTILESSTLPVGGAARRLKEAADHPEAGATIWRAGMDGAIISQLTDVGMGAYASYHHGWPTIAFQQPSPLSVHYPYVAQPPRGWCKPEQDATVATQSLQDLQQLHLGTAAHNYFQASSSSTVYNGGGVGGYQQGTGGNAFLMPASTVVDEQGHSSTAANQGSTCSYGDEEGKLNLGYDAMAMASTGADPYAAASTVSIARANGYSNNWSSPFSGM

SEQ ID NO: 55 - protein, 659 aa, Triticum aestivum
MASGNDWLGFSLSGQENPQPHQDSSPPADIDIAGASGFYGLPTQPAPDAQLGVPGHHQPSYGITEAFNRGAHEIHDWNMRGDLDYNGGASELSMLVGSSAVGGKRAAMEAETEPKLEDFLVGNSFVSEQDQAGGFLFSGVPMATSTNSNSRSNTMELSMIKSWLRDNQVPQAPHPQSQTGAPAQQPQPHEEMGTDASSFDPLGRNGALVVAGSSQSLALSMSTGSGSHLPMAVLGGSPSGGVSESTSSENKRASGAMDSPGGAVEAVARKSIDTFGQRTSIYRGVTRHRWTGRYEAHLWDNSCRREGQSRKGRQVYLGGYDKEDKAARAYDLAALKYWGTTTTTNFPINTYEKEVDEMKHMTRQEYIAYLRRNSSGFSRGASKYRGVTRHHQHGRWQARIGRVAGNKDLYLGTFSTEEEAAEAYDIAAIKFRGLNAVTNFDMSRYDVKTILESSTLPVGGAARRPKEVADHPEAGATIWRAGMDGGVISQLTDVGMGTYASYHHGWPTIAFQQPSPLSVHYPYVAQPSRGWCKPEQDASVATQSLQDLQQLHLGTAAHNYFQASSSSTVYNGGGYQQGTSGNAFLMPASTVVDEQGHSSTATNQGSTCSYGDEEGKLNLGYDAMAMASTGADPYAAAPTVSIARANGYSNNWSSPFNGM

SEQ ID NO: 56 - protein, 703 aa, Sorghum bicolor
MATVNNWLAFSLSPQELPPTQTDSTLISAATTDDVSGDVCFNIPQDWSMRGSELSALVAEPKLEDFLGGISFSEQHHKANCNMIPSTSSTACYASSGATAGYHHQLYHQPTSSALHFADSVMVASSAGGVHDGGAMLSAASANGSAGAGAASANGSGSIGLSMIKNWLRSQPAPMQPRVAAAESVQGLSLSMNMAGATQGAAGMPLLAGERGRAPESVSTSAQGGAVVTAPKEDSGGSGVAATGALVAVSTDTGGSGASADNTARKTVDTFGQRTSIYRGVTRHRWTGRYEAHLWDNSCRREGQTRKGRQVYLGGYDKEEKAARAYDLAALKYWGPTTTTNFPVNNYEKELEDMKHMTRQEFVASLRRKSSGFSRGASIYRGVTRHHQHGRWQARIGRVAGNKDLYLGTFSTQEEAAEAYDIAAIKFRGLNAVTNFDMSRYDVKSILDSSALPIGSAAKRLKEAEAAASAQHHAGVVSYDVGRIASQLGDGGALAAAYGAHYHGAWPTIAFQPSAATGLYHPYAQPMRGWCKQEQDHAVIAAAHSLQELHHLNLGAAAGAHDFFSAGQQAAMHGLGSMDNASLEHSTGSNSVVYNGVGDSNGSTVVGSGGYMMPMSAATATATTAMVSHEQVHARAQGDHHDEAKQAAQMGYESYLVNAENYGGGRMSAAWATVSAPPAASSNDNMADVGHGGAQLFSVWNDT

SEQ ID NO: 57 - protein, 308 aa, Triticum aestivum
MDKQSVMWRQLQHQHHQASGAAGGNAVAAAGAGATTTTAPVRPSGARWTPTPEQVKILKDLYYDCGIRSPTAEQIQRIAARLRQYGRIEGKNVFYWFQNHKARERQKKRLGVDVNGSPLATATAADVLALSPSGAAAGLYGAGSCAGRGAAVHPDASATTTTCWGDSTLQDYMGARSTAGAGNHGGAAAAAPTPWPAASFPFSTNQTPPMPPPRELPLFPTGGGRQESADDFNGSSYHLQPNSSQWWEAAAASNANAMAVVHHQLLQEQHEQQHSFYNSSGNQQQMMMMMPIQDAGTSPELTLRAPYM

SEQ ID NO: 58 - protein, 327 aa, Sorghum bicolor
MAANVGAGRSAVGGGGGGGAGGTGTAAASGSVATTAVCRPIGSRWTPTPEQIRILKEFYYGCGIRSPNSEQIQRITAMLRQHGKIEGKNVFYWFQNHKARERQKRRLTNLDVNVPTAAAAGAADASTHLGVLSLSSPSGAAPPSPTLGFYAGNGGAGSTVLLDTSSDCAAMATETCFLQDYMGVMGTGSAAAASPWACFSSSNTMAAAAARAPTVTRAPETLPLFPTGGDDSQPRRPRHGVPVPVAAGEAIRGGSSSSRYLPFWGAAPTTASATSIGIQQQHQLLQLQEQYSFYSNAMPGTGSQDASAASLELSLSSWCSPYPAGTM

SEQ ID NO: 59 - protein, 260 aa, Arabidopsis thaliana
MENEVNAGTASSSRWNPTKDQITLLENLYKEGIRTPSADQIQQITGRLRAYGHIEGKNVFYWFQNHKARQRQKQKQERMAYFNRLLHKTSRFFYPPPCSNVGCVSPYYLQQASDHHMNQHGSVYTNDLLHRNNVMIPSGGYEKRTVTQHQKQLSDIRTTAATRMPISPSSLRFDRFALRDNCYAGEDINVNSSGRKTLPLFPLQPLNASNADGMGSSSFALGSDSPVDCSSDGAGREQPFIDFFSGGSTSTRFDSNGNGL

SEQ ID NO: 60 - protein, 325 aa, Arabidopsis thaliana
MSSSNKNWPSMFKSKPCNNNHHHQHEIDTPSYMHYSNCNLSSSFSSDRIPDPKPRWNPKPEQIRILESIFNSGTINPPREEIQRIRIRLQEYGQIGDANVFYWFQNRKSRAKHKLRVHHKSPKMSKKDKTVIPSTDADHCFGFVNQETGLYPVQNNELVVTEPAGFLFPVHNDPSAAQSAFGFGDFVVPVVTEEGMAFSTVNNGVNLETNENFDKIPAINLYGGDGNGGGNCFPPLTVPLTINQSQEKRDVGLSGGEDVGDNVYPVRMTVFINEMPIEVVSGLFNVKAAFGNDAVLINSFGQPILTDEFGVTYQPLQNGAIYYLI

SEQ ID NO: 61 - DNA, 1592 bp, Medicago truncatula
  source 1..1592: mol_type = genomic DNA
actgttaataatttttaaacgtcagcgcactaaaaaaacgaaaagacggacacgtgaaaataaaaaacacacactagtttatgacgcaatactattttacttatgatttgggtacattagacaaaaccgtgaaagagatgtatcagctatgaaacctgtatacttcaatacagagacttactcatatcggatacgtacgcacgaagtatcatattaattattttaatttttaataaatattttatcggatacttatgtgatactctacatatacacaaggatatttctaagatactttatagatacgtatcctagaaaaacatgaagagtaaaaaagtgagacaatgttgtaaaaattcattataaatgtatatgattcaattttagatatgcatcagtataattgattctcgatgaaacacttaaaattatatttcttgtggaagaacgtagcgagagaggtgattcagttagacaacattaaataaaattaatgttaagttcttttaatgatgtttctctcaatatcacatcatatgaaaatgtaatatgatttataagaaaatttttaaaaaatttattttaataatcacatgtactattttttaaaaattgtatcttttataataatacaataataaagagtaatcagtgttaatttttcttcaaatataagttttattataaatcattgttaacgtatcataagtcattaccgtatcgtatcttaatttttttttaaaaaccgctaattcacgtacccgtattgtattgtacccgcacctgtatcacaatcgatcttagttagaagaattgtctcgaggcggtgcaagacagcatataatagacgtggactctcttataccaaacgttgtcgtatcacaaagggttaggtaacaagtcacagtttgtccacgtgtcacgttttaattggaagaggtgccgttggcgtaatataacagccaatcgatttttgctataaaagcaaatcaggtaaactaaacttcttcattcttttcttccccatcgctacaaaaccggttcctttggaaaagagattcattcaaacctagcacccaattccgtttcaaggtataatctactttctattcttcgattattttattattattagctactatcgtttaatcgatcttttcttttgatccgtcaaatttaaattcaattagggttttgttcttttctttcatctgattgaaatccttctgaattgaaccgtttacttgattttactgtttattgtatgatttaatcctttgtttttcaaagacagtctttagattgtgattaggggttcatataaatttttagatttggatttttgtattgtatgattcaaaaaatacgtcctttaattagattagtacatggatattttttacccgatttattgattgtcagggagaatttgatgagcaagtttttttgatgtctgttgtaaattgaattgattataattgctgatctgctgcttccagttttcataacccatattcttttaaccttgttgtacacacaatgaaaaattggtgattgattcatttgtttttctttgttttggattatacagg

SEQ ID NO: 62 - DNA, 2000 bp, Zea mays
  source 1..2000: mol_type = genomic DNA
gtcgtgcccctctctagagataaagagcattgcatgtctaaagtataaaaaattaccacatatttttttgtcacacttatttgaagtgtagtttatctatctctatacatatatttaaacttcactctacaaataatatagtctataatactaaaataatattagtgttttagaggatcatataaataaactgctagacatggtctaaaggataattgaatattttgacaatctacagttttatctttttagtgtgcatgtgatctctctgttttttttgcaaatagcttgacctatataatacttcatccattttattagtacatccatttaggatttagggttgatggtttctatagactaatttttagtacatccattttattctttttagtctctaaattttttaaaactaaaactctattttagttttttatttaataatttagatataaaatgaaataaaataaattgactacaaataaaacaaataccctttaagaaataaaaaaactaagcaaacatttttcttgtttcgagtagataatgacaggctgttcaacgccgtcgacgagtctaacggacaccaaccagcgaaccagcagcgtcgcgtcgggccaagcgaagcagacggcacggcatctctgtagctgcctctggacccctctcgagagttccgctccaccgttggacttgctccgctgtcggcatccagaaattgcgtggcggagcggcagacgtgaggcggcacggcaggcggcctcttcctcctctcacggcaccggcagctacgggggattcctttcccaccgctccttcgctttcccttcctcgcccgccgtaataaatagacaccccctccacaccctctttccccaacctcgtgttcgttcggagcgcacacacacgcaaccagatctcccccaaatccagccgtcggcacctccgcttcaaggtacgccgctcatcctccccccccccctctctctaccttctctagatcggcgatccggtccatggttagggcccggtagttctacttctgttcatgtttgtgttagagcaaacatgttcatgttcatgtttgtgatgatgtggtctggttgggcggtcgttctagatcggagtaggatactgtttcaagctacctggtggatttattaattttgtatctgtatgtgtgtgccatacatcttcatagttacgagtttaagatgatggatggaaatatcgatctaggataggtatacatgttgatgcgggttttactgatgcatatacagagatgctttttttctcgcttggttgtgatgatatggtctggttgggcggtcgttctagatcggagtagaatactgtttcaaactacctggtggatttattaaaggataaagggtcgttctagatcggagtagaatactgtttcaaactacctggtggatttattaaaggatctgtatgtatgtgcctacatcttcatagttacgagtttaagatgatggatggaaatatcgatctaggataggtatacatgttgatgcgggttttactgatgcatatacagagatgctttttttcgcttggttgtgatgatgtggtctggttgggcggtcgttctagatcggagtagaatactgtttcaaactacctggtggatttattaattttgtatctttatgtgtgtgccatacatcttcatagttacgagtttaagatgatggatggaaatattgatctaggataggtatacatgttgatgtgggttttactgatgcatatacatgatggcatatgcggcatctattcatatgctctaaccttgagtacctatctattataataaacaagtatgttttataattattttgatcttgatatacttggatgatggcatatgcagcagctatatgtggattttttagccctgccttcatacgctatttatttgcttggtactgtttcttttgtccgatgctcaccctgttgtttggtgatacttc

SEQ ID NO: 63 - DNA, 4101 bp, synthetic construct
  misc_feature 1..4101: note = Cas9
  source 1..4101: mol_type = other DNA
gacaagaagtacagcatcgggctggcgatcgggaccaactccgtcggctgggctgtgattaccgacgagtacaaggtgccatccaagaagttcaaggtcctcggcaacactgaccggcacagcattaagaagaacctgattggggcgctgctgttcgattcgggggagactgcggaggcgaccaggctgaagcggactgcgcgccggaggtacaccaggaggaagaatcggatctgctacctccaggagattttctcgaatgagatggccaaggtggacgattccttcttccatcgcctggaggagtcgttcctcgttgaggaggacaagaagcatgagaggcatcccattttcgggaatatcgttgacgaggtggcttaccatgagaagtacccgaccatctaccatctgcggaagaagctcgtcgattcgaccgataaggccgacctgcggctgatctacctggccctcgcgcacatgattaagttccggggccatttcctcatcgagggcgacctcaacccggacaactcggacgtggataagctcttcattcagctcgtgcagacatacaaccagctcttcgaggagaatcccattaacgcctcgggggtcgacgctaaggctattctctcggctcggctgtcgaagtcgcgccggctggagaatctcattgcccagctcccaggcgagaagaagaacggcctcttcggcaacctgattgccctgtcgctggggctcacaccgaatttcaagtcgaacttcgacctcgccgaggacgctaagctccagctcagcaaggatacttacgatgatgacctcgataacctgctcgcccagattggggatcagtacgcggatctgttcctcgcggccaagaatctcagcgatgctattctcctgtcggacattctccgcgtcaacacagagattactaaggccccactgtcggcgagcatgattaagaggtacgatgagcatcatcaggacctgacactgctcaaggcgctggtccggcagcagctccccgagaagtacaaggagattttcttcgatcagtcaaagaatgggtacgcgggctacattgatggcggcgcgtcccaggaggagttctacaagttcattaagcccatcctggagaagatggacgggaccgaggagctgctggtgaagctcaatcgggaggacctgctccggaagcagcgcacattcgacaatggctcgattcctcaccagattcacctgggcgagctgcacgccattctccgcaggcaggaggacttctacccgttcctcaaggacaaccgcgagaagatcgagaagatcctgaccttccggattccatactacgtggggccgctcgcgcgggggaactcccggttcgcgtggatgactcgcaagtccgaagaaacgattacaccgtggaatttcgaggaggtcgtcgacaagggcgctagtgcgcagtcattcattgagaggatgaccaatttcgataagaacctgcctaacgagaaggtgctgccgaagcattcgctgctctacgagtacttcaccgtttacaatgagctgaccaaggtgaagtatgtgactgagggcatgaggaagccagcgttcctgagcggcgagcagaagaaggctatcgtggacctgctcttcaagactaaccggaaggtgactgtgaagcagctcaaggaggactacttcaagaagattgagtgcttcgattccgttgagattagcggggtggaggatcggttcaatgcttcgctcgggacataccacgatctcctgaagatcattaaggataaggacttcctcgacaacgaggagaacgaggacattctcgaagatattgtcctgaccctcaccctcttcgaggatcgggagatgatcgaggagaggctcaagacatacgctcatctgttcgatgataaggtcatgaagcagctgaagcgcaggcggtacacagggtgggggcggctgagccggaagctgatcaacgggattcgggataagcagtccgggaagacaattctcgacttcctcaagtccgacgggttcgctaaccggaacttcatgcagctcattcatgatgactcgctgacattcaaggaggatattcagaaggcgcaggtttcggggcagggcgactcgctccacgagcatattgcgaatctggcgggctcccccgcgattaagaagggcattctgcaaaccgtcaaggtggttgatgagctggtcaaggtcatggggcggcataagccagagaatattgtcatcgagatggcgcgggagaatcagaccacacagaaggggcagaagaactcacgggagcggatgaagcgcatcgaggagggcatcaaggagctggggtcgcagatcctgaaggagcatcccgtggagaacactcagctgcaaaatgagaagctgtacctctactacctccagaacgggagggacatgtatgtggatcaggagctggatattaataggctgagcgattacgatgtcgaccacattgtcccacagtcgttcctgaaggacgacagcattgacaacaaggtgctgacccgctcggataagaacaggggcaagagcgataatgttccaagcgaggaggttgtgaagaagatgaagaactactggcggcagctcctgaacgcgaagctcatcacacagcggaagttcgacaacctcaccaaggctgagcgcgggggcctgagcgagctggacaaggcggggttcattaagaggcagctggtcgagacacggcagattacaaagcatgttgcgcagattctcgattcccggatgaacaccaagtacgatgagaacgataagctgattcgggaggtcaaggtaattaccctgaagtccaagctggtgtccgacttcaggaaggacttccagttctacaaggttcgggagatcaacaactaccaccacgcgcatgatgcctacctcaacgcggtcgtggggaccgctctcatcaagaagtacccaaagctggagtcagagttcgtctacggggattacaaggtttacgacgtgcggaagatgatcgctaagagcgagcaggagattggcaaggctaccgctaagtacttcttctactccaacatcatgaacttcttcaagacagagattaccctcgcgaatggcgagatccggaagaggcccctcatcgagacaaatggggagacaggggagattgtctgggataaggggcgggatttcgcgaccgtccggaaggtcctgtcgatgccccaggttaatattgtcaagaagactgaggtccagactggcggcttctcaaaggagtcgattctcccaaagaggaactccgataagctcattgctcggaagaaggattgggaccccaagaagtacgggggattcgactcccccactgttgcttactctgttctggttgttgctaaggtggagaaggggaagtcgaagaagctgaagagcgtgaaggagctgctcgggattacaattatggagaggtcatccttcgagaagaatcccatcgacttcctggaggccaagggctacaaggaggtgaagaaggacctgattattaagctgcccaagtactcgctcttcgagctggagaatgggcggaagcggatgctggcgtccgcgggggagctgcaaaaggggaacgagctggcgctcccctccaagtatgtgaacttcctctacctggcgtcgcactacgagaagctgaaggggtccccagaggataatgagcagaagcagctcttcgtcgagcagcataagcactacctggacgagattatcgagcagattagcgagttctcgaagcgggtcatcctcgcggatgcgaacctggataaggtgctcagcgcctacaataagcaccgggacaagccgattcgggagcaggcggagaatattattcacctcttcacactcaccaacctcggggcaccagctgcgttcaagtacttcgacactactatcgaccggaagcggtacacctcgacgaaggaggtgctcgacgccaccctcattcaccagtcgatcacaggcctgtacgagacacggattgacctgtcccagctcgggggcgac

SEQ ID NO: 64 - DNA, 4101 bp, synthetic construct
  misc_feature 1..4101: note = Cas9
  source 1..4101: mol_type = other DNA
gacaagaagtactccattggcctggcgattgggacaaactcggtggggtgggccgtgattacggatgagtacaaggttccaagcaagaagttcaaggtcctcgggaacacagatcggcattcgattaagaagaatctcattggggcgctcctcttcgactcgggggagacagcggaggctaccaggctcaagcggacagccaggcggcggtacacaaggcggaagaatcgcatctgctacctccaggagattttctcgaatgagatggcgaaggtggacgacagcttcttccatcggctggaggagtccttcctggtggaggaggataagaagcacgagaggcatccaattttcgggaacatcgtggacgaggttgcgtaccatgagaagtaccctacaatctaccatctgcggaagaagctggttgactccacagacaaggcggacctgaggctgatctacctcgctctggcccacatgattaagttccgcgggcatttcctgatcgagggggacctgaatcccgacaattcggatgtggacaagctcttcatccagctggtgcagacctacaaccagctgttcgaggagaatcccatcaatgcgtcgggcgttgacgctaaggccattctgtccgctaggctgtcgaagagcaggaggctggagaacctgatcgcccagctgccaggcgagaagaagaatgggctcttcgggaatctgattgcgctctccctggggctgacaccgaacttcaagagcaatttcgatctggctgaggacgcgaagctccagctctcgaaggacacttacgacgatgacctcgataacctcctcgcgcagatcggggaccagtacgctgatctcttcctcgccgctaagaacctctcggatgctatcctgctctccgacattctccgggttaataccgagattacaaaggccccactgtcggcgtccatgatcaagcggtacgatgagcatcatcaggatctcaccctgctcaaggccctcgtgcggcagcagctgcccgagaagtacaaggagattttcttcgaccagagcaagaatgggtacgctggctacattgacggcggggcctcacaggaggagttctacaagttcatcaagccaatcctggagaagatggatgggacagaggagctgctggtgaagctcaaccgggaggatctgctcaggaagcagcggacgttcgacaacgggtcgattccccatcagatccacctgggggagctgcacgcgatcctgcgccggcaggaggatttctaccctttcctgaaggataatcgggagaagatcgagaagattctcaccttccggattccctactacgtcgggccactcgcgcggggcaatagcaggttcgcctggatgacacggaagagcgaggagacaatcaccccctggaacttcgaggaggttgtcgacaagggggcgtccgcccagtcattcattgagcggatgaccaatttcgacaagaatctgccaaatgagaaggttctcccaaagcatagcctcctctacgagtacttcactgtttacaacgagctgaccaaggtgaagtatgtgaccgagggcatgcggaagcccgcgttcctgtccggcgagcagaagaaggccattgtggacctcctgttcaagaccaatcgcaaggtcacagtcaagcagctcaaggaggattacttcaagaagatcgagtgcttcgactcggttgagattagcggggtggaggatcggttcaacgcgagcctcggcacttaccacgacctcctgaagatcatcaaggataaggacttcctcgacaacgaggagaacgaggatattctggaggacatcgtgctcaccctgacgctgttcgaggatcgggagatgatcgaggagcgcctgaagacctacgctcatctcttcgatgataaggtcatgaagcagctgaagaggaggcggtacaccgggtggggccgcctgagcaggaagctcattaacgggatcagggacaagcagagcggcaagaccatcctggacttcctcaagagcgatggcttcgccaaccggaatttcatgcagctcatccacgacgactccctcaccttcaaggaggacattcagaaggctcaggtcagcggccagggcgactcgctgcatgagcacatcgctaacctggcgggcagcccagccatcaagaagggcatcctccagacagtgaaggtcgtggatgagctggtgaaggtcatgggccggcataagcccgagaatattgtgattgagatggcgcgggagaatcagaccactcagaagggccagaagaactcgcgggagcgcatgaagaggatcgaggaggggattaaggagctgggcagccagattctcaaggagcaccccgtggagaatacccagctccagaacgagaagctgtacctctactacctccagaatgggcgggacatgtatgttgatcaggagctggacatcaatcgcctctcggattacgacgtggaccacatcgtgccccagagcttcctgaaggatgatagcatcgacaataaggtcctgacccgctccgacaagaatcgcggcaagagcgacaacgtgccgagcgaggaggtcgtgaagaagatgaagaactactggcggcagctgctgaacgcgaagctcattacacagcggaagttcgataacctgacgaaggcggagaggggcggcctctccgagctggacaaggcgggcttcattaagaggcagctcgtggagactcgccagatcaccaagcacgtggctcagatcctcgatagccggatgaatacgaagtacgatgagaatgacaagctcatccgggaggtgaaggtaatcaccctgaagtcaaagctcgttagcgatttccggaaggacttccagttctacaaggtgcgggagattaacaactaccatcatgcgcacgatgcgtacctcaatgcggtggtgggcacagccctgattaagaagtaccccaagctggagagcgagttcgtctacggggactacaaggtgtacgatgttcggaagatgatcgccaagagcgagcaggagattgggaaggccaccgctaagtacttcttctactcgaatattatgaatttcttcaagaccgagatcacactcgctaatggggagattcggaagcggcccctcatcgagactaacggggagactggcgagattgtgtgggacaaggggcgcgacttcgctaccgtgcgcaaggtcctctcgatgccccaggttaatattgttaagaagacagaggtgcagacgggcgggttctccaaggagtctatcctgccgaagcggaactcggacaagctgatcgcccgcaagaaggattgggaccccaagaagtacgggggattcgatagcccaaccgtggcttacagcgtcctggtggtcgccaaggttgagaaggggaagtcgaagaagctcaagagcgttaaggagctgctgggcatcaccatcatggagcggtccagcttcgagaagaatcctatcgacttcctggaggctaaggggtacaaggaggtcaagaaggacctgatcattaagctgcccaagtactctctgttcgagctggagaacgggaggaagcggatgctggcgtctgctggcgagctacagaagggcaatgagctggcgctcccctcgaagtatgtcaacttcctctacctggcttcccattacgagaagctgaagggctcgcccgaggataatgagcagaagcagctcttcgtggagcagcacaagcactacctcgacgagatcattgagcagatttcggagttctcgaagcgggtcattctcgcggacgcgaacctcgacaaggtcctctcggcgtacaacaagcaccgggacaagcccatccgggagcaggccgagaacattatccacctcttcacactgaccaacctcggcgctcccgccgcgttcaagtacttcgacaccaccattgaccgcaagagatacacatccaccaaggaggtgctggacgcgaccctcatccaccagagcatcacaggcctctacgagacacggatcgacctctcgcagctcgggggcgat

SEQ ID NO: 65 - DNA, 4092 bp, synthetic construct
  misc_feature 1..4092: note = Cas9
  source 1..4092: mol_type = other DNA
gacaagaagtactcgatcggcctggcgattggcacaaacagcgtggggtgggctgtgatcactgatgagtacaaggtgccatcgaagaagttcaaggtgctggggaatacagaccggcattcgatcaagaagaatctcattggcgctctcctcttcgattccggcgagactgctgaggcgacccgcctgaagcgcaccgcccggcggcgctacactcggcggaagaataggatttgctacctccaggagattttctcgaatgagatggccaaggtggatgacagcttcttccaccgcctggaggagtcgttcctggtcgaggaggacaagaagcatgagcggcaccctatcttcgggaatatcgttgatgaggtcgcctaccacgagaagtaccccactatctaccatctccgcaagaagctcgtggacagcacagataaggccgacctccgcctgatctacctcgccctcgcgcacatgattaagttccgggggcacttcctcattgagggggatctgaatcccgataactccgacgtggacaagctgttcatccagctggtgcagacatacaaccagctgttcgaggagaatcccatcaacgcgagcggcgtggacgctaaggccattctgtcggctaggctctcgaagtcgaggcggctggagaacctgattgcgcagctccccggcgagaagaagaacgggctgttcgggaatctcatcgccctctccctcggcctcacaccaaacttcaagagcaatttcgacctggctgaggacgctaagctgcaactctcaaaggatacatacgatgacgacctggacaatctcctggctcagatcggcgaccagtacgctgacctgttcctcgcggccaagaatctgtcggacgcgattctcctcagcgacatcctgcgcgtcaataccgagattacgaaggctccactgtctgcgtcaatgattaagcggtacgatgagcatcaccaggatctgaccctcctgaaggcgctcgtgcggcagcagctgcccgagaagtacaaggagattttcttcgatcagagcaagaatggctacgccggctacatcgacgggggcgcgagccaggaggagttctacaagttcatcaagcccatcctggagaagatggacggcaccgaggagctactcgtgaagctcaatcgggaggatctcctccggaagcagcggacattcgataacgggtctatcccacaccagatccacctcggcgagctgcatgcgattctgcggcggcaggaggatttctaccctttcctgaaggacaaccgggagaagatcgagaagatcctcacattccggattccatactacgtcggccccctggcgaggggcaatagccggttcgcgtggatgacaaggaagtccgaggagactattaccccgtggaatttcgaggaggtggttgacaagggcgcttccgcgcagagcttcattgagcggatgacaaacttcgacaagaatctccccaacgagaaggtcctgccgaagcatagcctcctgtacgagtacttcaccgtctacaatgagctaactaaggtcaagtatgtgacagagggcatgaggaagccagccttcctctcaggcgagcagaagaaggccattgtggacctcctgttcaagacaaaccgcaaggtgacagtgaagcagctgaaggaggattacttcaagaagattgagtgcttcgactcagtggagatttcaggcgtggaggatcggttcaacgcgagcctggggacttaccacgacctgctgaagattattaaggacaaggacttcctggataacgaggagaatgaggacatcctggaggatattgtgctcaccctcaccctgttcgaggacagggagatgattgaggagaggctcaagacctacgcgcacctgttcgatgacaaggtcatgaagcagctgaagaggcggcgctacactgggtggggccgcctgtcgcggaagctgatcaacggcattcgggataagcagtccgggaagaccattctggatttcctgaagtcggacggcttcgccaacaggaatttcatgcagctgatccacgacgactccctcaccttcaaggaggacattcagaaggcccaggttagcggccagggggactcactccacgagcatattgccaatctggccggctctccagctatcaagaagggcatcctgcaaacagttaaggttgttgacgagctggttaaggtcatggggcggcataagcccgagaacattgtcatcgagatggctcgggagaaccagacaactcagaagggccagaagaactccagggagcgcatgaagcggattgaggagggcattaaggagctggggtcccagatcctcaaggagcaccctgtcgagaacactcagctgcaaaacgagaagctctacctgtactacctccagaacgggcgggatatgtatgtggatcaggagctggacatcaacaggctctccgactacgacgtggatcacattgtcccacagtctttcctcaaggatgattccatcgacaacaaggtgctgacgcgcagcgacaagaatagggggaagtcggacaacgttccgagcgaggaggtcgtgaagaagatgaagaattactggaggcagctcctgaatgcgaagctgatcactcagaggaagttcgacaatctgacaaaggcggagaggggcgggctctcggagctggataaggcgggcttcatcaagcggcagctcgttgaaacccggcagatcaccaagcatgtcgcccagatcctcgatagccgcatgaacaccaagtacgatgagaacgacaagctcattcgggaggttaaggtcattacgctgaagtccaagctcgtcagcgacttcaggaaggatttccagttctacaaggttcgggagattaacaactaccaccacgcgcatgatgcgtacctgaacgctgttgtcggcactgctctcatcaagaagtacccaaagctggagtccgagttcgtctacggggactacaaggtctacgatgtccggaagatgatcgccaagtcggagcaggagatcgggaaggctactgcgaagtacttcttctacagcaacattatgaatttcttcaagacggagattacgctggcgaacggggagattaggaagaggcccctcattgagactaatggggagacaggcgagattgtttgggacaagggccgcgacttcgcgactgtgcggaaggtcctgtccatgccacaggtgaatattgttaagaagacagaggtgcagactgggggcttctcgaaggagagcattctcccaaagcggaacagcgataagctcatcgcgcgcaagaaggattgggaccctaagaagtacggcggcttcgattctcccactgtggcctactccgttctcgtggttgccaaggttgagaaggggaagtcgaagaagctgaagtcggtcaaggagctgctcgggattacaatcatggagcggagcagcttcgagaagaaccctattgatttcctggaggccaagggctacaaggaggttaagaaggatctcattatcaagctccctaagtactctctgttcgagctggagaatggccggaagaggatgctggcctcggctggcgagctacagaaggggaatgagctggccctcccgtcgaagtatgtgaatttcctgtacctcgcgtcgcactacgagaagctcaagggcagcccggaggataatgagcagaagcagctcttcgtggagcagcataagcactacctggacgagatcattgagcagatcagcgagttctcgaagcgggttattctggctgatgctaacctggacaaggttctgagcgcctacaataagcatcgcgacaagccgattcgcgagcaggcggagaatattatccacctgttcaccctcactaacctcggggctcccgcggccttcaagtacttcgataccacaatagataggaagcggtacacctcgacgaaggaggtcctcgacgccacactcatccatcagtcgattacaggcctgtacgagacacggattgacctctcgcagctg

SEQ ID NO: 66 - DNA, 4101 bp, synthetic construct
  misc_feature 1..4101: note = Cas9
  source 1..4101: mol_type = other DNA
gacaagaagtattccataggcctggctatcggcaccaacagcgtgggctgggccgtcatcaccgacgagtacaaagtgccgagtaaaaagttcaaagtgctcggcaacaccgaccgccactccataaagaaaaacctgatcggggcgctcctgttcgacagcggcgagacggcggaggccacccgcttgaaacgcacggcccgacggcgctacacgcggcgcaagaaccggatctgttacctacaggagattttctctaacgagatggcgaaggtggacgactcgttctttcaccgcctcgaagagtccttcctcgtggaggaggacaagaaacacgagcgccacccgatcttcggcaacatcgtggacgaggtggcctaccacgagaagtacccgaccatctaccacctccggaagaaactcgtggacagcacggacaaggccgacctgaggctcatctacctcgccctggcgcacatgattaagttccggggccacttcctgatcgagggcgacctgaacccggacaacagcgacgtggacaagctgttcatccagctagtccagacctacaaccagcttttcgaggaaaaccccatcaacgccagcggggtggacgcgaaggcgatcctgtccgcccggctgagcaagtcccggcggctggagaacctcatcgcgcagttgcccggcgagaagaagaacgggctgttcgggaacctgatcgccctctccctggggctcaccccgaacttcaagtccaacttcgacctcgccgaggacgccaaactacagctgagcaaggacacctacgacgacgacctcgacaacctgctggcccagatcggggaccagtacgcagacctgttcctcgccgccaagaacctctccgacgccatcctgctgtcggacatcctgcgggtgaacacggagatcacgaaggccccgctctcggcctcgatgattaaacgctacgacgagcaccaccaggacttgaccctcctcaaggcgctggtccgccagcagcttcccgagaagtacaaggaaatctttttcgatcagagcaagaacgggtacgccgggtacatcgacggcggggcgtcccaggaggagttctacaagttcatcaagcccatcctggagaaaatggacgggaccgaggagctgctcgtgaagctcaaccgcgaagatttgctccgcaagcagcgcacgttcgacaacgggtcgatcccgcaccagatccacctgggcgagctgcacgcgatcctcaggcgtcaggaagacttctaccccttcctcaaggacaaccgcgagaagatagagaagattctgaccttcagaattccttattacgtgggcccgctggctcggggcaactcgcgcttcgcctggatgacgcgcaagtccgaggagaccatcaccccgtggaacttcgaggaggtggtggataagggtgcctcggcccagtccttcatcgagcggatgaccaacttcgacaagaacctgccgaacgagaaggtgctccccaagcacagcctgctctacgaatatttcacggtgtacaacgagctgacgaaggtcaagtacgtgaccgagggaatgaggaaacctgcattcctctccggggagcagaagaaagccatagtcgacctcctgttcaagaccaaccggaaggtcaccgtcaagcagctcaaggaggactacttcaagaagatcgagtgcttcgattcagtggagatcagcggcgtcgaggaccggttcaacgccagcctgggcacctaccacgacctgctcaagatcatcaaggacaaggacttcctcgacaacgaggagaacgaggacatcctggaggacatcgtgctgaccctgacgctcttcgaggaccgcgagatgatcgaggagcgcctcaagacctacgcccacctgttcgacgacaaggtgatgaagcagctcaagcggcggagatatactgggtggggccgcctctcccggaagctcattaacggtatcagggataagcagtccgggaagacgatcctcgacttcctcaagtcggacgggttcgccaaccgcaacttcatgcagctcatccacgacgactccctgacgttcaaggaggacatccagaaggcccaagtgtctggtcaaggtgactcgctccacgagcacatcgccaacctcgcgggcagcccggccatcaagaagggaatactccagaccgtcaaggtggtggacgagctggtgaaggtcatgggccgccacaagccggagaacatcgtcatcgagatggcgcgggagaaccagaccacgcagaaggggcagaaaaatagccgtgagcgcatgaagcgcatcgaggaggggattaaggagttgggcagccagatcctcaaggagcaccctgtggagaacacgcagttgcaaaacgagaagctctacctgtactacctccagaacgggagggatatgtacgtggaccaagaactggacatcaaccgcctgtccgactacgacgtggaccacatcgtgccgcagagcttcctcaaggacgacagcatcgacaacaaggtgctcacccggtccgacaagaatcggggcaagtccgacaacgtgcccagcgaggaggtcgtcaaaaagatgaaaaactactggcgacaactactgaacgccaagctcatcacccagcgcaagttcgacaacctcacaaaagccgagcgcggcgggttgagcgagctggacaaggccgggttcatcaagcgccagctcgtcgagacgcgccagatcacgaagcacgtcgcgcagatactcgacagccggatgaacaccaagtacgacgagaacgacaagctcatccgggaggtgaaggtcatcaccctcaagtcgaagctcgtgagcgacttccgcaaggacttccagttctacaaggtccgggagatcaacaactaccaccacgcccacgatgcttatcttaacgccgtggtggggacggccctcattaagaaatacccgaagctggagtcggagttcgtgtacggcgactacaaggtgtacgacgtcaggaagatgatcgccaagtccgaacaggagatcgggaaggccacggcgaaatacttcttctacagcaacatcatgaacttcttcaagaccgagatcaccctcgccaacggcgagatccgcaagcgcccgctcatcgagacgaacggggagaccggcgagatcgtctgggacaaggggcgcgacttcgccactgtgcggaaggtgctgtcgatgccccaggtcaacatcgtcaagaagacggaggtccagacgggcgggttcagcaaggagagcatcctgccgaagcgcaacagcgacaagctgatcgcccgcaaaaaggactgggatccaaaaaagtacggcggcttcgacagccccaccgtcgcctacagcgtcctcgtcgtcgctaaagtcgagaagggcaagtccaaaaagctcaagagcgtcaaggagctgctcgggatcaccatcatggagcggtccagcttcgagaagaacccaattgatttcctggaggcgaagggctacaaggaggtcaagaaagacctcatcataaagctgccgaagtactcactcttcgagctggagaacgggcgcaagcggatgctggcgtcggccggagagctccaaaagggcaacgagctggcgctgccgagcaagtacgtgaacttcctctacctggcgtcccactacgagaagctcaagggcagtccagaggataacgagcagaagcagctattcgtggagcagcacaagcactacctggacgagatcatcgagcagatcagcgagttctccaagcgcgtcatcctggcggacgccaacctggacaaggtgctgtccgcgtacaacaagcaccgcgacaagccgatccgcgagcaagccgagaacatcatccacctgttcaccctcacgaacctcggggcacccgccgccttcaaatatttcgacacgaccatcgaccgcaagcgctacaccagcacgaaggaggtgctcgacgccaccctgatccaccagagcatcaccgggctgtacgagacccgcatcgacctctcgcagctcggcggggac

SEQ ID NO: 67 - DNA, 4101 bp, synthetic construct
  misc_feature 1..4101: note = Cas9
  source 1..4101: mol_type = other DNA
gacaagaagtacagtattggattggccatcgggacgaacagcgtgggctgggccgtcatcaccgacgagtacaaggtgccatccaagaagtttaaggttctggggaataccgaccgccactcgatcaagaaaaatctcatcggggcgctgcttttcgacagcggcgagacggcggaagcgacgcggctcaagcggacggctcgtcgccgttacacccggcgtaagaaccgcatctgttacctccaggagatattcagcaacgagatggcgaaggtggacgactcctttttccaccgtcttgaggagtccttcctggtcgaggaggacaagaagcacgagcgccacccgatcttcgggaacatcgtggacgaggtggcctaccacgagaagtaccccacgatctaccacctccgcaaaaaactcgtggactcaactgacaaggccgatttgaggcttatctacctcgccctcgcccacatgattaagttccgtgggcacttcctaatcgagggtgacctcaaccccgacaactctgacgtggacaagctgttcatccagcttgtgcagacctacaatcagctctttgaggagaatccgatcaacgcatctggtgtggacgcaaaggccatcctcagcgcgcggctgagcaagtctaggcggttggagaacctgatcgcccaactgcccggcgagaagaaaaatggcctcttcggcaacctgatcgccctgtcgctggggctcacgccgaacttcaagagtaactttgacctggcggaggacgctaagctccagctatctaaggacacatacgacgacgacctggacaacctgctggcccagatcggcgaccagtacgccgacctcttcctagccgccaagaacctgtccgacgccatcctcctcagcgacatcctgcgcgtgaacacggagatcacgaaggctccgctcagcgcctccatgattaagcggtacgacgagcaccaccaagacctaactttactcaaagccctcgtgcggcagcagcttcccgagaagtacaaagagatattttttgatcagtccaagaacggttatgcgggctacatcgacggcggcgcgagccaggaggagttctacaagttcatcaagcccatcctggagaagatggacggcacggaggagctgctcgtgaagctcaaccgtgaagacctcctgcgaaagcagcgaaccttcgacaacggttcgatcccgcaccagatccacctcggggagctgcacgccatcctgaggcgacaggaggacttctaccctttcctaaaggacaaccgcgagaagattgaaaaaatcctgacgtttcgcataccctactacgtcggcccgctggcgcgcggcaactcccggttcgcctggatgacccgtaagagcgaggagacgatcaccccgtggaacttcgaggaggtcgtggacaagggcgcgagcgcgcagagcttcatcgagcgcatgaccaacttcgacaagaacctcccgaacgagaaggtgctcccaaagcactccctcctgtacgagtatttcaccgtgtacaacgagttgacaaaggtgaagtacgtgacggagggaatgcggaagcctgcgttcctctcgggcgagcagaagaaggcaatcgtggacctgctcttcaagaccaaccggaaggtgacggtgaagcagctcaaggaggactacttcaaaaaaatcgagtgcttcgactccgtggagataagcggcgtggaggaccgattcaacgcctccctcggcacctaccacgacctccttaagatcatcaaggacaaggacttcctggacaacgaggagaacgaggacatcctggaggacatcgtgctcaccctgaccctcttcgaggaccgggagatgatcgaggagcgcctcaagacgtacgcccacttgttcgacgacaaggtgatgaagcagctcaagcggcggcgatacaccgggtggggccgcctatcccgcaaacttatcaacggcatccgcgacaagcagtccggcaagacgatcctggatttcctcaagtcggacgggttcgccaaccggaacttcatgcagctcatccacgacgacagcctcacgttcaaggaggacatccagaaggcccaagtgagcggtcaaggggacagcctccacgagcacattgcgaaccttgctgggagccctgcgatcaagaaggggatattgcaaaccgtgaaggtcgtggacgagttggtgaaggtcatggggcgacacaagcccgagaacatcgtgatcgagatggccagggaaaatcagaccacgcagaagggccaaaaaaacagccgcgagcggatgaagcggatcgaggagggcatcaaggagctggggtcgcagatcctcaaggagcacccggtggagaacacgcagctccagaacgagaagctgtacctctattacctacagaacgggcgggatatgtacgtggaccaggagctagacatcaaccgcctgtccgactacgacgtggaccatatcgtcccgcagtcgttcttgaaggacgacagcatcgacaacaaggtgctcacaagatcggataagaatcgaggcaagtccgacaacgtgccctcggaggaggtggtcaagaaaatgaaaaactactggcggcagttgctgaacgccaagctcattacgcagcggaagttcgacaacctgacgaaggctgaacgtggtgggctcagcgagctagacaaggcggggttcatcaagcggcagctcgtcgagacccggcagatcaccaagcacgtggcgcagatcctggactcgcgcatgaacaccaagtacgacgagaacgacaagctcatccgtgaggtgaaggtcatcacccttaagtctaagctggtcagtgacttccgcaaggacttccagttctacaaggtccgggagatcaacaactaccaccacgcgcacgacgcctacctcaacgcggtggtggggacggcgcttattaagaaatatcccaagctggaaagcgagttcgtttacggcgactacaaggtgtacgacgtccgcaagatgatcgcaaagtcggaacaggaaatcggaaaggcgacggccaaatatttcttttactccaacatcatgaatttttttaagacggagatcaccctggcgaacggggagatccgcaagcggcccctcatcgagaccaacggggagacgggcgagatcgtctgggacaagggccgggacttcgccaccgtgcggaaggtgctttctatgcctcaagtcaatatcgtcaaaaagacagaggtgcagaccggcgggttcagcaaggagtctatcctgccgaagcgcaactcggacaagctcatcgcgcgcaagaaagactgggaccccaaaaaatatggcgggttcgactcgccgaccgtcgcctacagcgtcctcgtggtggctaaggtcgagaagggcaagagcaaaaagctaaagtcggtgaaggagctgctgggcatcaccatcatggagcgctcgtctttcgagaagaatccaatcgacttcctagaggcgaaggggtacaaggaggtcaaaaaggatcttatcatcaaactgccgaagtacagtctgttcgagctggagaacgggcggaagcggatgctggctagtgcgggcgagttgcagaagggcaacgagttggcactgccctccaagtacgtgaacttcctgtacctggcctcccactacgagaagctcaaggggagccccgaggacaacgagcagaagcagctattcgtcgagcagcacaagcactacctggacgagatcatcgagcagatcagtgagttctccaagcgggtcatcctcgcggacgccaacctggacaaggtgctgagcgcgtacaacaagcacagggacaagccaatcagggaacaggccgagaacatcatccacctgttcaccctgaccaacctgggtgcaccggctgccttcaagtactttgacacgaccatcgaccggaagcgctacacctccacgaaggaggtgctggacgccacgctgatccaccagagcatcaccgggctctacgagacacggatcgacctgagccagcttggcggggac

SEQ ID NO: 68 - DNA, 4092 bp, synthetic construct
  misc_feature 1..4092: note = Cas9
  source 1..4092: mol_type = other DNA
gacaaaaagtattccattggactcgctatcggcacgaacagcgtcgggtgggcggtcatcactgacgagtacaaggtgccgagcaagaagtttaaggtgctgggaaacaccgacaggcactcgatcaagaaaaatcttatcggggccctactcttcgactccggagaaaccgccgaggccacccggttgaagcgcacggcccgccgtcgctacaccaggcgcaagaaccggatctgctacctccaggagatattcagcaatgagatggcgaaggtggacgactcgttttttcacaggctagaggagtctttcctcgtggaggaggacaagaaacacgagcgccaccccatcttcggcaacatcgtggatgaggtggcatatcacgagaagtacccaaccatctaccacctccgcaaaaagctcgtggactctaccgacaaggccgacctccgtctgatctacctcgcgctggcccacatgattaagttccgaggacactttctgatcgagggcgacctgaacccagacaacagcgacgtggacaagctgttcatccaacttgtccagacctacaatcagctcttcgaggagaaccctatcaacgcctcgggcgtggacgcgaaggccatcctgtccgcccgcctgagcaagtcgcggcggctggagaacctgatcgcccagctccccggcgaaaaaaagaacggcctcttcggcaacctcatcgcgttgtcgctggggctcaccccgaacttcaagtccaacttcgacctggccgaggacgctaaactccagctctcgaaggatacctacgacgacgacctcgacaacctgctggcccagatcggcgaccagtacgcggaccttttcctggcggccaagaacctgagcgacgcgatcctccttagcgacatactccgtgtgaacaccgagatcacgaaggccccgctctccgcgtccatgattaagcgctacgacgagcaccaccaagaccttaccctgcttaaggcgctggtcaggcagcagttaccggagaagtacaaggagatcttttttgatcaatctaagaacggttacgccgggtacatcgacggcggcgcgtcccaggaggagttctacaagttcatcaagccgatcttggagaaaatggacgggaccgaggagctgctcgtgaagctcaaccgcgaagacctcctccgcaagcagcgcaccttcgacaacgggagcatcccgcaccagatccacctgggagagctgcacgcgatcctgcggagacaagaggacttctaccccttcctcaaggacaaccgggagaagattgaaaaaatacttacttttcgtatcccgtactacgtcgggccccttgcgaggggcaactccagattcgcgtggatgacccgcaagtccgaggagaccatcaccccgtggaacttcgaggaggtggtggacaagggcgcgtcggcccagtcgttcatcgagcgcatgaccaacttcgacaagaaccttccgaacgagaaggtgctcccgaagcacagcctgctctacgaatattttactgtgtacaacgagctgacgaaggtcaagtacgttacggaggggatgaggaagcccgccttcctctccggcgagcagaagaaagccattgtggatctcctgttcaagaccaaccgcaaggtgacggtgaaacagctcaaagaggactacttcaagaagatcgagtgcttcgactccgtagagatcagcggggtcgaggaccgcttcaacgcctcgctgggcacgtaccacgacctgctaaagattatcaaggacaaagacttcctagacaatgaggagaacgaggacattctggaggacatcgtgctgactctgacgctgttcgaagaccgcgagatgatcgaggagcggcttaagacgtacgcccacctgttcgacgacaaggtgatgaagcagttgaaacggcggcgctacaccgggtggggccgcctctcccgcaagctcatcaacggcatccgcgacaagcagtcggggaagacgatcctggacttcctcaagagcgacggcttcgccaaccgaaacttcatgcagctaatccacgacgacagcctgacgttcaaggaggacatccagaaggcccaagtgagcggccagggagactcgctacacgagcatatcgccaacctggctggcagcccggcgattaagaaaggaatcctccaaaccgtcaaagtggtggacgagctggtgaaggtgatgggccgccacaagcccgagaacattgtgatcgagatggcgcgggagaaccagacgacgcagaagggccaaaaaaatagcagggaaaggatgaagcgaatagaggaggggatcaaggagctggggagccagattctcaaagagcacccggtcgagaacacacagctccagaacgagaagctgtacctctactacctccaaaacggccgcgatatgtacgtggaccaggaactagacatcaaccggctgagcgactatgacgtggaccacatcgtgccgcagtccttcctcaaggacgactcgattgacaacaaagtgctcactagatccgacaagaacagaggcaagagcgataacgtcccgtcggaggaggtcgtcaagaaaatgaaaaactactggcggcagctcctaaacgccaagctcatcacgcagcgtaagttcgacaacctgacgaaggcggagcggggcgggctgagcgagctggacaaagcggggttcatcaagcggcagctcgttgagacgcggcagatcacaaagcacgtcgcgcaaatcctcgactcccgcatgaacaccaagtacgacgagaacgacaagctcatccgggaggtgaaggtcattacccttaaatcgaagctcgtcagcgactttcgtaaggacttccagttctacaaggtcagagagatcaacaactaccaccacgcccacgacgcctatctgaacgccgtggtgggcaccgcgcttattaagaagtaccccaagctggagtccgagttcgtgtacggcgactacaaggtttatgacgtcaggaagatgatcgccaagtcggaacaggagatcggaaaagctaccgccaaatatttcttctatagcaacatcatgaacttcttcaaaaccgagatcaccctcgccaacggcgagatccggaagcgcccgctcatcgagaccaacggggagaccggggagatcgtctgggacaaggggcgggacttcgctactgtccgaaaggtgctctccatgccacaagtgaatatcgtcaagaaaacagaggtgcagaccggagggttcagtaaggagtccatcctgcccaagcggaactccgacaagctaattgctcgcaaaaaggattgggatcctaaaaaatatggcggcttcgactcgcccacggtcgcctactctgtgctggtcgtggcgaaggtggagaagggcaagtccaagaagctcaagagcgtcaaggagctgctggggatcacgatcatggagcgtagttcgtttgagaagaatcccatcgacttcctggaggctaagggctacaaggaggtcaaaaaggacctcatcattaagctgccgaagtacagcctcttcgagctggagaacgggcggaagcgtatgctcgcctccgctggggagttacaaaaggggaacgagctggcgctgccgtctaagtacgtcaacttcctgtacctggcctcccactacgagaagctcaaggggtcgccggaggacaacgagcagaagcagctcttcgtagagcagcacaagcactacctggacgagatcatcgagcagatttcagagttctcaaagcgggtcatcctcgccgacgccaacctggacaaggtgctctcggcctacaacaagcaccgggacaagccgatccgcgaacaggccgaaaacatcatccacctgttcacgctcaccaacctcggtgccccggcggccttcaagtactttgacacgaccatcgaccggaagcgctatacctcgacgaaggaggtgctggacgccaccctgatccaccagtccatcaccgggctttacgagacccggatcgacctctcgcagcta

SEQ ID NO: 69 - DNA, 4101 bp, synthetic construct
  misc_feature 1..4101: note = Cas9
  source 1..4101: mol_type = other DNA
gacaagaagtatagtattggactcgccatcggaaccaactctgtggggtgggctgttattacagatgaatataaggtgccatccaaaaagtttaaagttctgggcaatactgatagacactcaatcaagaagaatctgataggtgcacttctgtttgatagtggagagactgccgaggcaaccagacttaaaaggactgcaagaagaagatataccagaagaaagaataggatttgctatttgcaggaaatcttcagcaacgaaatggccaaggttgatgactcatttttccataggttggaggagagttttcttgtggaggaagataagaagcacgaaagacacccaattttcgggaatatagtggacgaggtggcttatcatgagaagtatcccactatctaccacctgagaaagaaacttgtggactcaaccgataaggctgatcttaggcttatatacttggcccttgcacatatgatcaaattcaggggccattttcttatcgaaggcgatcttaatcccgataactcagatgtggacaagctgtttatacaacttgtgcaaacctacaatcaactcttcgaggagaatcccattaacgcctccggcgtggatgcaaaagccatactgtcagccagactgagcaaaagtaggagactggagaatcttatagcccaactgcccggtgaaaagaagaatgggctcttcggaaatctgatcgctctttcattggggttgacacccaactttaagagtaactttgacttggcagaagatgcaaagttgcagctcagtaaagacacatatgacgatgaccttgacaatctcttggcacaaataggggatcaatacgctgaccttttcctcgctgccaagaacctcagcgacgctatactgttgtccgacattcttagggttaataccgaaattacaaaggcccctcttagtgcaagtatgatcaaaaggtatgatgagcatcaccaagaccttacactgctgaaggctctggttagacagcaactccctgaaaagtataaggaaatattcttcgaccaaagtaagaacgggtacgccggttatattgatgggggcgcaagtcaagaagaattttacaaattcatcaagccaattcttgaaaagatggacgggactgaggaattgctggtgaaactgaatagagaggaccttcttagaaaacagaggacatttgacaatgggtccatcccacaccagattcatctgggggaactccacgcaatattgaggagacaagaagacttttacccattccttaaggataatagagagaaaatcgaaaaaatcctgactttcaggattccttactatgttgggccactggccagggggaactcaagattcgcttggatgacaaggaagtcagaagaaaccataaccccttggaattttgaagaggtggttgataagggggcatcagcccagtctttcatagagaggatgaccaactttgataaaaatcttccaaatgagaaggttttgccaaaacatagtcttttgtacgagtactttactgtttataacgaattgaccaaggtgaagtatgtgaccgagggaatgaggaagccagcatttttgtccggggagcaaaagaaagcaatcgttgatcttctcttcaagaccaacagaaaagtgaccgtgaaacaactgaaggaagactacttcaaaaagatagaatgtttcgattcagtggaaattagcggtgttgaagacaggttcaatgcttcattgggtacttaccacgacctgttgaagataatcaaagacaaggactttctcgataatgaggagaacgaagacatcttggaagacattgtgcttacactcactttgtttgaggacagggaaatgattgaggaaagactcaaaacttacgctcatttgtttgatgataaggttatgaaacaactaaaaagaagaaggtacaccggctggggaagattgagtaggaaactgatcaacggtattagagataaacaatccggaaagactatcctcgatttccttaagagtgatggctttgcaaataggaattttatgcagctgattcatgacgactcacttaccttcaaagaagacatccaaaaagctcaggtgtctgggcaaggcgacagtctgcatgaacatatagctaacttggctgggagtcccgccatcaagaaggggatacttcaaacagttaaagttgtggacgaattggtgaaggtaatgggaaggcacaagcctgaaaatatagtgatagaaatggcaagggaaaatcaaacaacccagaagggacagaagaacagtagggaaaggatgaaaaggatagaagaggggatcaaagagcttggtagccagatcctcaaggaacatccagtggagaatacccaacttcaaaacgagaaactctatttgtactacttgcagaacggaagagatatgtatgtggaccaagagcttgatattaacaggctgagcgattatgacgttgaccacatagtgccccaatcattcctcaaggatgactctattgataataaggtgctgacaaggagtgacaagaatagagggaaatccgacaacgttccatccgaggaagttgtgaagaagatgaagaactactggaggcagttgctgaacgctaagctcattacccagaggaaattcgataacctgaccaaagcagagagaggcgggctgagcgaactcgataaagcaggtttcatcaagagacaactcgtggagactaggcaaattactaagcacgtggctcaaatactcgacagcaggatgaacacaaagtacgacgagaacgacaagctcattagagaggttaaggttattactctgaaaagtaaattggttagcgatttcagaaaggatttccaattctataaggttagagagatcaacaattatcatcatgcacatgatgcctatctgaatgctgtggttggtacagcccttatcaagaagtaccctaagctagagagcgagtttgtgtacggagattataaggtgtatgatgtgaggaaaatgatcgctaaaagtgagcaagagattggaaaggctaccgccaaatacttcttttattccaatattatgaatttcttcaagacagaaatcaccctggctaacggcgagataaggaagaggccgcttatcgaaactaatggggagacaggcgaaatagtgtgggacaaagggagggatttcgcaactgtgaggaaggttttgagcatgcctcaggtgaatatcgttaagaaaaccgaagttcaaactggagggttctctaaggaaagcattctccccaagaggaactccgacaagctgattgctagaaagaaagactgggaccccaagaagtatggcggattcgactcacccactgtggcatatagcgttctcgtggtggcaaaggttgaaaagggtaaatccaaaaaactcaaatccgtgaaggaactccttggcataactattatggaaaggagtagctttgaaaagaatcccatcgactttctcgaagctaagggctataaggaagttaagaaggaccttataatcaaacttccaaaatactccctttttgagttggaaaacggcagaaagagaatgttggccagtgccggggagcttcaaaagggcaacgaactggctctgcctagcaaatatgtgaactttttgtatctggcatcacactacgagaaacttaaaggctctcctgaggacaacgagcaaaaacagctctttgttgaacagcataagcactacctcgacgagattattgagcagatcagcgagttctcaaagagagttattctggctgacgctaatcttgacaaggttttgtccgcttacaacaaacacagggataagccaatcagggagcaggcagaaaacataatccatctctttaccctgacaaacctcggtgcccccgctgctttcaagtattttgatactaccattgacaggaagagatatacttccactaaggaagtgctcgacgcaaccctcatacaccaaagtatcacaggcctctatgaaactaggatagatttgtctcaacttgggggcgat

SEQ ID NO: 70 - DNA, 4101 bp, synthetic construct
  misc_feature 1..4101: note = Cas9
  source 1..4101: mol_type = other DNA
gacaaaaagtattccatcgggcttgctatcggaaccaactctgtggggtgggcagttattaccgacgaatacaaggtgcccagcaagaagtttaaggttctggggaacacagatagacatagcataaagaaaaacctgataggcgcactgttgttcgactccggggaaacagccgaagctaccaggctgaagagaactgcaagaagaaggtacaccagaagaaaaaacagaatatgttatctccaagagattttctctaacgagatggccaaggtggacgactcattctttcacagactggaagaatctttccttgtggaagaagataagaaacacgagaggcaccctatttttggcaatatcgtggatgaggtggcttaccacgaaaaataccctacaatataccacctcaggaaaaaattggttgatagtacagacaaggccgacctcaggctcatctatttggccctggcccatatgattaaattcagggggcactttctcatcgagggagatttgaaccccgacaacagtgatgttgataagctctttattcagctcgtgcagacttacaatcagttgtttgaggaaaaccccattaatgcttccggggtggacgccaaggcaatcctttctgcaagactctcaaagtcaaggagactcgaaaatctgatagcacagcttccaggagagaagaagaacgggctctttggaaacctgatcgctctgtcactcggactcacacccaatttcaaaagcaattttgatttggcagaggacgctaagctgcaactcagtaaggatacctacgacgatgacttggataatctgctcgcacaaattggggaccagtatgcagacctgtttctcgcagctaagaacttgagtgacgccatattgctcagtgacatcctcagggttaataccgagattacaaaagctccactctctgcaagcatgatcaagaggtatgacgagcaccatcaagacctgacactccttaaggcgttggttaggcagcaacttcctgaaaagtataaggaaatcttcttcgatcaaagcaaaaacggctacgccggctatatagacgggggagcatcccaagaagaattttataagttcataaaacctatattggagaagatggacgggacagaggaattgctcgtgaaactgaacagggaggatctcctcaggaagcaaaggaccttcgacaatggctccatcccacatcagattcacctcggcgaactgcacgcaatactgagaagacaagaggacttttatcctttcctgaaggacaacagggagaaaatcgagaaaatcttgacattcagaatcccatactacgttgggcctctggccagaggtaacagtaggttcgcctggatgactaggaaatcagaggagactattacaccctggaactttgaagaagttgttgataagggagcttcagcacaatcattcatcgaaagaatgacaaactttgacaaaaatctgcctaatgagaaagtgctcccaaaacattccctgctgtatgagtattttaccgtttataacgagcttaccaaggtgaaatacgttactgaaggtatgagaaagccagcttttctttcaggggagcaaaagaaggctatcgtggatcttctctttaagaccaacagaaaggttaccgtgaagcagcttaaggaagactactttaaaaagatcgagtgttttgactcagtggaaataagcggtgttgaagatagattcaacgcatccttgggaacttatcatgatcttcttaagataatcaaggataaagactttctcgacaacgaggaaaacgaagatatactggaggacatagttctgacacttactttgttcgaggatagggagatgatcgaggaaagactgaaaacatatgctcaccttttcgacgacaaagttatgaaacaactcaagagaaggagatatacagggtgggggagattgagcaggaaactgattaatggtatcagagacaaacagtcaggaaaaacaatactcgactttttgaaatcagacgggttcgcaaataggaatttcatgcagcttatacacgacgattcacttacttttaaagaggacattcaaaaggctcaagttagtggacaaggtgactccctccacgaacacatcgcaaatctcgctggcagccctgcaattaagaagggtatactccagacagttaaggttgttgacgagctggttaaagtgatgggaagacacaaacccgagaacatagtgatagagatggccagggaaaaccaaaccactcaaaaagggcagaaaaattccagagagaggatgaaaaggattgaagaaggtatcaaggagctgggtagccaaattctgaaagaacatcctgtggaaaacactcaactccagaatgagaaactctatctgtactatctgcaaaatgggagagatatgtatgtggaccaggaactggacataaacaggctctcagattacgatgtggatcatatcgtgccacagtcctttcttaaggatgatagcatcgacaataaggtgcttaccaggtccgacaagaacaggggaaagtcagataacgtgccttctgaagaagttgttaaaaagatgaagaactactggagacagctgcttaacgctaagctcataacacagaggaagtttgacaacttgaccaaggccgagagaggcggactctcagaattggataaggcagggttcataaaaaggcagctggtggaaacaaggcagataactaaacatgtggctcagatcctcgatagtaggatgaatacaaaatacgatgagaacgacaagctcataagggaggttaaagtgataactctgaaatccaaactggttagcgattttaggaaggatttccagttttacaaagttagggagatcaacaattatcatcacgcccacgatgcctacttgaacgcagttgtgggtactgcacttatcaaaaagtaccctaagctggaatccgagtttgtttatggagactataaggtgtacgacgttagaaaaatgattgcaaagtcagagcaggagatagggaaagccactgcaaaatatttcttttatagcaatatcatgaatttctttaagacagaaatcacactggccaatggggaaataaggaagaggcccctgatcgaaactaatggcgagacaggggagattgtgtgggataaaggtagggactttgcaacagtgaggaaagtgctgagcatgccccaagttaatatcgttaaaaagaccgaggttcaaacagggggctttagtaaggaaagcattttgcccaagaggaatagtgacaaattgattgctaggaaaaaagattgggaccccaaaaagtatggcggatttgatagccccactgttgcttactccgtgctcgtggttgcaaaggtggagaagggaaagagcaagaaactgaagtcagttaaggaactccttggtatcactatcatggaaagaagctcctttgagaagaaccctattgacttcctggaggctaaagggtacaaagaggttaagaaagaccttatcattaaattgcccaaatatagtcttttcgagcttgaaaacggaagaaagaggatgcttgcatccgctggcgaattgcaaaagggcaatgagcttgctctcccttccaagtatgtgaacttcctttatcttgcctcacactatgaaaaactcaaaggttcacccgaagacaacgaacaaaagcaactatttgtggaacaacacaagcactacctggacgaaatcattgagcaaatttctgagttttcaaaaagggtaatcttggctgacgcaaatctcgacaaagttttgtcagcttacaacaaacatagagataagccaattagagagcaagctgagaatatcatccatctgtttaccctgactaaccttggagcgcctgctgcttttaaatatttcgacaccacaatcgacaggaagaggtacactagcactaaggaagttctcgacgccaccctcatccaccagagtattacaggcctgtacgagacaagaattgatctttctcaacttggtggtgac

SEQ ID NO: 71 - DNA, 4101 bp, synthetic construct
  misc_feature 1..4101: note = Cas9
  source 1..4101: mol_type = other DNA
gataagaagtactcaatcggtctggcaatcggaaccaactctgtgggttgggcagtgattacagatgagtataaggtgccaagcaaaaaattcaaggtgctgggtaataccgacagacacagcattaagaagaatttgattggagcactcctctttgactcaggggaaacagcagaggcaacaaggctgaagaggacagcaaggcggaggtacacaaggcggaaaaacaggatatgctacctccaggaaatctttagcaacgagatggctaaagtggatgatagctttttccatagactcgaagaatcctttcttgttgaagaggacaaaaagcatgaaaggcatcccatcttcggcaatatagttgatgaggttgcataccatgagaagtaccccacaatctaccacctcagaaagaaacttgtggactccacagataaagcagacctgaggctcatatacctcgcactcgcacacatgatcaagttcagagggcactttctcatcgaaggtgacctgaatccagataattcagatgtggataaactgtttatacagctggtgcaaacatacaaccaacttttcgaggaaaacccaatcaatgcctccggtgttgatgcaaaggccatcctgtcagcaagactcagcaaaagcaggcggctcgaaaacctcatcgcccagcttcccggtgaaaagaagaacgggctctttggtaatctcatcgcattgagccttggtcttactccaaacttcaagagcaattttgatctggcagaggatgctaaactgcaactctcaaaggacacatatgacgatgaccttgacaatctgttggcccagatcggggaccaatatgcagacctcttcctggccgcaaagaatctgtcagatgcaatcctcttgtccgacatactgagagttaacactgagatcacaaaggcacctctgtccgcctccatgattaagagatacgatgagcatcaccaggatctgactttgctcaaagccctcgttagacagcagttgccagaaaagtacaaagaaatattctttgatcaatcaaaaaacggatatgcagggtacatcgacggtggggcaagccaggaagagttctacaaattcatcaaacctatcctggaaaagatggatgggacagaagagctgctggttaagctgaatagggaagacctcctcagaaagcagaggacatttgataacgggagcatccctcatcaaatccacctcggtgaactccatgctatcctgagaaggcaggaagacttttatccatttttgaaggacaatagggagaaaatcgaaaaaatcctgacattcagaatcccatactacgttggtcctctggcaagaggtaacagtaggttcgcatggatgacaaggaaaagcgaggagacaatcacaccctggaattttgaggaagttgttgacaagggtgccagcgcacaatcctttatcgaaagaatgacaaatttcgacaagaatctgcctaacgaaaaggttctcccaaagcattcactcctgtacgaatattttacagtttataacgaactgactaaagttaaatacgttaccgagggtatgaggaagccagcattcctttccggggaacagaagaaagctattgtggacctcctgttcaagacaaatagaaaagtgacagttaagcaactcaaagaggattacttcaaaaagatcgaatgttttgactctgtggagatcagcggggtggaggatagattcaacgccagcctgggtacatatcatgatctcctgaaaatcattaaagacaaggacttccttgacaacgaggagaacgaggacattctggaagacattgttctgaccctcacactctttgaggatagggagatgattgaggaaagactgaagacctacgcccacctctttgacgataaagtgatgaaacagctcaagagaagaaggtatacaggttgggggagactgagcaggaagttgatcaatgggattagggacaaacagtccgggaaaacaatcctcgattttctgaagtcagacggtttcgcaaacagaaattttatgcagctcattcacgatgacagcttgacattcaaggaagacatccaaaaggctcaagtgagcggccaaggggatagcctccacgagcatattgcaaatctggcaggttcaccagccatcaaaaagggcatacttcagacagttaaggttgtggacgaattggttaaagttatgggcaggcataagccagagaatatcgttatcgaaatggcaagggagaaccaaacaactcaaaaagggcagaaaaatagcagagagaggatgaaaagaatcgaggaagggatcaaggaacttgggtcccaaatcctcaaggagcacccagttgaaaatactcaactgcaaaacgagaagctctatctctactatctccaaaacgggagggatatgtatgttgaccaggagctggatattaacagactgtcagattatgatgttgatcatatcgtgccccagtcattcctgaaggacgattccatcgacaacaaagttctcacaaggtccgataaaaacaggggcaagtccgataacgttccaagcgaagaagtggtgaaaaagatgaaaaactattggagacaacttctgaatgcaaagttgattactcagagaaagtttgacaacctcacaaaagcagaaagaggcgggcttagcgaactcgataaggcagggtttatcaaaagacagctggttgagacaaggcagatcacaaaacatgtggcacagatccttgactcaaggatgaataccaagtatgatgagaatgataagttgatcagggaggttaaagttatcacactcaaatccaaactggtgtcagacttcaggaaagactttcaattttataaggtgagggagatcaataactaccaccatgcacatgacgcctacctgaacgcagtggtgggtacagcattgattaaaaaataccctaagctggagtctgagtttgtgtacggggactacaaggtgtacgacgtgaggaaaatgatagccaagtccgagcaggagatcgggaaagcaacagctaagtatttcttttacagtaatatcatgaatttctttaaaactgagattactctggcaaacggggagatcaggaaaagacccctcatcgagactaatggtgaaacaggtgagatcgtttgggacaaggggagggattttgctactgttagaaaagttctgagtatgccacaagtgaatattgtgaaaaagacagaagttcagacaggtgggttctccaaagaatccatcctgcccaagagaaattcagacaagctcatcgcaagaaagaaggactgggaccctaagaagtacggaggatttgacagccccaccgtggcctattccgtgcttgttgtggcaaaggtggagaaagggaagagcaaaaaactgaaatccgtgaaagaactgctgggaattaccatcatggaaagaagctcctttgagaagaacccaatcgacttcctggaagcaaaaggatataaggaagtgaaaaaggacctcattatcaagctcccaaaatactcacttttcgagttggagaacggtagaaagaggatgctggcaagcgcaggggaacttcagaaaggcaatgagctggcattgccatcaaagtatgtgaacttcctctacttggccagccattacgagaaacttaaaggtagcccagaagataacgagcaaaaacagctctttgtggaacagcataagcattatctggatgagatcatagaacaaatctcagagttttccaagagagttatcctcgcagatgcaaacctggataaggttctctcagcctataataagcatagagacaagccaattagagagcaagcagagaacattatccacttgttcactcttacaaacctgggggcaccagccgccttcaaatatttcgatacaacaatagacagaaagaggtataccagcaccaaagaagttctcgacgccacactgatccatcaatcaatcacaggcctttacgaaactaggatcgacttgtcacaactgggtggggat

SEQ ID NO: 72 - DNA, 3307 bp, synthetic construct
  misc_feature 1..3307: note = Cas9
  source 1..3307: mol_type = other DNA
gagcaaggacacctacgacgacgacttggacaacctattggcccagataggtgaccagtatgcagacctcttccttgcggccaagaacttgagtgacgctatactgctcagtgacatcctgagggtgaacactgagatcactaaggcccctctctctgcctcaatgattaagcgttacgacgagcatcaccaggatctcaccctgcttaaggcccttgttcggcagcagctccctgagaagtacaaggagatattttttgaccagtctaagaacggctacgccggttacattgacggtggggcaagccaggaggagttctacaagttcatcaagccgatccttgagaagatggacggcaccgaggagctacttgtcaagttgaaccgggaagacctgctccggaaacagcgtacattcgacaacggcagcatccctcaccagatccacctgggcgaactacacgccatcctccgacgtcaggaggacttctatccattcttgaaagataacagggaaaaaatcgaaaaaatacttacgtttcgaataccttactacgtggggccccttgctcggggaaactccagattcgcatggatgaccaggaagtcagaggagaccatcacaccctggaactttgaggaggtggttgacaaaggtgcttctgcccagtccttcattgagcggatgactaacttcgacaagaacctgcccaacgagaaggtgctgccaaagcacagcctgctctacgaatactttactgtgtacaatgagctgacgaaggtgaagtacgtgacagaggggatgcggaagcccgctttcctgagcggcgagcaaaaaaaagcaatcgtggacctactgttcaagaccaaccgaaaggtgacagtgaagcagctcaaggaggactacttcaaaaaaatcgagtgcttcgactctgttgagataagcggcgtggaggaccgattcaacgcctcattgggaacctatcacgacctgctcaagatcattaaggacaaggacttcctggataatgaggagaatgaggacatcctggaggatattgtgctgacccttactctattcgaggacagggagatgatcgaggagcgactcaagacctacgctcacctgttcgacgacaaggttatgaagcaattgaagcgtaggcgatacacggggtggggaagactctcccgaaaactgataaacggcatcagggacaagcagtcagggaagacgatcttggacttcctgaaatccgacgggttcgccaaccgcaacttcatgcagctcattcacgacgactcactaacgttcaaagaggacattcagaaggctcaagtcagtggacaaggcgactccctgcacgagcacattgcaaaccttgcgggctccccggcgattaaaaagggcattctccaaacggttaaggtggtggacgagctggtgaaggtgatgggccgacacaagcctgagaacatcgtgatcgagatggccagggagaaccagactacccagaagggtcagaagaactctcgggaacgtatgaagcgtattgaggaggggattaaggagttgggctctcaaatcctcaaggagcaccctgtggagaacactcagctccaaaacgagaagctgtacctgtactacctgcaaaacgggcgcgatatgtacgtggatcaggagttggacatcaacaggcttagcgattacgacgtggaccacatcgtgccacagtcattcttaaaggacgacagcatcgacaacaaggttctgacgaggagcgacaagaatcgagggaaaagtgacaatgttccatccgaggaggtggtcaagaaaatgaagaactattggcgtcagcttctgaacgccaagctcatcacccagcggaaattcgacaacctgactaaggctgagcgaggcggactctccgagcttgacaaggctggcttcatcaagcggcagttggtcgaaacccgacagataacgaagcacgttgcccagatacttgactcccgtatgaacaccaagtacgacgagaacgacaagctcatcagggaggtgaaggtcattacccttaagtccaaactcgtcagcgactttcgtaaggacttccagttctacaaggtgcgcgagatcaataactaccaccacgcacacgacgcctacctgaacgcagtggttggaaccgcgttgattaaaaagtaccccaagttggagtcggagttcgtttacggggactacaaggtgtacgacgttcggaagatgatcgccaagtctgaacaggagatcgggaaagcaaccgccaagtatttcttctatagcaacatcatgaacttctttaaaaccgagatcacacttgccaatggcgagatccgtaagaggccgctgatcgagacaaatggggagactggcgagatcgtgtgggacaagggccgcgacttcgcaaccgttcggaaagtcttgtccatgcctcaagtcaacatcgtcaagaagactgaggtgcaaacaggcgggttctcgaaggagtccatactgcccaagaggaactcagacaagctcatagcacgcaaaaaagactgggatccaaagaaatacggcgggttcgactcgccgacagtcgcatactccgtgttagtggtggctaaagtggaaaaggggaagtccaagaagctcaagtccgtcaaggagttgctcgggatcaccattatggaacggtcctcattcgagaagaatcccattgacttcctagaggcgaagggctacaaagaggtcaaaaaggacctaattattaagctccccaagtattcactcttcgaacttgaaaatggtcgtaagcggatgttggcaagcgctggagagcttcagaaggggaacgagcttgcactgccttccaagtacgtgaacttcctgtacctcgcctctcattacgagaagttgaagggctcaccggaggacaacgagcagaagcagttgttcgtggagcagcacaagcactacctcgacgagatcattgagcagataagtgagttcagcaaacgggtgatccttgccgacgctaacctggacaaggtgctgagcgcctacaacaagcacagagacaagccgatccgagagcaagcggagaacatcatacacctgttcaccctcacgaacctcggggctcccgcagccttcaaatattttgacacgaccatcgaccgtaaacgctacactagcacgaaggaggtgctggacgctacccttatccaccagtccatcaccggcctgtacgagacgagaatcgacttgtcgcagctcggtggtgac

SEQ ID NO: 73 - DNA, 4101 bp, synthetic construct
  misc_feature 1..4101: note = Cas9
  source 1..4101: mol_type = other DNA
gacaaaaaatactcaattggtctggcaattgggaccaacagtgtcggatgggccgtgattaccgacgagtacaaggtgccgtccaaaaaattcaaggtgcttgggaacaccgaccgccactcgatcaagaaaaacctaatcggtgcgttgcttttcgacagtggggagaccgccgaggcaacacgcttaaaacgcacagctaggaggagatatacacggcgcaagaaccgaatatgctacttacaggagatattctccaatgagatggcgaaggtggacgactctttcttccatcggcttgaggaatccttcctggtcgaggaggacaagaagcacgagcgacacccgatattcgggaacatcgttgatgaggtggcgtaccacgagaagtacccaacgatataccacttacgcaagaagctcgtggactctacggacaaggccgacttgcgccttatctacttggcactggcccacatgattaagttccgaggccacttccttatcgagggtgacctgaaccccgataactccgacgtggacaagctcttcatccaactcgtccagacatacaaccagctattcgaggagaatcctatcaacgcctctggggtggacgctaaagctatcctctcagcccgcctgtcaaagtcgaggaggttggagaacctaatcgcccagcttccaggcgagaagaaaaatgggctgttcggaaaccttatcgcactctcactgggcctaaccccgaacttcaagtccaacttcgacctggcagaggacgcgaaattgcagttgtcgaaagacacctatgacgatgacctggacaacctgttggcccagataggggaccagtacgccgacctgttcctagcggccaagaacctgtccgacgccatcttgctgtcggatatactgcgggtgaacaccgagatcactaaagcacctctctccgccagcatgattaagcgttacgacgagcaccaccaagatttgaccctgctaaaggcacttgtacggcagcagcttcccgagaagtacaaggagatctttttcgaccaaagcaagaacggctacgccgggtacatcgacggaggtgccagccaggaggagttctacaagttcattaagcccatcctggagaagatggacgggactgaggaactacttgtgaagctgaaccgggaagacttactacggaagcagcgtaccttcgacaacggttctatcccacatcagatccatcttggggagttgcacgcgatcctgcgacgccaggaggacttttaccccttcctgaaagacaaccgcgagaaaatcgagaagatactgaccttcagaataccttactacgtcggaccccttgcgcgaggcaactcaagattcgcgtggatgaccaggaaatcagaggagaccatcacaccctggaatttcgaggaggtggttgacaagggtgcctccgcccagtcctttatcgaacgaatgaccaacttcgacaagaacttgcccaacgagaaggtgctccccaaacacagcctcctctacgaatatttcacagtgtacaacgagcttactaaagttaagtatgttactgagggcatgaggaaacccgccttcctgtcaggcgagcagaagaaagctattgtggacctccttttcaagaccaaccggaaggtgacagtgaagcagctcaaggaggactacttcaagaagatagagtgcttcgacagcgtggagatcagcggggtggaggacagattcaatgcctctctcggaacataccacgacttgcttaagatcatcaaggacaaggacttcctcgacaacgaggaaaacgaggatattctggaggatattgttctgactcttaccctgttcgaggaccgggagatgatcgaggagcgtctcaagacctacgcccacctgttcgacgacaaagttatgaagcagctcaagcgtcggagatataccggatggggccgtctgtctcggaagctcatcaacgggatcagggacaagcagtcagggaagacgatcttagacttccttaagtctgacggcttcgccaacaggaacttcatgcagttgatccacgacgacagccttaccttcaaggaggacatccagaaggcccaagtgagtggccagggtgacagcctccacgagcatattgctaatcttgcgggttccccagcgattaaaaagggcatacttcaaaccgttaaggtggtggacgagcttgtcaaggtgatggggcgacacaagcccgagaacatcgtgatcgagatggccagggagaaccagaccacccagaaggggcagaagaatagccgagaacgcatgaagcgcatcgaggaggggattaaggagctagggagccagatcctcaaggaacatcccgtcgagaacacccagctccagaacgagaagctatacctctactacttgcaaaacgggagggatatgtacgtggatcaggagttggacattaaccgcctaagcgactacgacgtagatcacatcgtgcctcagtcattcctcaaagacgacagcattgacaacaaagtcttgacccgatccgacaagaaccgaggaaaatccgacaatgtgccctcagaggaggtcgtcaagaaaatgaagaactattggaggcagctacttaacgccaaactcataacccagcggaagttcgacaacctgacaaaggctgagcggggtgggctcagcgagcttgacaaggctggcttcatcaagcggcagttggtggagacaagacagataacgaagcacgtggctcagatcctggactctcgcatgaacacgaagtacgacgagaacgacaaattgatccgcgaggtcaaggttattacgctcaagagcaaacttgtcagcgatttccgcaaggacttccagttctacaaggtgagggagattaacaactaccaccatgcacatgatgcctacttgaacgcagtggtggggaccgcgcttattaaaaagtaccctaagttggagtcagagttcgtttatggggactacaaggtgtacgacgtccggaagatgattgcaaagtctgaacaggaaatcgggaaggccaccgccaaatatttcttctacagtaacattatgaatttttttaagactgaaattactctcgcaaacggcgagatcaggaagcgtcccctcatcgagacaaacggggagaccggggagatagtctgggacaaggggcgggacttcgctacggtgaggaaggtgctctcgatgccacaagtgaacatcgtcaaaaagacagaggtgcagaccggtggcttctcaaaggagtcaatcctgccaaaacgtaacagcgacaagctcatcgcccgcaagaaagactgggaccctaagaagtatggtgggttcgactcaccgacggtcgcatactccgttctggtcgtggcaaaggtggaaaagggcaagtccaaaaaactgaaatccgtgaaggagttgcttggcattaccatcatggaacgcagcagcttcgagaagaaccccattgacttcctggaggctaaagggtacaaggaggtcaagaaagatttaattattaagctacctaagtacagcttgttcgagctggagaacggccgaaaacgaatgctcgcatccgccggggaacttcaaaagggcaacgagcttgcgctgccctccaagtacgtgaacttcctgtacttggcatcccactacgagaaactcaagggtagcccagaggacaacgagcagaagcagctattcgtggagcagcacaagcactacctcgacgagataatcgagcagatcagtgagttcagtaagcgggtgatactcgcggacgccaacttggacaaggtgcttagtgcctacaacaagcaccgtgacaagcccatccgagaacaggctgagaacatcatccaccttttcactctgacaaacctcggtgctcccgccgccttcaaatacttcgacactaccatcgacaggaagcgctacacatctacgaaggaagttcttgacgctacgcttattcatcagtctatcacagggctgtacgagacaaggatcgaccttagccaactcggcggggat

SEQ ID NO: 74 - protein, 1367 aa, synthetic construct
DKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 75 - protein, 1367 aa, synthetic construct
DKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLADDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPALESEFVYGDYKVYDVRKMIAKSEQEIGKATAKYFFYSNIMNFFKTEITLANGEIRKAPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 76 - protein, 228 aa, Rattus norvegicus
SSETGPVAVDPTLRRRIEPHEFEVFFDPRELRKETCLLYEINWGGRHSIWRHTSQNTNKHVEVNFIEKFTTERYFCPNTRCSITWFLSWSPCGECSRAITEFLSRYPHVTLFIYIARLYHHADPRNRQGLRDLISSGVTIQIMTEQESGYCWRNFVNYSPSNEAHWPRYPHLWVRLYVLELYCIILGLPPCLNILRRKQPQLTFFTIALQSCHYQRLPPHILWATGLK

SEQ ID NO: 77 - protein, 199 aa, Homo sapiens
MEASPASGPRHLMDPHIFTSNFNNGIGRHKTYLCYEVERLDNGTSVKMDQHRGFLHNQAKNLLCGFYGRHAELRFLDLVPSLQLDPAQIYRVTWFISWSPCFSWGCAGEVRAFLQENTHVRLRIFAARIYDYDPLYKEALQMLRDAGAQVSIMTYDEFKHCWDTFVDHQGCPFQPWDGLDEHSQALSGRLRAILQNQGN

SEQ ID NO: 78 - DNA, 621 bp, Petromyzon marinus
  source 1..621: mol_type = genomic DNA
acagatgcagagtatgtgagaattcacgaaaagctggacatctataccttcaagaagcagttctttaacaataagaagtctgtgagccataggtgctacgtgctgttcgagctgaagagaaggggtgaaagaagggcatgtttttgggggtatgctgtgaacaagccccagtctggaactgagagaggcattcacgccgaaattttcagcatcagaaaggtggaggaatacctgagggataaccctggacagtttacaattaattggtattctagctggtctccatgcgctgactgtgccgagaagatcctggaatggtacaaccaggagctgagaggaaatggccataccctgaagatttgggcctgcaagctgtactatgaaaagaacgcaagaaatcagatcggactgtggaacctgagggataatggtgtggggctgaacgtgatggtgtccgagcactatcagtgctgtagaaagattttcattcagtcctcacataatcagctgaacgagaatagatggctggaaaagactctgaagagggctgagaagagaaggtccgaactgtcaattatgatccaggtgaagatcctgcacaccactaagtcacctgccgtg

SEQ ID NO: 79 - protein, 160 aa, synthetic construct
FERNYDPRELRKETYLLYEIKWGKSGKLWRHWCQNNRTQHAEVYFLENIFNARRFNPSTHCSITWYLSWSPCAECSQKIVDFLKEHPNVLEIYVARLYYHEDERNRQGLRDLVNSGVTIRIMDLPDYNYCWKTFVSDQGGDEDYWPGHFAPWIKQYSLKL

SEQ ID NO: 80 - protein, 229 aa, Rattus norvegicus [sequence omitted]

SEQ ID NO: 81 - protein, 198 aa, Homo sapiens [sequence omitted]

SEQ ID NO: 82 - protein, 197 aa, synthetic construct
MDSLLMNRREFLYQFKNVRWAKGRRETYLCYVVKRRDSATSFSLDFGYLRNKNGCHVELLFLRYISDWDLDPGRCYRVTWFISWSPCYDCARHVADFLRGNPNLSLRIFTARLYFCEDRKAEPEGLRRLHRAGVQIAIMTFKDYFYCWNTFVENHGRTFKAWEGLHENSVRLSRQLRRILLPLYEVDDLRDAFRTCT

SEQ ID NO: 83 - protein, 207 aa, synthetic construct
TDAEYVRIHEKLDIYTFKKQFSNNKKSVSHRCYVLFELKRRGERRACFWGYAVNKPQSGTERGIHAEIFSIRKVEEYLRDNPGQFTINWYSSWSPCADCAEKILEWYNQELRGNGHTLKIWVCKLYYEKNARNQIGLWNLRDNGVGLNVMVSEHYQCCRKIFIQSSHNQLNENRWLEKTLKRAEKRRSELSIMFQVKILHTTKSPAV

SEQ ID NO: 84 - protein, 228 aa, synthetic construct
SSKTGPVAVDPTLRRRIEPHEFEVFFDPRELRKETCLLYEINWGGRHSIWRHTSQNTNKHVEVNFIEKFTTERYFCPNTRCSITWFLSWSPCGECSRAITEFLSRYPNVTLFIYIARLYHLANPRNRQGLRDLISSGVTIQIMTEQESGYCWHNFVNYSPSNESHWPRYPHLWVRLYVLELYCIILGLPPCLNILRRKQSQLTSFTIALQSCHYQRLPPHILWATGLK

SEQ ID NO: 85 - protein, 162 aa, synthetic construct
SFERNYDPRELRKETYLLYEIKWGKSGKLWRHWCQNNRTQHAEVYFLENIFNARRFNPSTHCSITWYLSWSPCAECSQKIVDFLKEHPNVNLEIYVARLYYPENERNRQGLRDLVNSGVTIRIMDLPDYNYCWKTFVSDQGGDEDYWPGHFAPWIKQYSLKL

SEQ ID NO: 86 - protein, 166 aa, Escherichia coli
SEVEFSHEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARDAKTGAAGSLMDVLHHPGMNHRVEITEGILADECAALLSDFFRMRRQEIKAQKKAQSSTD

SEQ ID NO: 87 - protein, 166 aa, synthetic construct
SEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHYPGMNHRVEITEGILADECAALLCYFFRMPRQVFNAQKKAQSSTD

SEQ ID NO: 88 - protein, 166 aa, synthetic construct
SEVEFSHEYWMRHALTLAKRAWDEREVPVGAVLVLNNRVIGEGWNRSIGLHDPTAHAEIMALRQGGLVMQNYRLIDATLYVTFEPCVMCAGAMIHSRIGRVVFGVRNAKTGAAGSLMDVLHYPGMNHRVEITEGILADECAALLCYFFRMRRQVFNAQKKAQSSTD

SEQ ID NO: 89 - protein, 166 aa, synthetic construct
SEVEFSHEYWMRHALTLAKRALDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHYPGMNHRVEITEGILADECNALLCYFFRMRRQVFNAQKKAQSSTD

SEQ ID NO: 90 - protein, 166 aa, synthetic construct
SEVEFSHEYWMRHALTLAKRALDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHYPGMNHRVEITEGILADECNALLCYFFRMPRQVFNAQKKAQSSTD

SEQ ID NO: 91 - protein, 1763 aa, unidentified
SEVEFSHEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARDAKTGAAGSLMDVLHHPGMNHRVEITEGILADECAALLSDFFRMRRQEIKAQKKAQSSTDSGGSSGGSSGSETPGTSESATPESSGGSSGGSSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHYPGMNHRVEITEGILADECAALLCYFFRMPRQVFNAQKKAQSSTDSGGSSGGSSGSETPGTSESATPESSGGSSGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 92 - protein, 1565 aa, unidentified
SEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSINSGGSSGGSSGSETPGTSESATPESSGGSSGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 93 - protein, 1565 aa, unidentified
SEVEFSHEYWMRHALTLAKRARDEREVPVGAVLVLNNRVIGEGWNRAIGLHDPTAHAEIMALRQGGLVMQNYRLYDATLYSTFEPCVMCAGAMIHSRIGRVVFGVRNAKTGAAGSLMDVLHHPGMNHRVEITEGILADECAALLCRFFRMPRRVFNAQKKAQSSTDSGGSSGGSSGSETPGTSESATPESSGGSSGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 94 - protein, 364 aa, unidentified
SEVEFSHEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARDAKTGAAGSLMDVLHHPGMNHRVEITEGILADECAALLSDFFRMRRQEIKAQKKAQSSTDSGGSSGGSSGSETPGTSESATPESSGGSSGGSSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHYPGMNHRVEITEGILADECAALLCYFFRMPRQVFNAQKKAQSSTD

SEQ ID NO: 95 - protein, 167 aa, unidentified
MSEVEFSHEYWMRHALTLAKRARDEREVPVGAVLVLNNRVIGEGWNRAIGLHDPTAHAEIMALRQGGLVMQNYRLYDATLYSTFEPCVMCAGAMIHSRIGRVVFGVRNAKTGAAGSLMDVLHHPGMNHRVEITEGILADECAALLCRFFRMPRRVFNAQKKAQSSTD

SEQ ID NO: 96 - protein, 167 aa, unidentified
MSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSIN

SEQ ID NO: 97 - protein, 83 aa, unidentified
  source 1..83: note = Bacillus phage AR9
TNLSDIIEKETGKQLVIQESILMLPEEVEEVIGNKPESDILVHTAYDESTDENVMLLTSDAPEYKPWALVIQDSNGENKIKML

SEQ ID NO: 98-100 - skipped (3 entries)

SEQ ID NO: 101 - DNA, 66 bp, unidentified
  source 1..66: mol_type = other DNA
ttcttgtcgtacttatagatcgctacgttatttcaattttgaaaatctgagtcctgggagtgcgga

SEQ ID NO: 102 - protein, 605 aa, unidentified
MSGWESYYKTEGDEEAEEEQEENLEASGDYKYSGRDSLIFLVDASKAMFESQSEDELTPFDMSIQCIQSVYISKIISSDRDLLAWFYGTEKDKNSVNFKIYVLQELDNPGAKRILELDQFKGQQGQKRFQDMMGHGSDYSLSEVLWVCANLFSDVQFKMSHKRIMLFTNEDNPHGNDSAKASRARTKAGDLRDTGIFLDLHLKKPGGFDISLFYRDIISIAEDEDLRVHFEESSKLEDLLRKVRAKETRKRALSRLKLKLNKDIVISVGIYNLVQKALKPPPIKLYRETNEPVKTKTRTFNTSTGGLLLPSDTKRSQIYGSRQIILEKEETEELKRFDDPGLMLMGFKPLVLLKKHHYLRPSLFVYPEESLVIGSSTLFSALLIKCLEKEVAALCRYTPRRNIPPYFVALVPQEEELDDQKIQVTPPGFQLVFLPFADDKRKMPFTEKIMATPEQVGKMKAIVEKLRFTYRSDSFENPVLQQHFRNLEALALDLMEPEQAVDLTLPKVEAMNKRLGSLVDEFKELVYPPDYNPEGKVTKRKHDNEGSGSKRPKVEYSEEELKTHISKGTLGKFTVPLKEACRAYGLKSGLKKQELLEALTKHFQD

SEQ ID NO: 103 - protein, 482 aa, synthetic construct
MVRSGNKAAWLCMDVGFTMSNSIPGIESPFEQAKKVITMFVQRQVFAENKDEIALVLFGTDGTDNPLSGGDQYQNITVHRHLMLPDFDLLEDIESKIQPGSQQADFLDALIVSMDVIQHETIGKKFEKRHIEIFTDLSSRFSKSQLDIIIHSLKKCDISERHSIHWPCRLTIGSNLSIRIAAYKSILQERVKKTTWDAKTLKKEDIQKETVYCLNDDDETEVLKEDIIQGFRYGSDIVPFSKVDEEQMKYKSEGKCFSVLGFCKSSQVQRRFFMGNQVLKVFAARDDEAAAVALSSLIHALDDLDIWAIVRYAYDKRANPQVGVAFPHIKHNYECLVYVQLPFMEDLRQYMFSSLKNSKKYAPTEAQLNAVDALIDSMSLAKKDEKTDTLEDLFPTTKIPNPRFQRLFQCLLHRALHPREPLPPIQQHIWNMLNPPAEVTTKSQIPLSKIKTLFPLIEAKKKDQVTAQEIFQDNHEDGPTAK

SEQ ID NO: 104 - DNA, 10 bp, unidentified
  source 1..10: mol_type = other DNA
aatttttgga

SEQ ID NO: 105 - protein, 83 aa, unidentified
GSVIDVSSQRVNVQRPLDALGNSLNSPVIIKLKGDREFRGVLKSFDLHMNLVLNDAEELEDGEVTRRLGTVLIRGDNIVYISP

SEQ ID NO: 106 - DNA, 25 bp, unidentified
  source 1..25: mol_type = other DNA; note = bacteriophage MS2
gcgcacatgaggatcacccatgtgc

SEQ ID NO: 107 - protein, 116 aa, unidentified
  source 1..116: note = bacteriophage MS2
MASNFTQFVLVDNGGTGDVTVAPSNFANGIAEISSNSRSQAYKVTCSVRQSSAQNRKYTIKVEVPKGAWRSYLNMELTIPIFATNSDCELIVKAMQGLLKDGNPIPSAIAANSGIY

SEQ ID NO: 108 - DNA, 26 bp, unidentified
  source 1..26: mol_type = other DNA; note = bacteriophage PP7
ataaggagtttatatggaaaccctta

SEQ ID NO: 109 - protein, 127 aa, unidentified
  source 1..127: note = bacteriophage PP7
MSKTIVLSVGEATRTLTEIQSTADRQIFEEKVGPLVGRLRLTASLRQNGAKTAYRVNLKLDQADWDCSTSVCGELPKVRYTQVWSHDVTIVANSTEASRKSLYDLTKSLVATSQVEDLVVNLVPLGR

SEQ ID NO: 110 - DNA, 19 bp, Zea mays
  source 1..19: mol_type = genomic DNA
ctgaatgcctgcgagcatc

SEQ ID NO: 111 - protein, 62 aa, Zea mays
MKSIRCKNCNKLLFKADSFDHIEIRCPRCKRHIIMLNACEHPTEKHCGKREKITHSDETVRY

SEQ ID NO: 112 - protein, 24 aa, synthetic construct
EELLSKNYHLENEVARLKKGSGSG

SEQ ID NO: 113 - protein, 241 aa, synthetic construct
EEELLSKNYHLENEVARLKKGSGSGEELLSKNYHLENEVARLKKGSGSGEELLSKNYHLENEVARLKKGSGSGEELLSKNYHLENEVARLKKGSGSGEELLSKNYHLENEVARLKKGSGSGEELLSKNYHLENEVARLKKGSGSGEELLSKNYHLENEVARLKKGSGSGEELLSKNYHLENEVARLKKGSGSGEELLSKNYHLENEVARLKKGSGSGEELLSKNYHLENEVARLKKGSGSG

SEQ ID NO: 114 - protein, 277 aa, synthetic construct
MGPDIVMTQSPSSLSASVGDRVTITCRSSTGAVTTSNYASWVQEKPGKLFKGLIGGTNNRAPGVPSRFSGSLIGDKATLTISSLQPEDFATYFCALWYSNHWVFGQGTKVELKRGGGGSGGGGSGGGGSSGGGSEVKLLESGGGLVQPGGSLKLSCAVSGFSLTDYGVNWVRQAPGRGLEWIGVIWGDGITDYNSALKDRFIISKDNGKNTVYLQMSKVRSDDTALYYCVTGLFDYWGQGTLVTVSSYPYDVPDYAGGGGGSGGGGSGGGGSGGGGS

SEQ ID NO: 115 - protein, 1227 aa, synthetic construct
SKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIARGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 116 - protein, 1307 aa, unidentified
  source 1..1307: note = Acidaminococcus sp.
MTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRN

SEQ ID NO: 117 - protein, 1241 aa, Butyrivibrio proteoclasticus
MLLYENYTKRNQITKSLRLELRPQGKTLRNIKELNLLEQDKAIYALLERLKPVIDEGIKDIARDTLKNCELSFEKLYEHFLSGDKKAYAKESERLKKEIVKTLIKNLPEGIGKISEINSAKYLNGVLYDFIDKTHKDSEEKQNILSDILETKGYLALFSKFLTSRITTLEQSMPKRVIENFEIYAANIPKMQDALERGAVSFAIEYESICSVDYYNQILSQEDIDSYNRLISGIMDEDGAKEKGINQTISEKNIKIKSEHLEEKPFRILKQLHKQILEEREKAFTIDHIDSDEEVVQVTKEAFEQTKEQWENIKKINGFYAKDPGDITLFIVVGPNQTHVLSQLIYGEHDRIRLLLEEYEKNTLEVLPRRTKSEDARYDKFVNAVPKKVAKESHTFDGLQKMTGDDRLFILYRDELARNYMRIKEAYGTFERDILKSRRGIKGNRDVQESLVSFYDELTKFRSALRIINSGNDEKADPIFYNTFDGIFEKANRTYKAENLCRNYVTKSPADDARIMASCLGTPARLRTHWWNGEENFAINDVAMIRRGDEYYYFVLTPDVKPVDLKTKDETDAQIFVQRKGAKSFLGLPKALFKCILEPYFESPEHKNDKNCVIEEYVSKPLTIDRRAYDIFKNGTFKKTNIGIDGLTEEKFKDDCRYLIDVYKEFIAVYTRYSCFNMSGLKRADEYNDIGEFFSDVDTRLCTMEWIPVSFERINDMVDKKEGLLFLVRSMFLYNRPRKPYERTFIQLFSDSNMEHTSMLLNSRAMIQYRAASLPRRVTHKKGSILVALRDSNGEHIPMHIREAIYKMKNNFDISSEDFIMAKAYLAEHDVAIKKANEDIIRNRRYTEDKFFLSLSYTKNADISARTLDYINDKVEEDTQDSRMAVIVTRNLKDLTYVAVVDEKNNVLEEKSLNEIDGVNYRELLKERTKIKYHDKTRLWQYDVSSKGLKEAYVELAVTQISKLATKYNAVVVVESMSSTFKDKFSFLDEQIFKAFEARLCARMSDLSFNTIKEGEAGSISNPIQVSNNNGNSYQDGVIYFLNNAYTRTLCPDTGFVDVFDKTRLITMQSKRQFFAKMKDIRIDDGEMLFTFNLEEYPTKRLLDRKEWTVKIAGDGSYFDKDKGEYVYVNDIVREQIIPALLEDKAVFDGNMAEKFLDKTAISGKSVELIYKWFANALYGIITKKDGEKIYRSPITGTEIDVSKNTTYNFGKKFMFKQEYRGDGDFLDAFLNYMQAQDIAV

SEQ ID NO: 118 - protein, 1238 aa, Methanoplasma termitum
MNNYDEFTKLYPIQKTIRFELKPQGRTMEHLETFNFFEEDRDRAEKYKILKEAIDEYHKKFIDEHLTNMSLDWNSLKQISEKYYKSREEKDKKVFLSEQKRMRQEIVSEFKKDDRFKDLFSKKLFSELLKEEIYKKGNHQEIDALKSFDKFSGYFIGLHENRKNMYSDGDEITAISNRIVNENFPKFLDNLQKYQEARKKYPEWIIKAESALVAHNIKMDIVFSLEYFNKVLNQEGIQRYNLALGGYVTKSGEKMMGLNDALNLAHQSEKSSKGRIHMTPLFKQILSEKESFSYIPDVFTEDSQLLPSIGGFFAQIENDKDGNIFDRALELISSYAEYDTERIYIRQADINRVSNVIFGEWGTLGGLMREYKADSINDINLERTCKKVDKWLDSKEFALSDVLEAIDRTGNNDAFNEYISKMRTAREKIDAARKEMKFISEKISGDEESIHIIKTLLDSVQQFLHFFNLFKARQDIPLDGAFYAEFDEVHSKLFAIVPLYNKVRNYLTKNNLNTKKIKLNFKNPTLANGWDQNKVYDYASLIFLRDGNYYLGIINPKRKKNIKFEQGSGNGPFYRKMVYKQIPGPNKNLRPVFLTSTKGKKEYKPSKEIIEGYEADKHIRGDKFDLDFCHKLIDFFKESIEKHKDWSKFNFYFSPTESYGDISEFYLDVEKQGYRMHFENISAETIDEYVEKGDLFLFQIYNKDFVKAATGKKDMHTIYWNAAFSPENLQDVVVKLNGEAELFYRDKSDIKEIVHREGEILVNRTYNGRTPVPDKIHKKLTDYHNGRTKDLGEAKEYLDKVRYFKAHYDITKDRRYLNDKIYFHVPLTLNFKANGKKNLNKMVIEKFLSDEKAHIIGIDRGERNLLYYSIIDRSGKIIDQQSLNVIDGFDYREKLNQREIEMKDARQSWNAIGKIKDLKEGYLSKAVHEITKMAIQYNAIVVMEELNYGFKRGRFKVEKQIYQKFENMLIDKMNYLVFKDAPDESPGGVLNAYQLTNPLESFAKLGKQTGILFYVPAAYTSKIDPTTGFVNLFNTSSKTNAQERKEFLQKFESISYSAKDGGIFAFAFDYRKFGTSKTDHKNVWTAYTNGERMRYIKEKKRNELFDPSKEIKEALTSSGIKYDGGQNILPDILRSNNNGLIYTMYSSFIAAIQMRVYDGKEDYIISPIKNSKGEFFRTDPKRRELPIDADANGAYNIALRGELTMRAIAEKFDPDSEKMAKLELKHKDWFEFMQTRGD

SEQ ID NO: 119 - protein, 1281 aa, Eubacterium eligens
MNGNRSIVYREFVGVIPVAKTLRNELRPVGHTQEHIIQNGLIQEDELRQEKSTELKNIMDDYYREYIDKSLSGVTDLDFTLLFELMNLVQSSPSKDNKKALEKEQSKMREQICTHLQSDSNYKNIFNAKLLKEILPDFIKNYNQYDVKDKAGKLETLALFNGFSTYFTDFFEKRKNVFTKEAVSTSIAYRIVHENSLIFLANMTSYKKISEKALDEIEVIEKNNQDKMGDWELNQIFNPDFYNMVLIQSGIDFYNEICGVVNAHMNLYCQQTKNNYNLFKMRKLHKQILAYTSTSFEVPKMFEDDMSVYNAVNAFIDETEKGNIIGKLKDIVNKYDELDEKRIYISKDFYETLSCFMSGNWNLITGCVENFYDENIHAKGKSKEEKVKKAVKEDKYKSINDVNDLVEKYIDEKERNEFKNSNAKQYIREISNIITDTETAHLEYDDHISLIESEEKADEMKKRLDMYMNMYHWAKAFIVDEVLDRDEMFYSDIDDIYNILENIVPLYNRVRNYVTQKPYNSKKIKLNFQSPTLANGWSQSKEFDNNAIILIRDNKYYLAIFNAKNKPDKKIIQGNSDKKNDNDYKKMVYNLLPGANKMLPKVFLSKKGIETFKPSDYIISGYNAHKHIKTSENFDISFCRDLIDYFKNSIEKHAEWRKYEFKFSATDSYSDISEFYREVEMQGYRIDWTYISEADINKLDEEGKIYLFQIYNKDFAENSTGKENLHTMYFKNIFSEENLDKIIKLNGQAELFYRRASVKNPVKHKKDSVLVNKTYKNQLDNGDVVRIPIPDDIYNEIYKMYNGYIKESDLSEAAKEYLDKVEVRTAQKDIVKDYRYTVDKYFIHTPITINYKVTARNNVNDMVVKYIAQNDDIHVIGIDRGERNLIYISVIDSHGNIVKQKSYNILNNYDYKKKLVEKEKTREYARKNWKSIGNIKELKEGYISGVVHEIAMLIVEYNAIIAMEDLNYGFKRGRFKVERQVYQKFESMLINKLNYFASKEKSVDEPGGLLKGYQLTYVPDNIKNLGKQCGVIFYVPAAFTSKIDPSTGFISAFNFKSISTNASRKQFFMQFDEIRYCAEKDMFSFGFDYNNFDTYNITMGKTQWTVYTNGERLQSEFNNARRTGKTKSINLTETIKLLLEDNEINYADGHDIRIDMEKMDEDKKSEFFAQLLSLYKLTVQMRNSYTEAEEQENGISYDKIISPVINDEGEFFDSDNYKESDDKECKMPKDADANGAYCIALKGLYEVLKIKSEWTEDGFDRNCLKLPHAEWLDFIQNKRYE

SEQ ID NO: 120 - protein, 1300 aa, Francisella tularensis
MSIYQEFVNKYSLSKTLRFELIPQGKTLENIKARGLILDDEKRAKDYKKAKQIIDKYHQFFIEEILSSVCISEDLLQNYSDVYFKLKKSDDDNLQKDFKSAKDTIKKQISEYIKDSEKFKNLFNQNLIDAKKGQESDLILWLKQSKDNGIELFKANSDITDIDEALEIIKSFKGWTTYFKGFHENRKVNYSSNDIPTSIIYRIVDDNLPKFLENKAKYESLKDKAPEAINYEQIKKDLAEELTFDIDYKTSEVNQRVFSLDEVFEIANFNNYLNQSGITKFNTIIGGKFVNGENTKRKGINEYINLYSQQINDKTLKKYKMSVLFKQILSDTESKSFVIDKLEDDSDVVTTMQSFYEQIAAFKTVEEKSIKETLSLLFDDLKAQKLDLSKIYFKNDKSLTDLSQQVFDDYSVIGTAVLEYITQQIAPKNLDNPSKKEQELIAKKTEKAKYLSLETIKLALEEFNKHRDIDKQCRFEEILANFAAIPMIFDEIAQNKDNLAQISIKYQNQGKKDLLQASAEDDVKAIKDLLDQTNNLLHKLKIFHISQSEDKANILDKDEHFYLVFEECYFELANIVPLYNKIRNYITQKPYSDEKFKLNFENSTLANGWDKNKEPDNTAILFIKDDKYYLGVMNKKNNKIFDDKAIKENKGEGYKKIVYKLLPGANKMLPKVFFSAKSIKFYNPSEDILRIRNHSTHTKNGSPQKGYEKFEFNIEDCRKFIDFYKQSISKHPEWKDFGFRFSDTQRYNSIDEFYREVENQGYKLTFENISESYIDSVVNQGKLYLFQIYNKDFSAYSKGRPNLHTLYWKALFDERNLQDVVYKLNGEAELFYRKQSIPKKITHPAKEAIANKNKDNPKKESVFEYDLIKDKRFTEDKFFFHCPITINFKSSGANKFNDEINLLLKEKANDVHILSIDRGERHLAYYTLVDGKGNIIKQDTFNIIGNDRMKTNYHDKLAAIEKDRDSARKDWKKINNIKEMKEGYLSQVVHEIAKLVIEYNAIVVFEDLNFGFKRGRFKVEKQVYQKLEKMLIEKLNYLVFKDNEFDKTGGVLRAYQLTAPFETFKKMGKQTGIIYYVPAGFTSKICPVTGFVNQLYPKYESVSKSQEFFSKFDKICYNLDKGYFEFSFDYKNFGDKAAKGKWTIASFGSRLINFRNSDKNHNWDTREVYPTKELEKLLKDYSIEYGHGECIKAAICGESDKKFFAKLTSVLNTILQMRNSKTGTELDYLISPVADVNGNFFDSRQAPKNMPQDADANGAYHIGLKGLMLLGRIKNNQEGKKLNLVIKNEEYFEFVQNRNN

SEQ ID NO: 121 - protein, 1206 aa, unidentified
  source 1..1206: note = Lachnospiraceae bacterium
MYYESLTKQYPVSKTIRNELIPIGKTLDNIRQNNILESDVKRKQNYEHVKGILDEYHKQLINEALDNCTLPSLKIAAEIYLKNQKEVSDREDFNKTQDLLRKEVVEKLKAHENFTKIGKKDILDLLEKLPSISEDDYNALESFRNFYTYFTSYNKVRENLYSDKEKSSTVAYRLINENFPKFLDNVKSYRFVKTAGILADGLGEEEQDSLFIVETFNKTLTQDGIDTYNSQVGKINSSINLYNQKNQKANGFRKIPKMKMLYKQILSDREESFIDEFQSDEVLIDNVESYGSVLIESLKSSKVSAFFDALRESKGKNVYVKNDLAKTAMSVIVFENWRTFDDLLNQEYDLANENKKKDDKYFEKRQKELKKNKSYSLEHLCNLSEDSCNLIENYIHQISDDIENIIINNETFLRIVINEHDRSRKLAKNRKAVKAIKDFLDSIKVLERELKLINSSGQELEKDLIVYSAHEELLVELKQVDSLYNMTRNYLTKKPFSTEKVKLNFNRSTLLNGWDRNKETDNLGVLLLKDGKYYLGIMNTSANKAFVNPPVAKTEKVFKKVDYKLLPVPNQMLPKVFFAKSNIDFYNPSSEIYSNYKKGTHKKGNMFSLEDCHNLIDFFKESISKHEDWSKFGFKFDTQASYNDISEFYREVEKQGYKLTYTDIDETYINDLIERNELYLFQIYNKDFSMYSKGKLNLHTLYFMMLFDQRNIDDVVYKLNGEAEVFYRPASISEDELIIHKAGEEIKNKNPNRARTKETSTFSYDIVKDKRYSKDKFTLHIPITMNFGVDEVKRFNDAVNSAIRIDENVNVIGIDRGERNLLYVVVIDSKGNILEQISLNSIINKEYDIETDYHALLDEREGGRDKARKDWNTVENIRDLKAGLYLQVVNVVAKLVLKYNAIICLEDLNFGFKRGRQKVEKQVYQKFEKMLIDKLNYLVIDKSREQTSPKELGGALNALQLTSKFKSFKELGKQSGVIYYVPAYLTSKIDPTTGFANLFYMKCENVEKSKRFFDGFDFIRFNALENVFEFGFDYRSFTQRACGINSKWTVCTNGERIIKYRNPDKNNMFDEKVVVVTDEMKNLFEQYKIPYEDGRNVKDMIISNEEAEFYRRLYRLLQQTLQMRNSTSDGTRDYIISPVKNKREAYFNSELSDGSVPKDADANGAYNIARKGLWVLEQIRQKSEGEKINLAMTNAEWLEYAQTHLL

SEQ ID NO: 122 - protein, 1233 aa, unidentified
  source 1..1233: note = Lachnospiraceae bacterium
MDYGNGQFERRAPLTKTITLRLKPIGETRETIREQKLLEQDAAFRKLVETVTPIVDDCIRKIADNALCHFGTEYDFSCLGNAISKNDSKAIKKETEKVEKLLAKVLTENLPDGLRKVNDINSAAFIQDTLTSFVQDDADKRVLIQELKGKTVLMQRFLTTRITALTVWLPDRVFENFNIFIENAEKMRILLDSPLNEKIMKFDPDAEQYASLEFYGQCLSQKDIDSYNLIISGIYADDEVKNPGINEIVKEYNQQIRGDKDESPLPKLKKLHKQILMPVEKAFFVRVLSNDSDARSILEKILKDTEMLPSKIIEAMKEADAGDIAVYGSRLHELSHVIYGDHGKLSQIIYDKESKRISELMETLSPKERKESKKRLEGLEEHIRKSTYTFDELNRYAEKNVMAAYIAAVEESCAEIMRKEKDLRTLLSKEDVKIRGNRHNTLIVKNYFNAWTVFRNLIRILRRKSEAEIDSDFYDVLDDSVEVLSLTYKGENLCRSYITKKIGSDLKPEIATYGSALRPNSRWWSPGEKFNVKFHTIVRRDGRLYYFILPKGAKPVELEDMDGDIECLQMRKIPNPTIFLPKLVFKDPEAFFRDNPEADEFVFLSGMKAPVTITRETYEAYRYKLYTVGKLRDGEVSEEEYKRALLQVLTAYKEFLENRMIYADLNFGFKDLEEYKDSSEFIKQVETHNTFMCWAKVSSSQLDDLVKSGNGLLFEIWSERLESYYKYGNEKVLRGYEGVLLSILKDENLVSMRTLLNSRPMLVYRPKESSKPMVVHRDGSRVVDRFDKDGKYIPPEVHDELYRFFNNLLIKEKLGEKARKILDNKKVKVKVLESERVKWSKFYDEQFAVTFSVKKNADCLDTTKDLNAEVMEQYSESNRLILIRNTTDILYYLVLDKNGKVLKQRSLNIINDGARDVDWKERFRQVTKDRNEGYNEWDYSRTSNDLKEVYLNYALKEIAEAVIEYNAILIIEKMSNAFKDKYSFLDDVTFKGFETKKLAKLSDLHFRGIKDGEPCSFTNPLQLCQNDSNKILQDGVIFMVPNSMTRSLDPDTGFIFAINDHNIRTKKAKLNFLSKFDQLKVSSEGCLIMKYSGDSLPTHNTDNRVWNCCCNHPITNYDRETKKVEFIEEPVEELSRVLEENGIETDTELNKLNERENVPGKVVDAIYSLVLNYLRGTVSGVAGQRAVYYSPVTGKKYDISFIQAMNLNRKCDYYRIGSKERGEWTDFVAQLIN

SEQ ID NO: 123 - protein, 1227 aa, unidentified
  source 1..1227: note = Lachnospiraceae bacterium
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIANINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIASNKEWLEYAQTSVKH

SEQ ID NO: 124 - protein, 1264 aa, unidentified
  source 1..1264: note = Lachnospiraceae bacterium
MEDYSGFVNIYSIQKTLRFELKPVGKTLEHIEKKGFLKKDKIRAEDYKAVKKIIDKYHRAYIEEVFDSVLHQKKKKDKTRFSTQFIKEIKEFSELYYKTEKNIPDKERLEALSEKLRKMLVGAFKGEFSEEVAEKYNKNLFSKELIRNEIEKFCETDEERKQVSNFKSFTTYFTGFHSNRQNIYSDEKKSTAIGYRIIHQNLPKFLDNLKIIESIQRRFKDFPWSDLKKNLKKIDKNIKLTEYFSIDGFVNVLNQKGIDAYNTILGGKSEESGEKIQGLNEYINLYRQKNNIDRKNPLNVKILFKQILGDRETKSFIPEAFPDDQSVLNSITEFAKYLKLDKKKKSIIAELKKFLSSFNRYELDGIYLANDNSLASISTFLFDDWSFIKKSVSFKYDESVGDPKKKIKSPLKYEKEKEKWLKQKYYTISFLNDAIESYSKSQDEKRVKIRLEAYFAEFKSKDDAKKQFDLLERIEEAYAIVEPLLGAEYPRDRNLKADKKEVGKIKDFLDSIKSLQFFLKPLLSAEIFDEKDLGFYNQLEGYYEEIDISGHLYNKVRNYLTGKIYSKEKFKLNFENSTLLKGWDENREVANLCVIFREDQKYYLGVMDKENNTILSDIPKVKPNELFYEKMVYKLIPTPHMQLPRIIFSSDNLSIYNPSKSILKIREAKSFKEGKNFKLKDCHKFIDFYKESISKNEDWSRFDFKFSKTSSYENISEFYREVERQGYNLDFKKVSKFYIDSLVEDGKLYLFQIYNKDFSIFSKGKPNLHTIYFRSLFSKENLKDVCLKLNGEAEMFFRKKSINYDEKKKREGHHPELFEKLKYPILKDKRYSEDKFQFHLPISLNFKSKERLNFNLKVNEFLKRNKDINIIGIDRGERNLLYLVMINQKGEILKQTLLDSMQSGKGRPEINYKEKLQEKEIERDKARKSWGTVENIKELKEGYLSIVIHQISKLMVENNAIVVLEDLNIGFKRGRQKVERQVYQKFEKMLIDKLNFLVFKENKPTEPGGVLKAYQLTDEFQSFEKLSKQTGFLFYVPSWNTSKIDPRTGFIDFLHPAYENIEKAKQWINKFDSIRFNSKMDWFEFTADTRKFSENLMLGKNRVWVICTTNVERYFTSKTANSSIQYNSIQITEKLKELFVDIPFSNGQDLKPEILRKNDAVFFKSLLFYIKTTLSLRQNNGKKGEEEKDFILSPVVDSKGRFFNSLEASDDEPKDADANGAYHIALKGLMNLLVLNETKEENLSRPKWKIKNKDWLEFVWERNR

SEQ ID NO: 125 - protein, 1373 aa, Moraxella bovoculi
MLFQDFTHLYPLSKTVRFELFIDRTLEHIHAKNFLSQDETMADMHQKVKVILDDYHRDFIADMMGEVKLTKLAEFYDVYLKFRKNPKDDELQKAQLKDLQAVLRKEIVKPIGNGGKYKAGYDRLFGAKLFKDGKELGDLAKFVIAQEGESSPKLAHLAHFEKFSTYFTGFHDNRKNMYSDEDKHTAIAYRLIHENLPRFIDNLQILTTIKQKHSALYDQIINELTASGLDVSLASHLDGYHKLLTQEGITAYNTLLGGISGEAGSPKIQGINELINSHHNQHCHKSERIAKLRPLHKQILSDGMSVSFLPSKFADDSEMCQAVNEFYRHYADVFAKVQSLFDGFDDHQKDGIYVEHKNLNELSKQAFGDFALLGRVLDGYYVDVVNPEFNERFAKAKTDNAKAKLTKEKDKFIKGVHSLASLEQAIEHYTARHDDESVQAGKLGQYFKHGLAGVDNPIQKIHNNHSTIKGFLERERPAGERALPKIKSGKNPEMTQLRQLKELLDNALNVAHFAKLLTTKTTLDNQDGNFYGEFGVLYDELAKIPTLYNKVRDYLSQKPFSTEKYKLNFGNPTLLNGWDLNKEKDNFGVILQKDGCYYLALLDKAHKKVFDNAPNTGKSIYQKMIYKYLEVRKQFPKVFFSKEAIAINYHPSKELVEIKDKGRQRSDDERLKLYRFILECLKIHPKYDKKFEGAIGDIQLFKKDKKGREVPISEKDLFKDINGIFSSKPKLEMEDFFIGEFKRYNPSQDLVDQYNIYKKIDSNDNRKKENFYNNHPKFKKDLVRYYYESMCKHEEWEESFEFSKKLQDIGCYVDVNELFTEIETRRLNYKISFCNINADYIDELVEQGQLYLFQIYNKDFSPKAHGKPNLHTLYFKALFSEDNLADPIYKLNGEAQIFYRKASLDMNETTIHRAGEVLENKNPDNPKKRQFVYDIIKDKRYTQKDFMLHVPITMNFGVQGMTIKEFNKKVNQSIQQYDEVNVIGIDRGERHLLYLTVINSKGEILEQCSLNDITTASANGTQMTTPYHKILDKREIERLNARVGWGEIETIKELKSGYLSHVVHQISQLMLKYNAIVVLEDLNFGFKRGRFKVEKQIYQNFENALIKKLNHLVLKDKADDEIGSYKNALQLTNNFTDLKSIGKQTGFLFYVPAWNTSKIDPETGFVDLLKPRYENIQASQAFFGKFDKICYNADKDYFEFHIDYAKFTDKAKNSRQIWTICSHGDKRYVYDKTANQNKGAAKGINVNDILKSLFARHHINEKQPNLVMDICQNNDKEFHKSLMYLLKTLLALRYSNASSDEDFILSPVANDEGVFFNSALADDTQPQNADANGAYHIALKGLWLLNELKNSDDLNKVKLAIDNQTWLNFAQNR

SEQ ID NO: 126 - protein, 1352 aa, unidentified
  source 1..1352: note = Parcubacteria bacterium
MENIFDQFIGKYSLSKTLRFELKPVGKTEDFLKINKVFEKDQTIDDSYNQAKFYFDSLHQKFIDAALASDKTSELSFQNFADVLEKQNKIILDKKREMGALRKRDKNAVGIDRLQKEINDAEDIIQKEKEKIYKDVRTLFDNEAESWKTYYQEREVDGKKITESKADLKQKGADFLTAAGILKVLKYEFPEEKEKEFQAKNQPSLFVEEKENPGQKRYIFDSFDKFAGYLTKFQQTKKNLYAADGTSTAVATRIADNFIIFHQNTKVFRDKYKNNHTDLGFDEENIFEIERYKNCLLQREIEHIKNENSYNKIIGRINKKIKEYRDQKAKDTKLTKSDFPFFKNLDKQILGEVEKEKQLIEKTREKTEEDVLIERFKEFIENNEERFTAAKKLMNAFCNGEFESEYEGIYLKNKAINTISRRWFVSDRDFELKLPQQKSKNKSEKNEPKVKKFISIAEIKNAVEELDGDIFKAVFYDKKIIAQGGSKLEQFLVIWKYEFEYLFRDIERENGEKLLGYDSCLKIAKQLGIFPQEKEAREKATAVIKNYADAGLGIFQMMKYFSLDDKDRKNTPGQLSTNFYAEYDGYYKDFEFIKYYNEFRNFITKKPFDEDKIKLNFENGALLKGWDENKEYDFMGVILKKEGRLYLGIMHKNHRKLFQSMGNAKGDNANRYQKMIYKQIADASKDVPRLLLTSKKAMEKFKPSQEILRIKKEKTFKRESKNFSLRDLHALIEYYRNCIPQYSNWSFYDFQFQDTGKYQNIKEFTDDVQKYGYKISFRDIDDEYINQALNEGKMYLFEVVNKDIYNTKNGSKNLHTLYFEHILSAENLNDPVFKLSGMAEIFQRQPSVNEREKITTQKNQCILDKGDRAYKYRRYTEKKIMFHMSLVLNTGKGEIKQVQFNKIINQRISSSDNEMRVNVIGIDRGEKNLLYYSVVKQNGEIIEQASLNEINGVNYRDKLIEREKERLKNRQSWKPVVKIKDLKKGYISHVIHKICQLIEKYSAIVVLEDLNMRFKQIRGGIERSVYQQFEKALIDKLGYLVFKDNRDLRAPGGVLNGYQLSAPFVSFEKMRKQTGILFYTQAEYTSKTDPITGFRKNVYISNSASLDKIKEAVKKFDAIGWDGKEQSYFFKYNPYNLADEKYKNSTVSKEWAIFASAPRIRRQKGEDGYWKYDRVKVNEEFEKLLKVWNFVNPKATDIKQEIIKKIKAGDLQGEKELDGRLRNFWHSFIYLFNLVLELRNSFSLQIKIKAGEVIAVDEGVDFIASPVKPFFTTPNPYIPSNLCWLAVENADANGAYNIARKGVMILKKIREHAKKDPEFKKLPNLFISNAEWDEAARDWGKYAGTTALNLDH

SEQ ID NO: 127 - protein, 1260 aa, Porphyromonas crevioricanis
MDSLKDFTNLYPVSKTLRFELKPVGKTLENIEKAGILKEDEHRAESYRRVKKIIDTYHKVFIDSSLENMAKMGIENEIKAMLQSFCELYKKDHRTEGEDKALDKIRAVLRGLIVGAFTGVCGRRENTVQNEKYESLFKEKLIKEILPDFVLSTEAESLPFSVEEATRSLKEFDSFTSYFAGFYENRKNIYSTKPQSTAIAYRLIHENLPKFIDNILVFQKIKEPIAKELEHIRADFSAGGYIKKDERLEDIFSLNYYIHVLSQAGIEKYNALIGKIVTEGDGEMKGLNEHINLYNQQRGREDRLPLFRPLYKQILSDREQLSYLPESFEKDEELLRALKEFYDHIAEDILGRTQQLMTSISEYDLSRIYVRNDSQLTDISKKMLGDWNAIYMARERAYDHEQAPKRITAKYERDRIKALKGEESISLANLNSCIAFLDNVRDCRVDTYLSTLGQKEGPHGLSNLVENVFASYHEAEQLLSFPYPEENNLIQDKDNVVLIKNLLDNISDLQRFLKPLWGMGDEPDKDERFYGEYNYIRGALDQVIPLYNKVRNYLTRKPYSTRKVKLNFGNSQLLSGWDRNKEKDNSCVILRKGQNFYLAIMNNRHKRSFENKMLPEYKEGEPYFEKMDYKFLPDPNKMLPKVFLSKKGIEIYKPSPKLLEQYGHGTHKKGDTFSMDDLHELIDFFKHSIEAHEDWKQFGFKFSDTATYENVSSFYREVEDQGYKLSFRKVSESYVYSLIDQGKLYLFQIYNKDFSPCSKGTPNLHTLYWRMLFDERNLADVIYKLDGKAEIFFREKSLKNDHPTHPAGKPIKKKSRQKKGEESLFEYDLVKDRRYTMDKFQFHVPITMNFKCSAGSKVNDMVNAHIREAKDMHVIGIDRGERNLLYICVIDSRGTILDQISLNTINDIDYHDLLESRDKDRQQEHRNWQTIEGIKELKQGYLSQAVHRIAELMVAYKAVVALEDLNMGFKRGRQKVESSVYQQFEKQLIDKLNYLVDKKKRPEDIGGLLRAYQFTAPFKSFKEMGKQNGFLFYIPAWNTSNIDPTTGFVNLFHVQYENVDKAKSFFQKFDSISYNPKKDWFEFAFDYKNFTKKAEGSRSMWILCTHGSRIKNFRNSQKNGQWDSEEFALTEAFKSLFVRYEIDYTADLKTAIVDEKQKDFFVDLLKLFKLTVQMRNSWKEKDLDYLISPVAGADGRFFDTREGNKSLPKDADANGAYNIALKGLWALRQIRQTSEGGKLKLAISNKEWLQFVQERSYEKD

SEQ ID NO: 128 - protein, 1324 aa, Prevotella disiens
MENYQEFTNLFQLNKTLRFELKPIGKTCELLEEGKIFASGSFLEKDKVRADNVSYVKKEIDKKHKIFIEETLSSFSISNDLLKQYFDCYNELKAFKKDCKSDEEEVKKTALRNKCTSIQRAMREAISQAFLKSPQKKLLAIKNLIENVFKADENVQHFSEFTSYFSGFETNRENFYSDEEKSTSIAYRLVHDNLPIFIKNIYIFEKLKEQFDAKTLSEIFENYKLYVAGSSLDEVFSLEYFNNTLTQKGIDNYNAVIGKIVKEDKQEIQGLNEHINLYNQKHKDRRLPFFISLKKQILSDREALSWLPDMFKNDSEVIDALKGFYIEDGFENNVLTPLATLLSSLDKYNLNGIFIRNNEALSSLSQNVYRNFSIDEAIDAQNAELQTFNNYELIANALRAKIKKETKQGRKSFEKYEEYIDKKVKAIDSLSIQEINELVENYVSEFNSNSGNMPRKVEDYFSLMRKGDFGSNDLIENIKTKLSAAEKLLGTKYQETAKDIFKKDENSKLIKELLDATKQFQHFIKPLLGTGEEADRDLVFYGDFLPLYEKFEELTLLYNKVRNRLTQKPYSKDKIRLCFNKPKLMTGWVDSKTEKSDNGTQYGGYLFRKKNEIGEYDYFLGISSKAQLFRKNEAVIGDYERLDYYQPKANTIYGSAYEGENSYKEDKKRLNKVIIAYIEQIKQTNIKKSIIESISKYPNISDDDKVTPSSLLEKIKKVSIDSYNGILSFKSFQSVNKEVIDNLLKTISPLKNKAEFLDLINKDYQIFTEVQAVIDEICKQKTFIYFPISNVELEKEMGDKDKPLCLFQISNKDLSFAKTFSANLRKKRGAENLHTMLFKALMEGNQDNLDLGSGAIFYRAKSLDGNKPTHPANEAIKCRNVANKDKVSLFTYDIYKNRRYMENKFLFHLSIVQNYKAANDSAQLNSSATEYIRKADDLHIIGIDRGERNLLYYSVIDMKGNIVEQDSLNIIRNNDLETDYHDLLDKREKERKANRQNWEAVEGIKDLKKGYLSQAVHQIAQLMLKYNAIIALEDLGQMFVTRGQKIEKAVYQQFEKSLVDKLSYLVDKKRPYNELGGILKAYQLASSITKNNSDKQNGFLFYVPAWNTSKIDPVTGFTDLLRPKAMTIKEAQDFFGAFDNISYNDKGYFEFETNYDKFKIRMKSAQTRWTICTFGNRIKRKKDKNYWNYEEVELTEEFKKLFKDSNIDYENCNLKEEIQNKDNRKFFDDLIKLLQLTLQMRNSDDKGNDYIISPVANAEGQFFDSRNGDKKLPLDADANGAYNIARKGLWNIRQIKQTKNKDDLNLSISSTEWLDFVREKPYLK

SEQ ID NO: 129 - protein, 1484 aa, unidentified
  VARIANT 1073: note = Xaa can be any naturally occurring amino acid
  source 1..1484: note = Peregrinibacteria bacterium
MSNFFKNFTNLYELSKTLRFELKPVGDTLTNMKDHLEYDEKLQTFLKDQNIDDAYQALKPQFDEIHEEFITDSLESKKAKEIDFSEYLDLFQEKKELNDSEKKLRNKIGETFNKAGEKWKKEKYPQYEWKKGSKIANGADILSCQDMLQFIKYKNPEDEKIKNYIDDTLKGFFTYFGGFNQNRANYYETKKEASTAVATRIVHENLPKFCDNVIQFKHIIKRKKDGTVEKTERKTEYLNAYQYLKNNNKITQIKDAETEKMIESTPIAEKIFDVYYFSSCLSQKQIEEYNRIIGHYNLLINLYNQAKRSEGKHLSANEKKYKDLPKFKTLYKQIGCGKKKDLFYTIKCDTEEEANKSRNEGKESHSVEEIINKAQEAINKYFKSNNDCENINTVPDFINYILTKENYEGVYWSKAAMNTISDKYFANYHDLQDRLKEAKVFQKADKKSEDDIKIPEAIELSGLFGVLDSLADWQTTLFKSSILSNEKLKIITDSQTPSEALLKMIFNDIEKNMESFLKETNDIITLKKYKGNKEGTEKIKQWFDYTLAINRMLKYFLVKENKIKGNSLDTNISEALKTLIYSDDAEWFKWYDALRNYLTQKPQDEAKENKLKLNFDNPSLAGGWDVNKECSNFCVILKDKNEKKYLAMIKKGENTLFQKEWTEGRGKNLTKKSNPLFEINNCEILSKMEYDFWADVSKMIPKCSTQLKAVVNHFKQSDNEFIFPIGYKVTSGEKFREECKISKQDFELNNKVFNKNELSVTAMRYDLSSTQEKQYIKAFQKEYWELLFKQEKRDTKLTNNEIFNEWINFCNKKYSELLSWERKYKDALTNWINFCKYFLSKYPKTTLFNYSFKESENYNSLDEFYRDVDICSYKLNINTTINKSILDRLVEEGKLYLFEIKNQDSNDGKSIGHKNNLHTIYWNAIFENFDNRPKLNGEAEIFYRKAISKDKLGIVKGKKTKNGTWIIKNYRFSKEKFILHVPITLNFCSNNEYVNDIVNTKFYNFSNLHFLGIDRGEKHLAYYSLVNKNGEIVDQGTLNLPFTDKDGNQRSIKKEKYFYNKQEDKWEAKEVDXWNYNDLLDAMASNRDMARKNWQRIGTIKEAKNGYVSLVIRKIADLAVNNERPAFIVLEDLNTGFKRSRQKIDKSVYQKFELALAKKLNFLVDKNAKRDEIGSPTKALQLTPPVNNYGDIENKKQAGIMLYTRANYTSQTDPATGWRKTIYLKAGPEETTYKKDGKIKNKSVKDQIIETFTDIGFDGKDYYFEYDKGEFVDEKTGEIKPKKWRLYSGENGKSLDRFRGEREKDKYEWKIDKIDIVKILDDLFVNFDKNISLLKQLKEGVELTRNNEHGTGESLRFAINLIQQIRNTGNNERDNDFILSPVRDENGKHFDSREYWDKETKGEKISMPSSGDANGAFNIARKGIIMNAHILANSDSKDLSLFVSDEEWDLHLNNKTEWKKQLNIFSSRKAMAKRKKKRPAATKK

SEQ ID NO: 130 - protein, 1245 aa, Porphyromonas macacae
MKTQHFFEDFTSLYSLSKTIRFELKPIGKTLENIKKNGLIRRDEQRLDDYEKLKKVIDEYHEDFIANILSSFSFSEEILQSYIQNLSISEARAKIEKTMRDTLAKAFSEDERYKSIFKKELVKKDIPVWCPAYKSLCKKFDNFTTSLVPFHENRKNLYTSNEITASIPYRIVHVNLPKFIQNIEALCELQKKMGADLYLEMMENLRNVWPSFVKTPDDLCNLKTYNHLMVQSSISEYNRFVGGYSTEDGTKHQGINEWINIYRQRNKEMRLPGLVFLHKQILAKVDSSSFISDTLENDDQVFCVLRQFRKLFWNTVSSKEDDAASLKDLFCGLSGYDPEAIYVSDAHLATISKNIFDRWNYISDAIRRKTEVLMPRKKESVERYAEKISKQIKKRQSYSLAELDDLLAHYSEESLPAGFSLLSYFTSLGGQKYLVSDGEVILYEEGSNIWDEVLIAFRDLQVILDKDFTEKKLGKDEEAVSVIKKALDSALRLRKFFDLLSGTGAEIRRDSSFYALYTDRMDKLKGLLKMYDKVRNYLTKKPYSIEKFKLHFDNPSLLSGWDKNKELNNLSVIFRQNGYYYLGIMTPKGKNLFKTLPKLGAEEMFYEKMEYKQIAEPMLMLPKVFFPKKTKPAFAPDQSVVDIYNKKTFKTGQKGFNKKDLYRLIDFYKEALTVHEWKLFNFSFSPTEQYRNIGEFFDEVREQAYKVSMVNVPASYIDEAVENGKLYLFQIYNKDFSPYSKGIPNLHTLYWKALFSEQNQSRVYKLCGGGELFYRKASLHMQDTTVHPKGISIHKKNLNKKGETSLFNYDLVKDKRFTEDKFFFHVPISINYKNKKITNVNQMVRDYIAQNDDLQHGIDRGERNLLYISRIDTRGNLLEQFSLNVIESDKGDLRTDYQKILGDREQERLRRRQEWKSIESIKDLKDGYMSQVVHKICNMVVEHKAIVVLENLNLSFMKGRKKVEKSVYEKFERMLVDKLNYLVVDKKNLSNEPGGLYAAYQLTNPLFSFEELHRYPQSGILFFVDPWNTSLTDPSTGFVNLLGRINYTNVGDARKFFDRFNAIRYDGKGNILFDLDLSRFDVRVETQRKLWTLTTFGSRIAKSKKSGKWMVERIENLSLCFLELFEQFNIGYRVEKDLKKAILSQDRKEFYVRLIYLFNLMMQIRNSDGEEDYILSPALNEKNLQFDSRLIEAKDLPVDADANGAYNVARKGLMVVQRIKRGDHESIHRIGRAQWLRYVQEGIVE

SEQ ID NO: 131 - protein, 1250 aa, Smithella sp.
MQTLFENFTNQYPVSKTLRFELIPQGKTKDFIEQKGLLKKDEDRAEKYKKVKNIIDEYHKDFIEKSLNGLKLDGLEKYKTLYLKQEKDDKDKKAFDKEKENLRKQIANAFRNNEKFKTLFAKELIKNDLMSFACEEDKKNVKEFEAFTTYFTGFHQNRANMYVADEKRTAIASRLIHENLPKFIDNIKIFEKMKKEAPELLSPFNQTLKDMKDVIKGTTLEEIFSLDYFNKTLTQSGIDIYNSVIGGRTPEEGKTKIKGLNEYINTDFNQKQTDKKKRQPKFKQLYKQILSDRQSLSFIAEAFKNDTEILEAIEKFYVNELLHFSNEGKSTNVLDAIKNAVSNLESFNLTKMYFRSGASLTDVSRKVFGEWSIINRALDNYYATTYPIKPREKSEKYEERKEKWLKQDFNVSLIQTAIDEYDNETVKGKNSGKVIADYFAKFCDDKETDLIQKVNEGYIAVKDLLNTPCPENEKLGSNKDQVKQIKAFMDSIMDIMHFVRPLSLKDTDKEKDETFYSLFTPLYDHLTQTIALYNKVRNYLTQKPYSTEKIKLNFENSTLLGGWDLNKETDNTAIILRKDNLYYLGIMDKRHNRIFRNVPKADKKDFCYEKMVYKLLPGANKMLPKVFFSQSRIQEFTPSAKLLENYANETHKKGDNFNLNHCHKLIDFFKDSINKHEDWKNFDFRFSATSTYADLSGFYHEVEHQGYKISFQSVADSFIDDLVNEGKLYLFQIYNKDFSPFSKGKPNLHTLYWKMLFDENNLKDVVYKLNGEAEVFYRKKSIAEKNTTIHKANESIINKNPDNPKATSTFNYDIVKDKRYTIDKFQFHIPITMNFKAEGIFNMNQRVNQFLKANPDINIIGIDRGERHLLYYALINQKGKILKQDTLNVIANEKQKVDYHNLLDKKEGDRATARQEWGVIETIKELKEGYLSQVIHKLTDLMIENNAIIVMEDLNFGFKRGRQKVEKQVYQKFEKMLIDKLNYLVDKNKKANELGGLLNAFQLANKFESFQKMGKQNGFIFYVPAWNTSKTDPATGFIDFLKPRYENLNQAKDFFEKFDSIRLNSKADYFEFAFDFKNFTEKADGGRTKWTVCTTNEDRYQWNRALNNNRGSQEKYDITAELKSLFDGKVDYKSGKDLKQQIASQESADFFKALMKNLSITLSLRHNNGEKGDNEQDYILSPVADSKGRFFDSRKADDDMPKNADANGAYHIALKGLWCLEQISKTDDLKKVKLAISNKEWLEFVQTLKG

SEQ ID NO: 132 - DNA, 3987 bp, synthetic construct
  source 1..3987: mol_type = other DNA
atggccgggagcaagaagcgccggataaagcaggacacgcagttcgagggcttcaccaacctgtaccaagtctccaagacgctccggttcgagcttatcccgcaagggaagaccctgaaacacatccaggaacaaggtttcatcgaggaggacaaggcccgcaacgaccactacaaggagctcaagcccataatcgatcggatctacaagacgtacgccgaccagtgcctccaactggtgcagctcgactgggagaacctgagcgccgccattgacagctaccgcaaggaaaagacggaggagacgcgcaacgcccttattgaggagcaagccacctaccgcaacgccatccacgactacttcatcgggcgcaccgacaacctgacggacgcgatcaacaagcgccacgcggaaatctacaagggccttttcaaggccgagctcttcaacgggaaggtcctaaaacagctcgggactgtcacgacaaccgagcatgagaacgccctccttcgcagcttcgacaagttcaccacatacttctcgggcttctaccggaaccgcaagaacgttttcagcgccgaggacatctccaccgccatcccgcacaggatcgtccaggacaacttccccaagttcaaggagaactgccacatcttcacgcgcctgattacagccgtaccttcacttcgtgagcacttcgagaacgtcaaaaaggccatcgggatcttcgtctccacgtccatcgaggaggtattctctttcccgttctataaccagctcctgacccagacgcagatcgacctctacaaccagctactgggcggcatcagccgggaggccgggaccgagaaaataaagggcctcaacgaagttctcaacctggccatccagaagaacgacgagaccgcgcatatcatcgcatccctgccgcatcgcttcattcctttgttcaagcagatattgagcgaccggaacaccctctcgttcatcctcgaagaattcaagagcgacgaggaggtcattcagtctttctgcaagtacaagacgctcctacggaatgagaatgtgctggagaccgcggaggcactcttcaatgagctgaactccattgacctgacccacatcttcattagccacaagaaactggagacgatctccagcgccctgtgcgaccactgggacactctccgcaacgccctctacgaacgccggatctccgaacttaccggcaagataactaagtcggctaaggagaaggtgcaacggagcctcaagcacgaggacatcaaccttcaggaaatcatctcagccgcgggcaaggagctgagcgaggcgtttaagcagaaaacatcggagatactgagccacgcgcacgcggccctggatcaaccgctgccgacgactctcaagaagcaagaggagaaggaaatccttaagtcccagctcgactcgctgctcggcctctatcacttgctcgactggttcgcggttgatgagtccaacgaggtggacccggagttctccgcgcgcctcacgggtattaagctggagatggagccaagcttaagcttctacaacaaggcccgcaactacgcgaccaaaaaaccgtactcagtcgagaaattcaagctgaatttccagatgcctacattggcgagggggtgggacgtgaaccgcgagaagaacaatggagccatcctgttcgtcaaaaatgggttgtactacctgggcatcatgcccaagcagaagggccgttacaaggccctgtcattcgagcctaccgagaagacctcggagggcttcgacaagatgtactacgactatttcccggacgccgccaagatgatcccgaagtgctccacgcagctcaaagccgtcacggcccacttccagacgcataccacgccgatacttctgagcaacaacttcattgagccgctagagatcacgaaggagatatacgacctaaacaaccccgaaaaggagcccaagaagttccagacagcctacgctaagaagacaggtgatcagaagggatatagggaggcactctgcaagtggatcgacttcacgcgcgacttcctgtcgaaatatacaaagacgaccagcattgacctaagttctctccgcccatcctcccagtacaaggatctgggcgagtattatgcggagctgaacccattgctgtaccacatcagcttccagaggatcgccgagaaggagattatggacgcggtggagacggggaaactatacctgttccaaatatataacaaggacttcgctaaagggcaccacgggaagcccaacctgcacacactctactggacgggcttgttttcgccagaaaatttggccaagacttcgatcaagctcaacggccaggcggagttgttttaccgtcccaagtctcgcatgaagcgcatggcgcatcgcctcggagagaaaatgcttaacaagaagctcaaggatcagaagacgcccatacctgatacgttgtaccaggaattgtacgactacgtgaaccaccgcctatcgcacgacctctcagacgaggcccgcgccctcctcccaaacgtgattactaaggaggtttcccatgaaataatcaaggaccgacggttcaccagcgacaaattttttttccacgtgcctatcacgctcaattaccaggcggccaactccccatcgaagttcaaccagcgcgtgaacgcctaccttaaggagcacccggagaccccaatcatcgggatcgaccgtggcgagcggaacctgatctatattacggtgatcgatagcaccgggaagatcctggagcagcgctccctgaacacaatccagcagtttgactaccagaagaaactcgacaaccgggagaaggagcgcgtcgcagcccggcaagcatggagtgtggtcggcaccataaaggacctgaaacagggttacctaagtcaagttatccacgagatcgttgacctgatgatacactatcaagccgtagtcgtgctggagaacctcaacttcgggtttaagtccaagcgcaccggcatcgcggagaaggcggtgtaccagcagttcgagaagatgctgatcgacaagctgaactgcctggtgctcaaggactaccctgcggagaaggtcggcggggtcttgaacccgtaccagctaaccgaccagttcacgagcttcgccaaaatgggcacgcagtccggattcttgttttatgtcccggctccatatacaagtaagatcgacccgctgacagggtttgttgacccattcgtgtggaagaccatcaagaaccacgagagcaggaaacacttcttagagggcttcgacttcctgcattacgacgttaagacaggcgacttcatcctgcacttcaagatgaaccgcaacctgtcgttccagaggggcctgcccggcttcatgcccgcctgggatatcgtctttgagaagaatgagacgcagttcgacgcgaaggggacgccgttcatcgctggaaagcggatcgtgccggtcatcgagaaccaccgcttcacgggtcgctaccgagatttataccccgccaacgaactaattgcgctgctggaggagaaggggatcgtgttccgagatggcagcaacattctcccgaagctgctggagaacgacgactcgcacgctattgacacgatggtcgccctcatacggagcgtgcttcagatgcggaacagtaacgctgccacgggcgaggactacattaactcccccgtccgcgacctcaacggggtctgcttcgatagccgcttccagaacccggagtggcctatggatgcggacgcgaacggggcctaccacatcgccctcaagggccaactcctgctcaaccacttgaaggaaagcaaagacctcaaattgcagaatggcatcagtaaccaggactggctcgcgtacatccaggaactgagaaacgggtccaagaagcggcgtatcaagcaagattga

SEQ ID NO: 133 - DNA, 3987 bp, synthetic construct
  source 1..3987: mol_type = other DNA
atggcgggaagcaaaaagcgccggattaagcaagacacgcagttcgagggcttcacgaacctctaccaagtcagcaagaccctccggttcgagctgataccacagggaaagacgctcaagcacatccaggaacagggcttcatcgaggaggacaaggcgcgcaacgaccactacaaggagttgaaaccgatcatcgaccgcatctacaagacgtacgccgaccagtgcctccagctcgtgcagctcgactgggagaacctctccgccgccattgactcgtaccggaaggagaagactgaggagacccgcaacgccctgatcgaggagcaagcaacctaccggaacgccatccacgactacttcatcggccgcaccgacaacctcaccgacgcgatcaacaagcggcacgcggagatatacaaagggctgttcaaggcggagctgttcaacggcaaggtgctcaagcagctagggacggtgaccacgaccgagcacgagaacgcgctcctccgcagcttcgacaagttcaccacctacttcagcggcttctaccggaaccgcaagaatgtgttcagcgcggaggacatcagcacggccatcccgcaccgcatcgtccaggacaacttcccgaagttcaaggagaactgccacatcttcacccgcctgataaccgccgtcccctccctgcgggagcacttcgagaacgtcaaaaaggcaattgggatcttcgtctcgaccagcattgaggaggtgttcagcttccccttctacaaccagctcctcacccagacgcagatcgacctgtacaatcagttgctcggcgggataagccgcgaggcgggaaccgaaaaaatcaaggggctgaacgaagtgttgaacctcgccatccagaagaacgacgagaccgcgcacatcatcgcctccctgccccaccggttcatcccgctgttcaagcagatcctctctgaccggaacaccctgtccttcattcttgaggagttcaagtcggacgaggaggtcatccagagcttctgcaagtacaagacgctgctacggaacgagaacgtgctggagacggcggaggcactgttcaacgagctaaacagcatcgacctcacgcacatcttcatcagtcacaagaaactggagaccatctcctccgcgctgtgcgaccactgggacacgctcaggaacgcgctctacgagcgccgaatcagtgagctgacgggcaagatcacgaagtccgcgaaggagaaggtgcagcggtccctcaagcacgaggacatcaacctccaggagatcatctcagcggctgggaaagagctgtccgaggcgttcaagcagaaaacgagcgaaatcctgtcccacgcgcacgcggccctggatcagcctctgccgacgaccctcaagaaacaagaagaaaaggaaatcctcaagtcgcagctcgactcgctgctgggcctgtaccatctcctcgactggttcgccgtggacgagagcaacgaggtggaccccgagttctccgcgcggcttacggggatcaagctggagatggagcccagcctgtccttctacaacaaggcgcgcaactacgccaccaagaagccctacagcgtggagaagttcaagctcaacttccagatgcccactctcgcacgtgggtgggacgtcaaccgcgaaaaaaataatggggcgatcctgttcgtcaagaacggcctgtactacttgggcatcatgccgaaacagaagggccgctacaaggccctgagcttcgaaccgaccgagaaaacgagcgaggggttcgacaagatgtactacgactacttccccgacgccgcgaagatgattccaaagtgctccacgcagcttaaggccgtgacggcccacttccagacgcacacgaccccgatcctcctcagcaacaacttcatcgagcccctggagatcacgaaggagatatacgacctgaacaacccggagaaggagcccaagaaattccagaccgcctacgccaagaagacaggcgaccaaaagggttacagggaggccctctgcaagtggatcgacttcactagggacttcctgtccaagtacaccaagactacctctatcgacctgtccagcctccgcccgtcgtcccagtacaaagatttgggcgagtattacgcggagctgaacccactgctctaccacatcagcttccagcgcatcgcggagaaggagatcatggacgcagtggagacgggcaagctatacctatttcagatatacaacaaagacttcgctaagggacaccacggcaagcctaacctgcacaccctctactggacggggctcttcagcccggagaacctcgccaagacctcgatcaagctcaacggccaggccgagctgttctaccggcccaagtcccgcatgaagcggatggcccaccggctcggggagaaaatgctcaacaagaaattgaaggaccaaaaaacgccgatacccgacaccctataccaggagctgtacgactatgtgaaccaccgcctgagccacgacctcagcgacgaggcgcgggccctcctgccgaacgtcatcacaaaggaggtcagccacgagatcatcaaggaccggcgcttcacctccgacaagtttttctttcacgtgcccatcacgctcaactaccaggccgccaactcgccgtccaagttcaaccagcgcgtgaacgcctacctcaaggagcaccccgagaccccgatcatcgggattgaccgaggggagcggaacctcatctacatcaccgtcatcgacagcaccgggaagatccttgaacagcggtcgctcaacaccatccagcagttcgactaccagaagaaactcgacaaccgggagaaggagagagtggcggcccgccaggcttggtccgtcgtcgggacgattaaggacttgaaacaaggttacctgtcgcaagtgatccacgagatcgttgacctgatgatccactaccaagccgtcgtggtcctggagaacctcaacttcggcttcaagagcaaacgaaccggcatcgcggagaaggccgtgtaccagcagttcgaaaaaatgctgatcgacaagctgaactgcctcgtgctcaaggactaccccgctgagaaggtcggcggggtgctgaacccgtaccagctcactgaccagttcaccagcttcgcaaagatgggcacccagtccggcttcctgttctacgtgcctgcgccatacacctcgaagatcgacccgctcaccgggttcgtggaccccttcgtctggaagaccatcaagaaccacgagagccgcaagcacttcctggagggcttcgacttcctccactacgacgtcaagaccggggacttcatcctgcacttcaagatgaaccgcaacctcagtttccagcgcggcctgccggggttcatgcccgcttgggatatagtcttcgagaagaatgagacgcagttcgacgcgaagggcaccccgttcatcgccgggaagcgcatcgtgccggtcatcgagaaccaccggttcaccgggcgctaccgcgacctatacccggcgaacgagttgatcgccctcctggaggagaagggcatcgtgttccgcgacggctccaacatcctcccgaagctgctcgaaaacgacgactcccacgccatcgacacgatggtcgcgctgatccggtcggtgctccagatgcggaactccaacgccgcgacgggcgaggactacatcaacagtccggtccgcgatctgaacggcgtctgcttcgactcccggttccagaaccccgagtggccgatggacgcggacgcgaacggcgcataccacatcgccctaaaagggcaattgctgctcaaccacctcaaggaatccaaagacctaaagctccagaacggcatctccaaccaggactggctggcgtacatccaggaactgcggaacgggagcaaaaaacgtcggatcaagcaagattga

SEQ ID NO: 134 - DNA, 3987 bp, synthetic construct
  source 1..3987: mol_type = other DNA
atggcgggctccaagaaacgccggattaagcaagatacccagttcgaggggttcacgaacctctaccaagtgagcaagaccctccgattcgaactgattcctcaggggaagaccctcaagcacatccaggagcaagggttcatcgaggaggacaaggcgcggaacgaccactacaaggaactcaaacccatcatcgaccgcatctacaagacctacgccgatcagtgcctccagctcgtgcagttggactgggagaacctcagcgcggccattgactcctaccggaaggagaaaacggaggagacgcgcaacgcgctcatcgaggaacaggcaacctatcgcaacgccatccacgactacttcatcgggaggactgacaacctcactgacgcgattaacaagcgccacgcggagatatacaagggactcttcaaagcggagctgtttaacggcaaggttctcaagcaactcggcactgtgaccacgaccgagcatgagaacgccctgctccgctccttcgacaagttcaccacctacttctccgggttctaccgcaaccgcaagaatgtcttcagcgcggaggacatcagcacggccattccacatcgaatcgtccaagataacttcccgaagttcaaggagaactgccacatcttcacccgactcattactgctgtaccgtcgttacgcgaacacttcgagaacgtcaagaaggcaattggaatcttcgtctctacgtcaatagaggaggtgttcagcttccctttctacaaccagctccttacgcagacccagatagacctgtacaatcagctcctcggtgggatcagccgggaggcggggactgagaagattaaagggctcaacgaggtcttgaacctggccatccaaaaaaacgatgagacggcgcacatcatcgcctcgctgccccaccggttcatcccgctgttcaagcagatcctcagtgacaggaacaccttgagctttatcctagaggagttcaagagcgacgaggaggtgatccagagcttctgcaagtacaaaaccctgctgaggaacgagaacgtcctggagacggcggaggcgctgttcaacgagctgaactctatcgacttaactcacatattcatctcgcacaagaagctggagactattagctctgcactctgcgaccactgggacaccctccgcaacgcgctctacgagcgccgcatctcggagctgaccgggaagatcaccaaatccgcgaaggaaaaggtccagcgttccctcaaacacgaggatattaacttacaggagattatctcagcggctgggaaggagttgtcagaggcgttcaagcagaaaacttccgagatcctgagccacgcgcacgcagcgctcgaccagcctctgcccaccaccctcaaaaagcaggaagaaaaagagatcctcaagagccagttggactccctgctggggctctatcaccttctcgactggttcgccgtcgatgagtcgaacgaggtggaccccgagttctccgcccggctgaccggcatcaagctagagatggagccgtccctcagcttctacaataaggcccgcaactacgcgaccaaaaaaccctacagcgtggagaagttcaagctgaacttccagatgccgaccttagcacgcggttgggacgtaaacagggagaagaacaatggagccatcctgttcgtcaagaacgggctttactacctcgggataatgcccaagcagaagggccgctacaaggccctttccttcgagccgacggagaaaacctccgaggggttcgacaagatgtactacgactacttccccgacgccgccaagatgatcccgaagtgctcaacgcagctaaaagccgtgaccgcccacttccagacccacacgacgccgatcctgctgagcaacaacttcatcgagccccttgagatcactaaggagatatacgacctgaacaaccccgagaaggagcccaagaagtttcaaaccgcctacgccaaaaaaactggcgaccaaaagggctacagggaggcgctgtgtaagtggatcgacttcacacgcgacttcctttcgaagtatacgaagacaacctctattgacctgagcagcctgcgtcctagctcccagtacaaagatttgggcgagtactacgcggagcttaatccactactctaccacatctcattccagcgcatcgctgagaaggaaatcatggacgcggtggagacaggcaaactgtacctcttccagatatacaacaaagacttcgctaaggggcaccacgggaagcccaaccttcatacgctctactggacgggcctattcagccccgaaaatctggccaagacctccatcaagctgaacggccaagcggagctgttctacagacccaagagccggatgaagcggatggcccacaggctcggcgagaaaatgcttaacaaaaagttgaaggaccagaaaacccctatccccgacaccctctaccaggaactgtacgactacgtgaaccacaggctctcgcacgacctttccgacgaggcccgtgccctactcccgaacgtcattaccaaagaggtttcgcacgagatcatcaaggaccggcggttcacgagcgacaagtttttctttcacgtccccatcacccttaactaccaggcggccaactccccatccaagttcaaccagcgtgtgaatgcctacctcaaggagcacccagagaccccgatcattgggatcgaccggggcgagcggaacctgatctacatcaccgtcatcgactcgacgggcaagattcttgagcagagatcgttgaataccatacagcagttcgactaccagaagaaactcgacaaccgcgagaaggagcgcgtggcggcccgccaggcgtggtccgtcgttgggacgattaaggacttgaaacaaggttatctgtcccaagtcatccacgagatcgttgatctgatgatccactatcaggcagtggtggtgctggagaatctcaacttcggcttcaagagtaagcggacgggaatcgccgagaaggccgtgtaccagcagttcgagaagatgctgatcgacaagctcaactgccttgtgctgaaagactacccggccgagaaggtcggcggcgtcctcaacccgtaccaacttaccgaccagttcacctccttcgccaagatgggcactcagtccgggttcttgttctacgtccccgcaccttacacctctaagatcgaccctctgactggcttcgtagatccattcgtgtggaagaccattaagaaccacgagagccgcaagcacttcctggagggcttcgacttcctgcactacgacgtgaagaccggggacttcatccttcacttcaagatgaaccggaacctcagcttccagcggggcctgccggggttcatgcccgcctgggacatcgtgttcgagaagaacgagacccagttcgacgcgaagggcacgcccttcatcgccgggaagcgtatcgtgccggtgatcgagaaccatcgtttcacgggtcgctaccgtgacctctacccggcgaacgagcttatcgcactcctggaggagaagggcatcgtcttccgggacggctccaacatcctcccgaaactgctggaaaacgacgactctcacgccatcgacacgatggtggccctcatccggtccgtgctccaaatgcggaacagcaacgccgccaccggtgaggactacatcaacagcccggtccgggatctgaacggggtgtgcttcgattcgcggttccagaatcctgagtggccgatggacgcggatgcaaacggggcgtaccacatcgcgctcaagggccagttacttctgaaccaccttaaggagtctaaagatttgaaactccagaacgggatctcgaaccaggactggctggcctacatccaagagttgcggaacggcagcaagaagcggcggattaagcaagattag

SEQ ID NO: 135 - protein, 9 aa, synthetic construct
LAGLIDADG

(19) United States
(12) Patent Application Publication
(10) Pub. No.: US 2025/0283098 A1
(43) Pub. Date: Sep. 11, 2025

(54) MORPHOGENIC REGULATORS AND METHODS OF USING THE SAME

(71) Applicant: Pairwise Plants Services, Inc., Durham, NC (US)

(72) Inventors: Ming Cheng, Cary, NC (US); Qingchun Shi, Cary, NC (US); Thomas J. Poorten, Durham, NC (US); Brian Charles Wilding Crawford, Cary, NC (US); Sathya Sheela Jali, Cary, NC (US); Doug Heckart, Wake Forest, NC (US); Yongjoo Kim, Durham, NC (US)

(21) Appl. No.: 19/218,804

(22) Filed: May 27, 2025

Related U.S. Application Data
(62) Division of application No. 17/630,973, filed on Jan. 28, 2022, now Pat. No. 12,344,850.
(60) Provisional application No. 62/880,131, filed on Jul. 30, 2019.

(51) Int. Cl. C12N 15/82

(52) U.S. Cl. CPC C12N 15/821; C12N 15/8216

(57) ABSTRACT

Methods and compositions for generating transgene-free plants are described. In particular, the transgene-free plants are regenerated by transient expression of morphogenic regulators in a plant cell that is precisely edited using a gene-editing methodology. Also, provided are transformed plants, plant cell, tissues, and seeds in which a target gene is edited, but non-transgenic.

### STATEMENT REGARDING ELECTRONIC FILING OF A SEQUENCE LISTING

A Sequence Listing in XML format, entitled 1499-39DV_ST26.xml, 283,095 bytes in size, generated on May 25, 2025, and filed herewith, is hereby incorporated by reference in its entirety for its disclosures.

### FIELD

The present disclosure relates to morphogenic regulators and methods of using the same. The present disclosure further relates to next generation plant breeding, including breeding for vegetative propagated crops, along with methods and compositions for producing transgene-free plants, optionally in which a target gene is edited.

### BACKGROUND

Recombinant DNA technology has emerged as a powerful tool for editing genes and genetic elements in vectors to produce novel recombinant DNA products. Due to the continuous improvement of DNA technology and the application of Agrobacterium for transgene delivery, transgenic plants have been successfully generated over two decades and genetically modified (GM) plant products have been commercialized to meet high demands of modern society. Not surprisingly, genetic transformation in plants has become a routine practice so that there are more than one hundred agricultural crops to have been genetically modified around the world.

The International Service for the Acquisition of Agri-Biotech Applications (ISAAA) reported that about 160 million hectares were used for cultivating transgenic crops in 2011, which was a 94-fold increase within five years from 1996. There is no doubt that GM technology has become an important plant breeding tool as an answer to a threat of food security in developing countries.

Along with rapid growth of transgenic crop cultivation and production, GM plants have raised concerns from regulatory agencies and the public in some countries, including Europe. One of main concerns is that a piece of foreign DNA and/or a selectable marker gene (usually an antibiotic- or herbicide-resistant gene) remains in the genomes of GM plants. There have been a few approaches to producing transgene-free transgenic plants. One approach is to segregate away integration of a transgene and/or a piece of foreign DNA by conventional breeding methods, for example, by backcrossing the transgenic plants with its parent and achieving offspring with a genetic identity which is closer to that of the parent. In this way, a desired trait in the transgenic plants can be transferred to the favored genetic background of another variety, while undesired foreign DNA can be eliminated.

The aforementioned segregating-away approach has been applied to row crops for removal of unwanted foreign DNA and/or transgene(s); however, they are not particularly adopted to vegetative propagated crops due to their heterozygotic nature and/or multiploidy. The recurrent crossing method to segregate away the transgene(s) is practically and economically undesirable for heterozygotic and/or multiploid plants because the crossings would be technically challenging and/or selection of progeny with only the desired traits and/or gene of interest (GOI) would require multiple rounds of crossing to generate pure and/or near-isogenic lines, which could result in genetic drift, such as loss of beneficial alleles. Also, conventional genetic engineering methods still have not given solutions to the technical challenges of identifying random mutations caused by integration of foreign DNAs in a host genome and removing them in a selective, efficient, and economic manner. Therefore, there is a great need in the art for a method of creating transgene-free plants with high heterozygosity and/or multiploidy, along with introduction of targeted genetic modification by employing new targeted genome engineering technologies.

### SUMMARY OF THE DISCLOSURE

One aspect of the present disclosure is directed to an isolated polynucleotide comprising a nucleotide sequence having at least 90%, 95%, or 98% sequence identity to the nucleotide sequence of any one of SEQ ID NOs: 1-18 or SEQ ID NOs: 28-49. In some embodiments, the isolated polynucleotide comprises a nucleotide sequence having at least 90%, 95%, or 98% sequence identity to the nucleotide sequence of any one of SEQ ID NOs: 1-18. In some embodiments, the isolated polynucleotide comprises the nucleotide sequence of any one of SEQ ID NOs: 1-18 or SEQ ID NOs: 28-49. In some embodiments, the isolated polynucleotide comprises the nucleotide sequence of any one of SEQ ID NOs: 1-18.

Another aspect of the present disclosure is directed to an isolated polynucleotide encoding an amino acid sequence having at least 90%, 95%, or 98% sequence identity to the polypeptide sequence of any one of SEQ ID NOs: 19-27 or SEQ ID NOs: 50-60. In some embodiments, the isolated polynucleotide encodes an amino acid sequence having at least 90%, 95%, or 98% sequence identity to the polypeptide sequence of any one of SEQ ID NOs: 19-27. In some embodiments, the isolated polynucleotide encodes the polypeptide sequence of any one of SEQ ID NOs: 19-27 or SEQ ID NOs: 50-60. In some embodiments, the isolated polynucleotide encodes the polypeptide sequence of any one of SEQ ID NOs: 19-27.

A further aspect of the present disclosure is directed to an isolated polypeptide comprising an amino acid sequence having at least 90%, 95%, or 98% sequence identity to the amino acid sequence of any one of SEQ ID NOs: 19-27 or SEQ ID NOs: 50-60. In some embodiments, the isolated polypeptide comprises an amino acid sequence having at least 90%, 95%, or 98% sequence identity to the amino acid sequence of any one of SEQ ID NOs: 19-27. In some embodiments, the isolated polypeptide comprises the amino acid sequence of any one of SEQ ID NOs: 19-27 or SEQ ID NOs: 50-60. In some embodiments, the isolated polypeptide comprises the amino acid sequence of any one of SEQ ID NOs: 19-27.

Another aspect of the present disclosure is directed to a method for transforming a plant cell, the method comprising: introducing a polynucleotide or a polypeptide comprising a heterologous morphogenic regulator into the plant cell, thereby transforming the plant cell.

A further aspect of the present disclosure is directed to a method of improving or increasing regeneration frequency in a group of plant cells, the method comprising: introducing a polynucleotide comprising a heterologous morphogenic regulator into a plant cell in the group of plant cells, thereby improving or increasing regeneration frequency in the group of plant cells.

An additional aspect of the present disclosure is directed to a plant cell, plant part, and/or plant comprising: an edited polynucleotide, wherein the plant cell, plant part, and/or plant is transgene-free.

A further aspect of the present disclosure is directed to a plant cell comprising: a polynucleotide comprising a heterologous morphogenic regulator.

Another aspect of the present disclosure is directed to a group of plant cells comprising: a first plant cell comprising a polynucleotide comprising a heterologous morphogenic regulator; and a second plant cell comprising a polynucleotide encoding gene editing machinery or a gene editing complex (e.g., a ribonucleoprotein) that is capable of modifying at least one target nucleic acid.

The present disclosure can provide a solution to the aforementioned problem of conventional breeding and/or genetic engineering methods to avoid integrating undesired transgenes and/or foreign DNA pieces from stably integrated transgenic plants. In some embodiments, the present disclosure provides a method of modulating and/or modifying a target nucleic acid using a gene-editing system. Furthermore, the disclosure teaches a method of stimulating regeneration of a gene-edited plant or plant part from a plant cell, optionally in which a target nucleic acid is stably and precisely edited. The regeneration of a gene-edited plant or plant part can be induced, as elaborated upon herein, by at least one transiently expressed morphogenic regulator taught in the present disclosure.

The present disclosure further provides expression cassettes and/or vectors comprising a polynucleotide and/or polypeptide of the present invention, and cells comprising a polynucleotide and/or polypeptide of the present invention.

It is noted that aspects of the invention described with respect to one embodiment, may be incorporated in a different embodiment although not specifically described relative thereto. That is, all embodiments and/or features of any embodiment can be combined in any way and/or combination. Applicant reserves the right to change any originally filed claim and/or file any new claim accordingly, including the right to be able to amend any originally filed claim to depend from and/or incorporate any feature of any other claim or claims although not originally claimed in that manner. These and other objects and/or aspects of the present invention are explained in detail in the specification set forth below. Further features, advantages and details of the present invention will be appreciated by those of ordinary skill in the art from a reading of the figures and the detailed description of the preferred embodiments that follow, such description being merely illustrative of the present invention.

### BRIEF DESCRIPTION OF THE FIGURES

FIG. 1 illustrates GUS assay results of one hundred and six shoots induced by the ipt gene expression. From one hundred six ipt-induced shoots, seven samples were GUS-positive, but the other ninety-nine samples were GUS-negative.

### DETAILED DESCRIPTION

The present disclosure now will be described hereinafter with reference to the accompanying drawings and examples, in which embodiments of the invention are shown. This description is not intended to be a detailed catalog of all the different ways in which the invention may be implemented, or all the features that may be added to the instant invention. For example, features illustrated with respect to one embodiment may be incorporated into other embodiments, and features illustrated with respect to a particular embodiment may be deleted from that embodiment. Thus, the invention contemplates that in some embodiments of the invention, any feature or combination of features set forth herein can be excluded or omitted. To illustrate, if the specification states that a composition comprises components A, B and C, it is specifically intended that any of A, B or C, or a combination thereof, can be omitted and disclaimed singularly or in any combination. In addition, numerous variations and additions to the various embodiments suggested herein will be apparent to those skilled in the art in light of the instant disclosure, which do not depart from the instant invention. Hence, the following descriptions are intended to illustrate some particular embodiments of the invention, and not to exhaustively specify all permutations, combinations and variations thereof.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

All publications, patent applications, patents and other references cited herein are incorporated by reference in their entireties for the teachings relevant to the sentence and/or paragraph in which the reference is presented.

As used in the description of the invention and the appended claims, the singular forms “a,” “an” and “the” are intended to include the plural forms as well, unless the context clearly indicates otherwise.

Also as used herein, “and/or” refers to and encompasses any and all possible combinations of one or more of the associated listed items, as well as the lack of combinations when interpreted in the alternative (“or”).

The term “about,” as used herein when referring to a measurable value such as an amount or concentration and the like, is meant to encompass variations of ±10%, ±5%, ±1%, ±0.5%, or even ±0.1% of the specified value as well as the specified value. For example, “about X” where X is the measurable value, is meant to include X as well as variations of ±10%, ±5%, ±1%, ±0.5%, or even ±0.1% of X. A range provided herein for a measureable value may include any other range and/or individual value therein.

As used herein, phrases such as “between X and Y” and “between about X and Y” should be interpreted to include X and Y. As used herein, phrases such as “between about X and Y” mean “between about X and about Y” and phrases such as “from about X to Y” mean “from about X to about Y.”

Recitation of ranges of values herein are merely intended to serve as a shorthand method of referring individually to each separate value falling within the range, unless otherwise indicated herein, and each separate value is incorporated into the specification as if it were individually recited herein. For example, if the range 10 to 15 is disclosed, then 11, 12, 13, and 14 are also disclosed.

The term “comprise,” “comprises” and “comprising” as used herein, specify the presence of the stated features, integers, steps, operations, elements, and/or components, but do not preclude the presence or addition of one or more other features, integers, steps, operations, elements, components, and/or groups thereof.

As used herein, the transitional phrase “consisting essentially of” means that the scope of a claim is to be interpreted to encompass the specified materials or steps recited in the claim and those that do not materially affect the basic and novel characteristic(s) of the claimed invention. Thus, the term “consisting essentially of” when used in a claim of this invention is not intended to be interpreted to be equivalent to “comprising.”

As used herein, the terms “increase,” “increasing,” “enhance,” “enhancing,” “improve” and “improving” (and grammatical variations thereof) describe an elevation in a specified parameter or value of at least about 5%, 10%, 15%, 20%, 25%, 30%, 35%, 40%, 45%, 50%, 55%, 60%, 65%, 70%, 75%, 80%, 85%, 90%, 95%, 100%, 150%, 200%, 300%, 400%, 500% or more as compared to a control.

As used herein, the terms “reduce,” “reduced,” “reducing,” “reduction,” “diminish,” and “decrease” (and grammatical variations thereof), describe, for example, a decrease in a specified parameter or value of at least about 5%, 10%, 15%, 20%, 25%, 30%, 35%, 40%, 45%, 50%, 55%, 60%, 65%, 70%, 75%, 80%, 85%, 90%, 95%, 97%, 98%, 99%, or 100% as compared to a control. In particular embodiments, the reduction can result in no or essentially no (i.e., an insignificant amount, e.g., less than about 10% or even 5%) detectable activity or amount.

A “heterologous” or a “recombinant” nucleotide sequence is a nucleotide sequence not naturally associated with a host cell into which it is introduced, including non-naturally occurring multiple copies of a naturally occurring nucleotide sequence.

A “native” or “wild type” nucleic acid, nucleotide sequence, polypeptide or amino acid sequence refer to a naturally occurring or endogenous nucleic acid, nucleotide sequence, polypeptide or amino acid sequence. Thus, for example, a “wild type mRNA” is an mRNA that is naturally occurring in or endogenous to the reference organism. A “homologous” nucleic acid sequence is a nucleotide sequence naturally associated with a host cell into which it is introduced.

As used herein, the terms “nucleic acid,” “nucleic acid molecule,” “nucleotide sequence” and “polynucleotide” refer to RNA or DNA that is linear or branched, single or double stranded, or a hybrid thereof. The term also encompasses RNA/DNA hybrids. When dsRNA is produced synthetically, less common bases, such as inosine, 5-methylcytosine, 6-methyladenine, hypoxanthine and others can also be used for antisense, dsRNA, and ribozyme pairing. For example, polynucleotides that contain C-5 propyne analogues of uridine and cytidine have been shown to bind RNA with high affinity and to be potent antisense inhibitors of gene expression. Other modifications, such as modification to the phosphodiester backbone, or the 2′-hydroxy in the ribose sugar group of the RNA can also be made.

As used herein, the term “nucleotide sequence” refers to a heteropolymer of nucleotides or the sequence of these nucleotides from the 5′ to 3′ end of a nucleic acid molecule and includes DNA or RNA molecules, including cDNA, a DNA fragment or portion, genomic DNA, synthetic (e.g., chemically synthesized) DNA, plasmid DNA, mRNA, and anti-sense RNA, any of which can be single stranded or double stranded. The terms “nucleotide sequence” “nucleic acid,” “nucleic acid molecule,” “nucleic acid construct,” “recombinant nucleic acid,” “oligonucleotide” and “polynucleotide” are also used interchangeably herein to refer to a heteropolymer of nucleotides. Nucleic acid molecules and/or nucleotide sequences provided herein are presented herein in the 5′ to 3′ direction, from left to right and are represented using the standard code for representing the nucleotide characters as set forth in the U.S. sequence rules, 37 CFR §§ 1.821-1.825 and the World Intellectual Property Organization (WIPO) Standard ST.25. A “5′ region” as used herein can mean the region of a polynucleotide that is nearest the 5′ end of the polynucleotide. Thus, for example, an element in the 5′ region of a polynucleotide can be located anywhere from the first nucleotide located at the 5′ end of the polynucleotide to the nucleotide located halfway through the polynucleotide. A “3′ region” as used herein can mean the region of a polynucleotide that is nearest the 3′ end of the polynucleotide. Thus, for example, an element in the 3′ region of a polynucleotide can be located anywhere from the first nucleotide located at the 3′ end of the polynucleotide to the nucleotide located halfway through the polynucleotide.

As used herein, the term “gene” refers to a nucleic acid molecule capable of being used to produce mRNA, antisense RNA, miRNA, anti-microRNA antisense oligodeoxyribonucleotide (AMO) and the like. Genes may or may not be capable of being used to produce a functional protein or gene product. Genes can include both coding and non-coding regions (e.g., introns, regulatory elements, promoters, enhancers, termination sequences and/or 5′ and 3′ untranslated regions).

A polynucleotide, gene, or polypeptide may be “isolated” by which is meant a nucleic acid or polypeptide that is substantially or essentially free from components normally found in association with the nucleic acid or polypeptide, respectively, in its natural state. In some embodiments, such components include other cellular material, culture medium from recombinant production, and/or various chemicals used in chemically synthesizing the nucleic acid or polypeptide.

The term “mutation” refers to point mutations (e.g., missense, or nonsense, or insertions or deletions of single base pairs that result in frame shifts), insertions, deletions, and/or truncations. When the mutation is a substitution of a residue within an amino acid sequence with another residue, or a deletion or insertion of one or more residues within a sequence, the mutations are typically described by identifying the original residue followed by the position of the residue within the sequence and by the identity of the newly substituted residue.

The terms “complementary” or “complementarity,” as used herein, refer to the natural binding of polynucleotides under permissive salt and temperature conditions by base-pairing. For example, the sequence “A-G-T” (5′ to 3′) binds to the complementary sequence “T-C-A” (3′ to 5′). Complementarity between two single-stranded molecules may be “partial,” in which only some of the nucleotides bind, or it may be complete when total complementarity exists between the single stranded molecules. The degree of complementarity between nucleic acid strands has significant effects on the efficiency and strength of hybridization between nucleic acid strands.

“Complement” as used herein can mean 100% complementarity with the comparator nucleotide sequence or it can mean less than 100% complementarity (e.g., “substantially complementary,” e.g., about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, and the like, complementarity).

A “portion” or “fragment” of a nucleotide sequence or polypeptide sequence will be understood to mean a nucleotide or polypeptide sequence of reduced length (e.g., reduced by 1, 2, 3, 4, 5, 6, 7, 8, 9, 10, 11, 12, 13, 14, 15, 16, 17, 18, 19, 20 or more residue(s) (e.g., nucleotide(s) or peptide(s)) relative to a reference nucleotide or polypeptide sequence, respectively, and comprising, consisting essentially of and/or consisting of a nucleotide or polypeptide sequence of contiguous residues, respectively, identical or almost identical (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical) to the reference nucleotide or polypeptide sequence. Such a nucleic acid fragment or portion according to the invention may be, where appropriate, included in a larger polynucleotide of which it is a constituent. As an example, a repeat sequence of guide nucleic acid of this invention may comprise a portion of a wild type CRISPR-Cas repeat sequence (e.g., a wild type Type V CRISPR Cas repeat, e.g., a repeat from the CRISPR Cas system that includes, but is not limited to, Cas12a (Cpf1), Cas12b, Cas12c (C2c3), Cas12d (CasY), Cas12e (CasX), Cas12g, Cas12h, Cas12i, C2c1, C2c4, C2c5, C2c8, C2c9, C2c10, Cas14a, Cas14b, and/or Cas14c, and the like).

Different nucleic acids or proteins having homology are referred to herein as “homologues.” The term homologue includes homologous sequences from the same and other species and orthologous sequences from the same and other species. “Homology” refers to the level of similarity between two or more nucleic acid and/or amino acid sequences in terms of percent of positional identity (i.e., sequence similarity or identity). Homology also refers to the concept of similar functional properties among different nucleic acids or proteins. Thus, the compositions and methods of the invention further comprise homologues to the nucleotide sequences and polypeptide sequences of this invention. “Orthologous” and “orthologs” as used herein, refers to homologous nucleotide sequences and/or amino acid sequences in different species that arose from a common ancestral gene during speciation. A homologue or ortholog of a nucleotide sequence of this invention has a substantial sequence identity (e.g., at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5% or 100%) to said nucleotide sequence of the invention.

As used herein “sequence identity” refers to the extent to which two optimally aligned polynucleotide or polypeptide sequences are invariant throughout a window of alignment of components, e.g., nucleotides or amino acids. “Identity” can be readily calculated by known methods including, but not limited to, those described in: Computational Molecular Biology (Lesk, A. M., ed.) Oxford University Press, New York (1988); Biocomputing: Informatics and Genome Projects (Smith, D. W., ed.) Academic Press, New York (1993); Computer Analysis of Sequence Data, Part I (Griffin, A. M., and Griffin, H. G., eds.) Humana Press, New Jersey (1994); Sequence Analysis in Molecular Biology (von Heinje, G., ed.) Academic Press (1987); and Sequence Analysis Primer (Gribskov, M. and Devereux, J., eds.) Stockton Press, New York (1991).

As used herein, the term “percent sequence identity” or “percent identity” refers to the percentage of identical nucleotides in a linear polynucleotide sequence of a reference (“query”) polynucleotide molecule (or its complementary strand) as compared to a test (“subject”) polynucleotide molecule (or its complementary strand) when the two sequences are optimally aligned. In some embodiments, “percent identity” can refer to the percentage of identical amino acids in an amino acid sequence as compared to a reference polypeptide.

As used herein, the phrase “substantially identical,” or “substantial identity” in the context of two nucleic acid molecules, nucleotide sequences or protein sequences, refers to two or more sequences or subsequences that have at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5% or 100% nucleotide or amino acid residue identity, when compared and aligned for maximum correspondence, as measured using one of the following sequence comparison algorithms or by visual inspection. In some embodiments of the invention, the substantial identity exists over a region of consecutive nucleotides of a nucleotide sequence of the invention that is about 10 nucleotides to about 20 nucleotides, about 10 nucleotides to about 25 nucleotides, about 10 nucleotides to about 30 nucleotides, about 15 nucleotides to about 25 nucleotides, about 30 nucleotides to about 40 nucleotides, about 50 nucleotides to about 60 nucleotides, about 70 nucleotides to about 80 nucleotides, about 90 nucleotides to about 100 nucleotides, or more nucleotides in length, and any range therein, up to the full length of the sequence. In some embodiments, the nucleotide sequences can be substantially identical over at least about 20 nucleotides (e.g., about 20, 21, 22, 23, 24, 25, 26, 27, 28, 29, 30, 31, 32, 33, 34, 35, 36, 37, 38, 39, 40 nucleotides). In some embodiments, a substantially identical nucleotide or protein sequence performs substantially the same function as the nucleotide (or encoded protein sequence) to which it is substantially identical.

For sequence comparison, typically one sequence acts as a reference sequence to which test sequences are compared. When using a sequence comparison algorithm, test and reference sequences are entered into a computer, subsequence coordinates are designated if necessary, and sequence algorithm program parameters are designated. The sequence comparison algorithm then calculates the percent sequence identity for the test sequence(s) relative to the reference sequence, based on the designated program parameters.

Optimal alignment of sequences for aligning a comparison window are well known to those skilled in the art and may be conducted by tools such as the local homology algorithm of Smith and Waterman, the homology alignment algorithm of Needleman and Wunsch, the search for similarity method of Pearson and Lipman, and optionally by computerized implementations of these algorithms such as GAP, BESTFIT, FASTA, and TFASTA available as part of the GCG WISCONSIN PACKAGE® (Accelrys Inc., San Diego, CA) software suite for sequence analysis. An “identity fraction” for aligned segments of a test sequence and a reference sequence is the number of identical components which are shared by the two aligned sequences divided by the total number of components in the reference sequence segment, e.g., the entire reference sequence or a smaller defined part of the reference sequence. Percent sequence identity is represented as the identity fraction multiplied by 100. The comparison of one or more polynucleotide sequences may be to a full-length polynucleotide sequence or a portion thereof, or to a longer polynucleotide sequence. For purposes of this invention “percent identity” may also be determined using BLASTX version 2.0 for translated nucleotide sequences and BLASTN version 2.0 for polynucleotide sequences.

Two nucleotide sequences may also be considered substantially complementary when the two sequences hybridize to each other under stringent conditions. In some representative embodiments, two nucleotide sequences considered to be substantially complementary hybridize to each other under highly stringent conditions.

“Stringent hybridization conditions” and “stringent hybridization wash conditions” in the context of nucleic acid hybridization experiments such as Southern and Northern hybridizations are sequence dependent, and are different under different environmental parameters. An extensive guide to the hybridization of nucleic acids is found in Tijssen Laboratory Techniques in Biochemistry and Molecular Biology-Hybridization with Nucleic Acid Probes part I chapter 2 “Overview of principles of hybridization and the strategy of nucleic acid probe assays” Elsevier, New York (1993). Generally, highly stringent hybridization and wash conditions are selected to be about 5° C. lower than the thermal melting point (Tₘ) for the specific sequence at a defined ionic strength and pH.

The Tₘ is the temperature (under defined ionic strength and pH) at which 50% of the target sequence hybridizes to a perfectly matched probe. Very stringent conditions are selected to be equal to the Tₘ for a particular probe. An example of stringent hybridization conditions for hybridization of complementary nucleotide sequences which have more than 100 complementary residues on a filter in a Southern or northern blot is 50% formamide with 1 mg of heparin at 42° C., with the hybridization being carried out overnight. An example of highly stringent wash conditions is 0.1 5M NaCl at 72° C. for about 15 minutes. An example of stringent wash conditions is a 0.2×SSC wash at 65° C. for 15 minutes (see, Sambrook, infra, for a description of SSC buffer). Often, a high stringency wash is preceded by a low stringency wash to remove background probe signal. An example of a medium stringency wash for a duplex of, e.g., more than 100 nucleotides, is 1×SSC at 45° C. for 15 minutes. An example of a low stringency wash for a duplex of, e.g., more than 100 nucleotides, is 4-6×SSC at 40° C. for 15 minutes. For short probes (e.g., about 10 to 50 nucleotides), stringent conditions typically involve salt concentrations of less than about 1.0 M Na ion, typically about 0.01 to 1.0 M Na ion concentration (or other salts) at pH 7.0 to 8.3, and the temperature is typically at least about 30° C. Stringent conditions can also be achieved with the addition of destabilizing agents such as formamide. In general, a signal to noise ratio of 2× (or higher) than that observed for an unrelated probe in the particular hybridization assay indicates detection of a specific hybridization. Nucleotide sequences that do not hybridize to each other under stringent conditions are still substantially identical if the proteins that they encode are substantially identical. This can occur, for example, when a copy of a nucleotide sequence is created using the maximum codon degeneracy permitted by the genetic code.

A polynucleotide and/or recombinant nucleic acid construct of this invention can be codon optimized for expression. In some embodiments, a polynucleotide, nucleic acid construct, expression cassette, and/or vector of the present invention (e.g., that comprises/encodes a nucleic acid binding domain (e.g., a DNA binding domain such as a sequence-specific DNA binding domain from a polynucleotide-guided endonuclease, a zinc finger nuclease, a transcription activator-like effector nuclease (TALEN), an Argonaute protein, and/or a CRISPR-Cas effector protein), a guide nucleic acid, a cytosine deaminase, adenine deaminase, and/or a heterologous morphogenic regulator) may be codon optimized for expression in an organism (e.g., an animal, a plant, a fungus, an archacon, or a bacterium). In some embodiments, the codon optimized nucleic acid constructs, polynucleotides, expression cassettes, and/or vectors of the invention have about 70% to about 99.9% (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5%. 99.9% or 100%) identity or more to the reference nucleic acid constructs, polynucleotides, expression cassettes, and/or vectors but which have not been codon optimized.

In any of the embodiments described herein, a polynucleotide or nucleic acid construct of the invention may be operatively associated with a variety of promoters and/or other regulatory elements for expression in a an organism or cell thereof (e.g., a plant and/or a cell of a plant). Thus, in some embodiments, a polynucleotide or nucleic acid construct of this invention may further comprise one or more promoters, introns, enhancers, and/or terminators operably linked to one or more nucleotide sequences. In some embodiments, a promoter may be operably associated with an intron (e.g., Ubi1 promoter and intron). In some embodiments, a promoter associated with an intron maybe referred to as a “promoter region” (e.g., Ubi1 promoter and intron).

By “operably linked” or “operably associated” as used herein in reference to polynucleotides, it is meant that the indicated elements are functionally related to each other, and are also generally physically related. Thus, the term “operably linked” or “operably associated” as used herein, refers to nucleotide sequences on a single nucleic acid molecule that are functionally associated. Thus, a first nucleotide sequence that is operably linked to a second nucleotide sequence means a situation when the first nucleotide sequence is placed in a functional relationship with the second nucleotide sequence. For instance, a promoter is operably associated with a nucleotide sequence if the promoter effects the transcription or expression of said nucleotide sequence. Those skilled in the art will appreciate that the control sequences (e.g., promoter) need not be contiguous with the nucleotide sequence to which it is operably associated, as long as the control sequences function to direct the expression thereof. Thus, for example, intervening untranslated, yet transcribed, nucleic acid sequences can be present between a promoter and the nucleotide sequence, and the promoter can still be considered “operably linked” to the nucleotide sequence.

As used herein, the term “linked,” or “fused” in reference to polypeptides, refers to the attachment of one polypeptide to another. A polypeptide may be linked or fused to another polypeptide (at the N-terminus or the C-terminus) directly (e.g., via a peptide bond) or through a linker (e.g., a peptide linker).

The term “linker” in reference to polypeptides is art-recognized and refers to a chemical group, or a molecule linking two molecules or moieties, e.g., two domains of a fusion protein, such as, for example, a CRISPR-Cas effector protein and a peptide tag and/or a polypeptide of interest. A linker may be comprised of a single linking molecule (e.g., a single amino acid) or may comprise more than one linking molecule. In some embodiments, the linker can be an organic molecule, group, polymer, or chemical moiety such as a bivalent organic moiety. In some embodiments, the linker may be an amino acid or it may be a peptide. In some embodiments, the linker is a peptide. In some embodiments, a peptide linker useful with this invention may be about 2 to about 100 or more amino acids in length. In some embodiments, a peptide linker may be a GS linker.

In some embodiments, two or more polynucleotide molecules may be linked by a linker that can be an organic molecule, group, polymer, or chemical moiety such as a bivalent organic moiety. A polynucleotide may be linked or fused to another polynucleotide (at the 5′ end or the 3′ end) via a covalent or non-covenant linkage or binding, including e.g., Watson-Crick base-pairing, or through one or more linking nucleotides. In some embodiments, a polynucleotide motif of a certain structure may be inserted within another polynucleotide sequence (e.g. extension of the hairpin structure in guide RNA). In some embodiments, the linking nucleotides may be naturally occurring nucleotides. In some embodiments, the linking nucleotides may be non-naturally occurring nucleotides.

A “promoter” is a nucleotide sequence that controls or regulates the transcription of a nucleotide sequence (e.g., a coding sequence) that is operably associated with the promoter. The coding sequence controlled or regulated by a promoter may encode a polypeptide and/or a functional RNA. Typically, a “promoter” refers to a nucleotide sequence that contains a binding site for RNA polymerase II and directs the initiation of transcription. In general, promoters are found 5′, or upstream, relative to the start of the coding region of the corresponding coding sequence. A promoter may comprise other elements that act as regulators of gene expression; e.g., a promoter region. These include a TATA box consensus sequence, and often a CAAT box consensus sequence (Breathnach and Chambon, (1981) Annu. Rev. Biochem. 50:349). In plants, the CAAT box may be substituted by the AGGA box (Messing et al., (1983) in Genetic Engineering of Plants, T. Kosuge, C. Meredith and A. Hollaender (eds.), Plenum Press, pp. 211-227). In some embodiments, a promoter region may comprise at least one intron (e.g., SEQ ID NO:61 or SEQ ID NO:62).

Promoters useful with this invention can include, for example, constitutive, inducible, temporally regulated, developmentally regulated, chemically regulated, tissue-preferred and/or tissue-specific promoters for use in the preparation of recombinant nucleic acid molecules, e.g., “synthetic nucleic acid constructs” or “protein-RNA complex.” These various types of promoters are known in the art.

The choice of promoter may vary depending on the temporal and spatial requirements for expression, and also may vary based on the host cell to be transformed. Promoters for many different organisms are well known in the art. Based on the extensive knowledge present in the art, the appropriate promoter can be selected for the particular host organism of interest. Thus, for example, much is known about promoters upstream of highly constitutively expressed genes in model organisms and such knowledge can be readily accessed and implemented in other systems as appropriate.

In some embodiments, a promoter functional in a plant may be used with the constructs of this invention. Non-limiting examples of a promoter useful for driving expression in a plant include the promoter of the RubisCo small subunit gene 1 (PrbcS1), the promoter of the actin gene (Pactin), the promoter of the nitrate reductase gene (Pnr) and the promoter of duplicated carbonic anhydrase gene 1 (Pdca1) (See, Walker et al. Plant Cell Rep. 23:727-735 (2005); Li et al. Gene 403:132-142 (2007); Li et al. Mol Biol. Rep. 37:1143-1154 (2010)). PrbcS1 and Pactin are constitutive promoters and Pnr and Pdca1 are inducible promoters. Pnr is induced by nitrate and repressed by ammonium (Li et al. Gene 403:132-142 (2007)) and Pdca1 is induced by salt (Li et al. Mol Biol. Rep. 37:1143-1154 (2010)).

Examples of constitutive promoters useful for plants include, but are not limited to, cestrum virus promoter (cmp) (U.S. Pat. No. 7,166,770), the rice actin 1 promoter (Wang et al. (1992) Mol. Cell. Biol. 12:3399-3406; as well as U.S. Pat. No. 5,641,876), CaMV 35S promoter (Odell et al. (1985) Nature 313:810-812), CaMV 19S promoter (Lawton et al. (1987) Plant Mol. Biol. 9:315-324), nos promoter (Ebert et al. (1987) Proc. Natl. Acad. Sci USA 84:5745-5749), Adh promoter (Walker et al. (1987) Proc. Natl. Acad. Sci. USA 84:6624-6629), sucrose synthase promoter (Yang & Russell (1990) Proc. Natl. Acad. Sci. USA 87:4144-4148), and the ubiquitin promoter. The constitutive promoter derived from ubiquitin accumulates in many cell types. Ubiquitin promoters have been cloned from several plant species for use in transgenic plants, for example, sunflower (Binet et al., 1991. Plant Science 79:87-94), maize (Christensen et al., 1989. Plant Molec. Biol. 12:619-632), and arabidopsis (Norris et al. 1993. Plant Molec. Biol. 21:895-906). The maize ubiquitin promoter (UbiP) has been developed in transgenic monocot systems and its sequence and vectors constructed for monocot transformation are disclosed in the European patent publication EP0342926. The ubiquitin promoter is suitable for the expression of the nucleotide sequences of the invention in transgenic plants, especially monocotyledons. Further, the promoter expression cassettes described by McElroy et al. (Mol. Gen. Genet. 231:150-160 (1991)) can be easily modified for the expression of the nucleotide sequences of the invention and are particularly suitable for use in monocotyledonous hosts.

In some embodiments, tissue specific/tissue preferred promoters can be used for expression of a heterologous polynucleotide in a plant cell. Tissue specific or preferred expression patterns include, but are not limited to, green tissue specific or preferred, root specific or preferred, stem specific or preferred, flower specific or preferred or pollen specific or preferred. Promoters suitable for expression in green tissue include many that regulate genes involved in photosynthesis and many of these have been cloned from both monocotyledons and dicotyledons. In one embodiment, a promoter useful with the invention is the maize PEPC promoter from the phosphoenol carboxylase gene (Hudspeth & Grula, Plant Molec. Biol. 12:579-589 (1989)). Non-limiting examples of tissue-specific promoters include those associated with genes encoding the seed storage proteins (such as β-conglycinin, cruciferin, napin and phaseolin), zein or oil body proteins (such as oleosin), or proteins involved in fatty acid biosynthesis (including acyl carrier protein, stearoyl-ACP desaturase and fatty acid desaturases (fad 2-1)), and other nucleic acids expressed during embryo development (such as Bcc4, see, e.g., Kridl et al. (1991) Seed Sci. Res. 1:209-219; as well as EP U.S. Pat. No. 255,378). Tissue-specific or tissue-preferential promoters useful for the expression of the nucleotide sequences of the invention in plants, particularly maize, include but are not limited to those that direct expression in root, pith, leaf or pollen. Such promoters are disclosed, for example, in WO 93/07278, incorporated by reference herein for its disclosure of promoters. Other non-limiting examples of tissue specific or tissue preferred promoters useful with the invention the cotton rubisco promoter disclosed in U.S. Pat. No. 6,040,504; the rice sucrose synthase promoter disclosed in U.S. Pat. No. 5,604,121; the root specific promoter described by de Framond (FEBS 290:103-106 (1991); European patent EP 0452269 to Ciba-Geigy); the stem specific promoter described in U.S. Pat. No. 5,625,136 (to Ciba-Geigy) and which drives expression of the maize trpA gene; the cestrum yellow leaf curling virus promoter disclosed in WO 01/73087; and pollen specific or preferred promoters including, but not limited to, ProOsLPS10 and ProOsLPS11 from rice (Nguyen et al. Plant Biotechnol. Reports 9(5):297-306 (2015)), ZmSTK2_USP from maize (Wang et al. Genome 60(6):485-495 (2017)), LAT52 and LAT59 from tomato (Twell et al. Development 109(3):705-713 (1990)), Zm13 (U.S. Pat. No. 10,421,972), PLA₂-δ promoter from arabidopsis (U.S. Pat. No. 7,141,424), and/or the ZmC5 promoter from maize (International PCT Publication No. WO1999/042587.

Additional examples of plant tissue-specific/tissue preferred promoters include, but are not limited to, the root hair-specific cis-elements (RHEs) (KIM ET AL. The Plant Cell 18:2958-2970 (2006)), the root-specific promoters RCc3 (Jeong et al. Plant Physiol. 153:185-197 (2010)) and RB7 (U.S. Pat. No. 5,459,252), the lectin promoter (Lindstrom et al. (1990) Der. Genet. 11:160-167; and Vodkin (1983) Prog. Clin. Biol. Res. 138:87-98), corn alcohol dehydrogenase 1 promoter (Dennis et al. (1984) Nucleic Acids Res. 12:3983-4000), S-adenosyl-L-methionine synthetase (SAMS) (Vander Mijnsbrugge et al. (1996) Plant and Cell Physiology, 37(8):1108-1115), corn light harvesting complex promoter (Bansal et al. (1992) Proc. Natl. Acad. Sci. USA 89:3654-3658), corn heat shock protein promoter (O'Dell et al. (1985) EMBO J. 5:451-458; and Rochester et al. (1986) EMBO J. 5:451-458), pea small subunit RuBP carboxylase promoter (Cashmore, “Nuclear genes encoding the small subunit of ribulose-1,5-bisphosphate carboxylase” pp. 29-39 In: Genetic Engineering of Plants (Hollaender ed., Plenum Press 1983; and Poulsen et al. (1986) Mol. Gen. Genet. 205:193-200), Ti plasmid mannopine synthase promoter (Langridge et al. (1989) Proc. Natl. Acad. Sci. USA 86:3219-3223), Ti plasmid nopaline synthase promoter (Langridge et al. (1989), supra), petunia chalcone isomerase promoter (van Tunen et al. (1988) EMBO J. 7:1257-1263), bean glycine rich protein 1 promoter (Keller et al. (1989) Genes Dev. 3:1639-1646), truncated CaMV 35S promoter (O'Dell et al. (1985) Nature 313:810-812), potato patatin promoter (Wenzler et al. (1989) Plant Mol. Biol. 13:347-354), root cell promoter (Yamamoto et al. (1990) Nucleic Acids Res. 18:7449), maize zein promoter (Kriz et al. (1987) Mol. Gen. Genet. 207:90-98; Langridge et al. (1983) Cell 34:1015-1022; Reina et al. (1990) Nucleic Acids Res. 18:6425; Reina et al. (1990) Nucleic Acids Res. 18:7449; and Wandelt et al. (1989) Nucleic Acids Res. 17:2354), globulin-1 promoter (Belanger et al. (1991) Genetics 129:863-872), α-tubulin cab promoter (Sullivan et al. (1989) Mol. Gen. Genet. 215:431-440), PEPCase promoter (Hudspeth & Grula (1989) Plant Mol. Biol. 12:579-589), R gene complex-associated promoters (Chandler et al. (1989) Plant Cell 1:1175-1183), and chalcone synthase promoters (Franken et al. (1991) EMBO J. 10:2605-2612).

Useful for seed-specific expression is the pea vicilin promoter (Czako et al. (1992) Mol. Gen. Genet. 235:33-40; as well as the seed-specific promoters disclosed in U.S. Pat. No. 5,625,136. Useful promoters for expression in mature leaves are those that are switched at the onset of senescence, such as the SAG promoter from Arabidopsis (Gan et al. (1995) Science 270:1986-1988).

In addition, promoters functional in chloroplasts can be used. Non-limiting examples of such promoters include the bacteriophage T3 gene 9 5′ UTR and other promoters disclosed in U.S. Pat. No. 7,579,516. Other promoters useful with the invention include but are not limited to the S-E9 small subunit RuBP carboxylase promoter and the Kunitz trypsin inhibitor gene promoter (Kti3).

Additional regulatory elements useful with this invention include, but are not limited to, introns, enhancers, termination sequences and/or 5′ and 3′ untranslated regions.

An intron useful with this invention can be an intron identified in and isolated from a plant and then inserted into an expression cassette to be used in transformation of a plant. As would be understood by those of skill in the art, introns can comprise the sequences required for self-excision and are incorporated into nucleic acid constructs/expression cassettes in frame. An intron can be used either as a spacer to separate multiple protein-coding sequences in one nucleic acid construct, or an intron can be used inside one protein-coding sequence to, for example, stabilize the mRNA. If they are used within a protein-coding sequence, they are inserted “in-frame” with the excision sites included. Introns may also be associated with promoters to improve or modify expression. As an example, a promoter/intron combination useful with this invention includes but is not limited to that of the maize Ubi1 promoter and intron.

Non-limiting examples of introns useful with the present invention include introns from the ADHI gene (e.g., Adh1-S introns 1, 2 and 6), the ubiquitin gene (Ubi1), the RuBisCO small subunit (rbcS) gene, the RuBisCO large subunit (rbcL) gene, the actin gene (e.g., actin-1 intron), the pyruvate dehydrogenase kinase gene (pdk), the nitrate reductase gene (nr), the duplicated carbonic anhydrase gene 1 (Tdca1), the psbA gene, the atpA gene, or any combination thereof.

“Gene editing machinery” and a “gene editing complex” as used herein can each be referred to as an editing system. “Gene editing machinery” and a “gene editing complex” useful with this invention can be any site-specific (sequence-specific) genome editing system now known or later developed, which system can introduce mutations in a target specific manner. For example, gene editing machinery or a gene editing complex can include, but are not limited to, a CRISPR-Cas editing system, a meganuclease editing system, a zinc finger nuclease (ZFN) editing system, a transcription activator-like effector nuclease (TALEN) editing system, a base editing system and/or a prime editing system, each of which can comprise one or more polypeptides and/or one or more polynucleotides that when present and/or expressed as a system in a cell can modify (mutate) a target nucleic acid in a sequence specific manner. In some embodiments, an editing system (e.g., site- or sequence-specific editing system) can comprise one or more polynucleotides and/or one or more polypeptides, including but not limited to a nucleic acid binding domain (DNA binding domain), a nuclease, and/or other polypeptide, and/or a polynucleotide.

In some embodiments, an editing system can comprise one or more sequence-specific nucleic acid binding domains (e.g., DNA binding domains) that can be from, for example, a polynucleotide-guided endonuclease, a CRISPR-Cas endonuclease (e.g., CRISPR-Cas effector protein), a zinc finger nuclease, a transcription activator-like effector nuclease (TALEN) and/or an Argonaute protein. In some embodiments, an editing system can comprise one or more cleavage domains (e.g., nucleases) including, but not limited to, an endonuclease (e.g., Fok1), a polynucleotide-guided endonuclease, a CRISPR-Cas endonuclease (e.g., CRISPR-Cas effector protein), a zinc finger nuclease, and/or a transcription activator-like effector nuclease (TALEN).

A “nucleic acid binding domain” as used herein includes a DNA binding domain and may be a site- or sequence-specific nucleic acid binding domain. In some embodiments, a nucleic acid binding domain may be a sequence-specific nucleic acid binding domain such as, but not limited to, a sequence-specific binding domain from, for example, a polynucleotide-guided endonuclease, a CRISPR-Cas effector protein (e.g., CRISPR-Cas endonuclease), a zinc finger nuclease, a transcription activator-like effector nuclease (TALEN) and/or an Argonaute protein. In some embodiments, a nucleic acid binding domain comprises a cleavage domain (e.g., a nuclease domain) such as, but not limited to, an endonuclease (e.g., Fok1), a polynucleotide-guided endonuclease, a CRISPR-Cas endonuclease, a zinc finger nuclease, and/or a transcription activator-like effector nuclease (TALEN). In some embodiments, the nucleic acid binding domain is a polypeptide that can associate (e.g., form a complex) with one or more nucleic acid molecules (e.g., form a complex with a guide nucleic acid as described herein) that can direct or guide the nucleic acid binding domain to a specific target nucleotide sequence (e.g., a gene locus of a genome) that is complementary to the one or more nucleic acid molecules (or a portion or region thereof), thereby causing the nucleic acid binding domain to bind to the nucleotide sequence at the specific target site. In some embodiments, the nucleic acid binding domain is a CRISPR-Cas effector protein as described herein. In some embodiments, reference is made to specifically to a CRISPR-Cas effector protein for simplicity, but a nucleic acid binding domain as described herein may be used.

In some embodiments, a gene editing complex comprises a ribonucleoprotein such as an assembled ribonucleoprotein complex (e.g., that comprises a CRISPR-Cas effector protein and a guide nucleic acid). A gene editing complex, as used herein, may be assembled when introduced into a plant cell or may assemble into a complex (e.g., a covalently and/or non-covalently bound complex) after and/or during introduction into a plant cell. Exemplary ribonucleoproteins and methods of use thereof include, but are not limited to, those described in Malnoy et al., (2016) Front. Plant Sci. 7:1904; Subburaj et al., (2016) Plant Cell Rep. 35:1535; Woo et al., (2015) Nat. Biotechnol. 33:1162; Liang et al., (2017) Nat. Commun. 8:14261; Svitashev et al., Nat. Commun. 7, 13274 (2016); Zhang et al., (2016) Nat. Commun. 7:12617; Kim et al., (2017) Nat. Commun. 8:14406.

In some embodiments, a polynucleotide and/or a nucleic acid construct of the invention can be an “expression cassette” or can be comprised within an expression cassette. As used herein, “expression cassette” means a recombinant nucleic acid molecule comprising, for example, a nucleic acid construct of the invention (e.g., a polynucleotide comprising a heterologous morphogenic regulator, a polynucleotide encoding a CRISPR-Cas effector protein, a polynucleotide encoding a CRISPR-Cas fusion protein, a polynucleotide encoding a cytosine deaminase, a polynucleotide encoding an adenine deaminase, and/or a polynucleotide comprising a guide nucleic acid), wherein the nucleic acid construct is operably associated with at least a control sequence (e.g., a promoter). Thus, some embodiments of the invention provide expression cassettes designed to express, for example, a nucleic acid construct of the invention. When an expression cassette comprises more than one polynucleotide, the polynucleotides may be operably linked to a single promoter that drives expression of all of the polynucleotides or the polynucleotides may be operably linked to one or more different promoters (e.g., three polynucleotides may be driven by one, two or three promoters in any combination). Thus, for example, a polynucleotide comprising a heterologous morphogenic regulator, a polynucleotide encoding a CRISPR-Cas effector protein, a polynucleotide encoding a deaminase, and a polynucleotide comprising a guide nucleic acid comprised in an expression cassette may each be operably associated with a single promoter or one or more of the polynucleotide(s) may be operably associated with separate promoters (e.g., two or three promoters) in any combination. In some embodiments, two or more different expression cassettes can be provided that one or more polynucleotides. For example, a first expression cassette may be provided that comprises a polynucleotide comprising a heterologous morphogenic regulator and a second expression cassette may be provided that comprises a polynucleotide encoding a CRISPR-Cas effector protein, a polynucleotide encoding a deaminase, and a polynucleotide comprising a guide nucleic acid that may each be operably associated with a single promoter or one or more of the polynucleotide(s) may be operably associated with separate promoters (e.g., two or three promoters) in any combination. The first expression cassette may comprise one or more polynucleotides that are the same as or different than one or more polynucleotides in the second expression cassette and vice versa.

In some embodiments, an expression cassette comprising the polynucleotides/nucleic acid constructs of the invention may be optimized for expression in an organism (e.g., an animal, a plant, a bacterium and the like).

An expression cassette comprising a nucleic acid construct of the invention may be chimeric, meaning that at least one of its components is heterologous with respect to at least one of its other components (e.g., a promoter from the host organism operably linked to a polynucleotide of interest to be expressed in the host organism, wherein the polynucleotide of interest is from a different organism than the host or is not normally found in association with that promoter). An expression cassette may also be one that is naturally occurring but has been obtained in a recombinant form useful for heterologous expression.

An expression cassette can optionally include a transcriptional and/or translational termination region (i.e., termination region) and/or an enhancer region that is functional in the selected host cell. A variety of transcriptional terminators and enhancers are known in the art and are available for use in expression cassettes. Transcriptional terminators are responsible for the termination of transcription and correct mRNA polyadenylation. A termination region and/or the enhancer region may be native to the transcriptional initiation region, may be native to a gene encoding a CRISPR-Cas effector protein or a gene encoding a deaminase, may be native to a host cell, or may be native to another source (e.g., foreign or heterologous to the promoter, to a gene encoding the CRISPR-Cas effector protein or a gene encoding the deaminase, to a host cell, or any combination thereof).

An expression cassette of the invention also can include a polynucleotide encoding a selectable marker, which can be used to select a transformed host cell. As used herein, “selectable marker” means a polynucleotide sequence that when expressed imparts a distinct phenotype to the host cell expressing the marker and thus allows such transformed cells to be distinguished from those that do not have the marker. Such a polynucleotide sequence may encode either a selectable or screenable marker, depending on whether the marker confers a trait that can be selected for by chemical means, such as by using a selective agent (e.g., an antibiotic and the like), or on whether the marker is simply a trait that one can identify through observation or testing, such as by screening (e.g., fluorescence). Many examples of suitable selectable markers are known in the art and can be used in the expression cassettes described herein.

The expression cassettes, the nucleic acid molecules/constructs and polynucleotide sequences described herein can be used in connection with vectors. The term “vector” refers to a composition for transferring, delivering or introducing a nucleic acid (or nucleic acids) into a cell. A vector comprises a nucleic acid construct comprising the nucleotide sequence(s) to be transferred, delivered or introduced. Vectors for use in transformation of host organisms are well known in the art. Non-limiting examples of general classes of vectors include viral vectors, plasmid vectors, phage vectors, phagemid vectors, cosmid vectors, fosmid vectors, bacteriophages, artificial chromosomes, minicircles, or Agrobacterium binary vectors in double or single stranded linear or circular form which may or may not be self transmissible or mobilizable. In some embodiments, a viral vector can include, but is not limited, to a retroviral, lentiviral, adenoviral, adeno-associated, or herpes simplex viral vector. A vector as defined herein can transform a prokaryotic or eukaryotic host either by integration into the cellular genome or exist extrachromosomally (e.g. autonomous replicating plasmid with an origin of replication). Additionally included are shuttle vectors by which is meant a DNA vehicle capable, naturally or by design, of replication in two different host organisms, which may be selected from actinomycetes and related species, bacteria and eukaryotic (e.g. higher plant, mammalian, yeast or fungal cells). In some embodiments, the nucleic acid in the vector is under the control of, and operably linked to, an appropriate promoter or other regulatory elements for transcription in a host cell. The vector may be a bi-functional expression vector which functions in multiple hosts. In the case of genomic DNA, this may contain its own promoter and/or other regulatory elements and in the case of cDNA this may be under the control of an appropriate promoter and/or other regulatory elements for expression in the host cell. Accordingly, a nucleic acid construct of this invention and/or expression cassettes comprising the same may be comprised in vectors as described herein and as known in the art.

As used herein, “contact,” “contacting,” “contacted,” and grammatical variations thereof, refer to placing the components of a desired reaction together under conditions suitable for carrying out the desired reaction (e.g., transformation, transcriptional control, genome editing, nicking, and/or cleavage). Thus, for example, a target nucleic acid may be contacted with a nucleic acid construct of the invention encoding, for example, a nucleic acid binding domain (e.g., a DNA binding domain such as a sequence-specific DNA binding protein (e.g., polynucleotide-guided endonuclease, a CRISPR-Cas effector protein (e.g., CRISPR-Cas endonuclease), a zinc finger nuclease, a transcription activator-like effector nuclease (TALEN) and/or an Argonaute protein)), a guide nucleic acid, a deaminase (e.g., a cytosine and/or adenine deaminase), and a heterologous morphogenic regulator, under conditions whereby the CRISPR-Cas effector protein and heterologous morphogenic regulator are expressed, and the CRISPR-Cas effector protein forms a complex with the guide nucleic acid, the complex hybridizes to the target nucleic acid, and optionally the deaminase is recruited to the CRISPR-Cas effector protein (and thus, to the target nucleic acid) or the deaminase is fused to the CRISPR-Cas effector protein, thereby modifying the target nucleic acid. In some embodiments, the deaminase and the CRISPR-Cas effector protein localize at the target nucleic acid, optionally through covalent and/or non-covalent interactions.

As used herein, “modifying” or “modification” in reference to a target nucleic acid includes editing (e.g., mutating), covalent modification, exchanging/substituting nucleic acids/nucleotide bases, deleting, cleaving, nicking, and/or transcriptional control of a target nucleic acid.

“Recruit,” “recruiting” or “recruitment” as used herein refer to attracting one or more polypeptide(s) or polynucleotide(s) to another polypeptide or polynucleotide (e.g., to a particular location in a genome) using protein-protein interactions, RNA-protein interactions, and/or chemical interactions. Protein-protein interactions can include, but are not limited to, peptide tags (epitopes, multimerized epitopes) and corresponding affinity polypeptides, RNA recruiting motifs and corresponding affinity polypeptides, and/or chemical interactions. Example chemical interactions that may be useful with polypeptides and polynucleotides for the purpose of recruitment can include, but are not limited to, rapamycin-inducible dimerization of FRB-FKBP; Biotin-streptavidin interaction; SNAP tag (Hussain et al. Curr Pharm Des. 19(30):5437-42 (2013)); Halo tag (Los et al. ACS Chem Biol. 3(6):373-82 (2008)); CLIP tag (Gautier et al. Chemistry & Biology 15:128-136 (2008)); DmrA-DmrC heterodimer induced by a compound (Tak et al. Nat Methods 14(12):1163-1166 (2017)); Bifunctional ligand approaches (fuse two protein-binding chemicals together) (Voß et al. Curr Opin Chemical Biology 28:194-201 (2015)) (e.g. dihyrofolate reductase (DHFR) (Kopyteck et al. Cell Chem Biol 7(5):313-321 (2000)).

“Introducing,” “introduce,” “introduced” (and grammatical variations thereof) in the context of a polynucleotide of interest, polypeptide of interest, and/or editing system means presenting a nucleotide sequence of interest (e.g., polynucleotide, a nucleic acid construct, and/or a guide nucleic acid), a polypeptide of interest, and/or an editing system (e.g., a polynucleotide, polypeptide, and/or ribonucleoprotein) to a host organism or cell of said organism (e.g., host cell; e.g., a plant cell) in such a manner that the nucleotide sequence of interest, polypeptide of interest, and/or editing system gains access to the interior of a cell. Thus, for example, a polynucleotide comprising a heterologous morphogenic regulator and/or a nucleic acid construct encoding a CRISPR-Cas effector protein, a guide nucleic acid, and a deaminase may be introduced into a cell of an organism, thereby transforming the cell with the polynucleotide comprising a heterologous morphogenic regulator and/or the CRISPR-Cas effector protein, guide nucleic acid, and deaminase. Another example is that a polynucleotide comprising a heterologous morphogenic regulator and/or an editing system comprising a ribonucleoprotein may be introduced into a cell of an organism, thereby transforming the cell with the polynucleotide comprising a heterologous morphogenic regulator and/or the ribonucleoprotein. The ribonucleoprotein may comprise a CRISPR-Cas effector domain, guide nucleic acid, and optionally a deaminase domain. In some embodiments, a polypeptide comprising a heterologous morphogenic regulator and/or an editing system comprising a ribonucleoprotein may be introduced into a cell of an organism, thereby transforming the cell with the polypeptide comprising a heterologous morphogenic regulator and/or the ribonucleoprotein.

The term “transformation” as used herein refers to the introduction of a heterologous nucleic acid, polypeptide, and/or ribonucleoprotein into a cell. Transformation of a cell may be stable or transient. Thus, in some embodiments, a host cell or host organism may be stably transformed with a polynucleotide/nucleic acid molecule of the invention. In some embodiments, a host cell or host organism may be transiently transformed with a nucleic acid construct, a polypeptide and/or a ribonucleoprotein of the invention.

“Transient transformation” or “transiently transformed” in the context of a polynucleotide, polypeptide, and/or ribonucleoprotein means that a polynucleotide, polypeptide, and/or ribonucleoprotein is introduced into the cell and does not integrate into the genome of the cell.

By “stably introducing” or “stably introduced” in the context of a polynucleotide that is introduced into a cell is intended that the introduced polynucleotide is stably incorporated into the genome of the cell, and thus the cell is stably transformed with the polynucleotide.

“Stable transformation” or “stably transformed” as used herein means that a nucleic acid molecule is introduced into a cell and integrates into the genome of the cell. As such, the integrated nucleic acid molecule is capable of being inherited by the progeny thereof, more particularly, by the progeny of multiple successive generations. “Genome” as used herein includes the nuclear and the plastid genome, and therefore includes integration of the nucleic acid into, for example, the chloroplast or mitochondrial genome. Stable transformation as used herein can also refer to a transgene that is maintained extrachromasomally, for example, as a minichromosome or a plasmid.

Transient transformation may be detected by, for example, an enzyme-linked immunosorbent assay (ELISA) or Western blot, which can detect the presence of a peptide or polypeptide encoded by one or more transgene introduced into an organism. Stable transformation of a cell can be detected by, for example, a Southern blot hybridization assay of genomic DNA of the cell with nucleic acid sequences which specifically hybridize with a nucleotide sequence of a transgene introduced into an organism (e.g., a plant). Stable transformation of a cell can be detected by, for example, a Northern blot hybridization assay of RNA of the cell with nucleic acid sequences which specifically hybridize with a nucleotide sequence of a transgene introduced into a host organism. Stable transformation of a cell can also be detected by, e.g., a polymerase chain reaction (PCR) or other amplification reactions as are well known in the art, employing specific primer sequences that hybridize with target sequence(s) of a transgene, resulting in amplification of the transgene sequence, which can be detected according to standard methods Transformation can also be detected by direct sequencing and/or hybridization protocols well known in the art.

Accordingly, in some embodiments, nucleotide sequences, polynucleotides, nucleic acid constructs, and/or expression cassettes of the invention may be expressed transiently and/or they can be stably incorporated into the genome of the host organism. Thus, in some embodiments, a nucleic acid construct of the invention may be transiently introduced into a cell with a guide nucleic acid and as such, no DNA maintained in the cell.

A nucleic acid construct, polypeptide, and/or ribonucleoprotein of the invention can be introduced into a cell by any method known to those of skill in the art. In some embodiments of the invention, transformation of a cell comprises nuclear transformation. In other embodiments, transformation of a cell comprises plastid transformation (e.g., chloroplast transformation). In some embodiments of the invention, transformation of a cell comprises particle bombardment. In still further embodiments, the recombinant nucleic acid construct of the invention can be introduced into a cell via conventional breeding techniques.

Procedures for transforming both eukaryotic and prokaryotic organisms are well known and routine in the art and are described throughout the literature (See, for example, Jiang et al. 2013. Nat. Biotechnol. 31:233-239; Ran et al. Nature Protocols 8:2281-2308 (2013)).

A nucleotide sequence, polypeptide, and/or ribonucleoprotein therefore can be introduced into a host organism or its cell in any number of ways that are well known in the art. The methods of the invention do not depend on a particular method for introducing one or more nucleotide sequence(s), polypeptide(s), and/or ribonucleoprotein(s) into the organism, only that they gain access to the interior of at least one cell of the organism. Where more than one nucleotide sequence, polypeptide, and/or ribonucleoprotein is to be introduced, they can be assembled as part of a single construct (e.g., a single nucleic acid construct,) or as separate constructs. Accordingly, a nucleotide sequence, polypeptide, and/or ribonucleoprotein can be introduced into the cell of interest in a single transformation event, and/or in separate transformation events, or, alternatively, where relevant, a nucleotide sequence can be incorporated into a plant, for example, as part of a breeding protocol.

The terms “genetically engineered host cell,” “recombinant host cell,” and “recombinant strain” are used interchangeably herein and refer to host cells that have been genetically engineered by a method of the present disclosure. Thus, the terms include a host cell (e.g., bacteria, yeast cell, fungal cell, CHO, human cell, plant cell, protoplast derived from plant, callus, etc.) that has been genetically altered, modified, or engineered, such that it exhibits an altered, modified, or different genotype and/or phenotype (e.g., when the genetic modification affects coding nucleic acid sequences), as compared to the naturally-occurring host cell from which it was derived. It is understood that the terms refer not only to the particular recombinant host cell in question, but also to the progeny or potential progeny of such a host cell.

The term “genetically engineered” may refer to any manipulation of a host cell's genome (e.g., by insertion or deletion of nucleic acids).

The term “next generation plant breeding” refers to a host of plant breeding tools and methodologies that are available to today's breeder. A key distinguishing feature of next generation plant breeding is that the breeder is no longer confined to relying upon observed phenotypic variation, in order to infer underlying genetic causes for a given trait. Rather, next generation plant breeding may include the utilization of molecular markers and marker assisted selection (MAS), such that the breeder can directly observe movement of alleles and genetic elements of interest from one plant in the breeding population to another, and is not confined to merely observing phenotype. Further, next generation plant breeding methods are not confined to utilizing natural genetic variation found within a plant population. Rather, the breeder utilizing next generation plant breeding methodology can access a host of modern genetic engineering tools that directly alter/change/edit the plant's underlying genetic architecture in a targeted manner, in order to bring about a phenotypic trait of interest. In aspects, the plants bred with a next generation plant breeding methodology are indistinguishable from a plant that was bred in a traditional manner, as the resulting end product plant could theoretically be developed by either method. In particular aspects, a next generation plant breeding methodology may result in a plant that comprises: a genetic modification that is a deletion of any size; a genetic modification that is a single base pair substitution; a genetic modification that is an introduction of nucleic acid sequences from within the plant's natural gene pool (e.g. any plant that could be crossed or bred with a plant of interest) or from editing of nucleic acid sequences in a plant to correspond to a sequence known to occur in the plant's natural gene pool; and offspring of said plants.

The term “traditional plant breeding” refers to the utilization of natural variation found within a plant population as a source for alleles and genetic variants that impart a trait of a interest to a given plant. Traditional breeding methods make use of crossing procedures that rely largely upon observed phenotypic variation to infer causative allele association. That is, traditional plant breeding relies upon observations of expressed phenotype of a given plant to infer underlying genetic cause. These observations are utilized to inform the breeding procedure in order to move allelic variation into germplasm of interest. Further, traditional plant breeding has also been characterized as comprising random mutagenesis techniques, which can be used to introduce genetic variation into a given germplasm. These random mutagenesis techniques may include chemical and/or radiation-based mutagenesis procedures. Consequently, one key feature of traditional plant breeding, is that the breeder does not utilize a genetic engineering tool that directly alters/changes/edits the plant's underlying genetic architecture in a targeted manner, in order to introduce genetic diversity and bring about a phenotypic trait of interest.

As used herein, the term “endogenous gene,” refers to a naturally occurring gene, in the location in which it is naturally found within the host cell genome. An endogenous gene as described herein can include alleles of naturally occurring genes that have been mutated according to any of a method of the present disclosure, i.e. an endogenous gene could have been modified at some point by traditional plant breeding methods and/or next generation plant breeding methods.

As used herein, the term “exogenous” refers to a substance coming from some source other than its native source. For example, the terms “exogenous protein,” or “exogenous gene” refer to a protein or gene from a non-native source, and that has been artificially supplied to a biological system. As used herein, the term “exogenous” is used interchangeably with the term “heterologous,” and refers to a substance coming from some source other than its native source.

As used herein, the term “heterologous” refers to a substance coming from some source or location other than its native source or location. In some embodiments, the term “heterologous nucleic acid” refers to a nucleic acid sequence that is not naturally found in the particular organism. For example, the term “heterologous promoter” may refer to a promoter that has been taken from one source organism and utilized in another organism, in which the promoter is not naturally found. However, the term “heterologous promoter” may also refer to a promoter that is from within the same source organism, but has merely been moved to a novel location, in which said promoter is not normally located.

As used herein, the term “nucleotide change” refers to, e.g., nucleotide substitution, deletion, and/or insertion, as is well understood in the art. For example, mutations contain alterations that produce single nucleotide substitutions, silent substitutions, additions, or deletions, but do not alter the properties or activities of the encoded protein or how the proteins are made.

As used herein, the term “protein modification” refers to, e.g., amino acid substitution, amino acid modification, deletion, and/or insertion, as is well understood in the art.

As used herein, the term “codon optimization” implies that the codon usage of a DNA or RNA is adapted to that of a cell or organism of interest to improve the transcription rate of said recombinant nucleic acid in the cell or organism of interest. The skilled person is well aware of the fact that a target nucleic acid can be modified at one position due to the codon degeneracy, whereas this modification will still lead to the same amino acid sequence at that position after translation, which is achieved by codon optimization to take into consideration the species-specific codon usage of a target cell or organism.

As used herein, the term “naturally occurring” as applied to a nucleic acid, a polypeptide, a cell, or an organism, refers to a nucleic acid, polypeptide, cell, or organism that is found in nature. The term “naturally occurring” may refer to a gene or sequence derived from a naturally occurring source. Thus, for the purposes of this disclosure, a “non-naturally occurring” sequence is a sequence that has been synthesized, mutated, engineered, edited, or otherwise modified to have a different sequence from known natural sequences. In some embodiments, the modification may be at the protein level (e.g., amino acid substitutions, deletions, or insertions). In other embodiments, the modification may be at the DNA level (e.g., nucleotide substitutions, deletions, or insertions).

As used herein, the phrases “recombinant construct”, “expression construct”, “chimeric construct”, and “recombinant DNA construct” are used interchangeably herein. A recombinant construct comprises an artificial combination of nucleic acid fragments, e.g., regulatory and coding sequences that are not found together in nature. For example, a chimeric construct may comprise regulatory sequences and coding sequences that are derived from different sources, or regulatory sequences and coding sequences derived from the same source, but arranged in a manner different than that found in nature. Such construct may be used by itself or may be used in conjunction with a vector. If a vector is used then the choice of vector is dependent upon the method that will be used to transform host cells as is well known to those skilled in the art. For example, a plasmid vector can be used. The skilled artisan is well aware of the genetic elements that must be present on the vector in order to successfully transform, select and propagate host cells comprising any of the isolated nucleic acid fragments of the disclosure. The skilled artisan will also recognize that different independent transformation events will result in different levels and patterns of expression (Jones et al., (1985) EMBO J. 4:2411-2418; De Almeida et al., (1989) Mol. Gen. Genetics 218:78-86), and thus that multiple events must be screened in order to obtain lines displaying the desired expression level and pattern. Such screening may be accomplished by Southern analysis of DNA, Northern analysis of mRNA expression, immunoblotting analysis of protein expression, or phenotypic analysis, among others. Vectors can be plasmids, viruses, bacteriophages, pro-viruses, phagemids, transposons, artificial chromosomes, and the like, that replicate autonomously or can integrate into a chromosome of a host cell. A vector can also be a naked RNA polynucleotide, a naked DNA polynucleotide, a polynucleotide composed of both DNA and RNA within the same strand, a poly-lysine-conjugated DNA or RNA, a peptide-conjugated DNA or RNA, a liposome-conjugated DNA, or the like, that is not autonomously replicating. As used herein, the term “expression” refers to the production of a functional end-product e.g., an mRNA or a protein (precursor or mature).

In some embodiments, an editing system of the present invention comprises a CRISPR-Cas effector protein. As used herein, a “CRISPR-Cas effector protein” is a protein or polypeptide or domain thereof that cleaves, cuts, or nicks a nucleic acid, binds a nucleic acid (e.g., a target nucleic acid and/or a guide nucleic acid), and/or that identifies, recognizes, or binds a guide nucleic acid as defined herein. In some embodiments, a CRISPR-Cas effector protein may be an enzyme (e.g., a nuclease, endonuclease, nickase, etc.) or portion thereof and/or may function as an enzyme. In some embodiments, a CRISPR-Cas effector protein refers to a CRISPR-Cas nuclease polypeptide or domain thereof that comprises nuclease activity or in which the nuclease activity has been reduced or eliminated, and/or comprises nickase activity or in which the nickase has been reduced or eliminated, and/or comprises single stranded DNA cleavage activity (ss DNAse activity) or in which the ss DNAse activity has been reduced or eliminated, and/or comprises self-processing RNAse activity or in which the self-processing RNAse activity has been reduced or eliminated. A CRISPR-Cas effector protein may bind to a target nucleic acid. A CRISPR-Cas effector protein may be a Type I, II, III, IV, V, or VI CRISPR-Cas effector protein. In some embodiments, a CRISPR-Cas effector protein may be from a Type I CRISPR-Cas system, a Type II CRISPR-Cas system, a Type III CRISPR-Cas system, a Type IV CRISPR-Cas system, Type V CRISPR-Cas system, or a Type VI CRISPR-Cas system. In some embodiments, a CRISPR-Cas effector protein of the invention may be from a Type II CRISPR-Cas system or a Type V CRISPR-Cas system. In some embodiments, a CRISPR-Cas effector protein may be a Type II CRISPR-Cas effector protein, for example, a Cas9 effector protein. In some embodiments, a CRISPR-Cas effector protein may be Type V CRISPR-Cas effector protein, for example, a Cas12 effector protein. In some embodiments, a CRISPR-Cas effector protein may be a Cas12a polypeptide or domain thereof and optionally may have an amino acid sequence of any one of SEQ ID NOs:115-131 and/or a nucleotide sequence of any one of SEQ ID NOs:132-134.

In some embodiments, a CRISPR-Cas effector protein may be or include, but is not limited to, a Cas9, C2c1, C2c3, Cas12a (also referred to as Cpf1), Cas12b, Cas12c, Cas12d, Cas12e, Cas13a, Cas13b, Cas13c, Cas13d, Cas1, Cas1B, Cas2, Cas3, Cas3′, Cas3″, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4 (dinG), and/or Csf5 nuclease, optionally wherein the CRISPR-Cas effector protein may be a Cas9, Cas12a (Cpf1), Cas12b, Cas12c (C2c3), Cas12d (CasY), Cas12e (CasX), Cas12g, Cas12h, Cas12i, C2c4, C2c5, C2c8, C2c9, C2c10, Cas14a, Cas14b, and/or Cas14c effector protein.

In some embodiments, a CRISPR-Cas effector protein useful with the invention may comprise a mutation in its nuclease active site (e.g., RuvC, HNH, e.g., RuvC site of a Cas12a nuclease domain; e.g., RuvC site and/or HNH site of a Cas9 nuclease domain). A CRISPR-Cas effector protein having a mutation in its nuclease active site, and therefore, no longer comprising nuclease activity, is commonly referred to as “dead,” e.g., dCas9. In some embodiments, a CRISPR-Cas effector protein domain or polypeptide having a mutation in its nuclease active site may have impaired activity or reduced activity as compared to the same CRISPR-Cas effector protein without the mutation, e.g., a nickase, e.g, Cas9 nickase, Cas12a nickase.

A CRISPR Cas9 effector protein or CRISPR Cas9 effector domain useful with this invention may be any known or later identified Cas9 nuclease. In some embodiments, a CRISPR Cas9 polypeptide can be a Cas9 polypeptide from, for example, Streptococcus spp. (e.g., S. pyogenes, S. thermophilus), Lactobacillus spp., Bifidobacterium spp., Kandleria spp., Leuconostoc spp., Oenococcus spp., Pediococcus spp., Weissella spp., and/or Olsenella spp. In some embodiments, a CRISPR-Cas effector protein may be a Cas9 polypeptide or domain thereof and optionally may have a nucleotide sequence of any one of SEQ ID NOs:63-73 and/or an amino acid sequence of any one of SEQ ID NOs:74-75.

In some embodiments, the CRISPR-Cas effector protein may be a Cas9 polypeptide derived from Streptococcus pyogenes and recognizes the PAM sequence motif NGG, NAG, NGA (Mali et al, Science 2013; 339(6121): 823-826). In some embodiments, the CRISPR-Cas effector protein may be a Cas9 polypeptide derived from Streptococcus thermophiles and recognizes the PAM sequence motif NGGNG and/or NNAGAAW (W=A or T) (See, e.g., Horvath et al, Science, 2010; 327(5962): 167-170, and Deveau et al, J Bacteriol 2008; 190(4): 1390-1400). In some embodiments, the CRISPR-Cas effector protein may be a Cas9 polypeptide derived from Streptococcus mutans and recognizes the PAM sequence motif NGG and/or NAAR (R=A or G) (See, e.g., Deveau et al, J BACTERIOL 2008; 190(4): 1390-1400). In some embodiments, the CRISPR-Cas effector protein may be a Cas9 polypeptide derived from Streptococcus aureus and recognizes the PAM sequence motif NNGRR (R=A or G). In some embodiments, the CRISPR-Cas effector protein may be a Cas9 protein derived from S. aureus, which recognizes the PAM sequence motif N GRRT (R=A or G). In some embodiments, the CRISPR-Cas effector protein may be a Cas9 polypeptide derived from S. aureus, which recognizes the PAM sequence motif N GRRV (R=A or G). In some embodiments, the CRISPR-Cas effector protein may be a Cas9 polypeptide that is derived from Neisseria meningitidis and recognizes the PAM sequence motif N GATT or N GCTT (R=A or G, V=A, G or C) (See, e.g., Hou et ah, PNAS 2013, 1-6). In the aforementioned embodiments, N can be any nucleotide residue, e.g., any of A, G, C or T. In some embodiments, the CRISPR-Cas effector protein may be a Cas13a protein derived from Leptotrichia shahii, which recognizes a protospacer flanking sequence (PFS) (or RNA PAM (rPAM)) sequence motif of a single 3′ A, U, or C, which may be located within the target nucleic acid.

A Type V CRISPR-Cas effector protein useful with embodiments of the invention may be any Type V CRISPR-Cas nuclease. A Type V CRISPR-Cas nuclease useful with this invention as an effector protein can include, but is not limited, to Cas12a (Cpf1), Cas12b, Cas12c (C2c3), Cas12d (CasY), Cas12e (CasX), Cas12g, Cas12h, Cas12i, C2c1, C2c4, C2c5, C2c8, C2c9, C2c10, Cas14a, Cas14b, and/or Cas14c nuclease. In some embodiments, a Type V CRISPR-Cas nuclease polypeptide or domain useful with embodiments of the invention may be a Cas12a polypeptide or domain. In some embodiments, a Type V CRISPR-Cas effector protein or domain useful with embodiments of the invention may be a nickase, optionally, a Cas12a nickase.

In some embodiments, the CRISPR-Cas effector protein may be derived from Cas12a, which is a Type V Clustered Regularly Interspaced Short Palindromic Repeats (CRISPR)-Cas nuclease. Cas12a differs in several respects from the more well-known Type II CRISPR Cas9 nuclease. For example, Cas9 recognizes a G-rich protospacer-adjacent motif (PAM) that is 3′ to its guide RNA (gRNA, sgRNA, crRNA, crDNA, CRISPR array) binding site (protospacer, target nucleic acid, target DNA) (3′-NGG), while Cas12a recognizes a T-rich PAM that is located 5′ to the target nucleic acid (5′-TTN, 5′-TTTN. In fact, the orientations in which Cas9 and Cas12a bind their guide RNAs are very nearly reversed in relation to their N and C termini. Furthermore, Cas12a enzymes use a single guide RNA (gRNA, CRISPR array, crRNA) rather than the dual guide RNA (sgRNA (e.g., crRNA and tracrRNA)) found in natural Cas9 systems, and Cas12a processes its own gRNAs. Additionally, Cas12a nuclease activity produces staggered DNA double stranded breaks instead of blunt ends produced by Cas9 nuclease activity, and Cas12a relies on a single RuvC domain to cleave both DNA strands, whereas Cas9 utilizes an HNH domain and a RuvC domain for cleavage.

A CRISPR Cas12a effector protein/domain useful with this invention may be any known or later identified Cas12a polypeptide (previously known as Cpf1) (see, e.g., U.S. Pat. No. 9,790,490, which is incorporated by reference for its disclosures of Cpf1 (Cas12a) sequences). The term “Cas12a”, “Cas12a polypeptide” or “Cas12a domain” refers to an RNA-guided nuclease comprising a Cas12a polypeptide, or a fragment thereof, which comprises the guide nucleic acid binding domain of Cas12a and/or an active, inactive, or partially active DNA cleavage domain of Cas12a. In some embodiments, a Cas12a useful with the invention may comprise a mutation in the nuclease active site (e.g., RuvC site of the Cas12a domain). A Cas12a domain or Cas12a polypeptide having a mutation in its nuclease active site, and therefore, no longer comprising nuclease activity, is commonly referred to as deadCas12a (e.g., dCas12a). In some embodiments, a Cas12a domain or Cas12a polypeptide having a mutation in its nuclease active site may have impaired activity, e.g., may have nickase activity.

In some embodiments, a CRISPR-Cas effector protein may be optimized for expression in an organism, for example, in an animal, a plant, a fungus, an archaeon, or a bacterium. In some embodiments, a CRISPR-Cas effector protein (e.g., Cas12a polypeptide/domain or a Cas9 polypeptide/domain) may be optimized for expression in a plant.

Any deaminase domain/polypeptide useful for base editing may be used with this invention. A “cytosine deaminase” and “cytidine deaminase” as used herein refer to a polypeptide or domain thereof that catalyzes or is capable of catalyzing cytosine deamination in that the polypeptide or domain catalyzes or is capable of catalyzing the removal of an amine group from a cytosine base. Thus, a cytosine deaminase may result in conversion of cystosine to a thymidine (through a uracil intermediate), causing a C to T conversion, or a G to A conversion in the complementary strand in the genome. Thus, in some embodiments, the cytosine deaminase encoded by the polynucleotide of the invention generates a C→T conversion in the sense (e.g., “+”; template) strand of the target nucleic acid or a G→A conversion in antisense (e.g., “−”, complementary) strand of the target nucleic acid. In some embodiments, a cytosine deaminase encoded by a polynucleotide of the invention generates a C to T, G, or A conversion in the complementary strand in the genome.

A cytosine deaminase useful with this invention may be any known or later identified cytosine deaminase from any organism (see, e.g., U.S. Pat. No. 10,167,457 and Thuronyi et al. Nat. Biotechnol. 37:1070-1079 (2019), each of which is incorporated by reference herein for its disclosure of cytosine deaminases). Cytosine deaminases can catalyze the hydrolytic deamination of cytidine or deoxycytidine to uridine or deoxyuridine, respectively. Thus, in some embodiments, a deaminase or deaminase domain useful with this invention may be a cytidine deaminase domain, catalyzing the hydrolytic deamination of cytosine to uracil. In some embodiments, a cytosine deaminase may be a variant of a naturally-occurring cytosine deaminase, including, but not limited to, a primate (e.g., a human, monkey, chimpanzee, gorilla), a dog, a cow, a rat or a mouse. Thus, in some embodiments, an cytosine deaminase useful with the invention may be about 70% to about 100% identical to a wild type cytosine deaminase (e.g., about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical, and any range or value therein, to a naturally occurring cytosine deaminase).

In some embodiments, a cytosine deaminase useful with the invention may be an apolipoprotein B mRNA-editing complex (APOBEC) family deaminase. In some embodiments, the cytosine deaminase may be an APOBEC1 deaminase, an APOBEC2 deaminase, an APOBEC3A deaminase, an APOBEC3B deaminase, an APOBEC3C deaminase, an APOBEC3D deaminase, an APOBEC3F deaminase, an APOBEC3G deaminase, an APOBEC3H deaminase, an APOBEC4 deaminase, a human activation induced deaminase (hAID), an rAPOBEC1, FERNY, and/or a CDA1, optionally a pmCDA1, an atCDA1 (e.g., At2g19570), and evolved versions of the same. In some embodiments, the cytosine deaminase may be an APOBEC1 deaminase having the amino acid sequence of SEQ ID NO:76. In some embodiments, the cytosine deaminase may be an APOBEC3A deaminase having the amino acid sequence of SEQ ID NO:77. In some embodiments, the cytosine deaminase may be an CDA1 deaminase, optionally a CDA1 having the amino acid sequence of SEQ ID NO:78. In some embodiments, the cytosine deaminase may be a FERNY deaminase, optionally a FERNY having the amino acid sequence of SEQ ID NO:79. In some embodiments, the cytosine deaminase may be a rAPOBEC1 deaminase, optionally a rAPOBEC1 deaminase having the amino acid sequence of SEQ ID NO:80. In some embodiments, the cytosine deaminase may be a hAID deaminase, optionally a hAID having the amino acid sequence of SEQ ID NO:81 or SEQ ID NO:82. In some embodiments, a cytosine deaminase useful with the invention may be about 70% to about 100% identical (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5% or 100% identical) to the amino acid sequence of a naturally occurring cytosine deaminase (e.g., “evolved deaminases”) (see, e.g., SEQ ID NO:83, SEQ ID NO:84, SEQ ID NO:85). In some embodiments, a cytosine deaminase useful with the invention may be about 70% to about 99.5% identical (e.g., about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.5% identical) to the amino acid sequence of any one of SEQ ID NOs:76-85 (e.g., at least 80%, at least 85%, at least 90%, at least 92%, at least 95%, at least 96%, at least 97%, at least 98%, at least 99%, or at least 99.5% identical to the amino acid sequence of any one of SEQ ID NOs:76-85). In some embodiments, a polynucleotide encoding a cytosine deaminase may be codon optimized for expression in a plant and the codon optimized polypeptide may be about 70% to 99.5% identical to the reference polynucleotide.

An “adenine deaminase” and “adenosine deaminase” as used herein refer to a polypeptide or domain thereof that catalyzes or is capable of catalyzing the hydrolytic deamination (e.g., removal of an amine group from adenine) of adenine or adenosine. In some embodiments, an adenine deaminase may catalyze the hydrolytic deamination of adenosine or deoxyadenosine to inosine or deoxyinosine, respectively. In some embodiments, the adenosine deaminase may catalyze the hydrolytic deamination of adenine or adenosine in DNA. In some embodiments, an adenine deaminase encoded by a nucleic acid construct of the invention may generate an A→G conversion in the sense (e.g., “+”; template) strand of the target nucleic acid or a T→C conversion in the antisense (e.g., “−”, complementary) strand of the target nucleic acid. An adenine deaminase useful with this invention may be any known or later identified adenine deaminase from any organism (see, e.g., U.S. Pat. No. 10,113,163, which is incorporated by reference herein for its disclosure of adenine deaminases).

In some embodiments, an adenosine deaminase may be a variant of a naturally-occurring adenine deaminase. Thus, in some embodiments, an adenosine deaminase may be about 70% to 100% identical to a wild type adenine deaminase (e.g., about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical, and any range or value therein, to a naturally occurring adenine deaminase). In some embodiments, the deaminase or deaminase does not occur in nature and may be referred to as an engineered, mutated or evolved adenosine deaminase. Thus, for example, an engineered, mutated or evolved adenine deaminase polypeptide or an adenine deaminase domain may be about 70% to 99.9% identical to a naturally occurring adenine deaminase polypeptide/domain (e.g., about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8% or 99.9% identical, and any range or value therein, to a naturally occurring adenine deaminase polypeptide or adenine deaminase domain). In some embodiments, the adenosine deaminase may be from a bacterium, (e.g., Escherichia coli, Staphylococcus aureus, Haemophilus influenzae, Caulobacter crescentus, and the like). In some embodiments, a polynucleotide encoding an adenine deaminase polypeptide/domain may be codon optimized for expression in a plant.

In some embodiments, an adenine deaminase domain may be a wild type tRNA-specific adenosine deaminase domain, e.g., a tRNA-specific adenosine deaminase (TadA) and/or a mutated/evolved adenosine deaminase domain, e.g., mutated/evolved tRNA-specific adenosine deaminase domain (TadA*). In some embodiments, a TadA domain may be from E. coli. In some embodiments, the TadA may be modified, e.g., truncated, missing one or more N-terminal and/or C-terminal amino acids relative to a full-length TadA (e.g., 1, 2, 3, 4, 5, 6, 7, 8, 9, 10, 11, 12, 13, 14, 15, 6, 17, 18, 19, or 20 N-terminal and/or C terminal amino acid residues may be missing relative to a full length TadA. In some embodiments, a TadA polypeptide or TadA domain does not comprise an N-terminal methionine. In some embodiments, a wild type E. coli TadA comprises the amino acid sequence of SEQ ID NO:86. In some embodiments, a mutated/evolved E. coli TadA* comprises the amino acid sequence of SEQ ID NOs:87-90 (e.g., SEQ ID NOs: 87, 88, 89, or 90). In some embodiments, a polynucleotide encoding a TadA/TadA* may be codon optimized for expression in a plant. In some embodiments, an adenine deaminase may comprise all or a portion of an amino acid sequence of any one of SEQ ID NOs:91-96. In some embodiments, an adenine deaminase may comprise all or a portion of an amino acid sequence of any one of SEQ ID NOs:86-96.

In some embodiments, a nucleic acid construct of this invention may further encode a glycosylase inhibitor (e.g., a uracil glycosylase inhibitor (UGI) such as uracil-DNA glycosylase inhibitor). Thus, in some embodiments, a nucleic acid construct encoding a CRISPR-Cas effector protein and a cytosine deaminase and/or adenine deaminase may further encode a glycosylase inhibitor, optionally wherein the glycosylase inhibitor may be codon optimized for expression in a plant. In some embodiments, the invention provides fusion proteins comprising a CRISPR-Cas effector polypeptide and a UGI and/or one or more polynucleotides encoding the same, optionally wherein the one or more polynucleotides may be codon optimized for expression in a plant. In some embodiments, the invention provides fusion proteins comprising a CRISPR-Cas effector polypeptide, a deaminase domain (e.g., an adenine deaminase domain and/or a cytosine deaminase domain) and optionally a UGI and/or one or more polynucleotides encoding the same, optionally wherein the one or more polynucleotides may be codon optimized for expression in a plant. In some embodiments, the invention provides fusion proteins, wherein a CRISPR-Cas effector polypeptide, a deaminase domain, and/or a UGI may be fused to any combination of peptide tags and affinity polypeptides as described herein, which may thereby recruit the deaminase domain and/or UGI to the CRISPR-Cas effector polypeptide and to a target nucleic acid. In some embodiments, a guide nucleic acid may be linked to a recruiting RNA motif and one or more of the deaminase domain and/or UGI may be fused to an affinity polypeptide that is capable of interacting with the recruiting RNA motif, thereby recruiting the deaminase domain and UGI to a target nucleic acid.

A “uracil glycosylase inhibitor” useful with the invention may be any protein or polypeptide that is capable of inhibiting a uracil-DNA glycosylase base-excision repair enzyme. In some embodiments, a UGI domain comprises a wild type UGI or a fragment thereof. In some embodiments, a UGI domain useful with the invention may be about 70% to about 100% identical (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5% or 100% identical and any range or value therein) to the amino acid sequence of a naturally occurring UGI domain. In some embodiments, a UGI domain may comprise the amino acid sequence of SEQ ID NO:97 or a polypeptide having about 70% to about 99.5% identity to the amino acid sequence of SEQ ID NO:97 (e.g., at least 80%, at least 85%, at least 90%, at least 92%, at least 95%, at least 96%, at least 97%, at least 98%, at least 99%, or at least 99.5% identical to the amino acid sequence of SEQ ID NO:97). For example, in some embodiments, a UGI domain may comprise a fragment of the amino acid sequence of SEQ ID NO:97 that is 100% identical to a portion of consecutive nucleotides (e.g., 10, 15, 20, 25, 30, 35, 40, 45, 50, 55, 60, 65, 70, 75, 80 consecutive nucleotides; e.g., about 10, 15, 20, 25, 30, 35, 40, 45, to about 50, 55, 60, 65, 70, 75, 80 consecutive nucleotides) of the amino acid sequence of SEQ ID NO:97. In some embodiments, a UGI domain may be a variant of a known UGI (e.g., SEQ ID NO:97) having about 70% to about 99.5% identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5% identity, and any range or value therein) to the known UGI. In some embodiments, a polynucleotide encoding a UGI may be codon optimized for expression in a plant (e.g., a plant) and the codon optimized polypeptide may be about 70% to about 99.5% identical to the reference polynucleotide.

The nucleic acid constructs of the invention comprising a CRISPR-Cas effector protein or a fusion protein thereof may be used in combination with a guide nucleic acid (e.g., guide RNA (gRNA), CRISPR array, CRISPR RNA, crRNA), designed to function with the encoded CRISPR-Cas effector protein or domain, to modify a target nucleic acid. A guide nucleic acid useful with this invention may comprise at least one spacer sequence and at least one repeat sequence. The guide nucleic acid is capable of forming a complex with the CRISPR-Cas nuclease domain encoded and expressed by a nucleic acid construct of the invention and the spacer sequence is capable of hybridizing to a target nucleic acid, thereby guiding the complex to the target nucleic acid, wherein the target nucleic acid may be modified (e.g., cleaved or edited) and/or modulated (e.g., modulating transcription) by a deaminase (e.g., a cytosine deaminase and/or adenine deaminase, optionally present in and/or recruited to the complex).

As an example, a nucleic acid construct encoding a Cas9 domain linked to a cytosine deaminase domain (e.g., a fusion protein) may be used in combination with a Cas9 guide nucleic acid to modify a target nucleic acid, wherein the cytosine deaminase domain of the fusion protein deaminates a cytosine base in the target nucleic acid, thereby editing the target nucleic acid. In a further example, a nucleic acid construct encoding a Cas9 domain linked to an adenine deaminase domain (e.g., a fusion protein) may be used in combination with a Cas9 guide nucleic acid to modify a target nucleic acid, wherein the adenine deaminase domain of the fusion protein deaminates an adenosine base in the target nucleic acid, thereby editing the target nucleic acid. In some embodiments, a CRISPR-Cas effector protein (e.g., Cas9) is not fused to a cytosine deaminase and/or adenine deaminase.

Likewise, a nucleic acid construct encoding a Cas12a domain (or other selected CRISPR-Cas nuclease, e.g., C2c1, C2c3, Cas12b, Cas12c, Cas12d, Cas12e, Cas13a, Cas13b, Cas13c, Cas13d, Cas1, Cas1B, Cas2, Cas3, Cas3′, Cas3″, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4 (dinG), and/or Csf5) may be linked to a cytosine deaminase domain or adenine deaminase domain (e.g., fusion protein) and may be used in combination with a Cas12a guide nucleic acid (or the guide nucleic acid for the other selected CRISPR-Cas nuclease) to modify a target nucleic acid, wherein the cytosine deaminase domain or adenine deaminase domain of the fusion protein deaminates a cytosine base or adenosine base, respectively, in the target nucleic acid, thereby editing the target nucleic acid.

A “guide nucleic acid,” “guide RNA,” “gRNA,” “CRISPR RNA/DNA” “crRNA” or “crDNA” as used herein means a nucleic acid that comprises at least one spacer sequence, which is complementary to (and hybridizes to) a target DNA (e.g., protospacer), and at least one repeat sequence (e.g., a repeat of a Type V Cas12a CRISPR-Cas system, or a fragment or portion thereof; a repeat of a Type II Cas9 CRISPR-Cas system, or fragment thereof; a repeat of a Type V C2c 1 CRISPR Cas system, or a fragment thereof; a repeat of a CRISPR-Cas system of, for example, C2c3, Cas12a (also referred to as Cpf1), Cas12b, Cas12c, Cas12d, Cas12e, Cas13a, Cas13b, Cas13c, Cas13d, Cas1, Cas1B, Cas2, Cas3, Cas3′, Cas3″, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4 (dinG), and/or Csf5, or a fragment thereof), wherein the repeat sequence may be linked to the 5′ end and/or the 3′ end of the spacer sequence. In some embodiments, the guide nucleic acid comprises DNA. In some embodiments, the guide nucleic acid comprises RNA (e.g., is a guide RNA). The design of a gRNA of this invention may be based on a Type I, Type II, Type III, Type IV, Type V, or Type VI CRISPR-Cas system.

In some embodiments, a Cas12a gRNA may comprise, from 5′ to 3′, a repeat sequence (full length or portion thereof (“handle”); e.g., pseudoknot-like structure) and a spacer sequence.

In some embodiments, a guide nucleic acid may comprise more than one repeat sequence-spacer sequence (e.g., 2, 3, 4, 5, 6, 7, 8, 9, 10, or more repeat-spacer sequences) (e.g., repeat-spacer-repeat, e.g., repeat-spacer-repeat-spacer-repeat-spacer-repeat-spacer-repeat-spacer, and the like). The guide nucleic acids of this invention are synthetic, human-made and not found in nature. A gRNA can be quite long and may be used as an aptamer (like in the MS2 recruitment strategy) or other RNA structures hanging off the spacer.

A “repeat sequence” as used herein, refers to, for example, any repeat sequence of a wild-type CRISPR Cas locus (e.g., a Cas9 locus, a Cas12a locus, a C2c1 locus, etc.) or a repeat sequence of a synthetic crRNA that is functional with the CRISPR-Cas effector protein encoded by the nucleic acid constructs of the invention. A repeat sequence useful with this invention can be any known or later identified repeat sequence of a CRISPR-Cas locus (e.g., Type I, Type II, Type III, Type IV, Type V or Type VI) or it can be a synthetic repeat designed to function in a Type I, II, III, IV, V or VI CRISPR-Cas system. A repeat sequence may comprise a hairpin structure and/or a stem loop structure. In some embodiments, a repeat sequence may form a pseudoknot-like structure at its 5′ end (i.e., “handle”). Thus, in some embodiments, a repeat sequence can be identical to or substantially identical to a repeat sequence from wild-type Type I CRISPR-Cas loci, Type II, CRISPR-Cas loci, Type III, CRISPR-Cas loci, Type IV CRISPR-Cas loci, Type V CRISPR-Cas loci and/or Type VI CRISPR-Cas loci. A repeat sequence from a wild-type CRISPR-Cas locus may be determined through established algorithms, such as using the CRISPRfinder offered through CRISPRdb (see, Grissa et al. Nucleic Acids Res. 35(Web Server issue):W52-7). In some embodiments, a repeat sequence or portion thereof is linked at its 3′ end to the 5′ end of a spacer sequence, thereby forming a repeat-spacer sequence (e.g., guide nucleic acid, guide RNA/DNA, crRNA, crDNA).

In some embodiments, a repeat sequence comprises, consists essentially of, or consists of at least 10 nucleotides depending on the particular repeat and whether the guide nucleic acid comprising the repeat is processed or unprocessed (e.g., about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 to 100 or more nucleotides, or any range or value therein; e.g., about). In some embodiments, a repeat sequence comprises, consists essentially of, or consists of about 10 to about 20, about 10 to about 30, about 10 to about 45, about 10 to about 50, about 15 to about 30, about 15 to about 40, about 15 to about 45, about 15 to about 50, about 20 to about 30, about 20 to about 40, about 20 to about 50, about 30 to about 40, about 40 to about 80, about 50 to about 100 or more nucleotides.

A repeat sequence linked to the 5′ end of a spacer sequence can comprise a portion of a repeat sequence (e.g., 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 or more contiguous nucleotides of a wild type repeat sequence). In some embodiments, a portion of a repeat sequence linked to the 5′ end of a spacer sequence can be about five to about ten consecutive nucleotides in length (e.g., about 5, 6, 7, 8, 9, 10 nucleotides) and have at least 90% sequence identity (e.g., at least about 90%, 91%, 92%, 93%, 94%, 95%, 96%, 97%, 98%, 99%, or more) to the same region (e.g., 5′ end) of a wild type CRISPR Cas repeat nucleotide sequence. In some embodiments, a portion of a repeat sequence may comprise a pseudoknot-like structure at its 5′ end (e.g., “handle”).

A “spacer sequence” as used herein is a nucleotide sequence that is complementary to a target nucleic acid (e.g., target DNA) (e.g, protospacer). The spacer sequence can be fully complementary or substantially complementary (e.g., at least about 70% complementary (e.g., about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more)) to a target nucleic acid. Thus, in some embodiments, the spacer sequence can have one, two, three, four, or five mismatches as compared to the target nucleic acid, which mismatches can be contiguous or noncontiguous. In some embodiments, the spacer sequence can have 70% complementarity to a target nucleic acid. In other embodiments, the spacer nucleotide sequence can have 80% complementarity to a target nucleic acid. In still other embodiments, the spacer nucleotide sequence can have 85%, 90%, 95%, 96%, 97%, 98%, 99% or 99.5% complementarity, and the like, to the target nucleic acid (protospacer). In some embodiments, the spacer sequence is 100% complementary to the target nucleic acid. A spacer sequence may have a length from about 15 nucleotides to about 30 nucleotides (e.g., 15, 16, 17, 18, 19, 20, 21, 22, 23, 24, 25, 26, 27, 28, 29, or 30 nucleotides, or any range or value therein). Thus, in some embodiments, a spacer sequence may have complete complementarity or substantial complementarity over a region of a target nucleic acid (e.g., protospacer) that is at least about 15 nucleotides to about 30 nucleotides in length. In some embodiments, the spacer is about 20 nucleotides in length. In some embodiments, the spacer is about 21, 22, or 23 nucleotides in length.

In some embodiments, the 5′ region of a spacer sequence of a guide nucleic acid may be identical to a target DNA, while the 3′ region of the spacer may be substantially complementary to the target DNA (e.g., Type V CRISPR-Cas), or the 3′ region of a spacer sequence of a guide nucleic acid may be identical to a target DNA, while the 5′ region of the spacer may be substantially complementary to the target DNA (e.g., Type II CRISPR-Cas), and therefore, the overall complementarity of the spacer sequence to the target DNA may be less than 100%. Thus, for example, in a guide for a Type V CRISPR-Cas system, the first 1, 2, 3, 4, 5, 6, 7, 8, 9, 10 nucleotides in the 5′ region (i.e., seed region) of, for example, a 20 nucleotide spacer sequence may be 100% complementary to the target DNA, while the remaining nucleotides in the 3′ region of the spacer sequence are substantially complementary (e.g., at least about 70% complementary) to the target DNA. In some embodiments, the first 1 to 8 nucleotides (e.g., the first 1, 2, 3, 4, 5, 6, 7, 8, nucleotides, and any range therein) of the 5′ end of the spacer sequence may be 100% complementary to the target DNA, while the remaining nucleotides in the 3′ region of the spacer sequence are substantially complementary (e.g., at least about 50% complementary (e.g., 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more)) to the target DNA.

As a further example, in a guide for a Type II CRISPR-Cas system, the first 1, 2, 3, 4, 5, 6, 7, 8, 9, 10 nucleotides in the 3′ region (i.e., seed region) of, for example, a 20 nucleotide spacer sequence may be 100% complementary to the target DNA, while the remaining nucleotides in the 5′ region of the spacer sequence are substantially complementary (e.g., at least about 70% complementary) to the target DNA. In some embodiments, the first 1 to 10 nucleotides (e.g., the first 1, 2, 3, 4, 5, 6, 7, 8, 9, 10 nucleotides, and any range therein) of the 3′ end of the spacer sequence may be 100% complementary to the target DNA, while the remaining nucleotides in the 5′ region of the spacer sequence are substantially complementary (e.g., at least about 50% complementary (e.g., at least about 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more or any range or value therein)) to the target DNA. A recruiting guide RNA further comprises one or more recruiting motifs as described herein, which may be linked to the 5′ end of the guide or the 3′ end or it may be inserted into the recruiting guide nucleic acid (e.g., within the hairpin loop).

In some embodiments, a seed region of a spacer may be about 8 to about 10 nucleotides in length, about 5 to about 6 nucleotides in length, or about 6 nucleotides in length.

A “target nucleic acid”, “target DNA,” “target nucleotide sequence,” “target region,” and a “target region in the genome” are used interchangeably herein and refer to a region of a plant's genome that is targeted by an editing system (or a component thereof) as described herein. In some embodiments, a target nucleic acid is fully complementary (100% complementary) or substantially complementary (e.g., at least 70% complementary (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more)) to a spacer sequence in a guide nucleic acid of this invention. A target region useful for a CRISPR-Cas system may be located immediately 3′ (e.g., Type V CRISPR-Cas system) or immediately 5′ (e.g., Type II CRISPR-Cas system) to a PAM sequence in the genome of the organism (e.g., a plant genome). A target region may be selected from any region of at least 15 consecutive nucleotides (e.g., 16, 17, 18, 19, 20, 21, 22, 23, 24, 25, 26, 27, 28, 29, 30 nucleotides, and the like) located immediately adjacent to a PAM sequence.

A “protospacer sequence” refers to the target double stranded DNA and specifically to the portion of the target DNA (e.g., or target region in the genome) that is fully or substantially complementary (and hybridizes) to the spacer sequence of the CRISPR repeat-spacer sequences (e.g., guide nucleic acids, CRISPR arrays, crRNAs).

In the case of Type V CRISPR-Cas (e.g., Cas12a) systems and Type II CRISPR-Cas (Cas9) systems, the protospacer sequence is flanked by (e.g., immediately adjacent to) a protospacer adjacent motif (PAM). For Type IV CRISPR-Cas systems, the PAM is located at the 5′ end on the non-target strand and at the 3′ end of the target strand (see below, as an example).

| | | |
|---|---|---|
| 5′-NNNNNNNNNNNNNNNNNNN-3′ | RNA Spacer | (SEQ ID NO: 98) |
| \|\|\|\|\|\|\|\|\|\|\|\|\|\|\|\|\|\|\|\| | | |
| 3′AAANNNNNNNNNNNNNNNNNNN-5′ | Target strand | (SEQ ID NO: 99) |
| \|\|\|\| | | |
| 5′TTTNNNNNNNNNNNNNNNNNNN-3′ | Non-target strand | (SEQ ID NO: 100) |

In the case of Type II CRISPR-Cas (e.g., Cas9) systems, the PAM is located immediately 3′ of the target region. The PAM for Type I CRISPR-Cas systems is located 5′ of the target strand. There is no known PAM for Type III CRISPR-Cas systems. Makarova et al. describes the nomenclature for all the classes, types and subtypes of CRISPR systems (Nature Reviews Microbiology 13:722-736 (2015)). Guide structures and PAMs are described in by R. Barrangou (Genome Biol. 16:247 (2015)).

Canonical Cas12a PAMs are T rich. In some embodiments, a canonical Cas12a PAM sequence may be 5′-TTN, 5′-TTTN, or 5′-TTTV. In some embodiments, canonical Cas9 (e.g., S. pyogenes) PAMs may be 5′-NGG-3′. In some embodiments, non-canonical PAMs may be used but may be less efficient.

Additional PAM sequences may be determined by those skilled in the art through established experimental and computational approaches. Thus, for example, experimental approaches include targeting a sequence flanked by all possible nucleotide sequences and identifying sequence members that do not undergo targeting, such as through the transformation of target plasmid DNA (Esvelt et al. 2013. Nat. Methods 10:1116-1121; Jiang et al. 2013. Nat. Biotechnol. 31:233-239). In some aspects, a computational approach can include performing BLAST searches of natural spacers to identify the original target DNA sequences in bacteriophages or plasmids and aligning these sequences to determine conserved sequences adjacent to the target sequence (Briner and Barrangou. 2014. Appl. Environ. Microbiol. 80:994-1001; Mojica et al. 2009. Microbiology 155:733-740).

In some embodiments, the present invention provides expression cassettes and/or vectors comprising the nucleic acid constructs of the invention (e.g., one or more components of an editing system of the invention). In some embodiments, expression cassettes and/or vectors comprising the nucleic acid constructs of the invention and/or one or more guide nucleic acids may be provided. In some embodiments, a nucleic acid construct of the invention comprising a heterologous morphogenic regulator and encoding a base editor (e.g., a construct comprising a CRISPR-Cas effector domain and a deaminase domain (e.g., a fusion protein)) or the components for base editing (e.g., a CRISPR-Cas effector domain fused to a peptide tag or an affinity polypeptide, a deaminase domain fused to a peptide tag or an affinity polypeptide, and/or a UGI fused to a peptide tag or an affinity polypeptide), may be comprised on the same or on a separate expression cassette or vector from that comprising the one or more guide nucleic acids. When the nucleic acid construct encoding a base editor or the components for base editing is/are comprised on separate expression cassette(s) or vector(s) from that comprising the guide nucleic acid, a target nucleic acid may be contacted with (e.g., provided with) the expression cassette(s) or vector(s) encoding the base editor or components for base editing in any order from one another and the guide nucleic acid, e.g., prior to, concurrently with, or after the expression cassette comprising the guide nucleic acid is provided (e.g., contacted with the target nucleic acid).

Fusion proteins of the invention may comprise a sequence-specific DNA binding domain, a CRISPR-Cas effector protein, and/or a deaminase fused to a peptide tag or an affinity polypeptide that interact with the peptide tag, as known in the art, for use in recruiting the deaminase to the target nucleic acid. Methods of recruiting may also comprise a guide nucleic acids linked to an RNA recruiting motif and a deaminase fused to an affinity polypeptide capable of interacting with the RNA recruiting motif, thereby recruiting the deaminase to the target nucleic acid. Alternatively, chemical interactions may be used to recruit a polypeptide (e.g., a deaminase) to a target nucleic acid.

A peptide tag (e.g., epitope) useful with this invention may include, but is not limited to, a GCN4 peptide tag (e.g., Sun-Tag), a c-Myc affinity tag, an HA affinity tag, a His affinity tag, an S affinity tag, a methionine-His affinity tag, an RGD-His affinity tag, a FLAG octapeptide, a strep tag or strep tag II, a V5 tag, and/or a VSV-G epitope. Any epitope that may be linked to a polypeptide and for which there is a corresponding affinity polypeptide that may be linked to another polypeptide may be used with this invention as a peptide tag. In some embodiments, a peptide tag may comprise 1 or 2 or more copies of a peptide tag (e.g., repeat unit, multimerized epitope (e.g., tandem repeats)) (e.g., 1, 2, 3, 4, 5, 6, 7, 8, 9, 10, 11, 12, 13, 14, 15, 16, 17, 18, 19, 20, 21, 22, 23, 24, 25 or more repeat units. In some embodiments, an affinity polypeptide that interacts with/binds to a peptide tag may be an antibody. In some embodiments, the antibody may be a scFv antibody. In some embodiments, an affinity polypeptide that binds to a peptide tag may be synthetic (e.g., evolved for affinity interaction) including, but not limited to, an affibody, an anticalin, a monobody and/or a DARPin (see, e.g., Sha et al., Protein Sci. 26(5):910-924 (2017)); Gilbreth (Curr Opin Struc Biol 22(4):413-420 (2013)), U.S. Pat. No. 9,982,053, each of which are incorporated by reference in their entireties for the teachings relevant to affibodies, anticalins, monobodies and/or DARPins.

In some embodiments, a guide nucleic acid may be linked to an RNA recruiting motif, and a polypeptide to be recruited (e.g., a deaminase) may be fused to an affinity polypeptide that binds to the RNA recruiting motif, wherein the guide binds to the target nucleic acid and the RNA recruiting motif binds to the affinity polypeptide, thereby recruiting the polypeptide to the guide and contacting the target nucleic acid with the polypeptide (e.g., deaminase). In some embodiments, two or more polypeptides may be recruited to a guide nucleic acid, thereby contacting the target nucleic acid with two or more polypeptides (e.g., deaminases).

In some embodiments of the invention, a guide RNA may be linked to one or to two or more RNA recruiting motifs (e.g., 1, 2, 3, 4, 5, 6, 7, 8, 9, 10 or more motifs; e.g., at least 10 to about 25 motifs), optionally wherein the two or more RNA recruiting motifs may be the same RNA recruiting motif or different RNA recruiting motifs. In some embodiments, an RNA recruiting motif and corresponding affinity polypeptide may include, but is not limited, to a telomerase Ku binding motif (e.g., Ku binding hairpin) and the corresponding affinity polypeptide Ku (e.g., Ku heterodimer), a telomerase Sm7 binding motif and the corresponding affinity polypeptide Sm7, an MS2 phage operator stem-loop and the corresponding affinity polypeptide MS2 Coat Protein (MCP), a PP7 phage operator stem-loop and the corresponding affinity polypeptide PP7 Coat Protein (PCP), an SfMu phage Com stem-loop and the corresponding affinity polypeptide Com RNA binding protein, a PUF binding site (PBS) and the affinity polypeptide Pumilio/fem-3 mRNA binding factor (PUF), and/or a synthetic RNA-aptamer and the aptamer ligand as the corresponding affinity polypeptide. In some embodiments, the RNA recruiting motif and corresponding affinity polypeptide may be an MS2 phage operator stem-loop and the affinity polypeptide MS2 Coat Protein (MCP). In some embodiments, the RNA recruiting motif and corresponding affinity polypeptide may be a PUF binding site (PBS) and the affinity polypeptide Pumilio/fem-3 mRNA binding factor (PUF). Exemplary RNA recruiting motifs and corresponding affinity polypeptides that may be useful with this invention can include, but are not limited to, SEQ ID NOs:101-111.

In some embodiments, the components for recruiting polypeptides and nucleic acids may include those that function through chemical interactions that may include, but are not limited to, rapamycin-inducible dimerization of FRB-FKBP; Biotin-streptavidin; SNAP tag; Halo tag; CLIP tag; DmrA-DmrC heterodimer induced by a compound; bifunctional ligand (e.g., fusion of two protein-binding chemicals together; e.g. dihyrofolate reductase (DHFR).

In some embodiments, the nucleic acid constructs, expression cassettes or vectors of the invention that are optimized for expression in a plant may be about 70% to 100% identical (e.g., about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5% or 100%) to the nucleic acid constructs, expression cassettes or vectors comprising the same polynucleotide(s) but which have not been codon optimized for expression in a plant.

As described herein, a “peptide tag” may be employed to recruit one or more polypeptides. A peptide tag may be any polypeptide that is capable of being bound by a corresponding affinity polypeptide. A peptide tag may also be referred to as an “epitope” and when provided in multiple copies, a “multimerized epitope.” Example peptide tags can include, but are not limited to, a GCN4 peptide tag (e.g., Sun-Tag), a c-Myc affinity tag, an HA affinity tag, a His affinity tag, an S affinity tag, a methionine-His affinity tag, an RGD-His affinity tag, a FLAG octapeptide, a strep tag or strep tag II, a V5 tag, and/or a VSV-G epitope. In some embodiments, a peptide tag may also include phosphorylated tyrosines in specific sequence contexts recognized by SH2 domains, characteristic consensus sequences containing phosphoserines recognized by 14-3-3 proteins, proline rich peptide motifs recognized by SH3 domains, PDZ protein interaction domains or the PDZ signal sequences, and an AGO hook motif from plants. Peptide tags are disclosed in WO2018/136783 and U.S. Patent Application Publication No. 2017/0219596, which are incorporated by reference for their disclosures of peptide tags. Peptide tags that may be useful with this invention can include, but are not limited to, SEQ ID NO:112 and SEQ ID NO:113. An affinity polypeptide useful with peptide tags includes, but is not limited to, SEQ ID NO:114.

A peptide tag may comprise or be present in one copy or in 2 or more copies of the peptide tag (e.g., multimerized peptide tag or multimerized epitope) (e.g., about 2, 3, 4, 5, 6, 7, 8, 9, 10, 11, 12, 13, 14, 15, 16, 17, 18, 9, 20, 21, 22, 23, 24, or 25 or more peptide tags). When multimerized, the peptide tags may be fused directly to one another or they may be linked to one another via one or more amino acids (e.g., 1, 2, 3, 4, 5, 6, 7, 8, 9, 10, 11, 12, 13, 14, 15, 16, 17, 18, 19, 20 or more amino acids, optionally about 3 to about 10, about 4 to about 10, about 5 to about 10, about 5 to about 15, or about 5 to about 20 amino acids, and the like, and any value or range therein. Thus, in some embodiments, a CRISPR-Cas effector protein of the invention may comprise a CRISPR-Cas effector protein domain fused to one peptide tag or to two or more peptide tags, optionally wherein the two or more peptide tags are fused to one another via one or more amino acid residues. In some embodiments, a peptide tag useful with the invention may be a single copy of a GCN4 peptide tag or epitope or may be a multimerized GCN4 epitope comprising about 2 to about 25 or more copies of the peptide tag (e.g., about 2, 3, 4, 5, 6, 7, 8, 9, 10, 11, 12, 13, 14, 15, 16, 17, 18, 19, 20, 21, 22, 23, 24, 25 or more copies of a GCN4 epitope or any range therein).

In some embodiments, a peptide tag may be fused to a CRISPR-Cas polypeptide or domain. In some embodiments, a peptide tag may be fused or linked to the C-terminus of a CRISPR-Cas effector protein to form a CRISPR-Cas fusion protein. In some embodiments, a peptide tag may be fused or linked to the N-terminus of a CRISPR-Cas effector protein to form a CRISPR-Cas fusion protein. In some embodiments, a peptide tag may be fused within a CRISPR-Cas effector protein (e.g., a peptide tag may be in a loop region of a CRISPR-Cas effector protein). In some embodiments, peptide tag may be fused to a cytosine deaminase and/or to an adenine deaminase.

An “affinity polypeptide” (e.g., “recruiting polypeptide”) refers to any polypeptide that is capable of binding to its corresponding peptide tag, peptide tag, or RNA recruiting motif. An affinity polypeptide for a peptide tag may be, for example, an antibody and/or a single chain antibody that specifically binds the peptide tag, respectively. In some embodiments, an antibody for a peptide tag may be, but is not limited to, an scFv antibody. In some embodiments, an affinity polypeptide may be fused or linked to the N-terminus of a deaminase (e.g., a cytosine deaminase or an adenine deaminase). In some embodiments, the affinity polypeptide is stable under the reducing conditions of a cell or cellular extract.

The nucleic acid constructs of the invention and/or guide nucleic acids may be comprised in one or more expression cassettes as described herein. In some embodiments, a nucleic acid construct of the invention may be comprised in the same or in a separate expression cassette or vector from that comprising a guide nucleic acid and/or a recruiting guide nucleic acid.

When used in combination with guide nucleic acids and recruiting guide nucleic acids, the nucleic acid constructs of the invention (and expression cassettes and vectors comprising the same) may be used to modify a target nucleic acid and/or its expression. A target nucleic acid may be contacted with a nucleic acid construct of the invention and/or expression cassettes and/or vectors comprising the same prior to, concurrently with or after contacting the target nucleic acid with the guide nucleic acid/recruiting guide nucleic acid (and/or expression cassettes and vectors comprising the same.

The term “seed region” refers to the critical portion of a crRNA's or guide RNA's guide sequence that is most susceptible to mismatches with their targets. In some embodiments, a single mismatch in the seed region of a crRNA/gRNA can render a CRISPR complex inactive at that binding site. In some embodiments, the seed regions for Cas9 endonucleases are located along the last ˜12 nts of the 3′ portion of the guide sequence, which correspond (hybridize) to the portion of the protospacer target sequence that is adjacent to the PAM. In some embodiments, the seed regions for Cpf1 endonucleases are located along the first ˜5 nts of the 5′ portion of the guide sequence, which correspond (hybridize) to the portion of the protospacer target sequence adjacent to the PAM.

The term “modified” refers to a substance or compound (e.g., a cell, a polynucleotide sequence, and/or a polypeptide sequence) that has been altered or changed as compared to the corresponding unmodified substance or compound. For example, a modified polynucleotide can be a nucleic acid that has been edited (e.g., mutated such as by substituting and/or deleting a nucleic acid) and/or changed compared to its unmodified or native sequence and/or structure.

An “edited polynucleotide” as used herein refers to a polynucleotide that has been edited (e.g., mutated such as by substituting and/or deleting a nucleic acid) and/or changed compared to its unmodified or native sequence and/or structure. The term “gene edited plant, part or cell” as used herein refers to a plant, part or cell that comprises one or more endogenous genes that are edited by an editing system. An editing system of the present disclosure may comprise a targeting element and/or an editing element. The targeting element may be capable of recognizing and/or may be configured to recognize a target genomic sequence. The editing element may be capable of modifying and/or may be configured to modify a target nucleic acid, e.g., by substitution or insertion of one or more nucleotides, deletion of one or more nucleotides, alteration of a nucleotide sequence to include a regulatory sequence, insertion of a transgene at a safe harbor genomic site or other specific location in the genome, or any combination thereof. The targeting element and the editing element can be on the same nucleic acid molecule or different nucleic acid molecules. In some embodiments, the editing element is capable of precise genome editing by insertion and/or deletion of at least one nucleotide using a CRISPR/Cas system. In some embodiments, the editing element is capable of precise genome editing by substitution of a single nucleotide using a base editor, such cytosine base editor (CBE) and/or adenine base editor (ABE), which is directly or indirectly fused to a CRISPR-associated effector protein.

The terms “transgene” or “transgenic” as used herein refer to at least one nucleic acid sequence that is taken from the genome of one organism, or produced synthetically, and which is then introduced into a host cell or organism or tissue of interest and which is subsequently integrated into the host's genome by means of “stable” transformation or transfection approaches. In contrast, the term “transient” transformation or transfection or introduction refers to a way of introducing molecular tools including at least one nucleic acid (DNA, RNA, single-stranded or double-stranded or a mixture thereof) and/or at least one amino acid sequence, optionally comprising suitable chemical or biological agents, to achieve a transfer into at least one compartment of interest of a cell, including, but not restricted to, the cytoplasm, an organelle, including the nucleus, a mitochondrion, a vacuole, a chloroplast, or into a membrane, resulting in transcription and/or translation and/or association and/or activity of the at least one molecule introduced without achieving a stable integration or incorporation and thus inheritance of the respective at least one molecule introduced into the genome of a cell. The terms “transgene-free” refers to a condition in which a transgene is not present or found in the genome of a host cell or tissue or organism of interest.

As used herein, the term “tissue culture” indicates a composition comprising isolated cells of the same or a different type or a collection of such cells organized into parts of a plant. Exemplary types of tissue cultures are protoplasts, calli, plant clumps, and plant cells that can generate tissue culture that are intact in plants or parts of plants, such as embryos, pollen, flowers, seeds, leaves, stems, roots, root tips, anthers, pistils, meristematic cells, axillary buds, ovaries, seed coat, endosperm, hypocotyls, cotyledons and the like. The term “plant organ” refers to plant tissue or a group of tissues that constitute a morphologically and functionally distinct part of a plant. “Progeny” comprises any subsequent generation of a plant.

General methods in molecular and cellular biochemistry can be found in such standard textbooks as Molecular Cloning: A Laboratory Manual, 3rd Ed. (Sambrook et al., HaRBor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998), the disclosures of which are incorporated herein by reference.

As mentioned above, segregating to eliminate transgenes in vegetative propagated crops such as caneberries and cherries is not a preferred practice after a target gene is edited. Particularly, the heterozygotic nature of these crops requires several years or decades in the case of woody crops such as cherries, to breed newly edited traits to a desired elite cultivar for commercial production using traditional breeding methods. The present disclosure provides a solution to obtain transgene-free edited plants such as by transiently expressing the gene editing machinery (optionally, a Cas9 endonuclease and gRNA) in the plant cells and also by co-expressing at least one morphogenic regulator to regenerate edited cells without the aid of selection. The present disclosure also teaches methods for culturing plant tissues for transformation and regeneration as well as identification of the gene-edited event in the transformed plant, plant tissue, and plant cell.

A method of the present invention may comprise modifying a target nucleic acid using a nucleic acid construct of the invention, and/or an expression cassette and/or vector comprising the same. A method of the present invention may be carried out in an in vivo system (e.g., in a cell or in an organism) or in an in vitro system (e.g., cell free). A method, composition, and/or system of the present invention may generate and/or provide allelic diversity, optionally in a semi-random way. In some embodiments, a method of the present invention comprises determining a desired or preferred phenotype using and/or based on the modified target nucleic acid. A method of the present invention may provide one or more modified target nucleic acid(s), and the one or more modified target nucleic acid(s) may be analyzed for a desired or preferred phenotype.

According to some embodiments, a morphogenic regulator is provided. A morphogenic regulator may be present in and/or used in a composition, system, and/or method of the present invention. A “morphogenic regulator” as used herein refers to a nucleic acid sequence, gene, or polypeptide that can cause and/or stimulate morphogenesis in a cell in which it is present and expressed. A morphogenic regulator of the present invention may be used to regenerate a plant from a transformed plant cell. In some embodiments, ectopic overexpression of a morphogenic regulator involved in either embryo and/or meristem development stimulates growth of transgenic plants. Due to pleiotropic deleterious effect of some morphogenic regulators under a condition of constitutive expression, the present disclosure teaches the initial stimulating of morphogenic regulators while later restricting or eliminating their expression in the plant. In some embodiments, methods of controlling ectopic overexpression include, but are not limited to the use of transient expression, inducible promoters, tissue-specific promoters, and excision of the morphogenic regulators.

In some embodiments, ectopic overexpression of morphogenic regulators induce morphogenic growth response to enhance and/or improve transformation efficiencies, which may allow for transformation of numerous recalcitrant crops and/or successful regeneration of transformed plants. In some embodiments, transient expression of morphogenic regulators enhances and/or improves transformation efficiencies. In some embodiments, transient expression of a morphogenic regulator as described herein provides an increase and/or improvement in plant transformation efficiency and/or rate. In other embodiments, transient expression of morphogenic regulators provides an increase and/or improvement in a plant regeneration process and/or rate. In some embodiments, introduction of a polypeptide comprising a morphogenic regulator onto and/or into a plant cell provides an increase and/or improvement in plant transformation efficiency and/or rate and/or a plant regeneration process and/or rate. In some embodiments, the use of morphogenic regulators can solve current issues with plant genome modification and regeneration of genetically-modified plants and/or gene-edited plants. In some embodiments, plant transformation and regeneration improvements can be distinguished by their impact on either improving transformation efficiencies, or improving the regeneration process to recover transgenic plants.

Exemplary morphogenic regulators include, but are not limited to, a Wuschel (WUS) gene (e.g., from blackberry and/or cherry), Baby boom (BBM, e.g., from blackberry and/or cherry), Knotted1 (KN1) gene (e.g., from maize, blackberry, and/or cherry), isopentenyl transferase (ipt) gene (e.g., from Agrobacterium tumefaciens, blackberry, and/or cherry), CLAVATA3 (CLV3) mutant, miR156 (e.g., from citrus), AtGRF5 (e.g., from Arabidopsis thaliana), NTT (e.g., from Arabidopsis thaliana, blackberry, and/or cherry), HDZipII (e.g., from Arabidopsis thaliana, blackberry, and/or cherry), and orthologs or polypeptides thereof. Further exemplary morphogenic regulators include, but are not limited to those described in U.S. Pat. No. 7,256,322; Plant Cell 14:1737-1749 (2002); Plant Cell Physiol. 41(5) 583-590 (2000); Plant Physiol. 161(3):1076-1085 (2013); J Exp Bot. 69(12):2979-2993 (2018); Plant Cell 27(2):349-360 (2015); and Plant Physiol. 167(3):817-832 (2015).

In some embodiments, homologs and orthologs of WUS, BBM, KN1, IPT, CLV3, miR156, GRF5, NTT, and HDZipII genes are identified and used for improving plant transformation and/regeneration in a plant of interest.

Further exemplary morphogenic regulators include, but are not limited to, those having a nucleotide sequence having at least 90%, 91%, 92%, 93%, 94%, 95%, 96%, 97%, 98%, 99%, or 100% sequence identity to the nucleotide sequence of any one of SEQ ID NOs:1-18 or SEQ ID NOs:28-49. In some embodiments, a nucleotide sequence of the present invention may encode an amino acid sequence having at least 90%, 91%, 92%, 93%, 94%, 95%, 96%, 97%, 98%, 99%, or 100% sequence identity to the polypeptide sequence of any one of SEQ ID NOs:19-27 or SEQ ID NOs:50-60. Exemplary morphogenic regulators may have an amino acid sequence having at least 90%, 91%, 92%, 93%, 94%, 95%, 96%, 97%, 98%, 99%, or 100% sequence identity to the polypeptide sequence of any one of SEQ ID NOs:19-27 or SEQ ID NOs:50-60. A polynucleotide comprising a morphogenic regulator may be operably associated with a heterologous promoter. Compositions, systems, and/or methods of the present invention may provide a cell, plant part, plant, expression cassette, and/or vector comprising a polynucleotide comprising a morphogenic regulator as described herein and/or a polypeptide comprising a morphogenic regulator as described herein. In some embodiments, the cell, plant part, plant, expression cassette, and/or vector also includes an editing system (e.g., a polynucleotide encoding gene editing machinery or a gene editing complex (e.g., a ribonucleoprotein)), and/or a polynucleotide encoding a selectable marker.

The catabolic enzyme isopentenyl transferase (IPT) directs the synthesis of cytokinins and plays a major role in controlling cytokinin levels in plant tissues. Multiple routes have been proposed for cytokinin biosynthesis. Transfer RNA degradation has been suggested to be a source of cytokinin, because some tRNA molecules contain an isopentenyladenosine (iPA) residue at the site adjacent to the anticodon (Swaminathan, et al., (1977) Biochemistry 16:1355-1360). The modification is catalyzed by tRNA isopentenyl transferase (tRNA IPT; EC 2.5.1.8), which has been identified in various organisms such as Escherichia coli, Saccharomyces cerevisiae, Lactobacillus acidophilus, Homo sapiens, and Zea mays (Bartz, et al., (1972) Biochemie 54:31-39; Kline, et al., (1969) Biochemistry 8:4361-4371; Holtz, et al., (1975) Hoppe-Seyler's Z. Physiol. Chem. 356:1459-1464; Golovko, et al., (2000) Gene 258:85-93; and, Holtz, et al., (1979) Hoppe-Seyler's Z. Physiol. Chem. 359:89-101). However, this pathway is not considered to be the main route for cytokinin synthesis (Chen, et al., (1997) Physiol. Plant 101:665-673 and McGraw, et al., (1995) Plant Hormones, Physiology, Biochemistry and Molecular Biology. Ed. Davies, 98-117, Kluwer Academic Publishers, Dordrecht).

The current knowledge of cytokinin biosynthesis in plants is largely deduced from studies on a possible analogous system in Agrobacterium tumefaciens. Cells of A. tumefaciens are able to infect certain plant species by inducing tumor formation in host plant tissues (Van Montagu, et al., (1982) Curr Top Microbiol Immunol 96:237-254; Hansen, et al., (1999). Curr Top Microbiol Immunol 240:21-57). To do so, the A. tumefaciens cells synthesize and secrete cytokinins which mediate the transformation of normal host plant tissues into tumors or calli. This process is facilitated by the A. tumefaciens tumor-inducing plasmid which contains genes encoding the necessary enzyme and regulators for cytokinin biosynthesis. Biochemical and genetic studies revealed that Gene 4 of the tumor-inducing plasmid encodes an isopentenyl transferase (IPT), which converts AMP and DMAPP into isopentenyladenosine-5′-monophosphate (iPMP), the active form of cytokinins (Akiyoshi, et al., (1984) Proc. Natl. Acad. Sci. USA 81:5994-5998). Overexpression of the Agrobacterium ipt gene in a variety of transgenic plants has been shown to cause an increased level of cytokinins and elicit typical cytokinin responses in the host plant (Hansen, et al., (1999) Curr Top Microbiol Immunol 240:21-57). Therefore, it has been postulated that plant cells use machinery similar to that of A. tumefaciens cells for cytokinin biosynthesis. Arabidopsis IPT homologs have recently been identified in Arabidopsis and Petunia (Takei, et al., (2001) J. Biol. Chem. 276:26405-26410 and Kakimoto, (2001) Plant Cell Physiol. 42:677-685). Overexpression of the Arabidopsis IPT homologs in plants elevated cytokinin levels and elicited typical cytokinin responses in planta and under tissue culture conditions (Kakimoto, (2001) Plant Cell Physiol. 42:677-685).

In view of the influence of cytokinins on a wide variety of plant developmental processes, including root architecture, shoot and leaf development, and seed set, the ability to manipulate cytokinin levels in higher plant cells, and thereby drastically affect plant growth and productivity, offers significant commercial value (Mok, et al., (1994) Cytokinins. Chemistry, Action and Function. CRC Press, Boca Raton, Fla., pp. 155-166).

In some embodiments, the morphogenic regulator is an ipt gene encoding isopentenyl transferase. Isopentenyl transferase (ipt) gene is a cytokinin biosynthetic gene isolated from Agrobacterium tumefaciens.

In some embodiments, overexpression of a morphogenic regulator (e.g., an ipt gene) in transgenic plant cells induces shoot formation in media without additional plant growth regulators. In some embodiments, non-transgenic plants may be regenerated from the hormone-free media and they may be morphologically normal. In some embodiments, these non-transgenic plants are derived from cells where a morphogenic regulator was transiently expressed. In some embodiments, these non-transgenic plants are from the cells adjacent to the transformed cells which redistributed the cytokinin to the adjacent wild type cells.

Compositions, systems, and/or methods of the present invention may produce non-transgenic plants that are derived from cells under a condition of in which a morphogenic regulator is transiently expressed. In some embodiments, an expression cassette comprising gene editing machinery (Cas variants, Cpf1, gRNA targeting PDS or other genes) is transformed or co-transformed with another expression cassette comprising a morphogenic regulator. In some embodiments, the resultant transgenic (with an edited polynucleotide, but without expression of the morphogenic regulator) and non-transgenic (with anedited polynucleotide, and with expression of the morphogenic regulator) plants can be analyzed for transgene integration and gene editing efficiency.

A method of the present invention may comprise introducing a polynucleotide or a polypeptide comprising a heterologous morphogenic regulator into a plant cell, thereby transforming the plant cell. A “heterologous morphogenic regulator” as used herein refers to a morphogenic regulator as described herein that is heterologous to the plant cell in which the morphogenic regulator is being or has been introduced, or to a morphogenic regulator as described herein that is present at a different protein concentration than it would normally be present in the plant cell to which the morphogenic regulator is being or has been introduced. Compared to a control, the method may improve or increase regeneration frequency in a group of plant cells in which at least one of the plant cells is transformed with the polynucleotide or polypeptide comprising a heterologous morphogenic regulator. A “group of plant cells” as used herein refers to two more plant cells. In some embodiments, regeneration frequency may be improved or increased in a group of plant cells that are not transformed with a polynucleotide or polypeptide comprising a heterologous morphogenic regulator, but are in proximity to a cell comprising the polynucleotide or polypeptide comprising a heterologous morphogenic regulator. The method may include introducing an editing system into the same plant cell or a different plant cell as the heterologous morphogenic regulator. The step of introducing the polynucleotide or polypeptide comprising the heterologous morphogenic regulator and the step of introducing the editing system may be performed concurrently or sequentially in any order. A plant cell may be stably transformed with a polynucleotide comprising a heterologous morphogenic regulator and/or may be stably transformed with an editing system (e.g., a polynucleotide encoding a gene editing machinery or a gene editing complex such as a ribonucleoprotein). A plant cell may be transiently transformed with a polynucleotide or polypeptide comprising a heterologous morphogenic regulator and/or may be stably transformed with an editing system (e.g., a polynucleotide encoding a gene editing machinery or a gene editing complex).

A method of the present invention may not introduce (i.e., may be devoid of introducing) a polynucleotide encoding a selectable marker. Accordingly, a plant cell comprising the polynucleotide or polypeptide comprising a heterologous morphogenic regulator and/or editing system may not comprise a polynucleotide encoding a selectable marker. In some embodiments, a method of the present invention comprises introducing a polynucleotide encoding a selectable marker into the same plant cell or a different plant cell as the heterologous morphogenic regulator and/or editing system. A selectable marker may be stably or transiently transformed in a cell and/or method of the present invention.

A single polynucleotide may comprise a polynucleotide comprising a heterologous morphogenic regulator, a polynucleotide encoding an editing system, and/or a polynucleotide encoding a selectable marker, or two or more polynucleotides may include one or more of a polynucleotide comprising a heterologous morphogenic regulator, a polynucleotide encoding an editing system, and/or a polynucleotide encoding a selectable marker. In some embodiments, a first polynucleotide comprises a polynucleotide comprising a heterologous morphogenic regulator and a second polynucleotide comprises a polynucleotide encoding editing system.

A method of the present invention may stimulate embryogenesis and/or organogenesis in a plant cell. In some embodiments, a method of the present invention stimulates embryogenesis and/or organogenesis in a plant cell comprising a polynucleotide or polypeptide comprising a heterologous morphogenic regulator and optionally an editing system and/or selectable marker. In some embodiments, a plant cell and/or method of the present invention stimulates embryogenesis and/or organogenesis in a plant cell that does not comprise (i.e., is devoid of) a polynucleotide or polypeptide comprising a heterologous morphogenic regulator or does not have the heterologous morphogenic regulator at a same concentration (e.g., protein concentration) in the plant cell. For example, a method of the present invention may introduce a polynucleotide comprising a heterologous morphogenic regulator into a plant cell and the plant cell comprising the polynucleotide comprising a heterologous morphogenic regulator may stably or transiently express the polynucleotide comprising a heterologous morphogenic regulator. In stably or transiently expressing the polynucleotide comprising a heterologous morphogenic regulator, the plant cell may stimulate embryogenesis and/or organogenesis in a plant cell that does not comprise (i.e., is devoid of) a polynucleotide comprising a heterologous morphogenic regulator. The plant cell devoid of the polynucleotide comprising a heterologous morphogenic regulator may be a neighboring plant cell.

A “neighboring plant cell” as used herein refers to the location of a plant cell relative to another plant cell (i.e., the reference plant cell), and a neighboring plant cell is one that is in physical contact with, adjacent to, and/or in chemical communication with (e.g., receives cytokines from) the reference plant cell. The neighboring plant cell may be a plant cell that is from a different plant species or genotype than that of the transformed cell comprising the heterologous morphogenic regulator as described herein (e.g., the reference plant cell). In some embodiments, a neighboring plant cell is a plant cell in which a plasmodesmata (e.g., a primary and/or secondary plasmodesmata) connects the plant cell to the reference plant cell and/or in which a plasmodesmata traverses or passes through the cell wall of both the reference plant cell and neighboring plant cell. In some embodiments, a neighboring plant cell is the plant cell that is immediately adjacent to the reference plant call and middle lamella contacts or joins the cell walls of the two plant cells. In some embodiments, a neighboring plant cell is a plant cell in which an apoplast is formed by a portion of the cell wall of the reference plant cell and by a portion of the cell wall of the neighboring plant cell. In some embodiments, a neighboring plant cell is a plant cell in which 0, 1, 2, 3, 4, 5, 6, 7, 8, 9, 10, 11, 12, 13, 14, 15, 16, 17, 18, 19, 20, or more plant cells are between the cell wall of the neighboring plant cell and the cell wall of the reference plant cell. In some embodiments, a neighboring plant cell is one that is in the same explant as the reference plant cell and/or all cells in an explant are considered to be a neighboring plant cell. A neighboring plant cell may be devoid of a polynucleotide or a polypeptide comprising a heterologous morphogenic regulator, devoid of an editing system, and/or devoid of a selectable marker. In some embodiments, a first plant cell is transiently transformed with a polynucleotide or a polypeptide comprising a heterologous morphogenic regulator and the first plant cell stimulates a neighboring plant cell that is transiently transformed with an editing system to multiply and/or regenerate.

According to some embodiments, a composition, system, and/or method of the present invention causes or induces one or more plant cell(s) to multiply to provide one or more transgene-free plant cell(s), optionally using a plant cell in which a polynucleotide or a polypeptide comprising a heterologous morphogenic regulator has been introduced. The one or more transgene-free plant cell(s) may be undifferentiated plant cells or tissue or may be a callus. The one or more transgene-free plant cell(s) may be cultured explants. The one or more transgene-free cell(s) may form and/or may be grown to form one or more organ(s) and/or plant part(s). In some embodiments, a composition, system, and/or method of the present invention regenerates a transgene-free plant part or transgene-free plant, optionally using a plant cell in which a polynucleotide or a polypeptide comprising a heterologous morphogenic regulator has been introduced. In some embodiments, a transgene-free plant cell, a transgene-free plant part, and/or transgene-free plant is produced (e.g., regenerated) from the plant cell in which a polynucleotide or a polypeptide comprising a heterologous morphogenic regulator has been introduced. In some embodiments, the plant cell in which a polynucleotide or polypeptide comprising a heterologous morphogenic regulator has been introduced stimulates and/or initiates one or more neighboring plant cell(s) to multiply and/or regenerate to provide a transgene-free plant cell, a transgene-free plant part, or transgene-free plant. A method of the present invention may include, responsive to a step of introducing a polynucleotide or a polypeptide comprising a heterologous morphogenic regulator into a plant cell and optionally responsive to introducing an editing system into the same plant cell or a different plant cell (e.g., a neighboring plant cell), producing a plant cell, plant part and/or plant that is non-transgenic. A transgene-free plant cell, plant part and/or plant of the present invention may comprise an edited polynucleotide. The edited polynucleotide may be responsive to introducing an editing system into the same plant cell or a different plant cell (e.g., a neighboring plant cell) as the plant cell in which a polynucleotide or a polypeptide comprising a heterologous morphogenic regulator is introduced.

A composition, system, and/or method of the present invention may induce shoot formation, optionally using a plant cell in which a polynucleotide or a polypeptide comprising a heterologous morphogenic regulator and/or editing system has been introduced. In some embodiments, the method comprises expressing the heterologous morphogenic regulator in the plant cell and inducing shoot formation from the plant cell or a neighboring plant cell. The shoot formation may comprise apical dominance and/or roots.

A method of the present invention may include growing (e.g., culturing) a plant cell in the presence of and/or in contact with (e.g., on) media devoid of plant growth regulators and/or hormones (e.g., hormone-free media). The plant cell may comprise a polynucleotide or a polypeptide comprising a heterologous morphogenic regulator, editing system, and/or a selectable marker. In some embodiments, growing the plant cell comprises growing the plant cell in the presence of, in contact with, and/or on media devoid of plant growth regulators and/or hormones and producing a plant part and/or plant from the plant cell.

In some embodiments, a method of the present invention comprises transiently expressing an editing system in a plant cell and regenerating plant cells comprising an edited polynucleotide, optionally without the aid of selection (e.g., without using or introducing a selection marker). A polynucleotide encoding the editing system may be introduced into the plant cell or a neighboring plant cell, and/or the editing system may be introduced into the plant cell or a neighboring plant cell as an assembled ribonucleoprotein complex. In some embodiments, the editing system comprises a CRISPR-Cas effector protein (e.g., Cas9/Cas12a) and a guide nucleic acid.

In some embodiments, a method of the present invention comprises introducing (e.g., delivering) a polynucleotide or a polypeptide comprising a heterologous morphogenic regulator and a polynucleotide encoding a selectable marker to a plant cell, and regenerating a stably transformed plant under herbicide and/or antibiotic selection. In some embodiments, the method comprises introducing an editing system. In some embodiments, an editing system is not introduced.

In some embodiments, a method of the present invention comprises introducing (e.g., delivering) a polynucleotide or a polypeptide comprising a heterologous morphogenic regulator to a plant cell and does not include introducing a selectable marker to the plant cell. The method further includes regenerating a stably transformed plant without selection. In some embodiments, the method comprises introducing an editing system. In some embodiments, an editing system is not introduced.

In some embodiments, a composition, system and/or method of the present invention provides a genetic trigger for embryogenesis, organogenesis, and/or regeneration. In some embodiments, a composition, system and/or method of the present invention stimulates or causes transformed cells to multiply and/or regenerate faster and/or more efficiently than non-transformed cells. This may allow for transformed shoots to be more efficiently found among the regenerated shoots even without a selection system to inhibit the regeneration of untransformed cells.

In some embodiments, a method of the present invention comprises introducing (e.g., delivering) a polynucleotide or a polypeptide comprising a heterologous morphogenic regulator to a plant cell, and inducing a neighboring plant cell to multiply and/or regenerate. Certain types of morphogenic regulators (e.g., wuschel or WUS) organize embryo development on a concentration gradient from high to low. For this reason, cells that are transiently or stably transformed can induce their neighboring plant cells to organize an embryo and regenerate. Accordingly, a polynucleotide or a polypeptide comprising a heterologous morphogenic regulator may be introduced separately from another transgene and/or separately from an editing system. In some embodiments, the cells that receive the polynucleotide or polypeptide comprising a heterologous morphogenic regulator cause or induce neighboring plant cells, some of which have received the separate transgene and/or editing system, to multiply and/or regenerate. This can be with or without editing tools (e.g., an editing system) and/or with or without a selectable marker in the neighboring plant cells.

In some embodiments, a method of the present invention directly expresses transgenes encoding polynucleotides that initiate and/or define embryo identity. A composition, system, and/or method of the present invention may reduce or eliminate the dependence of a plant cell on media growth regulators and/or hormone supplements for regeneration. Media hormone supplementation enables many cells in a tissue, whether transformed or not, the potential to regenerate. However, an advantage of a composition, system, and/or method of the present invention can be that by introducing a polynucleotide or a polypeptide comprising a heterologous morphogenic regulator into a plant cell, the potential to regenerate is now tied to the transformation identity itself, representing a powerful approach to selectively encourage transformed cells to regenerate preferentially over untransformed cells. A polypeptide, polynucleotide, composition, system, and/or method of the present invention may increase or improve regeneration efficiency by about 2-, 4-, 6-, 8-, 10-, 20-, 30-, 40-, 50-, 60-, 70-, 80-, 90-100-, 200-, 300-, 400-, 500-, 600-, 700-, 800-, 900-, or 1000-fold compared to a control (e.g., a composition, system, cell (e.g., plant cell), and/or method that is devoid of a polypeptide and/or polynucleotide of the present invention).

A composition, system and/or method of the present invention may provide and/or produce a plant part and/or plant such as responsive to a step of introducing a polynucleotide or a polypeptide comprising a heterologous morphogenic regulator into a plant cell and optionally responsive to introducing an editing system into the same plant cell or a different plant cell (e.g., a neighboring plant cell) and responsive to growing plant cells. The plant part and/or plant may be morphologically normal compared to a control plant part and/or plant (e.g., compared to a non-transformed plant part or plant). Methods for determining morphological structures are known to those of skill in the art.

A composition, system and/or method of the present invention may comprise and/or introduce a morphogenic regulator as described herein. In some embodiments, the heterologous morphogenic regulator is a Knotted 1(KN1), Baby Boom (BBM), Wuschel (WUS) or Wuschel-related homeobox (WOX) gene, or an ortholog thereof. In some embodiments, the morphogenic regulator is ipt or an ortholog thereof. In some embodiments, the morphogenic regulator is kn1or an orthology thereof, optionally kn1 homeobox gene of maize. The maize transcription factor KN1 functions in plant meristems, self-renewing structures consisting of stem cells and their immediate daughters, which is associated with cytokinins accumulation through the positive regulation of cytokinin synthesis genes. In some embodiments, kn1 gene can be assessed to see if its transient expression is able to enhance plant regeneration and consequently increase gene editing occurrences. In some embodiments, the morphogenic regulator is WUS. In some embodiments, the morphogenic regulator is BBM. In some embodiments, the morphogenic regulator is CLV3. In some embodiments, the morphogenic regulator is miR156. In some embodiments, the morphogenic regulator is GRF5. In some embodiments, the morphogenic regulator is NTT. In some embodiments, the morphogenic regulator is HDZipII.

In some embodiments, the polynucleotide comprising the heterologous morphogenic regulator has at least 90%, 91%, 92%, 93%, 94%, 95%, 96%, 97%, 98%, 99%, or 100% sequence identity to the nucleotide sequence of any one of SEQ ID NOs:1-18 or SEQ ID NOs:28-49, optionally wherein the polynucleotide comprising the heterologous morphogenic regulator comprises the nucleotide sequence of any one of SEQ ID NOs:1-18 or SEQ ID NOs:28-49. In some embodiments, the polynucleotide comprising the heterologous morphogenic regulator has at least 90%, 91%, 92%, 93%, 94%, 95%, 96%, 97%, 98%, 99%, or 100% sequence identity to the nucleotide sequence of any one of SEQ ID NOs:1-18, optionally wherein the polynucleotide comprising the heterologous morphogenic regulator comprises the nucleotide sequence of any one of SEQ ID NOs:1-18. A polynucleotide comprising the heterologous morphogenic regulator may be operably associated with a promoter (e.g., a heterologous promoter). In some embodiments, the polynucleotide comprising the heterologous morphogenic regulator encodes an amino acid sequence having at least 90%, 91%, 92%, 93%, 94%, 95%, 96%, 97%, 98%, 99%, or 100% sequence identity to the polypeptide sequence of any one of SEQ ID NOs:19-27 or SEQ ID NOs:50-60, optionally wherein the polynucleotide comprising the heterologous morphogenic regulator encodes the polypeptide sequence of any one of SEQ ID NOs:19-27 or SEQ ID NOs:50-60. In some embodiments, the polynucleotide comprising the heterologous morphogenic regulator encodes an amino acid sequence having at least 90%, 91%, 92%, 93%, 94%, 95%, 96%, 97%, 98%, 99%, or 100% sequence identity to the polypeptide sequence of any one of SEQ ID NOs:19-27, optionally wherein the polynucleotide comprising the heterologous morphogenic regulator encodes the polypeptide sequence of any one of SEQ ID NOs:19-27. In some embodiments, the polypeptide comprising the heterologous morphogenic regulator has an amino acid sequence having at least 90%, 91%, 92%, 93%, 94%, 95%, 96%, 97%, 98%, 99%, or 100% sequence identity to the polypeptide sequence of any one of SEQ ID NOs:19-27 or SEQ ID NOs:50-60, optionally wherein the polypeptide comprising the heterologous morphogenic regulator has the polypeptide sequence of any one of SEQ ID NOs:19-27 or SEQ ID NOs:50-60. In some embodiments, the polypeptide comprising the heterologous morphogenic regulator has an amino acid sequence having at least 90%, 91%, 92%, 93%, 94%, 95%, 96%, 97%, 98%, 99%, or 100% sequence identity to the polypeptide sequence of any one of SEQ ID NOs:19-27, optionally wherein the polypeptide comprising the heterologous morphogenic regulator has the polypeptide sequence of any one of SEQ ID NOs:19-27. In some embodiments, the present disclosure provides a delivery of one or more components of an editing system directly to the plant cell. This is of interest, inter alia, for the generation of non-transgenic plants. In some embodiments, one or more of the components of the editing system is prepared outside the plant or plant cell and delivered to the cell. In some embodiments, the editing system is prepared in vitro prior to introduction to the plant cell. A base-editing fusion protein may be prepared by various methods known by one of skill in the art and include recombinant production. After expression, the CRISPR-Cas effector protein may be isolated, refolded if needed, purified and optionally treated to remove any purification tags if a tag such as a His-tag is present in the fusion protein. Once crude, partially purified, or more completely purified CRISPR-Cas effector protein is obtained, the protein may be introduced to the plant cell.

In some embodiments, the CRISPR-Cas effector protein is mixed with a guide nucleic acid that targets a target nucleic acid to form a pre-assembled ribonucleoprotein.

The individual components or pre-assembled ribonucleoprotein can be introduced into the plant cell via electroporation, by bombardment with targeted gene-editing system coated particles, by chemical transfection and/or by some other means of transport across a cell membrane. For instance, transfection of a plant protoplast with a pre-assembled CRISPR-Cas9 ribonucleoprotein has been demonstrated to ensure targeted modification of the plant genome (as described by Woo et al. Nature Biotechnology, 2015; 33(11):1162-1164).

In some embodiments, an editing system and/or one or more component(s) thereof may be introduced into a plant cell using nanoparticles. The editing system and/or components thereof, either as protein or nucleic acid or in a combination thereof, can be uploaded onto and/or packaged in nanoparticles and applied to the plants (such as for instance described in WO2008/042156 and US2013/0185823). In particular, the disclosure teaches nanoparticles uploaded with or packed with DNA molecule(s) encoding the CRISPR-Cas effector protein, DNA molecule(s) encoding cytosine deaminase (which may be fused to the CRISPR-Cas protein or a linker), and DNA molecules encoding the guide RNA and/or isolated guide RNA as described in WO2015/089419.

Further means of introducing one or more components of an editing system to a plant cell is by using cell penetrating peptides (CPP). Accordingly, some embodiments of the disclosure comprise compositions comprising a cell penetrating peptide linked to the gene-editing fusion protein. In some embodiments, a sequence-specific nucleic acid binding domain (e.g., a CRISPR Cas effector protein) and/or guide nucleic acid is coupled to one or more CPPs to effectively transport them inside plant protoplasts. Ramakrishna (Genome Res. 2014 June; 24(6): 1020-7 for Cas9 in human cells). In some embodiments, a sequence-specific nucleic acid binding domain (e.g., a CRISPR Cas effector protein) and/or guide nucleic acid are encoded by one or more circular or non-circular DNA molecule(s) which are coupled to one or more CPPs for plant protoplast delivery. The plant protoplasts are then regenerated to plant cells and further to plants. CPPs are generally described as short peptides of fewer than 35 amino acids either derived from proteins or from chimeric sequences which are capable of transporting biomolecules across cell membrane in a receptor independent manner. CPP can be cationic peptides, peptides having hydrophobic sequences, amphipathic peptides, peptides having proline-rich and anti-microbial sequence, and chimeric or bipartite peptides (Pooga and Langel 2005). CPPs are able to penetrate biological membranes and as such trigger the movement of various biomolecules across cell membranes into the cytoplasm and to improve their intracellular routing, and hence facilitate interaction of the biomolecule with the target.

In some embodiments, the methods described herein are used to modify a endogenous nucleic acid and/or gene and/or to modify their expression without the permanent introduction of any foreign gene including those encoding the editing system components into the genome of the plant, so as to avoid the presence of foreign DNA in the genome of the plant. In some embodiments, this may be provided by transient expression of the editing system components. In some embodiments, one or more of the components are expressed on one or more viral vectors which produce sufficient a sequence-specific nucleic acid binding domain (e.g., a CRISPR Cas effector protein), and guide nucleic acid to consistently steadily ensure modification of a nucleic acid of interest according to a method described herein. In some embodiments, transient expression of the editing system components is ensured in plant protoplasts and thus not integrated into the genome. The limited window of expression can be sufficient to allow the editing system to ensure modification of a target nucleic acid as described herein.

In some embodiments, the different components of the editing system are introduced in the plant cell, protoplast or plant tissue either separately or in mixture, with the aid of delivering molecules such as nanoparticles or CPP molecules as described herein above.

The expression of the editing system components can induce targeted modification of the genome. The different strategies described herein can allow CRISPR-mediated targeted genome editing without requiring the introduction of the editing system components into the plant genome. Components which are transiently introduced into the plant cell are used for editing a target nucleic acid and become non-functional naturally as they are stably integrated into a host genome.

In some embodiments, plant cells which have an edited polynucleotide and that are produced or obtained by any of the methods described herein, can be cultured to regenerate a whole plant that possesses the transformed or modified genotype and thus the desired phenotype. Conventional regeneration techniques are well known to those skilled in the art. Particular examples of such regeneration techniques rely on manipulation of certain phytohormones in a tissue culture growth medium, and typically relying on a biocide and/or herbicide marker which has been introduced together with the desired nucleotide sequences. In some embodiments, plant regeneration is obtained from cultured protoplasts, plant callus, explants, organs, pollens, embryos or parts thereof (see e.g. Evans et al. (1983), Handbook of Plant Cell Culture, Klee et al (1987) Ann. Rev. of Plant Phys.). However, the regeneration efficiency and ratio is very low.

In some embodiments, transformed or improved plants as described herein can be self-pollinated to provide seed for homozygous trait-improved plants of the disclosure, which have a desired trait such as seedlessness, reduced seed size, reduced endocarp tissue, or less lignified endocarp (homozygous for the DNA modification) or crossed with non-transgenic plants or different trait-improved plants to provide seed for heterozygous plants. Where a recombinant DNA was introduced into the plant cell, the resulting plant of such a crossing is a plant which is heterozygous for the recombinant DNA molecule. Both such homozygous and heterozygous plants obtained by crossing from the trait-improved plants and comprising the genetic modification (which can be a recombinant DNA) are referred to herein as “progeny”. Alternatively, gene-edited plants can be obtained by one of the methods described herein using an editing system whereby no foreign DNA is incorporated into the genome using transient expression/delivery or whereby foreign DNA is incorporated into the genome using stable transformation but removed/segregated away upon crossing. Progeny of such plants, obtained by further breeding may also contain the genetic modification such as nucleotide substitutions. Breedings may be performed by any breeding methods that are commonly used for different crops (e.g., Allard, Principles of Plant Breeding, John Wiley & Sons, NY, U. of CA, Davis, CA, 50-98 (1960). However, this approach cannot be applied to plants and/or crops with high heterozygosity and/or multiploidy.

Developing a method to generate CRISPR-mediated non-transgenic events is highly desirable for many applications of genome editing, particularly for asexually propagated, heterozygous, perennial crop plants. It has been reported that pre-assembled CRISPR/Cas9 ribonucleoproteins can be delivered into protoplasts to induce mutations, without the need for stable integration of CRISPR/Cas9 genes into the host-plant genome (Malnoy et al. 2016, Subburaj et al. 2016, Woo et al. 2015). Particle bombardment has also been used to deliver CRISPR/Cas9 ribonucleoproteins to wheat and maize cells, producing non-transgenic mutants (Liang et al. 2017, Svitashev et al 2016, Zhang et al. 2016, Kim 2017).

A method for production and expedient screening of CRISPR/Cas9-mediated non-transgenic mutant plants via transient T-DNA expression has been reported using tobacco as a model species (Che et al 2018). The mutant rate of non-transgenic plant from total regenerated plants was <0.5% when no selection was applied. The present disclosure teaches that transient expression of a morphogenic regulator in dicot species is demonstrated to enhance regeneration and may subsequently increase gene editing efficiency.

As mentioned above, transient expression of an editing system via Agrobacterium-mediated T-DNA delivery was reported to generate edited events without transgene integration, the overall efficiency was low due to the lack of selection (Chen et al. 2018). The present disclosure teaches use of transient expression of morphogenic regulator(s) to enhance the regeneration of edited cells without transgene integration. In some embodiments, morphogenic regulators are transiently transformed using Agrobacterium-mediated T-DNA delivery along with gene editing machinery that generates edited events.

In some embodiments, a gene-edited plant cell produced according to a method of the present is grown, multiplied, and/or regenerated in a medium without growth hormones, in which case, for example, at most 2%, at most 1%, at most 0.9%, at most 0.8%, at most 0.7%, at most 0.6%, at most 0.5%, at most 0.4%, at most 0.3%, at most 0.2%, at most 0.1%, at most 0.05%, at most 0.01%, at most 0.005%, at most 0.001%, at most 0.0005%, or at most 0.0001% or lower of the transformed cells are regenerated into a whole plants. In some embodiments, a gene-edited plant cell produced according to a method of the present invention is transformed with an expression cassette comprising one or more (e.g., 1, 2, 3, 4, 5, or more) morphogenic regulator(s) (such as WUS, BBN, KN1, IPT, CLV3, miR156, GRF5, NTT, and/or HDZipII) and regenerated in a medium without growth hormones, in which case, for example, at least 1%, at least 2%, at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or higher, of the transformed cells are regenerated into a whole plants.

In some embodiments of the present invention, transient expression of at least one morphogenic regulator in a transformed cell is capable of regenerating a plant without regeneration-inducing hormones added.

In some embodiments, transient expression of at least one morphogenic regulator in a transformed cell is capable of regenerating a plant in combination with addition of regeneration-inducing hormones in an improved, enhanced and/or synergistic manner.

In some embodiments, a plant cell of interest can be co-transformed with an expression cassette comprising one or more gene editing components/machinery and another expression cassette comprising at least one morphogenic regulator. In embodiments, the transient expression of at least one morphogenic regulator increases, enhances, improves transformation efficiency of the gene-editing system. In further embodiments, the transient expression of at least one morphogenic regulator increases, enhances, improves regeneration of the gene-edited plant cell. In further embodiments, the transient expression of at least one morphogenic regulator increases, enhances, improves regeneration of the gene-edited plant cell even in a culture medium without hormones such as regeneration-inducing hormones.

In other embodiments, the morphogenic regulators taught in the present disclosure can be used as a positive selection marker that gives rise to plant regeneration in a hormone-less culture medium. This allows for avoidance of use of a selectable marker gene (usually an antibiotic- or herbicide-resistant gene), which can make the transformed cells and/or regenerated plants transgenic due to undesired DNA pieces that is stably integrated and inherited.

In some embodiments, transient expression of morphogenic regulators in a plant cell transformed with the gene-editing machinery taught in the present disclosure increases, improves, and/or enhance plant transformation efficiency at least 0.5%, at least 1%, at least 2%, at least 3%, at least 4%, at least 5%, at least 6%, at least 7%, at least 8%, at least 9%, at least 10%, at least 12%, at least 15%, at least 18%, at least 20%, at least 30%, at least 40%, at least 50%, at least 60%, at least 70%, at least 80%, at least 90%, at least 100%, at least 150%, at least 200%, at least 250%, at least 300%, at least 400%, at least 500% or higher than a plant cell transformed with the gene-editing machinery, but without morphogenic regulator expression.

In some embodiments, transient expression of morphogenic regulators in a plant cell transformed with the gene-editing machinery taught in the present disclosure increases, improves, and/or enhance plant regeneration at least 0.5%, at least 1%, at least 2%, at least 3%, at least 4%, at least 5%, at least 6%, at least 7%, at least 8%, at least 9%, at least 10%, at least 12%, at least 15%, at least 18%, at least 20%, at least 30%, at least 40%, at least 50%, at least 60%, at least 70%, at least 80%, at least 90%, at least 100%, at least 150%, at least 200%, at least 250%, at least 300%, at least 400%, at least 500% or higher than a plant cell transformed with the gene-editing machinery, but without morphogenic regulator expression.

In some embodiments, the modified plant cells described herein demonstrate an altered expression and/or function of one or more endogenous target genes.

In some embodiments, the expression of an endogenous target gene in a particular pathway is reduced in the modified plant cells. In some embodiments, the expression of a plurality (e.g., two or more) of endogenous target genes in a particular pathway are reduced in the modified plant cells. For example, the expression of 2, 3, 4, 5, 6, 7, 8, 9, 10, or more endogenous target genes in a particular pathway may be reduced. In some embodiments, the expression of an endogenous target gene in one pathway and the expression of an endogenous target genes in another pathway is reduced in the modified plant cells. In some embodiments, the expression of a plurality of endogenous target genes in one pathway and the expression of a plurality of endogenous target genes in another pathway are reduced in the modified plant cells. For example, the expression of 2, 3, 4, 5, 6, 7, 8, 9, 10, or more endogenous target genes in one pathway may be reduced and the expression of 2, 3, 4, 5, 6, 7, 8, 9, 10, or more endogenous target genes in another particular pathway may be reduced. In some embodiments, the expression of a plurality of endogenous target genes in a plurality of pathways is reduced. For example, the expression of one endogenous gene from each of a plurality of pathways (e.g., 2, 3, 4, 5, 6, 7, 8, 9, 10, or more pathways) may be reduced. In additional aspects, the expression of a plurality of endogenous genes (e.g., 2, 3, 4, 5, 6, 7, 8, 9, 10, or more genes) from each of a plurality of pathways (e.g., 2, 3, 4, 5, 6, 7, 8, 9, 10, or more pathways) may be reduced.

In some embodiments, the function of a protein encoded by an endogenous target gene in a particular pathway is altered in the modified plant cells. In some embodiments, the functions of proteins encoded by a plurality (e.g., two or more) of endogenous target genes in a particular pathway are altered in the modified plant cells. For example, the function of proteins encoded by 2, 3, 4, 5, 6, 7, 8, 9, 10, or more endogenous target genes in a particular pathway may be altered. In some embodiments, the function of a protein encoded by an endogenous target gene in one pathway and the function of an endogenous target genes in another pathway is altered in the modified plant cells. In some embodiments, the functions of proteins encoded by a plurality of endogenous target genes in one pathway and the function of proteins encoded by a plurality of endogenous target genes in another pathway are altered in the modified plant cells. For example, the function of proteins encoded by 2, 3, 4, 5, 6, 7, 8, 9, 10, or more endogenous target genes in one pathway may be altered and the function of proteins encoded by 2, 3, 4, 5, 6, 7, 8, 9, 10, or more endogenous target genes in another particular pathway may be altered. In some embodiments, the functions of proteins encoded by a plurality of endogenous target genes in a plurality of pathways are altered. For example, the function of a protein encoded by one endogenous gene from each of a plurality of pathways (e.g., 2, 3, 4, 5, 6, 7, 8, 9, 10, or more pathways) may be altered. In additional aspects, the function of proteins encoded by a plurality of endogenous genes (e.g., 2, 3, 4, 5, 6, 7, 8, 9, 10, or more genes) from each of a plurality of pathways (e.g., 2, 3, 4, 5, 6, 7, 8, 9, 10, or more pathways) may be altered.

Gene editing generally refers to the process of modifying the nucleotide sequence of a genome, preferably in a precise or pre-determined manner. Examples of methods of gene editing described herein include methods of using site-directed nucleases to cut deoxyribonucleic acid (DNA) at precise target locations in the genome, thereby creating single-strand or double-strand DNA breaks at particular locations within the genome. Such breaks can be and regularly are repaired by natural, endogenous cellular processes, such as homology-directed repair (HDR) and non-homologous end joining (NHEJ). These two main DNA repair processes consist of a family of alternative pathways. NHEJ directly joins the DNA ends resulting from a double-strand break, sometimes with the loss or addition of nucleotide sequence, which may disrupt or enhance gene expression. HDR utilizes a homologous sequence, or donor sequence, as a template for inserting a defined DNA sequence at the break point. The homologous sequence can be in the endogenous genome, such as a sister chromatid. Alternatively, the donor can be an exogenous nucleic acid, such as a plasmid, a single-strand oligonucleotide, a double-stranded oligonucleotide, a duplex oligonucleotide or a virus, that has regions of high homology with the nuclease-cleaved locus, but which can also contain additional sequence or sequence changes including deletions that can be incorporated into the cleaved target locus. A third repair mechanism can be microhomology-mediated end joining (MMEJ), also referred to as “Alternative NHEJ,” in which the genetic outcome is similar to NHEJ in that small deletions and insertions can occur at the cleavage site. MMEJ can make use of homologous sequences of a few basepairs flanking the DNA break site to drive a more favored DNA end joining repair outcome, and recent reports have further elucidated the molecular mechanism of this process; see, e.g., Cho and Greenberg, Nature 518, 174-76 (2015); Kent et al., Nature Structural and Molecular Biology, Adv. Online doi:10.1038/nsmb.2961(2015); Mateos-Gomez et al., Nature 518, 254-57 (2015); Ceccaldi et al., Nature 528, 258-62 (2015). In some instances, it may be possible to predict likely repair outcomes based on analysis of potential microhomologies at the site of the DNA break.

“Recombination” refers to a process of exchange of genetic information between two polynucleotides, including but not limited to, donor capture by non-homologous end joining (NHEJ) and homologous recombination. For the purposes of this disclosure, “homologous recombination (HR)” refers to the specialized form of such exchange that takes place, for example, during repair of double-strand breaks in cells via homology-directed repair (HDR) mechanisms. This process requires nucleotide sequence homology, uses a “donor” molecule as a template to repair a “target” molecule (i.e., the one that experienced the double-strand break), and is variously known as “non-crossover gene conversion” or “short tract gene conversion,” because it leads to the transfer of genetic information from the donor to the target. Without wishing to be bound by any particular theory, such transfer can involve mismatch correction of heteroduplex DNA that forms between the broken target and the donor, and/or synthesis-dependent strand annealing, in which the donor is used to resynthesize genetic information that will become part of the target, and/or related processes. Such specialized HR often results in an alteration of the sequence of the target molecule such that part or all of the sequence of the donor polynucleotide is incorporated into the target polynucleotide.

Gene editing methods contemplated in various embodiments comprise engineered nucleases, designed to bind and cleave a target DNA sequence in a gene of interest. The engineered nucleases contemplated in particular embodiments, can be used to introduce a double-strand break in a target polynucleotide sequence, which may be repaired (i) by non-homologous end joining (NHEJ) in the absence of a polynucleotide template, e.g., a donor repair template, or (ii) by homology directed repair (HDR), i.e., homologous recombination, in the presence of a donor repair template. Engineered nucleases contemplated in certain embodiments, can also be engineered as nickases, which generate single-stranded DNA breaks that can be repaired using the cell's base-excision-repair (BER) machinery or homologous recombination in the presence of a donor repair template.

Gene editing via sequence-specific nucleases is known in the art. See references (1) Carroll, D. (2011) Genome engineering with zinc-finger nucleases. Genetics, 188, 773-82; (2) Wood, A. J. et al. (2011) Targeted gene editing across species using ZFNs and TALENs. Science (New York, N.Y.), 333, 307; (3) Perez-Pinera, P. et al. (2012) Advances in targeted gene editing. Current opinion in chemical biology, 16, 268-77, each of which is hereby incorporated by reference in their entireties.

A nuclease-mediated double-stranded DNA (dsDNA) break in the genome can be repaired by two main mechanisms: Non-Homologous End Joining (NHEJ), which frequently results in the introduction of non-specific insertions and deletions (indels), or homology directed repair (HDR), which incorporates a homologous strand as a repair template. See Symington, L. S. and Gautier, J. (2011) Double-strand break end resection and repair pathway choice. Annual review of genetics, 45, 247-71, which is hereby incorporated by reference in its entirety.

When a sequence-specific nuclease is delivered along with a homologous donor DNA construct containing the desired mutations, gene targeting efficiencies are increased by 1000-fold compared to just the donor construct alone. See Urnov et al. (2005) Highly efficient endogenous human gene correction using designed zinc-finger nucleases. Nature, 435, 646-51, which is hereby incorporated by reference in its entirety.

In some embodiments, the gene editing techniques of the present disclosure are used for plants that are modified using any gene editing tool, including, but not limited to: ZFNs, TALENS, CRISPR, and Mega nuclease technologies. In some embodiments, the gene editing tools of the present disclosure comprise proteins or polynucleotides which have been custom designed to target and cut at specific deoxyribonucleic acid (DNA) sequences. In some embodiments, gene editing proteins are capable of directly recognizing and binding to selected DNA sequences. In other embodiments, the gene editing tools of the present disclosure form complexes, wherein nuclease components rely on nucleic acid molecules for binding and recruiting the complex to the target DNA sequence.

In some embodiments, the single component gene editing tools comprise a binding domain capable of recognizing specific DNA sequences in the genome of the plant and a nuclease that cuts double-stranded DNA. The rationale of gene editing technology taught in the present disclosure is the use of a tool that allows the introduction of site-specific mutations in the plant genome or the site-specific integration of genes.

Many methods are available for delivering genes into plant cells, e.g. transfection, electroporation, viral vectors and Agrobacterium mediated transfer. Genes can be expressed transiently from a plasmid vector. Once expressed, the genes generate the targeted mutation that will be stably inherited, even after the degradation of the plasmid containing the gene.

Customizable nucleases can be used to make targeted double-stranded breaks (DSB) in living cells, the repair of which can be exploited to induce desired sequence changes. Two competing pathways effect repairs in most cells, including plant cells. Repair of a nuclease-induced DSB by non-homologous end joining (NHEJ) leads to the introduction of insertion/deletion mutations (indels) with high frequencies. By contrast, DSB repair by homology directed repair (HDR) with a user-supplied “donor template” DNA can lead to the introduction of specific alterations (e.g., point mutations and insertions) or the correction of mutant sequences back to wild-type.

In some embodiments, a plant cell of interest is generated by gene editing accomplished with engineered nucleases targeting one or more loci that contributes to a target gene of interest. Without wishing to be bound to any particular theory, it is contemplated that engineered nucleases are designed to precisely disrupt one or more target genes of interest through gene editing and, once nuclease activity and specificity are validated, lead to predictable disruption of target gene expression and/or function.

The engineered nucleases described herein generate single-stranded DNA nicks or double-stranded DNA breaks (DSB) in a target sequence. Furthermore, a DSB can be achieved in the target DNA by the use of two nucleases generating single-stranded micks (nickases). Each nickase cleaves one strand of the DNA and the use of two or more nickases can create a double strand break (e.g., a staggered double-stranded break) in a target DNA sequence. In other embodiments, the nucleases are used in combination with a donor repair template, which is introduced into the target sequence at the DNA break-site via homologous recombination at a DSB.

Engineered nucleases described herein that are suitable for gene editing comprise one or more DNA binding domains and one or more DNA cleavage domains (e.g., one or more endonuclease and/or exonuclease domains), and optionally, one or more linkers contemplated herein. An “engineered nuclease” refers to a nuclease comprising one or more DNA binding domains and one or more DNA cleavage domains, wherein the nuclease has been designed and/or modified to bind a DNA binding target sequence adjacent to a DNA cleavage target sequence. The engineered nuclease may be designed and/or modified from a naturally occurring nuclease or from a previously engineered nuclease.

Illustrative examples of nucleases that may be engineered to bind and cleave a target sequence include, but are not limited to homing endonucleases (meganucleases), megaTALs, transcription activator-like effector nucleases (TALENs), zinc finger nucleases (ZFNs), and clustered regularly-interspaced short palindromic repeats (CRISPR)/Cas nuclease systems.

In some embodiments, the nucleases contemplated herein comprise one or more heterologous DNA-binding and cleavage domains (e.g., ZFNs, TALENs, megaTALs), (Boissel et al., 2014; Christian et al., 2010). In other embodiments, the DNA-binding domain of a naturally-occurring nuclease may be altered to bind to a selected target site (e.g., a meganuclease that has been engineered to bind to site different than the cognate binding site). For example, meganucleases have been designed to bind target sites different from their cognate binding sites (Boissel et al., 2014). In particular embodiments, a nuclease requires a nucleic acid sequence to target the nuclease to a target site (e.g., CRISPR/Cas).

### (i) TALEN

Transcription activator-like effector nucleases (TALENs) comprise a nonspecific DNA-cleaving nuclease (e.g., a Fok I cleavage domain) fused to a DNA-binding domain that can be easily engineered so that TALENs can target essentially any sequence (See, e.g., Joung and Sander, Nature Reviews Molecular Cell Biology 14:49-55 (2013)). Methods for generating engineered TALENs are known in the art, see, e.g., the fast ligation-based automatable solid-phase high-throughput (FLASH) system described in U.S. Ser. No. 61/610,212, and Reyon et al., Nature Biotechnology 30,460-465 (2012); as well as the methods described in Bogdanove & Voytas, Science 333, 1843-1846 (2011); Bogdanove et al., Curr Opin Plant Biol 13, 394-401 (2010); Scholze & Boch, J. Curr Opin Microbiol (2011); Boch et al., Science 326, 1509-1512 (2009); Moscou & Bogdanove, Science 326, 1501 (2009); Miller et al., Nat Biotechnol 29, 143-148 (2011); Morbitzer et al., T. Proc Natl Acad Sci USA 107, 21617-21622 (2010); Morbitzer et al., Nucleic Acids Res 39, 5790-5799 (2011); Zhang et al., Nat Biotechnol 29, 149-153 (2011); Geissler et al., PLoS ONE 6, e19509 (2011); Weber et al., PLoS ONE 6, e19722 (2011); Christian et al., Genetics 186, 757-761 (2010); Li et al., Nucleic Acids Res 39, 359-372 (2011); Mahfouz et al., Proc Natl Acad Sci USA 108, 2623-2628 (2011); Mussolino et al., Nucleic Acids Res (2011); Li et al., Nucleic Acids Res 39, 6315-6325 (2011); Cermak et al., Nucleic Acids Res 39, e82 (2011); Wood et al., Science 333, 307 (2011); Hockemeye et al. Nat Biotechnol 29, 731-734 (2011); Tesson et al., Nat Biotechnol 29, 695-696 (2011); Sander et al., Nat Biotechnol 29, 697-698 (2011); Huang et al., Nat Biotechnol 29, 699-700 (2011); and Zhang et al., Nat Biotechnol 29, 149-153 (2011); all of which are incorporated herein by reference in their entirety.

In some embodiments, a TALEN that binds to and cleaves a target region of a locus that contributes to a target gene of interest. A “TALEN” refers to an engineered nuclease comprising an engineered TALE DNA binding domain contemplated elsewhere herein and an endonuclease domain (or endonuclease half-domain thereof), and optionally comprise one or more linkers and/or additional functional domains, e.g., an end-processing enzymatic domain of an end-processing enzyme that exhibits 5-3′ exonuclease, 5-3′ alkaline exonuclease, 3-5′exonuclease (e.g., Trex2), 5′ flap endonuclease, helicase or template-independent DNA polymerases activity.

In some embodiments, plants of interest are modified through Transcription activator-like (TAL) effector nucleases (TALENs). TALENS are polypeptides with repeat polypeptide arms capable of recognizing and binding to specific nucleic acid regions. By engineering the polypeptide arms to recognize selected target sequences, the TAL nucleases can be use to direct double stranded DNA breaks to specific genomic regions. These breaks can then be repaired via recombination to edit, delete, insert, or otherwise modify the DNA of a host organism. In some embodiments, TALENSs are used alone for gene editing (e.g., for the deletion or disruption of a gene). In other embodiments, TALs are used in conjunction with donor sequences and/or other recombination factor proteins that will assist in the Non-homologous end joining (NHEJ) process to replace the targeted DNA region. For more information on the TAL-mediated gene editing compositions and methods of the present disclosure, see U.S. Pat. Nos. 8,440,432; 8,440,432; 8,450,471; 8,586,526; 8,586,363; 8,592,645; 8,697,853; 8,704,041; 8,921,112; and 8,912,138, each of which is hereby incorporated in its entirety for all purposes.

### (ii) MegaTALs

Various illustrative embodiments contemplate a megaTAL nuclease that binds to and cleaves a target region of a locus that contributes to a target gene of interest. A “megaTAL” refers to an engineered nuclease comprising an engineered TALE DNA binding domain and an engineered meganuclease, and optionally comprise one or more linkers and/or additional functional domains, e.g., an end-processing enzymatic domain of an end-processing enzyme that exhibits 5-3′ exonuclease, 5-Y alkaline exonuclease, 3-5′exonuclease, 5′ flap endonuclease, helicase or template-independent DNA polymerases activity.

A “TALE DNA binding domain” is the DNA binding portion of transcription activator-like effectors (TALE or TAL-effectors), which mimics plant transcriptional activators to manipulate the plant transcriptome (see e.g., Kay et al., 2007. Science 318:648-651). TALE DNA binding domains contemplated in particular embodiments are engineered de novo or from naturally occurring TALEs. Illustrative examples of TALE proteins for deriving and designing DNA binding domains are disclosed in U.S. Pat. No. 9,017,967, and references cited therein, all of which are incorporated herein by reference in their entireties.

In some embodiments, plants of interest are modified through megaTALs. In some embodiments, megaTALs are engineered endonucleases capable of targeting selected DNA sequences and inducing DNA breaks.

(iii) Meganucleases/Homing Endonucleases (HE)

Meganucleases are sequence-specific endonucleases originating from a variety of organisms such as bacteria, yeast, algae and plant organelles. A number of Meganucleases are known in the art, see, e.g., WO 2012010976 (Meganuclease variants cleaving DNA target sequences of the TERT gene); U.S. Pat. Nos. 8,021,867; 8,119,361 and 8,119,381 (I-CreI meganucleases); U.S. Pat. No. 7,897,372 (I-CreI Meganuclease Variants with Modified Specificity).

In some embodiments, a homing endonuclease or meganuclease is engineered to bind to, and to introduce single-stranded nicks or double-strand breaks (DSBs) in, one or more loci that contribute to a target gene of interest. “Homing endonuclease” and “meganuclease” are used interchangeably and refer to naturally-occurring nucleases or engineered meganucleases that recognize 12-45 base-pair cleavage sites and are commonly grouped into five families based on sequence and structure motifs: LAGLIDADG (SED ID NO:135), GIY-YIG, HNH, His-Cys box, and PD-(D/E)XK.

Engineered HEs do not exist in nature and can be obtained by recombinant DNA technology or by random mutagenesis. Engineered HEs may be obtained by making one or more amino acid alterations, e.g., mutating, substituting, adding, or deleting one or more amino acids, in a naturally occurring HE or previously engineered HE. In particular embodiments, an engineered HE comprises one or more amino acid alterations to the DNA recognition interface. Engineered HEs contemplated in particular embodiments may further comprise one or more linkers and/or additional functional domains, e.g., an end-processing enzymatic domain of an end-processing enzyme that exhibits 5-3′ exonuclease, 5-3′ alkaline exonuclease, 3-5′exonuclease, 5′ flap endonuclease, helicase or template-independent DNA polymerases activity.

In some embodiments, plants of interest are modified through meganucleases. In some embodiments, meganucleases are engineered endonucleases capable of targeting selected DNA sequences and inducing DNA breaks. In some embodiments, new meganucleases targeting specific regions are developed through recombinant techniques which combine the DNA binding motifs from various other identified nucleases. In other embodiments, new meganucleases are created through semi-rational mutational analysis, which attempts to modify the structure of existing binding domains to obtain specificity for additional sequences. For more information on the use of meganucleases for genome editing, see Silva et al., 2011 Current Gene Therapy 11 pg 11-27; and Stoddard et al., 2014 Mobile DNA 5 pg 7, each of which is hereby incorporated in its entirety for all purposes.

### (iv) ZFN

Zinc-finger nucleases (ZFNs) are composed of programmable, sequence-specific zinc finger DNA-binding modules (see above) linked to a nonspecific DNA cleavage domain, e.g., a Fok I cleavage domain. Methods for making and using ZFNs are known in the art, see, e.g., (Maeder et al., 2008, Mol. Cell, 31:294-301; Joung et al., 2010, Nat. Methods, 7:91-92; Isalan et al., 2001, Nat. Biotechnol., 19:656-660; Sander et al., Nat Methods. 8(1):67-9, 2011; Bhakta et al., Genome Res. 23(3):530-8, 2013). In some embodiments, the ZFNs are described in, or are generated as described in, WO 2011/017293 or WO 2004/099366. Additional suitable ZFNs are described in U.S. Pat. Nos. 6,511,808, 6,013,453, 6,007,988, and 6,503,717 and U.S. patent application 2002/0160940.

In some embodiments, a zinc finger nuclease (ZFN) that binds to and cleaves a target region of a locus that contributes to a target gene of interest. A “ZFN” refers to an engineered nuclease comprising one or more zinc finger DNA binding domains and an endonuclease domain (or endonuclease half-domain thereof), and optionally comprise one or more linkers and/or additional functional domains, e.g., an end-processing enzymatic domain of an end-processing enzyme that exhibits 5-3′ exonuclease, 5-3′ alkaline exonuclease, 3-5′exonuclease, 5′ flap endonuclease, helicase or template-independent DNA polymerases activity.

In one embodiment, targeted double-stranded cleavage is achieved using two ZFNs, each comprising an endonuclease half-domain can be used to reconstitute a catalytically active cleavage domain. In another embodiment, targeted double-stranded cleavage is achieved with a single polypeptide comprising one or more zinc finger DNA binding domains and two endonuclease half-domains.

In one embodiment, a ZFN comprises a TALE DNA binding domain contemplated elsewhere herein, a zinc finger DNA binding domain, and an endonuclease domain (or endonuclease half-domain) contemplated elsewhere herein.

In one embodiment, a ZFN comprises a zinc finger DNA binding domain, and a meganuclease contemplated elsewhere herein.

In particular embodiments, the ZFN comprises a zinger finger DNA binding domain that has one, two, three, four, five, six, seven, or eight or more zinger finger motifs and an endonuclease domain (or endonuclease half-domain). Typically, a single zinc finger motif is about 30 amino acids in length. Zinc fingers motifs include both canonical C₂H₂ zinc fingers, and non-canonical zinc fingers such as, for example, C₃H zinc fingers and C₄ zinc fingers.

Zinc finger binding domains can be engineered to bind any DNA sequence. Candidate zinc finger DNA binding domains for a given 3 bp DNA target sequence have been identified and modular assembly strategies have been devised for linking a plurality of the domains into a multi-finger peptide targeted to the corresponding composite DNA target sequence. Other suitable methods known in the art can also be used to design and construct nucleic acids encoding zinc finger DNA binding domains, e.g., phage display, random mutagenesis, combinatorial libraries, computer/rational design, affinity selection, PCR, cloning from cDNA or genomic libraries, synthetic construction and the like. (See, e.g., U.S. Pat. No. 5,786,538; Wu et al., PNAS 92:344-348 (1995); Jamieson et al., Biochemistry 33:5689-5695 (1994); Rebar & Pabo, Science 263:671-673 (1994); Choo & Klug, PNAS 91:11163-11167 (1994); Choo & Klug, PNAS 91: 11168-11172 (1994); Desjarlais & Berg, PNAS 90:2256-2260 (1993); Desjarlais & Berg, PNAS 89:7345-7349 (1992); Pomerantz et al., Science 267:93-96 (1995); Pomerantz et al., PNAS 92:9752-9756 (1995); Liu et al., PNAS 94:5525-5530 (1997); Griesman & Pabo, Science 275:657-661 (1997); Desjarlais & Berg, PNAS 91:11-99-11103 (1994)).

Individual zinc finger motifs bind to a three or four nucleotide sequence. The length of a sequence to which a zinc finger binding domain is engineered to bind (e.g., a target sequence) will determine the number of zinc finger motifs in an engineered zinc finger binding domain. For example, for ZFNs in which the zinc finger motifs do not bind to overlapping subsites, a six-nucleotide target sequence is bound by a two-finger binding domain; a nine-nucleotide target sequence is bound by a three-finger binding domain, etc. In particular embodiments, DNA binding sites for individual zinc fingers motifs in a target site need not be contiguous, but can be separated by one or several nucleotides, depending on the length and nature of the linker sequences between the zinc finger motifs in a multi-finger binding domain.

In some embodiments, plants of interest are modified through Zinc Finger Nucleases. Three variants of the ZFN technology are recognized in plant genome engineering (with applications ranging from producing single mutations or short deletions/insertions in the case of ZFN-1 and -2 techniques up to targeted introduction of new genes in the case of the ZFN-3 technique):

ZFN-1: Genes encoding ZFNs are delivered to plant cells without a repair template. The ZFNs bind to the plant DNA and generate site specific double-strand breaks (DSBs). The natural DNA-repair process (which occurs through nonhomologous end-joining, NHEJ) leads to site specific mutations, in one or only a few base pairs, or to short deletions or insertions.

ZFN-2: Genes encoding ZFNs are delivered to plant cells along with a repair template homologous to the targeted area, spanning a few kilo base pairs. The ZFNs bind to the plant DNA and generate site-specific DSBs. Natural gene repair mechanisms generate site-specific point mutations e.g. changes to one or a few base pairs through homologous recombination and the copying of the repair template.

ZFN-3: Genes encoding ZFNs are delivered to plant cells along with a stretch of DNA which can be several kilo base pairs long and the ends of which are homologous to the DNA sequences flanking the cleavage site. As a result, the DNA stretch is inserted into the plant genome in a site specific manner.

### (v) FokI

FokI is a type IIs restriction endonuclease that includes a DNA recognition domain and a catalytic (endonuclease) domain. The fusion proteins described herein can include all of FokI or just the catalytic endonuclease domain, e.g., amino acids 388-583 or 408-583 of GenBank Acc. No. AAA24927.1, e.g., as described in WO95/09233, Li et al., Nucleic Acids Res. 39(1): 359-372 (2011); Cathomen and Joung, Mol. Ther. 16: 1200-1207 (2008), or a mutated form of FokI as described in Miller et al. Nat Biotechnol 25: 778-785 (2007); Szczepek et al., Nat Biotechnol 25: 786-793 (2007); or Bitinaite et al., Proc. Natl. Acad. Sci. USA. 95:10570-10575 (1998). See also Tsai et al., Nat Biotechnol. 2014 June; 32(6):569-76

In some embodiments, plants of interest are modified through FokI endonucleases.

Herein, the term “targeted gene-editing system” refers to a protein, nucleic acid, or combination thereof that is capable of substituting, inserting, or deleting one or more nucleotides at a target site and modifying an endogenous target DNA sequence when introduced into a cell, thereby causing one or more amino acid substitutions, insertions, or deletions. In some embodiments, the editing system taught in the present disclosure refers to the targeted gene editing system.

More specifically, the term “targeted base-editing system” refers to a protein, nucleic acid, or combination thereof that is capable of substituting one or more nucleotides at a target site and modifying an endogenous target DNA sequence when introduced into a cell, thereby causing one or more amino acid substitutions.

Numerous gene editing systems suitable for use in the methods of the present disclosure, include, but are not limited to, zinc-finger nuclease systems, TALEN systems, and CRISPR/Cas systems.

In some embodiments, a nuclease-inactivated CRISPR/Cas system having a base deaminase activity is utilized for a targeted base-editing. In other aspects, a nickase is used.

In some embodiments, the targeted base-editing system can mediate a change in the sequence of the endogenous target gene, for example, by introducing one or more point mutations into the endogenous target sequence, such as by substituting C with T (or G with A) or A with G (or T with C) in the endogenous target sequence.

In some embodiments, the targeted gene-editing system may mediate a change in the expression of the protein encoded by the endogenous target gene. In such embodiments, the targeted gene-editing system may regulate the expression of the encoded protein by modifications of the endogenous target DNA sequence, or by acting on the mRNA product encoded by the DNA sequence. In some embodiments, the targeted gene-editing system may result in the expression of a modified endogenous protein. In some embodiments, the modifications to the endogenous DNA sequence mediated by the targeted gene-editing system result in an altered function of the modified endogenous protein as compared to the corresponding endogenous protein in an unmodified plant cell. In such embodiments, the expression level of the modified endogenous protein may be increased, decreased or may be the same, or substantially similar to, the expression level of the corresponding endogenous protein in an unmodified plant cell.

The present disclosure provides a targeted gene-editing system to edit a target nucleotide sequence in the genome of a plant, comprising at least one of the followings; i) a gene editing fusion protein, and a guide RNA; ii) an expression construct comprising a nucleotide sequence encoding a gene editing fusion protein, and a guide RNA; iii) a gene editing fusion protein, and an expression construction comprising a nucleotide sequence encoding a guide RNA; iv) an expression construct comprising a nucleotide sequence encoding a gene editing fusion protein, and an expression construct comprising a nucleotide sequence encoding a guide RNA; v) an expression construct comprising a nucleotide sequence encoding gene editing fusion protein and a nucleotide sequence encoding guide RNA; wherein said gene editing fusion protein contains a CRISPR-associated effector domain and optionally a deaminase domain, said guide RNA can target said gene editing fusion protein to the target sequence in the plant genome.

In some embodiments, the present disclosure provides a polynucleotide encoding a gRNA. In some embodiments, a gRNA-encoding nucleic acid is comprised in an expression vector, e.g., a recombinant expression vector. In some embodiments, the present disclosure provides a polynucleotide encoding a site-directed modifying polypeptide. In some embodiments, the polynucleotide encoding a site-directed modifying polypeptide is comprised in an expression vector, e.g., a recombinant expression vector.

In some embodiments, the targeted gene-editing system comprises a Cas protein as a CRISPR-associated effector domain. Cas molecules of a variety of species can be used in the methods and compositions described herein, including Cas molecules derived from S. pyogenes, S. aureus, N. meningitidis, S. thermophiles, Acidovorax avenae, Actinobacillus pleuropneumoniae, Actinobacillus succinogenes, Actinobacillus suis, Actinomyces sp., Cycliphilusdenitrificans, Aminomonas paucivorans, Bacillus cereus, Bacillus smithii, Bacillus thuringiensis, Bacteroides sp., Blastopirellula marina, Bradyrhizobium sp., Brevibacillus laterospoxus, Campylobacter coli, Campylobacter jejuni, Campylobacter lari, Candidatus puniceispirillum, Clostridium cellulolyticum, Clostridium perfringens, Corynebacterium accolens, Corynebacterium diphtheria, Corynebacterium matruchotii, Dinoroseobacter shibae, Eubacterium dolichum, Gammaproteobacterium, Gluconacetobacter diazotrophicus, Haemophilus parainfluenzae, Haemophilus sputomm, Helicobacter canadensis, Helicobacter cinaedi, Helicobacter mustelae, Ilyobacter polytropus, Kingella kingae, Lactobacillus crispatus, Listeria ivanovii, Listeria monocytogenes, Listeriaceae bacterium, Methylocystis sp., Methylosinus trichosporium, Mobiluncus mulieris, Neisseria bacilliformis, Neisseria cinerea, Neisseria flavescens, Neisseria lactamica, Neisseria meningitidis, Neisseria sp., Neisseria wadsworthii, Nitrosomonas sp., Parvibaculum lavamentivorans, Pasteurella multocida, Phascolarctobacterium succinatutens, Ralstonia syzygii, Rhodopseudomonas palustris, Rhodovulum sp., Simonsiella muelleri, Sphingomonas sp., Sporolactobacillus vineae, Staphylococcus aureus, Staphylococcus lugdunensis, Streptococcus sp., Subdoligranulum sp., Tistrella mobilis, Treponema sp., or Verminephrobacter eiseniae.

In some embodiments, the Cas protein is a naturally-occurring Cas protein. In other embodiments, the Cas protein is an engineered Cas protein. In some embodiments, the Cas endonuclease is selected from the group consisting of C2C1, C2C3, Cpf1 (also referred to as Cas12a), Cas12b, Cas12c, Cas12d, Cas12e, Cas13a, Cas13b, Cas13c, Cas13d, Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, and Csf4.

In some embodiments, the Cas protein is an endoribonuclease such as a Cas13 protein. In some embodiments, the Cas13 protein is a Cas13a (Abudayyeh et al., Nature 550 (2017), 280-284), Cas13b (Cox et al., Science (2017) 358:6336, 1019-1027), Cas13c (Cox et al., Science (2017) 358:6336, 1019-1027), or Cas13d (Zhang et al., Cell 175 (2018), 212-223) protein.

In some embodiments, the Cas protein is a wild type or naturally occurring Cas9 protein or a Cas9 ortholog. Wild type Cas9 is a multi-domain enzyme that uses an HNH nuclease domain to cleave the target strand of DNA and a RuvC-like domain to cleave the non-target strand. Binding of WT Cas9 to DNA based on gRNA specificity results in double-stranded DNA breaks that can be repaired by non-homologous end joining (NHEJ) or homology-directed repair (HDR).

In some embodiments, the naturally occurring Cas9 polypeptide is selected from the group consisting of SpCas9, SpCas9-HF1, SpCas9-HF2, SpCas9-HF3, SpCas9-HF4, SaCas9, FnCpf, FnCas9, eSpCas9, and NmeCas9. In some embodiments, the Cas9 protein comprises an amino acid sequence having at least 60%, 65%, 70%, 75%, 80%, 85%, 90%, 95%, 96%, 97%, 98%, 99%, or 100% sequence identity to a Cas9 amino acid sequence described in Chylinski et al., RNA Biology 2013 10:5, 727-737; Hou et al., PNAS Early Edition 2013, 1-6).

In some embodiments, the Cas polypeptide comprises one or more of the following activities: (a) a nickase activity, i.e., the ability to cleave a single strand, e.g., the non-complementary strand or the complementary strand, of a nucleic acid molecule; (b) a double stranded nuclease activity, i.e., the ability to cleave both strands of a double stranded nucleic acid and create a double stranded break, which in an embodiment is the presence of two nickase activities; (c) an endonuclease activity; (d) an exonuclease activity; and/or (e) a helicase activity, i.e., the ability to unwind the helical structure of a double stranded nucleic acid. In other embodiments, the Cas protein may be dead or inactive (e.g. dCas).

In some embodiments, the Cas polypeptide is fused to heterologous polypeptide/proteins that has base deaminase activity.

In some embodiments, different Cas proteins (i.e., Cas9 proteins from various species) may be advantageous to use in the various provided methods in order to capitalize on various enzymatic characteristics of the different Cas proteins (e.g., for different PAM sequence preferences; for increased or decreased enzymatic activity; for an increased or decreased level of cellular toxicity; to change the balance between NHEJ, homology-directed repair, single strand breaks, double strand breaks, etc.).

In some embodiments, the Cas protein is a Cas9 protein derived from S. pyogenes and recognizes the PAM sequence motif NGG, NAG, NGA (Mali et al, Science 2013; 339(6121): 823-826). In some embodiments, the Cas protein is a Cas9 protein derived from S. thermophiles and recognizes the PAM sequence motif NGGNG and/or NNAGAAW (W=A or T) (See, e.g., Horvath et al, Science, 2010; 327(5962): 167-170, and Deveau et al, J Bacteriol 2008; 190(4): 1390-1400). In some embodiments, the Cas protein is a Cas9 protein derived from S. mutans and recognizes the PAM sequence motif NGG and/or NAAR (R=A or G) (See, e.g., Deveau et al, J BACTERIOL 2008; 190(4): 1390-1400). In some embodiments, the Cas protein is a Cas9 protein derived from S. aureus and recognizes the PAM sequence motif NNGRR (R=A or G). In some embodiments, the Cas protein is a Cas9 protein derived from S. aureus and recognizes the PAM sequence motif N GRRT (R=A or G). In some embodiments, the Cas protein is a Cas9 protein derived from S. aureus and recognizes the PAM sequence motif N GRRV (R=A or G). In some embodiments, the Cas protein is a Cas9 protein derived from N. meningitidis and recognizes the PAM sequence motif N GATT or N GCTT (R=A or G, V=A, G or C) (See, e.g., Hou et ah, PNAS 2013, 1-6). In the aforementioned embodiments, N can be any nucleotide residue, e.g., any of A, G, C or T. In some embodiments, the Cas protein is a Cas13a protein derived from Leptotrichia shahii and recognizes the PFS sequence motif of a single 3′ A, U, or C.

In embodiments, a Cas protein as a CRISPR-associated effector domain is codon-optimized based on plant genomes of the present disclosure.

In some embodiments, the at least one CRISPR-associated effector is selected from at least one of a nuclease, comprising a CRISPR nuclease, including Cas or Cpf1 nucleases.

In some embodiments, the at least one CRISPR-associated effector is a Cas polypeptide. In some embodiments, the at least one CRISPR-associated effector is a Cas polypeptide, wherein the Cas polypeptide comprises a site-specific DNA binding domain linked to at least one base editor. The CRISPR-associated effector or the nucleic acid sequence encoding the same, is selected from the group comprising (i) Cas9, including SpCas9, SaCas9, SaKKH-Cas9, VQR-Cas9, StlCas9, (ii) Cpf1, including AsCpf1, LbCpf1, FnCpf1, (iii) CasX, or (iv) CasY, or any variant or derivative of the aforementioned CRISPR-associated effector, preferably wherein the at least one CRISPR-associated effector comprises a mutation in comparison to the respective wild type sequence so that the resulting CRISPR-associated effector is converted to a single-strand specific DNA nickase, or to a DNA binding effector lacking all DNA cleavage ability, as described below.

Therefore, according to the present disclosure, artificially modified CRISPR nucleases are envisaged, which might indeed not be any “nucleases” in the sense of double-strand cleaving enzymes, but which are nickases or nuclease-dead variants, which still have inherent DNA recognition and thus binding ability.

In some embodiments, the Cas protein described above can be engineered to alter one or more properties of the Cas polypeptide. For example, in some embodiments, the Cas polypeptide comprises altered enzymatic properties, e.g., altered nuclease activity, (as compared with a naturally occurring or other reference Cas molecule) or altered helicase activity. In some embodiments, an engineered Cas polypeptide can have an alteration that alters its size, e.g., a deletion of amino acid sequence that reduces its size without significant effect on another property of the Cas polypeptide. In some embodiments, an engineered Cas polypeptide comprises an alteration that affects PAM recognition. For example, an engineered Cas polypeptide can be altered to recognize a PAM sequence other than the PAM sequence recognized by the corresponding wild type Cas protein. In some embodiments, the targeted gene-editing system comprises a Cas protein as a CRISPR-associated effector domain.

Cas polypeptides with desired properties can be made in a number of ways, including alteration of a naturally occurring Cas polypeptide or parental Cas polypeptide, to provide a mutant or altered Cas polypeptide having a desired property. For example, one or more mutations can be introduced into the sequence of a parental Cas polypeptide (e.g., a naturally occurring or engineered Cas polypeptide). Such mutations and differences may comprise substitutions (e.g., conservative substitutions or substitutions of non-essential amino acids); insertions; or deletions. In some embodiments, a mutant Cas polypeptide comprises one or more mutations (e.g., at least 1, 2, 3, 4, 5, 10, 15, 20, 30, 40 or 50 mutations) relative to a parental Cas polypeptide.

In an embodiment, a mutant Cas polypeptide comprises a cleavage property that differs from a naturally occurring Cas polypeptide. In some embodiments, the Cas is a deactivated Cas (dCas) mutant, which is catalytically dead. In such embodiments, the Cas polypeptide does not comprise any intrinsic enzymatic activity and is unable to mediate target nucleic acid cleavage. In such embodiments, the dCas is fused with a heterologous protein that is capable of modifying the target nucleic acid in a non-cleavage based manner. In some embodiments, the targeted gene-editing system comprises a Cas protein as a CRISPR-associated effector domain.

In some embodiments, the dCas is a dCas9 mutant. In some embodiments, a dCas protein is fused to base deaminase domains (e.g., cytidine deaminase, or adenosine deaminase). In some such cases, the dCas fusion protein is targeted by the gRNA to a specific location (i.e., sequence) in the target nucleic acid and exerts locus-specific modification such as replacing C with T (or G with A) if the fusion protein has cytidine deaminase activity) or replacing A with G (or T with C) if the fusion protein has adenosine deaminase activity.

In some embodiments, the dCas is a dCas13 mutant (Konermann et al., Cell 173 (2018), 665-676). These dCas13 mutants can then be fused to enzymes that modify RNA, including adenosine deaminases (e.g., ADAR1 and ADAR2). Adenosine deaminases convert adenine to inosine, which the translational machinery treats like guanine, thereby creating a functional A to G change in the target sequence.

In some embodiments, the CRISPR-associated effector protein is Cas9 endonuclease. In some embodiments, the CRISPR-associated effector protein is a CRISPR-Cas variant, which is dCas9 mutant or nCas9 nickase mutant. The Cas9 endonuclease has a DNA cleavage domain containing two subdomains: i) the RuvC subdomain cleaving the non-complementary single-stranded chain and ii) the HNH nuclease subdomain cleaving the chain that is complementary to gRNA. Mutations in these subdomains can inactivate Cas9 endonuclease to form deactivated Cas9 (dCas9), which is interchangeably used with “catalytically dead Cas9”. The nuclease-inactivated Cas9 retains DNA binding capacity directed by gRNA. Thus, in principle, when fused with an additional protein, the dCas9 can simply target said additional protein to almost any DNA sequence through co-expression with appropriate guide RNA. For example, catalytically dead Cas9 (dCas9), which contains Asp10Ala (D10A) and His840Ala (H840A) mutations that inactivate its nuclease activity, retains its ability to bind DNA in a guide RNA-programmed manner, but does not cleave the DNA backbone (Komor et al., nature (2016), Vol 533:420-424). In some embodiments, conjugation of dCas9 with an enzymatic or chemical catalyst that mediates the direct conversion of one base to another could enable RNA-programmed DNA base editing.

In some embodiments, the mutant Cas9 is a Cas9 nickase (nCas9) mutant. Cas9 nickase mutants comprise only one catalytically active domain (either the RuvC domain (D10A) or the HNH domain (H840A)). The Cas9 nickase mutants retain DNA binding based on gRNA specificity, but are capable of cutting only one strand of DNA resulting in a single-strand break (e.g. a “nick”). In some embodiments, two complementary Cas9 nickase mutants (e.g., one Cas9 nickase mutant with an inactivated RuvC domain, and one Cas9 nickase mutant with an inactivated HNH domain) are expressed in the same cell with two gRNAs corresponding to two respective target sequences; one target sequence on the sense DNA strand, and one on the antisense DNA strand. This dual-nickase system results in staggered double stranded breaks and can increase target specificity, as it is unlikely that two off-target nicks will be generated close enough to generate a double stranded break. In some embodiments, a Cas9 nickase mutant is co-expressed with a nucleic acid repair template to facilitate the incorporation of an exogenous nucleic acid sequence by homology-directed repair.

The dCas9 of the present disclosure can be derived from Cas9 of different species, for example, derived from S. pyogenes Cas9 (SpCas9). Mutations in both the RuvC subdomain and the HNH nuclease subdomain of the SpCas9 (includes, for example, D10A and H840A mutations) inactivate S. pyogenes Cas9 nuclease, resulting in a nuclease-dead/catalytically dead Cas9 (dCas9). In some embodiments of the present disclosure, the nuclease-inactivated Cas9 comprises the dCas9. In some preferred embodiments, the nuclease-inactivated Cas9 comprises.

Inactivation of one of the subdomains by mutation allows Cas9 to gain nickase activity, i.e., resulting in a Cas9 nickase (nCas9), for example, nCas9 with a D10A mutation only.

In some embodiments, the nuclease-inactivated Cas9 comprises amino acid substitutions D10A and/or H840A relative to wild type Cas9. In some embodiments, the nuclease-inactivated Cas9 of the present disclosure loses nuclease activity completely, which is catalytically dead. In such embodiments, the nuclease-inactivated Cas9 is the dCas9 with D10A and H840A. Therefore, the term “nuclease-inactivated Cas9” refers to dCas9 and/or nCas9.

In other embodiments, the nuclease-inactivated Cas9 of the present disclosure has nickase activity. In some embodiments, the nuclease-inactivated Cas9 is a Cas9 nickase that retains the cleavage activity of the HNH subdomain of Cas9, whereas the cleavage activity of the RuvC subdomain is inactivated. For example, the nuclease-inactivated Cas9 contains an amino acid substitution D10A relative to wild type Cas9. In such embodiments, the nuclease-inactivated Cas9 is the nCas9 with D10A only. In some embodiments of the present disclosure, the nuclease-inactivated Cas9 comprises the nCas9.

In some embodiments, the Cas polypeptides described herein can be engineered to alter the PAM/PFS specificity of the Cas polypeptide. In some embodiments, a mutant Cas polypeptide has a PAM/PFS specificity that is different from the PAM/PFS specificity of the parental Cas polypeptide. For example, a naturally occurring Cas protein can be modified to alter the PAM/PFS sequence that the mutant Cas polypeptide recognizes to decrease off target sites, improve specificity, or eliminate a PAM/PFS recognition requirement. In some embodiments, a Cas protein can be modified to increase the length of the PAM/PFS recognition sequence. In some embodiments, the length of the PAM recognition sequence is at least 4, 5, 6, 7, 8, 9, 10 or 15 amino acids in length. Cas polypeptides that recognize different PAM/PFS sequences and/or have reduced off-target activity can be generated using directed evolution. Exemplary methods and systems that can be used for directed evolution of Cas polypeptides are described, e.g., in Esvelt et al. Nature 2011, 472(7344): 499-503.

Exemplary Cas mutants are described in International PCT Publication No. WO 2015/161276 and Konermann et al., Cell 173 (2018), 665-676, which are incorporated herein by reference in their entireties.

In some embodiments, the deaminase domain is fused to the N-terminus of the nuclease-inactivated Cas9 domain. In some embodiments, the deaminase domain is fused to the C-terminus of the nuclease-inactivated Cas9 domain. In some embodiments, the deaminase domain and the nuclease inactivated Cas9 domain are fused through a linker. The linker can be a non-functional amino acid sequence having no secondary or higher structure, N-terminus and one or more NLSs at the C-terminus. Where there are more than one NLS, each NLS may be selected as independent from other NLSs. In some embodiments, the targeted base-editing fusion protein comprises two NLSs, for example, the two NLSs are located at the N-terminus and the C-terminus, respectively.

In some embodiment, a targeted base modification is a conversion of any nucleotide C, A, T, or G, to any other nucleotide. Any one of a C, A, T or G nucleotide can be exchanged in a site-directed way as mediated by a base editor, or a catalytically active fragment thereof, to another nucleotide. A base editing complex can comprise any base editor, or a base editor domain or catalytically active fragment thereof, which can convert a nucleotide of interest into any other nucleotide of interest in a targeted way.

A base editing domain according to the present disclosure can comprise at least one cytidine deaminase, or a catalytically active fragment thereof. The at least one base editing complex can comprise the cytidine deaminase, or a domain thereof in the form of a catalytically active fragment, as base editor.

According to the present disclosure, cytidine deaminases that can be used in connection with the present disclosure include, but are not limited to, members of the enzyme family known as apolipoprotein B mRNA-editing complex (APOBEC) family deaminase, an activation-induced deaminase (AID), or a cytidine deaminase 1 (CDA1). In particular embodiments, the deaminase in an APOBEC1 deaminase, an APOBEC2 deaminase, an APOBEC3A deaminase, an APOBEC3B deaminase, an APOBEC3C deaminase, and APOBEC3D deaminase, an APOBEC3E deaminase, an APOBEC3F deaminase an APOBEC3G deaminase, an APOBEC3H deaminase, or an APOBEC4 deaminase.

In the methods and systems of the present disclosure, the cytidine deaminase is capable of targeting Cytosine in a DNA single strand. In certain example embodiments the cytodine deaminase may edit on a single strand present outside of the binding component e.g. bound Cas9 and/or Cas13. In other example embodiments, the cytodine deaminase may edit at a localized bubble, such as a localized bubble formed by a mismatch at the target edit site but the guide sequence.

In some embodiments, the cytidine deaminase protein recognizes and converts one or more target cytosine residue(s) in a single-stranded bubble of a DNA-RNA heteroduplex into uracil residues (s). In some embodiments, the cytidine deaminase protein recognizes a binding window on the single-stranded bubble of a DNA-RNA heteroduplex. In some embodiments, the binding window contains at least one target cytosine residue(s). In some embodiments, the binding window is in the range of about 3 bp to about 100 bp. In some embodiments, the binding window is in the range of about 5 bp to about 50 bp. In some embodiments, the binding window is in the range of about 10 bp to about 30 bp. In some embodiments, the binding window is about 1 bp, 2 bp, 3 bp, 5 bp, 7 bp, 10 bp, 15 bp, 20 bp, 25 bp, 30 bp, 40 bp, 45 bp, 50 bp, 55 bp, 60 bp, 65 bp, 70 bp, 75 bp, 80 bp, 85 bp, 90 bp, 95 bp, or 100 bp.

In some embodiments, the cytidine deaminase protein comprises one or more deaminase domains. Not intended to be bound by theory, it is contemplated that the deaminase domain functions to recognize and convert one or more target cytosine (C) residue(s) contained in a single-stranded bubble of a DNA-RNA heteroduplex into (an) uracil (U) residue (s). In some embodiments, the deaminase domain comprises an active center. In some embodiments, the active center comprises a zinc ion. In some embodiments, amino acid residues in or near the active center interact with one or more nucleotide(s) 5′ to a target cytosine residue. In some embodiments, amino acid residues in or near the active center interact with one or more nucleotide(s) 3′ to a target cytosine residue.

In some embodiments, the cytidine deaminase comprises human APOBEC1 full protein (hAPOBECl) or the deaminase domain thereof (hAPOBECl-D) or a C-terminally truncated version thereof (hAPOBEC-T). In some embodiments, the cytidine deaminase is an APOBEC family member that is homologous to hAPOBECl, hAPOBEC-D or hAPOBEC-T. In some embodiments, the cytidine deaminase comprises human AID1 full protein (hAID) or the deaminase domain thereof (hAID-D) or a C-terminally truncated version thereof (hAID-T). In some embodiments, the cytidine deaminase is an AID family member that is homologous to hAID, hAID-D or hAID-T. In some embodiments, the hAIDT is a hAID which is C-terminally truncated by about 20 amino acids.

In some embodiments, the cytidine deaminase comprises the wild type amino acid sequence of a cytosine deaminase. In some embodiments, the cytidine deaminase comprises one or more mutations in the cytosine deaminase sequence, such that the editing efficiency, and/or substrate editing preference of the cytosine deaminase is changed according to specific needs.

Certain mutations of APOBEC 1 and APOBEC3 proteins have been described in Kim et al., Nature Biotechnology (2017) 35(4):371-377 and Harris et al. Mol. Cell (2002) 10:1247-1253, each of which is incorporated herein by reference in its entirety.

Additional embodiments of the cytidine deaminase are disclosed in WO2017/070632 and WO2018/213726, each of which is incorporated herein by reference in its entirety.

In some embodiments, at least one CRISPR-associated effector is temporarily or permanently linked to at least one base editor to form a targeted base editing complex, which is a base editing fusion protein, wherein the base editing complex mediates the at least one targeted base modification. The at least one CRISPR-associated effector can be non-covalently (temporarily) or covalently (permanently) be attached to at least one base editor. Any component of the at least one base editor can be temporarily or permanently linked to the at least one CRISPR-associated effector.

The adenosine deaminases (e.g. engineered adenosine deaminases, evolved adenosine deaminases) provided herein may be from any organism, such as a bacterium. In some embodiments, the deaminase or deaminase domain is a variant of a naturally-occurring deaminase from an organism. In some embodiments, the deaminase or deaminase domain does not occur in nature. For example, in some embodiments, the deaminase or deaminase domain is at least 50%, at least 55%, at least 60%, at least 65%, at least 70%, at least 75% at least 80%, at least 85%, at least 90%, at least 95%, at least 96%, at least 97%, at least 98%, at least 99%, or at least 99.5% identical to a naturally-occurring deaminase. In some embodiments, the adenosine deaminase is from a bacterium, such as, E. coli, S. aureus, S. typhi, S. putrefaciens, H. influenzae, or C. crescentus. In some embodiments, the adenosine deaminase is a TadA deaminase. In some embodiments, the TadA deaminase is an E. coli TadA deaminase (ecTadA). In some embodiments, the TadA deaminase is a truncated E. coli TadA deaminase. For example, the truncated ecTadA may be missing one or more N-terminal amino acids relative to a full-length ecTadA. In some embodiments, the truncated ecTadA may be missing 1, 2, 3, 4, 5, 6, 7, 8, 9, 10, 11, 12, 13, 14, 15, 6, 17, 18, 19, or 20 N-terminal amino acid residues relative to the full length ecTadA. In some embodiments, the truncated ecTadA may be missing 1, 2, 3, 4, 5, 6, 7, 8, 9, 10, 11, 12, 13, 14, 15, 6, 17, 18, 19, or 20 C-terminal amino acid residues relative to the full length ecTadA. In some embodiments, the ecTadA deaminase does not comprise an N-terminal methionine.

Some aspects of the disclosure provide adenosine deaminases. In some embodiments, the adenosine deaminases provided herein are capable of deaminating adenine. In some embodiments, the adenosine deaminases provided herein are capable of deaminating adenine in a deoxyadenosine residue of DNA. The adenosine deaminase may be derived from any suitable organism (e.g., E. coli). In some embodiments, the adenine deaminase is a naturally-occurring adenosine deaminase that includes one or more mutations corresponding to any of the mutations provided herein (e.g., mutations in ecTadA). One of skill in the art will be able to identify the corresponding residue in any homologous protein and in the respective encoding nucleic acid by methods well known in the art, e.g., by sequence alignment and determination of homologous residues. Accordingly, one of skill in the art would be able to generate mutations in any naturally-occurring adenosine deaminase (e.g., having homology to ecTadA) that corresponds to any of the mutations described herein, e.g., any of the mutations identified in ecTadA. In some embodiments, the adenosine deaminase is from a prokaryote. In some embodiments, the adenosine deaminase is from a bacterium. In some embodiments, the adenosine deaminase is from Escherichia coli, Staphylococcus aureus, Salmonella typhi, Shewanella putrefaciens, Haemophilus influenzae, Caulobacter crescentus, or Bacillus subtilis. In some embodiments, the adenosine deaminase is from E. coli.

In other embodiments, adenosine deaminases that can be used that include, but are not limited to, members of the enzyme family known as adenosine deaminases that act on RNA (ADARs), members of the enzyme family known as adenosine deaminases that act on tRNA (ADATs), and other adenosine deaminase domain-containing (ADAD) family members. According to the present disclosure, the adenosine deaminase is capable of targeting adenine in a RNA/DNA heteroduplex. Indeed, Zheng et al. (Nucleic Acids Res. 2017, 45(6): 3369-3377) has demonstrated that ADARs can carry out adenosine to inosine editing reactions on RNA/DNA heteroduplexes. In particular embodiments, the adenosine deaminase has been modified to increase its ability to edit DNA in a RNA/DNA heteroduplex as detailed herein below.

In some embodiments, the adenosine deaminase is derived from one or more metazoa species, including but not limited to, mammals, birds, frogs, squids, fish, flies and worms. In some embodiments, the adenosine deaminase is a human, squid or Drosophila adenosine deaminase.

In some embodiments, the adenosine deaminase is a human ADAR, including hADAR1, hADAR2, hADAR3. In some embodiments, the adenosine deaminase is a Caenorhabditis elegans ADAR protein, including ADR-1 and ADR-2. In some embodiments, the adenosine deaminase is a Drosophila ADAR protein, including dAdar. In some embodiments, the adenosine deaminase is a squid Loligo pealeii ADAR protein, including sqADAR2a and sqADAR2b. In some embodiments, the adenosine deaminase is a human ADAT protein. In some embodiments, the adenosine deaminase is a Drosophila ADAT protein. In some embodiments, the adenosine deaminase is a human ADAD protein, including TE R (hADAD1) and TE RL (hADAD2).

In some embodiments, the adenosine deaminase is a TadA protein such as E. coli TadA. See Kim et al., Biochemistry 45:6407-6416 (2006); Wolf et al., EMBO J. 21:3841-3851 (2002), each of which is incorporated herein by reference in its entirety. In some embodiments, the adenosine deaminase is mouse ADA (See Grunebaum et al., Curr. Opin. Allergy Clin. Immunol. 13:630-638 (2013)) or human ADAT2 (See Fukui et al., J. Nucleic Acids 2010:260512 (2010)), each of which is incorporated herein by reference in its entirety.

Additional embodiments of the adenosine deaminase are disclosed in U.S. Pat. No. 10,113,163 and WO2018/213708, each of which is incorporated herein by reference in its entirety.

In some embodiments, at least one CRISPR-associated effector is temporarily or permanently linked to at least one base editor to form a targeted base editing complex, which is a base editing fusion protein, wherein the base editing complex mediates the at least one targeted base modification. The at least one CRISPR-associated effector can be non-covalently (temporarily) or covalently (permanently) be attached to at least one base editor. Any component of the at least one base editor can be temporarily or permanently linked to the at least one CRISPR-associated effector.

In one aspect the present disclosure provides methods for targeted deamination of adenine in a DNA, more particularly in a locus of interest. The disclosure teaches the adenosine deaminase (AD) protein is recruited specifically to the relevant Adenine in the locus of interest by a CRISPR-Cas complex which can specifically bind to a target sequence. In order to achieve this, the adenosine deaminase protein can either be covalently linked to the CRISPR-Cas enzyme or be provided as a separate protein, but adapted so as to ensure recruitment thereof to the CRISPR-Cas complex.

In particular embodiments, recruitment of the adenosine deaminase to the target locus is ensured by fusing the adenosine deaminase or catalytic domain thereof to the CRISPR-Cas protein, which is a Cas or Cpf1 protein. Methods of generating a fusion protein from two separate proteins are known in the art and typically involve the use of spacers or linkers. The CRISPR-Cas protein can be fused to the adenosine deaminase protein or catalytic domain thereof on either the N- or C-terminal end thereof. In particular embodiments, the CRISPR-Cas protein is a Cas or Cpf1 protein and is linked to the N-terminus of the deaminase protein or its catalytic domain.

In the present disclosure, linker refers to a molecule which joins two entities such as two domains to form a fusion protein. Generally, such molecules have no specific biological activity other than to join or to preserve some minimum distance or other spatial relationship between the proteins. However, in certain embodiments, the linker may be selected to influence some property of the linker and/or the fusion protein such as the folding, net charge, or hydrophobicity of the linker.

Suitable linkers for use in the methods of the present disclosure are well known to those of skill in the art and include, but are not limited to, straight or branched-chain carbon linkers, heterocyclic carbon linkers, or peptide linkers. However, as used herein the linker may also be a covalent bond (carbon-carbon bond or carbon-heteroatom bond). In particular embodiments, the linker is used to separate the CRISPR-Cas protein and the cytidine deaminase by a distance sufficient to ensure that each protein retains its required functional property. Preferred peptide linker sequences adopt a flexible extended conformation and do not exhibit a propensity for developing an ordered secondary structure.

In some embodiments, the linker can be a chemical moiety which can be monomeric, dimeric, multimeric or polymeric. Exemplary linkers are disclosed in Maratea et al. (1985), Gene 40: 39-46; Murphy et al. (1986) Proc. Nat'l. Acad. Sci. USA 83: 8258-62; U.S. Pat. Nos. 4,935,233; and 4,751,180. For example, GlySer linkers GGS, GGGS or GSG can be used. GGS, GSG, GGGS or GGGGS linkers can be used in repeats of 3 such as (GGS)₃. In other embodiments, 1, 2, 3, 4, 5, 6, 7, 8, 9, 10, 11, 12 or more of (GGGGS), to provide suitable lengths. In some embodiments, linkers such as (GGGGS)₁, (GGGGS)₂, (GGGGS)₃, (GGGGS)₄, (GGGGS)₅, (GGGGS)₆, (GGGGS)₇, (GGGGS)₈, (GGGGS)₉, (GGGGS)₁₀, (GGGGS)₁₁, or (GGGGS)₁₂ may be used. In other embodiments, the linker is XTEN linker. In particular embodiments, the nuclease-inactivated CRISPR-associated effector protein such as dCas9 or nCas9 is linked to the deaminase protein or its catalytic domain by means of an XTEN linker. In some embodiments, the nuclease-inactivated Cas mutant is linked C-terminally to the N-terminus of a deaminase protein or its catalytic domain by means of an XTEN linker. In addition, N- and C-terminal NLSs can also function as linker.

The present disclosure provides guide RNAs (gRNAs) that direct a site-directed modifying polypeptide to a specific target nucleic acid sequence. A gRNA comprises a nucleic acid-targeting segment and protein-binding segment. The nucleic acid-targeting segment of a gRNA comprises a nucleotide sequence that is complementary to a sequence in the target nucleic acid sequence. As such, the nucleic acid-targeting segment of a gRNA interacts with a target nucleic acid in a sequence-specific manner via hybridization (i.e., base pairing), and the nucleotide sequence of the nucleic acid-targeting segment determines the location within the target nucleic acid that the gRNA will bind. The nucleic acid-targeting segment of a gRNA can be modified (e.g., by genetic engineering) to hybridize to any desired sequence within a target nucleic acid sequence.

The protein-binding segment of a guide RNA interacts with a site-directed modifying polypeptide (e.g. a Cas protein) to form a complex. The guide RNA guides the bound polypeptide to a specific nucleotide sequence within target nucleic acid via the above-described nucleic acid-targeting segment. The protein-binding segment of a guide RNA comprises two stretches of nucleotides that are complementary to one another and which form a double stranded RNA duplex.

In some embodiments, a gRNA comprises two separate RNA molecules. In such embodiments, each of the two RNA molecules comprises a stretch of nucleotides that are complementary to one another such that the complementary nucleotides of the two RNA molecules hybridize to form the double-stranded RNA duplex of the protein-binding segment. In some embodiments, a gRNA comprises a single guide RNA molecule (sgRNA).

The specificity of a gRNA for a target loci is mediated by the sequence of the nucleic acid-binding segment, which comprises about 20 nucleotides that are complementary to a target nucleic acid sequence within the target locus. In some embodiments, the corresponding target nucleic acid sequence is approximately 20 nucleotides in length. In some embodiments, the nucleic acid-binding segments of the gRNA sequences of the present disclosure are at least 90% complementary to a target nucleic acid sequence within a target locus. In some embodiments, the nucleic acid-binding segments of the gRNA sequences of the present disclosure are at least 95%, 96%, 97%, 98%, or 99% complementary to a target nucleic acid sequence within a target locus. In some embodiments, the nucleic acid-binding segments of the gRNA sequences of the present disclosure are 100% complementary to a target nucleic acid sequence within a target locus.

In some embodiments, the target nucleic acid sequence is an RNA target sequence. In some embodiments, the target nucleic acid sequence is a DNA target sequence.

In some embodiments, the targeted gene-editing system comprises two or more gRNA molecules each comprising a DNA-binding segment, wherein at least one of the nucleic acid-binding segments binds to a target DNA sequence of a target gene.

In some embodiments, the guide RNA is a single guide RNA (sgRNA). Methods of constructing suitable sgRNAs according to a given target sequence are known in the art. See e.g., Wang, Y. et al. Simultaneous editing of three homoeoalleles in hexaploid bread wheat confers heritable resistance to powdery mildew. Nat. Biotechnol. 32, 947-951 (2014); Shan, Q. et al. Targeted genome modification of crop plants using a CRISPR-Cas system. Nat. Biotechnol. 31, 686-688 (2013); Liang, Z. et al. Targeted mutagenesis in Zea mays using TALENs and the CRISPR/Cas system. J Genet Genomics. 41, 63-68 (2014).

The addition of a uracil DNA glycosylase (UGI) domain further increased the base-editing efficiency. In some embodiments, the targeted base-editing system further comprises a base excision repair (BER) inhibitor. Cellular DNA-repair response to the presence of a U:G pairing in DNA may be responsible for a decrease in nucleobase editing efficiency in plant cells. Uracil DNA glycosylase catalyzes removal of uracil from DNA in plant cells, which may initiate base excision repair, such that the U:G pair is reversed to C:G. In some embodiments, the BER inhibitor is an uracyl glycosylase inhibitor or an active domain thereof.

In some embodiments, the BER inhibitor is an inhibitor of uracil DNA glycosylase (UDG). In some embodiments, the BER inhibitor is an inhibitor of UDG. In some embodiments, the BER inhibitor is a polypeptide inhibitor. In some embodiments, the BER inhibitor is a protein that binds single-stranded DNA. For example, the BER inhibitor may be a Erwinia tasmaniensis single-stranded binding protein. In some embodiments, the BER inhibitor is a protein that binds uracil. In some embodiments, the BER inhibitor is a protein that binds uracil in DNA. In some embodiments, the BER inhibitor is a catalytically inactive UDG or binding domain thereof. In some embodiments, the BER inhibitor is a catalytically inactive UDG or binding domain thereof that does not excise uracil from the DNA. Other proteins that are capable of inhibiting (e.g., sterically blocking) UDG are within the scope of this disclosure. Additionally, any proteins that block or inhibit base-excision repair as also within the scope of this disclosure.

Base excision repair may be inhibited by molecules that bind the edited strand, block the edited base, inhibit uracil DNA glycosylase, inhibit base excision repair, protect the edited base, and/or promote fixing of the non-targeted strand. Accordingly, the use of the BER inhibitor described herein can increase the editing efficiency of a cytidine deaminase that is capable of catalyzing a C to U change.

In particular embodiments, the uracil glycosylase inhibitor (UGI) is the uracil DNA glycosylase inhibitor of Bacillus subtilis bacteriophage PBS1 or an active fragment thereof, such as an 83 residue protein of Bacillus subtilis bacteriophage PBS1.

Suitable UGI protein and nucleotide sequences are provided herein and additional suitable UGI sequences are known to those in the art, and include, for example, those published in Wang et al., Uracil-DNA glycosylase inhibitor gene of bacteriophage PBS2 encodes a binding protein specific for uracil-DNA glycosylase. J. Biol. Chem. 264: 1 163-1 171(1989); Lundquist et al., Site-directed mutagenesis and characterization of uracil-DNA glycosylase inhibitor protein. Role of specific carboxylic amino acids in complex formation with Escherichia coli uracil-DNA glycosylase. J. Biol. Chem. 272:21408-21419(1997); Ravishankar et al., X-ray analysis of a complex of Escherichia coli uracil DNA glycosylase (EcUDG) with a proteinaceous inhibitor. The structure elucidation of a prokaryotic UDG. Nucleic Acids Res. 26:4880-4887(1998); and Putnam et al., Protein mimicry of DNA from crystal structures of the uracil-DNA glycosylase inhibitor protein and its complex with Escherichia coli uracil-DNA glycosylase. J. Mol. Biol. 287:331-346(1999), each of which incorporated herein by reference. Additional embodiments of the uracil glycosylase inhibitor (UGI) are disclosed in WO2018/086623, WO2018/205995, WO2017/70632, and WO2018/213726, each of which is incorporated herein by reference in its entirety.

In some embodiments, the UGI domain comprises a wild type UGI or a UGI. In some embodiments, the UGI proteins provided herein include fragments of UGI and proteins homologous to a UGI or a UGI fragment.

Additional proteins may be uracil glycosylase inhibitors. For example, other proteins that are capable of inhibiting (e.g., sterically blocking) a uracil-DNA glycosylase base-excision repair enzyme are within the scope of this disclosure. Additionally, any proteins that block or inhibit base-excision repair as also within the scope of this disclosure. In some embodiments, a protein that binds DNA is used. In another embodiment, a substitute for UGI is used. In some embodiments, a uracil glycosylase inhibitor is a protein that binds single-stranded DNA. For example, a uracil glycosylase inhibitor may be a Erwinia tasmaniensis single-stranded binding protein. In some embodiments, a uracil glycosylase inhibitor is a protein that binds uracil. In some embodiments, a uracil glycosylase inhibitor is a protein that binds uracil in DNA. In some embodiments, a uracil glycosylase inhibitor is a catalytically inactive uracil DNA-glycosylase protein. In some embodiments, a uracil glycosylase inhibitor is a catalytically inactive uracil DNA-glycosylase protein that does not excise uracil from the DNA. As another example, a uracil glycosylase inhibitor is a catalytically inactive UDG.

In some embodiments, the base editing system comprises the following domains; i) the CRISPR-Cas protein (dCas9 or nCas9) and ii) the cytidine deaminase, which can be fused to or linked to a BER inhibitor (e.g., an inhibitor of uracil DNA glycosylase).

Uracil-DNA glycosylase (UDG) is an enzyme that reverts mutations in DNA. The most common mutation is the deamination of cytosine to uracil. UDG repairs these mutations and UDG is crucial in DNA repair. Various uracil-DNA glycosylases and related DNA glycosylases (EC) are present such as uracil-DNA glycosylase, thermophilic uracil-DNA glycosylase, G:T/U mismatch-specific DNA glycosylase (Mug), and single-strand selective monofunctional uracil-DNA glycosylase (SMUG1).

Uracil DNA glycosylases remove uracil from DNA, which can arise either by spontaneous deamination of cytosine or by the misincorporation of dU opposite dA during DNA replication. The prototypical member of this family is E. coli UDG, which was among the first glycosylases discovered. Four different uracil-DNA glycosylase activities have been identified in mammalian cells, including UNG, SMUG1, TDG, and MBD4, which vary in substrate specificity and subcellular localization. SMUG1 prefers single-stranded DNA as substrate, but also removes U from double-stranded DNA. In addition to unmodified uracil, SMUG1 can excise 5-hydroxyuracil, 5-hydroxymethyluracil and 5-formyluracil bearing an oxidized group at ring C5.[13] TDG and MBD4 are strictly specific for double-stranded DNA. TDG can remove thymine glycol when present opposite guanine, as well as derivatives of U with modifications at carbon 5.

TDG and SMUG1 are the major enzymes responsible for the repair of the U:G mispairs caused by spontaneous cytosine deamination, whereas uracil arising in DNA through dU misincorporation is mainly dealt with by UNG. MBD4 is thought to correct T:G mismatches that arise from deamination of 5-methylcytosine to thymine in CpG sites.

Uracil arising in DNA either from misincorporation of dUMP or from deamination of cytosine is actively removed through the multistep base excision repair (BER) pathway. BER of uracil is initiated by a uracil DNA glycosylase (UDG) activity that cleaves the N-glycosidic bond and excises uracil as a free base, generating an abasic (apurinic/apyrimidinic, AP) site in the DNA. Repair is completed through subsequent steps that include incision at the AP site, gap tailoring, repair synthesis, and ligation.

In some embodiments, the addition of a Uracil-DNA glycosylase (UDG) such as uracil N-glycosylase (UNG) can induce various mutations at targeted base. In some embodiments, the targeted base-editing system further comprises a Uracil-DNA glycosylase (UDG). Cellular DNA-repair response to the presence of a U:G pairing in DNA may be responsible for a decrease in nucleobase editing efficiency in plant cells. Uracil DNA glycosylase catalyzes removal of uracil from DNA in plant cells, which may initiate base excision repair, such that the U:G pair is reversed to C:G. In other embodiments, removal of uracil from DNA in plant cells are not always reversed to C for C:G paring, but randomized to other bases such as T, A, and G.

In some embodiments, a Uracil-DNA glycosylase (UDG) is fused to the targeted base editing system taught in the present disclosure to introduce a stable and targeted, but randomized single nucleotide substitution in a target gene of interest.

The use of the UDG described herein can increase the base randomization in a targeted single nucleotide of a target gene.

In some embodiments, a UDG is provided in cis. In some embodiments, a UDG is provided in trans. In some embodiments, a UDG is fused to a base editor (or a base editing system) described in the present disclosure. In other embodiments, a UDG trigger a stall DNA replication for base randomization. In other embodiments, a UDG triggers a DNA repair through DNA replication, thereby including base randomization. In further embodiments, naturally occurring UDG variants can be used as a UDG domain. In further embodiments, non-naturally occurring UDG variants can be used as a UDG domain. In further embodiments, a UDG can be genetically engineered to enhance a functional UDG activity.

A nuclear localization signal (NLS), or any other organelle targeting signal, can be further required to ensure proper targeting of the complex. The present disclosure relate to modifying an cytosine in a target locus of interest, whereby the target locus is within a plant cell. In order to improve targeting of the CRISPR-Cas protein and/or the cytidine deaminase protein or catalytic domain thereof used in the methods of the present disclosure to the nucleus, it may be advantageous to provide one or both of these components with one or more nuclear localization sequences (NLSs).

In some embodiments, the NLSs used in the context are heterologous to the proteins. In general, NLS consists of one or more short sequences of positively charged lysine or arginine exposed on the surface of a protein, but other types of NLS are also known in the art.

In some embodiments, the N-terminus of the gene editing fusion protein comprises an NLS with an amino acid sequence. In some embodiments, the C-terminus of the gene-editing fusion protein comprises an NLS.

In addition, the gene editing fusion protein may also include other localization sequences, such as cytoplasmic localization sequences, chloroplast localization sequences, mitochondrial localization sequences, and the like, depending on the location of the DNA to be edited. In order to obtain efficient expression in plants, in some embodiments, the nucleotide sequence encoding the gene editing fusion protein is codon optimized for the plant to be gene edited.

Codon optimization refers to a process of modifying a nucleic acid sequence for enhanced expression in the host cells of interest by replacing at least one codon (e.g. about or more than about 1, 2, 3, 4, 5, 10, 15, 20, 25, 50, or more codons) of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. Codon usage tables are readily available, for example, at the “Codon Usage Database” available at www.kazusa.orjp/codon/ and these tables can be adapted in a number of ways. See Nakamura, Y., et al. “Codon usage tabulated from the international DNA sequence databases: status for the year 2000” Nucl. Acids Res. 28:292 (2000).

In some embodiments, the codon-optimized nucleotide sequence encoding the gene editing fusion protein is provided herein. In some embodiments, the guide RNA is a single guide RNA (sgRNA). Methods of constructing suitable sgRNAs according to a given target sequence are known in the art. See e.g., Wang, Y. et al. Simultaneous editing of three homeoalleles in hexaploid bread wheat confers heritable resistance to powdery mildew. Nat. Biotechnol. 32, 947-951 (2014); Shan, Q. et al. Targeted genome modification of crop plants using a CRISPR-Cas system. Nat. Biotechnol. 31, 686-688 (2013); Liang, Z. et al. Targeted mutagenesis in Zea mays using TALENs and the CRISPR/Cas system. J Genet Genomics. 41, 63-68 (2014).

In order to ensure appropriate expression in a plant cell, the components of the targeted gene-editing system described herein are typically placed under control of a plant promoter, i.e. a promoter operable in plant cells. The use of different types of promoters is envisaged.

A constitutive plant promoter is a promoter that is able to express the open reading frame (ORF) that it controls in all or nearly all of the plant tissues during all or nearly all developmental stages of the plant (referred to as “constitutive expression”). One non-limiting example of a constitutive promoter is the cauliflower mosaic virus 35S promoter. “Regulated promoter” refers to promoters that direct gene expression not constitutively, but in a temporally- and/or spatially-regulated manner, and includes tissue-specific, tissue-preferred and inducible promoters. Different promoters may direct the expression of a gene in different tissues or cell types, or at different stages of development, or in response to different environmental conditions. In particular embodiments, one or more of the components of the targeted gene-editing system described herein are expressed under the control of a constitutive promoter, such as the cauliflower mosaic virus 35S promoter. Tissue-preferred promoters can be utilized to target enhanced expression in certain cell types within a particular plant tissue, for instance vascular cells in leaves or roots or in specific cells of the seed. Examples of particular promoters for use of the present disclosure can be found in Kawamata et al., (1997) Plant Cell Physiol 38:792-803; Yamamoto et al., (1997) Plant J 12:255-65; Hire et al, (1992) Plant Mol Biol 20:207-18,Kuster et al, (1995) Plant Mol Biol 29:759-72, and Capana et al., (1994) Plant Mol Biol 25:681-91.

Inducible promoters can be of interest to express one or more of the components of the targeted gene-editing system described herein under limited circumstances to avoid non-specific activity of the deaminase. In particular embodiments, one or more elements of the targeted gene-editing system described herein are expressed under control of an inducible promoter.

Examples of promoters that are inducible and that allow for spatiotemporal control of gene editing or gene expression may use a form of energy. The form of energy may include but is not limited to sound energy, electromagnetic radiation, chemical energy and/or thermal energy. Examples of inducible systems include tetracycline inducible promoters (Tet-On or Tet-Off), small molecule two-hybrid transcription activations systems (FKBP, ABA, etc), or light inducible systems (Phytochrome, LOV domains, or cryptochrome), such as a Light Inducible Transcriptional Effector (LITE) that direct changes in transcriptional activity in a sequence-specific manner. The components of a light inducible system may include a fusion protein of the targeted gene-editing system and a light-responsive cytochrome heterodimer (e.g. from Arabidopsis thaliana). Further examples of inducible DNA binding proteins and methods for their use are provided in U.S. 61/736,465 and U.S. 61/721,283, which is hereby incorporated by reference in its entirety.

In particular embodiments, transient or inducible expression can be achieved by using, for example, chemical-regulated promotors, i.e. whereby the application of an exogenous chemical induces gene expression. Modulating of gene expression can also be obtained by a chemical-repressible promoter, where application of the chemical represses gene expression. Chemical-inducible promoters include, but are not limited to, the maize ln2-2 promoter, activated by benzene sulfonamide herbicide safeners (De Veylder et al., (1997) Plant Cell Physiol 38:568-77), the maize GST promoter (GST-11-27, WO93/01294), activated by hydrophobic electrophilic compounds used as pre-emergent herbicides, and the tobacco PR-1 a promoter (Ono et al., (2004) Biosci Biotechnol Biochem 68:803-7) activated by salicylic acid. Promoters which are regulated by antibiotics, such as tetracycline-inducible and tetracycline-repressible promoters (Gatz et al., (1991) Mol Gen Genet 227:229-37; U.S. Pat. Nos. 5,814,618 and 5,789,156) can also be used herein.

In some embodiments, the nucleotide sequence encoding the gene-edited fusion protein and/or the nucleotide sequence encoding the guide RNA is operably linked to a plant expression regulatory element, such as a promoter. Examples of promoters that can be used in the present disclosure include, but are not limited to the cauliflower mosaic virus 35S promoter (Odell et al. (1985) Nature 313: 810-812), a maize Ubi-1 promoter, a wheat U6 promoter, a rice U3 promoter, a maize U3 promoter, a rice actin promoter, a TrpPro5 promoter (U.S. patent application Ser. No. 10/377,318; filed on Mar. 16, 2005), a pEMU promoter (Last et al. Theor. Appl. Genet. 8 1: 581-588), a MAS promoter (Velten et al. (1984) EMBO J. 3:2723-2730), a maize H3 histone promoter (Lepetit et al. Mol. Gen. Genet. 231:276-285 and Atanassova et al. (1992) Plant J. 2 (3): 291-300), and a Brassica napus ALS3 (PCT Application WO 97/41228) promoters. Promoters that can be used in the present disclosure also include the commonly used tissue specific promoters as reviewed in Moore et al. (2006) Plant J. 45 (4): 651-683.

In the present disclosure, the plants are intended to comprise without limitation angiosperm and gymnosperm plants such as acacia, alfalfa, amaranth, apple, apricot, artichoke, ash tree, asparagus, avocado, banana, barley, beans, beet, birch, beech, blackberry, black raspberry, blueberry, broccoli, Brussel's sprouts, cabbage, cane berry, canola, cantaloupe, carrot, cassava, cauliflower, cedar, a cereal, celery, chestnut, cherry, Chinese cabbage, citrus, Clementine, clover, coffee, corn, cotton, cowpea, cucumber, cypress, eggplant, elm, endive, eucalyptus, fennel, figs, fir, geranium, grape, grapefruit, groundnuts, ground cherry, gum hemlock, hickory, kale, kiwifruit, kohlrabi, larch, lettuce, leek, lemon, lime, locust, pine, maidenhair, maize, mango, maple, melon, millet, mushroom, mustard, nuts, oak, oats, oil palm, okra, onion, orange, an ornamental plant or flower or tree, papaya, palm, parsley, parsnip, pea, peach, peanut, pear, peat, pepper, persimmon, pigeon pea, peach, pine, pineapple, plantain, plum, pomegranate, potato, pumpkin, radicchio, radish, rapeseed, raspberry, rice, rye, sorghum, safflower, sallow, soybean, spinach, spruce, squash, strawberry, sugar beet, sugarcane, sunflower, sweet potato, sweet corn, tangerine, tea, tobacco, tomato, trees, triticale, turf grasses, turnips, vine, walnut, watercress, watermelon, wheat, wild strawberry, yams, yew, and zucchini.

The methods for targeted gene-editing system as described herein can be used to confer desired traits on essentially any plant. A wide variety of plants and plant cell systems may be engineered for the desired physiological and agronomic characteristics described herein using the nucleic acid constructs of the present disclosure and the various transformation methods. In preferred embodiments, target plants and plant cells for engineering include, but are not limited to, those monocotyledonous and dicotyledonous plants, such as crops including grain crops (e.g., wheat, maize, rice, millet, barley), fruit crops (e.g., tomato, apple, grape, peach, pear, plum, raspberry, black raspberry, blackberry, cane berry, cherry, avocado, strawberry, wild strawberry, orange), forage crops (e.g., alfalfa), root vegetable crops (e.g., carrot, potato, sugar beets, yam), leafy vegetable crops (e.g., lettuce, spinach); flowering plants (e.g., petunia, rose, chrysanthemum), conifers and pine trees (e.g., pine fir, spruce); plants used in phytoremediation (e.g., heavy metal accumulating plants); oil crops (e.g., sunflower, rape seed) and plants used for experimental purposes (e.g., Arabidopsis). In some embodiments, fruit crops such as tomato, apple, peach, pear, plum, raspberry, black raspberry, blackberry, cane berry, cherry, avocado, strawberry, wild strawberry, grape and orange.

The present disclosure provides methods for targeted editing in a plant cell, tissue, organ or plant. In one aspect, the present disclosure provides methods for producing a gene-edited plant by transient expression of a gene editing system. In another aspect, the present disclosure provides methods for a gene-edited and non-transgenic plant by transient expression of a gene editing system along with transient expression of at least one morphogenic regulator taught in the present disclosure. Therefore, the present disclosure teaches a gene-edited plant that is also transgene-free without any integration of foreign DNAs.

In some embodiments, an editing system as described herein is used to introduce targeted mutations, such as insertions, deletions, or substitutions, thereby causing a nonsense mutation (e.g., premature stop codon) or a missense mutation (e.g., encoding different amino acid residue). This is of interest where the single nucleotide mutations in certain endogenous genes can confer or contribute to a desired trait.

The methods described herein may result in the generation of gene-edited plants that have one or more desirable traits compared to the wild type plant.

In some embodiments, non-transgenic but gene-edited plants, plant parts, or cells are obtained, in that no exogenous DNA sequence is incorporated into the genome of any of the plant cells of the plant. In such embodiments, the gene-edited plants are non-transgenic. Where only the modification of an endogenous gene is ensured and no foreign genes are introduced or maintained in the plant genome, the resulting genetically modified crops contain no foreign genes and can thus basically be considered non-transgenic.

In some embodiments, modification of the target sequence can be accomplished simply by introducing or producing the gene editing protein and guide RNA in plant cells, and the modification can be stably inherited without the need of stable transformation of plants with an editing system. This avoids potential off-target effects of a stable editing system, and also avoids the integration of exogenous nucleotide sequences into the plant genome, and thereby resulting in higher biosafety. In some embodiments, the editing system is the targeted gene editing system. In further embodiments, the plant cells in which the genetic modification is stably inherited without stable transformation is edited by the gene editing system (i.e. the targeted gene editing system) are transformed and regenerated into a whole plant by the transient expression of one or more morphogenic regulators.

In other embodiments, the polynucleotides are delivered into the cell by a DNA virus (e.g., a geminivirus) or an RNA virus (e.g., a tobravirus). In other embodiments, the introducing steps include delivering to the plant cell a T-DNA containing one or more polynucleotide sequences encoding an encoding system (e.g., a CRISPR-Cas effector protein and a guide nucleic acid), where the delivering is via Agrobacterium. The polynucleotide sequence encoding the components of CRISPR/Cas system can be operably linked to a promoter, such as a constitutive promoter (e.g., a cauliflower mosaic virus 35S promoter), or a cell specific or inducible promoter in a plant cell of interest described herein. In other embodiments, the polynucleotide is introduced by microprojectile bombardment.

In some embodiments, the gene editing system can be introduced into plants by various methods well known to people skilled in the art. Methods that can be used to introduce the gene editing system of the present disclosure into plants include but not limited to particle bombardment, PEG-mediated protoplast transformation, Agrobacterium-mediated transformation, plant virus-mediated transformation, pollen tube approach, and ovary injection approach.

In some embodiments, the introduction is performed in the absence of a selective pressure, thereby avoiding the integration of exogenous nucleotide sequences in the plant genome. In some embodiments, the introduction comprises transforming the gene editing system into isolated plant cells or tissues, and then regenerating the transformed plant cells or tissues into an intact plant by the help of morphogenic regulator expression taught in the present disclosure. Preferably, the regeneration is performed in the absence of a selective pressure, i.e., no selective agent against the selective gene carried on the expression vector is used during the tissue culture. Without the use of a selective agent, the regeneration efficiency of the plant can be increased to obtain a modified plant that does not contain exogenous nucleotide sequences. To increase, enhance, improve the transformation and regeneration of a non-transgenic plant with a desired modification inherited, the morphogenic regulator(s) is transiently expressed in the plant cell.

In other embodiments, the editing system of the present disclosure can be transformed to a particular site on an intact plant, such as leaf, shoot tip, pollen tube, young ear, or hypocotyl. This is particularly suitable for the transformation of plants that are difficult to regenerate by tissue culture. In some embodiments, proteins expressed in vitro and/or RNA molecules transcribed in vitro are directly transformed into the plant. The proteins and/or RNA molecules are capable of achieving gene-editing in plant cells, and are subsequently degraded by the cells to avoid the integration of exogenous nucleotide sequences into the plant genome. Plants that can be gene-edited by the methods includes monocotyledon and dicotyledon. For example, the plant may be a crop plant such as wheat, rice, maize, soybean, sunflower, sorghum, rape, alfalfa, cotton, barley, millet, sugar cane, tomato, tobacco, cassava, or potato. For another example, the plant may be a fruit crops such as tomato, apple, peach, pear, plum, raspberry, black raspberry, blackberry, cane berry, cherry, avocado, strawberry, wild strawberry, grape and orange.

In some embodiments, the target sequence is associated with plant traits such as desired traits, and thereby the gene editing results in the plant having altered traits relative to a wild type plant. In the present disclosure, the target sequence to be modified (e.g., a target nucleic acid) may be located anywhere in the genome, for example, within a functional gene such as a protein-coding gene or, for example, may be located in a gene expression regulatory region such as a promoter region or an enhancer region, and thereby accomplish the functional modification of said gene or accomplish the modification of a gene expression.

In some embodiments, the method further comprises obtaining progeny of the gene-edited plant, which is also non-transgenic. In a further aspect, the disclosure also provides a gene-edited plant or progeny thereof or parts thereof, wherein the plant is obtained by the method described above.

In another aspect, the present disclosure also provides a plant breeding method comprising crossing a first gene-edited plant obtained by the above-mentioned method of the present disclosure with a second plant not containing said genetic modification, thereby introducing said genetic modification into said second plant.

In particular embodiments, the method further includes screening the plant cell after the introducing steps to determine whether the expression of the gene of interest has been modified. In particular embodiments, the methods include the step of regenerating a plant from the plant cell. In further embodiments, the methods include cross breeding the plant to obtain a genetically desired plant lineage.

In some embodiments, significant impact of transient expression of morphogenic regulators on plant transformation and regeneration can be applied for generation of gene-edited plant without foreign DNA integration. The regenerated plant from a gene-edited plant cell is transgene-free and/or DNA integration-free. Transient expression of at least one morphogenic regulators gives rise to successful regeneration of a plant cell of interest.

In other embodiments, significant impact of transient expression of morphogenic regulators on plant transformation and regeneration can be applied for plant regeneration of dicot species, especially those with heterozygotic nature and/or polyploidy.

In some embodiments, transient expression of morphogenic regulators can used to regenerate plants from protoplast cells. In other embodiments, transient expression of morphogenic regulator can be used to regenerate plants from edited callus by the gene-editing system taught in the present disclosure.

As described herein, the nucleic acids of the invention and/or expression cassettes and/or vectors comprising the same may be codon optimized for expression in an organism. An organism useful with this invention may be any organism or cell thereof for which a morphogenic regulator and/or nucleic acid modification may be useful. An organism can include, but is not limited to, any animal, any plant, any fungus, any archaeon, or any bacterium. In some embodiments, the organism may be a plant or cell thereof.

A target nucleic acid of any cell, plant, or plant part may be modified using a composition, system, and/or method of the invention. Any plant (or groupings of plants, for example, into a genus or higher order classification) may be modified using a nucleic acid construct, composition, system, and/or method of this invention including an angiosperm, a gymnosperm, a monocot, a dicot, a C3, C4, CAM plant, a bryophyte, a fern and/or fern ally, a microalgae, and/or a macroalgae. A plant and/or plant part useful with this invention may be a plant and/or plant part of any plant species/variety/cultivar. The term “plant part,” as used herein, includes but is not limited to, embryos, pollen, ovules, seeds, leaves, stems, shoots, flowers, branches, fruit, kernels, ears, cobs, husks, stalks, roots, root tips, anthers, plant cells including plant cells that are intact in plants and/or parts of plants, plant protoplasts, plant tissues, plant cell tissue cultures, plant calli, plant clumps, and the like. As used herein, “shoot” refers to the above ground parts including the leaves and stems. Further, as used herein, “plant cell” refers to a structural and physiological unit of the plant, which comprises a cell wall and also may refer to a protoplast. A plant cell can be in the form of an isolated single cell or can be a cultured cell or can be a part of a higher-organized unit such as, for example, a plant tissue or a plant organ.

Non-limiting examples of plants useful with the present invention include turf grasses (e.g., bluegrass, bentgrass, ryegrass, fescue), feather reed grass, tufted hair grass, miscanthus, arundo, switchgrass, vegetable crops, including artichokes, kohlrabi, arugula, leeks, asparagus, lettuce (e.g., head, leaf, romaine), malanga, melons (e.g., muskmelon, watermelon, crenshaw, honeydew, cantaloupe), cole crops (e.g., brussels sprouts, cabbage, cauliflower, broccoli, collards, kale, chinese cabbage, bok choy), cardoni, carrots, napa, okra, onions, celery, parsley, chick peas, parsnips, chicory, peppers, potatoes, cucurbits (e.g., marrow, cucumber, zucchini, squash, pumpkin, honeydew melon, watermelon, cantaloupe), radishes, dry bulb onions, rutabaga, eggplant, salsify, escarole, shallots, endive, garlic, spinach, green onions, squash, greens, beet (sugar beet and fodder beet), sweet potatoes, chard, horseradish, tomatoes, turnips, and spices; a fruit crop such as apples, apricots, cherries, nectarines, peaches, pears, plums, prunes, cherry, quince, fig, nuts (e.g., chestnuts, pecans, pistachios, hazelnuts, pistachios, peanuts, walnuts, macadamia nuts, almonds, and the like), citrus (e.g., clementine, kumquat, orange, grapefruit, tangerine, mandarin, lemon, lime, and the like), blueberries, black raspberries, boysenberries, cranberries, currants, gooseberries, loganberries, raspberries, strawberries, blackberries, grapes (wine and table), avocados, bananas, kiwi, persimmons, pomegranate, pineapple, tropical fruits, pomes, melon, mango, papaya, and lychee, a field crop plant such as clover, alfalfa, timothy, evening primrose, meadow foam, corn/maize (field, sweet, popcorn), hops, jojoba, buckwheat, safflower, quinoa, wheat, rice, barley, rye, millet, sorghum, oats, triticale, sorghum, tobacco, kapok, a leguminous plant (beans (e.g., green and dried), lentils, peas, soybeans), an oil plant (rape, canola, mustard, poppy, olive, sunflower, coconut, castor oil plant, cocoa bean, groundnut, oil palm), duckweed, Arabidopsis, a fiber plant (cotton, flax, hemp, jute), Cannabis (e.g., Cannabis sativa, Cannabis indica, and Cannabis ruderalis), lauraceae (cinnamon, camphor), or a plant such as coffee, sugar cane, tea, and natural rubber plants; and/or a bedding plant such as a flowering plant, a cactus, a succulent and/or an ornamental plant (e.g., roses, tulips, violets), as well as trees such as forest trees (broad-leaved trees and evergreens, such as conifers; e.g., elm, ash, oak, maple, fir, spruce, cedar, pine, birch, cypress, eucalyptus, willow), as well as shrubs and other nursery stock. In some embodiments, the nucleic acid constructs of the invention and/or expression cassettes and/or vectors encoding the same may be used to modify maize, soybean, wheat, canola, rice, tomato, pepper, sunflower, raspberry, blackberry, black raspberry and/or cherry.

In some embodiments, a plant cell of the present invention and/or that is used in a composition, system and/or method of the present invention is a plant cell is from an asexually propagated, heterozygous, perennial plant. In some embodiments, a plant cell of the present invention and/or that is used in a composition, system and/or method of the present invention is a dicot plant cell, optionally wherein the plant cell is a blackberry, raspberry, or cherry plant cell.

In some embodiments, the invention provides cells (e.g., plant cells, animal cells, bacterial cells, archaeon cells, and the like) comprising a polypeptide, polynucleotide, nucleic acid construct, expression cassette and/or vector of the invention.

The present invention further comprises a kit or kits to carry out the methods of this invention. A kit of this invention can comprise reagents, buffers, and apparatus for mixing, measuring, sorting, labeling, etc, as well as instructions and the like as would be appropriate for modifying a target nucleic acid.

In some embodiments, the invention provides a kit for comprising one or more nucleic acid construct(s) of the invention, expression cassette(s), vector(s), and/or cells comprising the same as described herein, with optional instructions for the use thereof. In some embodiments, a kit may further comprise an editing system (e.g., a CRISPR-Cas guide nucleic acid (corresponding to a CRISPR-Cas effector protein encoded by a polynucleotide of the invention) and/or an expression cassette, vector, and/or cell comprising the same Accordingly, in some embodiments, kits are provided comprising a nucleic acid construct comprising (a) a polynucleotide(s) as provided herein and (b) a promoter that drives expression of the polynucleotide(s) of (a). In some embodiments, the kit may further comprise a nucleic acid construct encoding a guide nucleic acid, wherein the construct comprises a cloning site for cloning of a nucleic acid sequence identical or complementary to a target nucleic acid sequence into backbone of the guide nucleic acid.

In some embodiments, the nucleic acid construct of the invention may be an mRNA that may encode one or more introns within the encoded polynucleotide(s). In some embodiments, the nucleic acid constructs of the invention, and/or an expression cassettes and/or vectors comprising the same, may further encode one or more selectable markers useful for identifying transformants (e.g., a nucleic acid encoding an antibiotic resistance gene, herbicide resistance gene, and the like).

The invention will now be described with reference to the following examples. It should be appreciated that these examples are not intended to limit the scope of the claims to the invention, but are rather intended to be exemplary of certain embodiments. Any variations in the exemplified methods that occur to the skilled artisan are intended to fall within the scope of the invention.

### EXAMPLES

The following examples are given for the purpose of illustrating various embodiments of the disclosure and are not meant to limit the present disclosure in any fashion. Changes therein and other uses which are encompassed within the spirit of the disclosure, as defined by the scope of the claims, will occur to those skilled in the art.

### Example 1. Transformation of Tobacco Leaves

Transformation can be accomplished by various methods known to be effective in plants, including particle-mediated delivery, Agrobacterium-mediated transformation, PEG-mediated delivery, and electroporation.

The tobacco leaves are prepared for transformation with plasmids containing the gene-editing system and/or plasmids containing at least one morphogenic regulator. The expression vector containing the morphogenic regulators such as WUS, BBN, KN1, IPT, CLV3, miR156, GRF5, NTT, and HDZipII is co-bombarded or co-transformed with another expression vector containing the targeted gene editing system, for example, CRISPR/Cas 9 endonuclease with gRNA.

The tobacco leaf transformation can be achieved by the simultaneous expression of the morphogenic regulator and the gene editing machinery after the vacuum infiltration of tobacco leaves with two populations of recombinant Agrobacterium.

### Example 2. Transient Expression of Morphogenic Regulators

Parameters of the transformation protocol can be modified to ensure that the activity of morphogenic regulators (including, but are not limited to, WUS, BBN, KN1, IPT, CLV3, miR156, GRF5, NTT, and HDZipII) is transient.

One method for transient expression can involve precipitating the expression vector in a manner that allows for transcription and expression, but precludes subsequent release of the DNA, for example, by using the chemical polyethlenimine.

In this example, three expression vectors were constructed to test integration efficiency of a morphogenic regulator; 1) GUS marker gene driven by a constitutive promoter, 2) ipt gene fused with GUS marker gene under control of a native promoter, 3) ipt gene fused with GUS marker gene under control of a constitutive promoter. Each vector was transformed into tobacco leaves (Nicotiana benthamiana).

The effect of an isopentenyl transferase (ipt) gene expression under a constitutive promoter could clearly be seen. For example, ipt gene expression induced multiple shoot formation in a hormone-free media when compared to two controls (i) without ipt gene expressed as well as (ii) with ipt gene expressed under a native promoter. The ipt transformed leaf explants showed darker green color and enlargement at 2-4 wks and numerous shoots emerged as early as 4 weeks after inoculation on hormone-free and nonselective medium.

The ipt gene under a constitutive promoter produced significantly more plants that were regenerated in a hormone-free media than the control where a construct without ipt gene was used, indicating that ipt gene enhanced plant regeneration in tobacco.

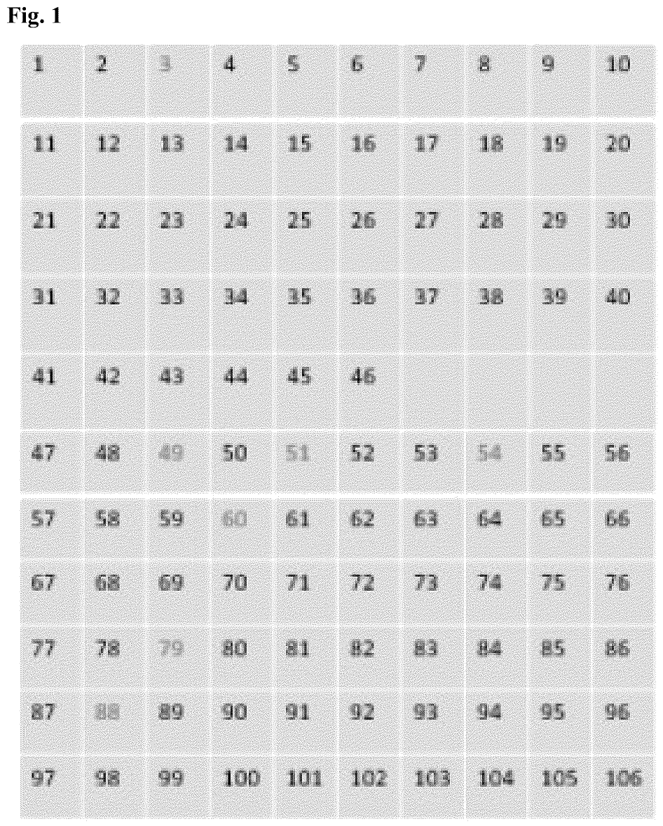

Over 90% of the plants regenerated were GUS negative in the first experiment. As shown in FIG. 1, GUS assay results of one hundred six shoots induced by the ipt gene expression indicate that about 93.4% (99 out of 106 ipt-induced shoots) were GUS negative, suggesting either that the GUS-negative shoots would not be stably integrated but transiently expressed. However, transgenic nature has not been determined. Thus, molecular analyses are being performed to determine the transgenic nature of these GUS negative plants is in progress.

On the other hand, seven samples were GUS-positive, which may indicate stable integration of ipt-GUS fusion cassette. Furthermore, the GUS-positive ipt-induced shoots generated too many shoots that could be explained by too high level of cytokinin accumulation due to the constitutive ipt gene expression. Thus, the GUS positive samples are considered to be stably integrated into a tobacco genome.

Importantly, molecular analyses are being performed to determine the transgenic nature of these GUS negative plants is in progress.

### Example 3. Constructions of Expression Vectors for Gene-Editing System and/or Morphogenic Regulators

In this example, the construction of expression vector contains a marker-free T-DNA. Especially, three constructs are being generated and tested for efficiency of gene-editing and/or plant regeneration.
1) Locate both ipt (Morphogenic regulator; Morgen) and gene-editing machinery outside of T-DNA Left Border
2) Locate editing machinery inside of T-DNA, but ipt/Morgen outside of T-DNA Left Border
3) Locate both ipt/Morgen and editing machinery inside of T-DNA

An expression vector comprising 1) expression cassette for ipt gene and 2) expression cassette for gene editing system (CRISPR/Cas9 and gRNA) will be transformed into a plant cell of interest in order to generate mutations at the corresponding targeted sites. DNA delivery (such as recombinant DNA, plasmid DNA) for targeted mutagenesis and gene editing in plants are presented herewith.

The gene-editing expression vectors will be transformed into plants of interest such as: black raspberry, blackberry, cane berry, cherry, peach, avocado, strawberry, wild strawberry, apple, tomato, grape, and peach as well as model plants such as Arabidopsis and tobacco. The disclosure teaches all types of transformation methods, including using agrobacterium-mediated protocols that are known in the art and/or developed by the inventors, as well as biolistic transformation methods. Tissue culture and regeneration of transformed plants will be performed accordingly.

### Example 4: Molecular Analysis for Transformed Plants

In this example, the activity of the gene-editing machinery will be examined with efficiency of plant transformation and regeneration by control of transient ipt gene expression on the transformed plants of interest including Arabidopsis, black raspberry, cane berry, blackberry, cherry, avocado, strawberry, wild strawberry, peach, grape, apple, tomato and/or tobacco.

### Example 5: Inducible Expression of Morphogenic Regulators

In this example, expression vectors were constructed to test inducible ipt expression (GR-LHG4-pOP4::ipt) in tobacco transformation. The vector contained two expression cassettes. The ipt was driven by a pOP4 promoter, whose transcription activity is controlled by the transcription factor LHG4. LHG4 was fused with glucocorticoid receptor (GR), constitutively expressed and located in cytoplasm. GR-LHG4 protein translocates to a cell nucleus upon the application of inducing chemical dexamethasone (Dex). Each vector was transformed into tobacco leaves (Nicotiana benthamiana). Leaf segments at about 1 square centimetre were prepared from tobacco plants maintained in vitro. An Agrobacterium suspension containing the plasmid was prepared at OD 0.2 as the inoculum. Leaf segments were inoculated by the Agrobacterium by submerging in the suspension for 10 minutes and then were transferred to sterile filter paper (1 mL of MS liquid added) for a 2-day co-culture. The leaf explants were then transferred to MS-based agar medium containing no plant growth regulators or selection agent for regeneration. Medium with and without inducing chemical Dex (μM) were compared for the effect of induced ipt expression.

The explants showed darker green color and enlargement at 2-4 wks than the control; and numerous shoots emerged as early as 4 weeks after inoculation on hormone-free and nonselective medium. In addition, numerous shoots emerged as early as 4 weeks on hormone-free nonselective medium supplemented with inducing chemical dexamethasone (Dex). As the control, no shoots were regenerated from the same medium without Dex.

### Example 6: Non-Transgenic Editing Using Morphogenic Regulators and a Gene-Editing System

The efficacy of non-transgenic editing using ipt in the same T-DNA with PDS editing machinery was examined. The vectors contained a sequence encoding LbCpf1 nuclease and gRNA targeting tobacco PDS genes to create deletion mutations. In the same vector, an ipt constitutive expression cassette was built in the same T-DNA with the PDS editing machinery. Agrobacterium containing the vector was used for tobacco transformation. Leaf segments at about 1 square centimetre were prepared from tobacco plants maintained in vitro. An Agrobacterium suspension containing the plasmid was prepared at OD 0.2 as the inoculum. Leaf segments were inoculated by the Agrobacterium by submerging in the suspension for 10 minutes and then were transferred to sterile filter paper (1 mL of MS liquid added) for a 2-day co-culture. The leaf explants were then transferred to MS-based agar medium containing no plant growth regulators or selection agent for regeneration.

The plants that were regenerated with ipt included many shoots when regenerated on the hormone-free nonselective medium. A population of 200 shoots was subjected to molecular determination of transgene integration and gene editing. A total of 7 samples had transgene integration among the 200 (providing 96.5% nontransgenic, which is similar with Example 2). Four samples showed deletion mutations targeted by gRNA, including 3 with transgene integration and 1 without the integration. As the control, 20 shoots regenerated from medium with selection and BAP were used for the same molecular determinations, and photobleaching phenotype was observed on some regenerated shoots.

### Example 7: Strong Constitutive Expression

RoKN1 and PavKN1 (strong constitutive expression) in tobacco transformation was tested. Leaf segments at about 1 square centimeter were prepared from tobacco plants maintained in vitro. An Agrobacterium suspension containing the plasmid (RoKN1 or PavKN1 expression) was prepared at OD 0.2 as the inoculum. Leaf segments were inoculated by the Agrobacterium by submerging in the suspension for 10 minute and transferred to sterile filter paper (1 mL of MS liquid added) for a 2-day co-culture. Then the leaf explants were transferred to MS-based agar medium containing 1 mg/L of BAP and 100 mg/L of spectinomycin as the selective agent. In contrast to expression with ipt, no different phenotype was observed on hormone-free nonselective medium but strong a shooty phenotype was observed on regeneration medium (BAP+selection).

### Example 8: Expression of Morphogenic Regulators with Various Promoters

Various morphogenic regulators were tested together with different promoters in tobacco transformation. RoWUS, SorbiWUS, PavWUS, AtBBM, TaBBM-1, TaBBM-2, SobiBBM, RoBBM, and PavBBM were tested. Each gene was driven by a double viral strong constitutive promoter or a weaker promoter (pNOS) for a comparative test. Leaf segments at about 1 square centimetre were prepared from tobacco plants maintained in vitro. An Agrobacterium suspension containing each of the plasmids was prepared at OD 0.2 as the inoculum. Leaf segments were inoculated by the Agrobacterium by submerging in the suspension for 10 minutes and were then transferred to sterile filter paper (1 mL of MS liquid added) for a 2-day co-culture. Then the leaf explants were transferred to four types of MS-based agar medium for regeneration: 1) hormone-free and selection-free; 2) BAP 1 mg/L and selection free; 3) BAP 1 mg/L and selection; 4) 2, 4D 0.2 mg/L and selection.

The medium conditions post inoculation include 1) hormone-free nonselective 2) BAP w/o selection 3) BAP+selection 4) 2,4 D+selection. There was no significant morphogenic changes for all tested genes on medium conditions 1), 2) or 3). On the 2,4 D containing medium, somatic embryogenesis was observed from RoWUS, PavWUS and SobiWUS transformed explants, as showed by early globular callus (both promoters), and bipolar structures (strong promoters) and proliferation of shoots from regenerated structure (strong promoter). The level of WUS expression appeared to affect the normal development as informed by visual reporter ZsGreen expression.

### Example 9

WOX2-GR and WOX8-GR in tobacco transformation were tested. Results: shoot regeneration was observed from WOX2-GR inoculated explants at the presence of Dex on medium with BAP and selection (image below). Strong shoot proliferations were observed from WOX8-GR inoculated explants at the presence of Dex on medium with BAP and selection.

### Example 10

RoWUS was tested in blackberrry transformation. Results: higher efficiency of regeneration was observed in RoWUS transformed leaf petiole explants, as shown by higher number of shoots regenerated per petiole. Both promoter types (constitutive strong and constitutive weak) showed similar gene effect. Two Agrobacterium strains were mixed at different ratios and co-infiltrated to blackberry explants. One strain delivered a T-DNA containing the RoWUS expression cassette and the other strain delivered a T-DNA containing a Cas12a expression cassette for genome editing. Many regenerated shoots were obtained due to RoWUS regeneration enhancing capacity. When delivered at a 1:1 ratio of RoWUS T-DNA to Cas12a T-DNA, 36 shoots were recovered and 2 were positive in a qPCR test for presence of a hygromycin selectable marker gene (5.6% efficiency). When delivered at a 1:9 ratio of RoWUS T-DNA to Cas12a T-DNA, 50 shoots were recovered and 5 were positive in a qPCR test for presence of a hygromycin selectable marker gene (10% efficiency). In contrast to these results, a control sample group transformed only with the T-DNA containing the Cas12a expression cassette yielded 41 shoots and only 1 was positive in a qPCR test for presence of a hygromycin selectable marker gene (2.4% efficiency). This indicates that transgenic shoots can be recovered at a higher efficiency when transforming with RoWUS than without it.

### REFERENCES

All references, articles, publications, patents, patent publications, and patent applications cited herein are incorporated by reference in their entireties for all purposes. However, mention of any reference, article, publication, patent, patent publication, and patent application cited herein is not, and should not, be taken as an acknowledgment or any form of suggestion that they constitute valid prior art or form part of the common general knowledge in any country in the world.
U.S. Pat. No. 7,256,322 B2
US20180273960 A1
Gordon-Kamm B., Sardesai N., Arling M., Lowe K., Hoerster G., Betts S., and Jones T. Using morphogenic genes to improve recovery and regeneration of transgenic plants. Plants 8(2). Pii: E38 (2019)
Chen et al. A method for the production and expedient screening of CRISPR/Cas9-mediated non-transgenic mutant plants. Hort Research 5:13 (2018)
Carimi F., De Pasquale F. Crescimanno F. G. Somatic embryogenesis and plant regeneration from pistil thin cell layers of citrus. Plant Cell Rep. 18, 935-940 (1999).
Paul H., Belaizi M., Sangwan-Norreel B.S. Somaticembryogenesisinapple. J. Plant Physiol. 143, 78-86 (1994).
Fischer R., Emans, N. Molecular farming of pharmaceutical proteins. Transgenic Res. 9, 279-299 (2000).
Pogue G. P. et al. Production of pharmaceutical-grade recombinant aprotinin and a monoclonal antibody product using plant-based transient expression systems. Plant Biotechnol. J. 8, 638-654 (2010).
Richael C. M., Kalyaeva M., Chretien R. C., Yan H., Adimulam S., Stivison A., Weeks J. T., Rommens C. M., Cytokinin vectors mediate marker-free and backbone-free plant transformation. Transgenic Res. 17:905-917 (2008).
Boutilier K, Offringa R, Sharma V K, Kieft H, Ouellet T, Zhang L, Hattori J, Liu C M, van Lammeren A A, Miki B L, Custers J B, van Lookeren Campagne MMEctopic expression of BABY BOOM triggers a conversion from vegetative to embryonic growth. Plant Cell 14:1737-1749 (2002)
Nishimura A., Tamaoki M., Sakamoto T., Matsuoka M., Over-Expression of Tobacco knotted1-Type Classl Homeobox Genes Alters Various Leaf Morphology. Plant Cell Physiol. 41(5) 583-590 (2000)
Song X F, Guo P., Ren S.-C, Xu T.-T., and Liu C.-M. Antagonistic Peptide Technology for Functional Dissection of CLV3/ESR Genes in Arabidopsis. Plant Physiol. 161(3):1076-1085 (2013)
Long J. M., Liu C. Y., Feng M. Q., Liu Y., Wu X. M., Guo W. W., miR156-SPLs Module Regulates Somatic Embryogenesis Induction in Citrus Callus. J Exp Bot. 69(12):2979-2993 (2018)
Zhang T. Q., Lian H., Tang H., Dolezal K., Zhou C. M., Yu S., Chen J. H., Chen Q., Liu H., Ljung K., Wang J. W. An intrinsic microRNA timer regulates progressive decline in shoot regenerative capacity in plant. Plant Cell 27(2):349-360 (2015)
Vercruyssen L, Tognetti V. B., Gonzalez N., Van Dingenen J., De Milde L., Bielach A., De Rycke R., Van Breusegem F., Inzé D. GROWTH REGULATING FACTOR5 Stimulates Arabidopsis Chloroplast Division, Photosynthesis, and Leaf Longevity. Plant Physiol. 167(3):817-832 (2015)
Malnoy, M. et al. DNA-free genetically edited grapevine and apple protoplast using CRISPR/Cas9 ribonucleoproteins. Front. Plant Sci. 7, 1904 (2016).
Subburaj, S. et al. Site-directed mutagenesis in Petunia×hybrida protoplast system using direct delivery of purified recombinant Cas9 ribonucleoproteins. Plant Cell Rep. 35, 1535-1544 (2016).
Woo, J. W. et al. DNA-free genome editing in plants with preassembled CRISPR-Cas9 ribonucleoproteins. Nat. Biotechnol. 33, 1162-1164 (2015).
Liang, Z. et al. Ef ficient DNA-free genome editing of bread wheat using CRISPR/Cas9 ribonucleoprotein complexes. Nat. Commun. 8, 14261 (2017).
Svitashev, S., Schwartz, C., Lenderts, B., Young, J. K. & Cigan, A. M. Genome editing in maize directed by CRISPR-Cas9 ribonucleoprotein complexes. Nat. Commun. 7, 13274 (2016).
Zhang, Y. et al. Efficient and transgene-free genome editing in wheat through transient expression of CRISPR/Cas9 DNA or RNA. Nat. Commun. 7, 12617 (2016).
Kim, H. et al. CRISPR/Cpf1-mediated DNA-free plant genome editing. Nat. Commun. 8, 14406 (2017).

What is claimed is:

1. An isolated polynucleotide encoding an amino acid sequence having at least 95% sequence identity to the polypeptide sequence of SEQ ID NO:26, wherein the isolated polynucleotide is operably associated with a heterologous promoter.

2. A cell, plant part, or plant comprising the isolated polynucleotide of claim 1.

3. The cell, plant part, or plant of claim 2, further comprising a polynucleotide encoding gene editing machinery and/or a polynucleotide encoding a selectable marker.

4. An expression cassette and/or vector comprising the isolated polynucleotide of claim 1.

5. The expression cassette and/or vector of claim 4, further comprising a polynucleotide encoding gene editing machinery and/or a polynucleotide encoding a selectable marker.
